(19) United States
(12) Patent Application Publication
(10) Pub. No.: US 2022/0177508 A1
(43) Pub. Date: Jun. 9, 2022

(54) C10-Cyclic Substituted 13-Membered Macrolides and Uses Thereof

(71) Applicants: Zikani Therapeutics, Inc., Watertown, MA (US); President and Fellows of Harvard College, Cambridge, MA (US)

(72) Inventors: Roger B. Clark, Lexington, MA (US); Richard Alm, Berlin, MA (US); Wesley Francis Austin, Cambridge, MA (US); Vijaya Gondi, Westford, MA (US); Philip Hogan, Brighton, MA (US); Ivan Jewett, Boston, MA (US); Sushmita D. Lahiri, Lexington, MA (US); Jonathan F. Lawrence, Somerville, MA (US); Xiben Li, Lexington, MA (US); Shuhao Shi, Madison, CT (US); Wenying Wang, Boston, MA (US); Yoshitaka Ichikawa, Cambridge, MA (US); Andrew G. Myers, Boston, MA (US); Ziyang Zhang, San Francisco, CA (US); Peter Niels Carlsen, Claymont, DE (US); Md. Ataur Rahman, Arlington, MA (US)

(21) Appl. No.: 17/294,918

(22) PCT Filed: Nov. 18, 2019
(86) PCT No.: PCT/US2019/062045

§ 371 Date: May 18, 2021

Related U.S. Application Data
(60) Provisional application No. 62/769,413, filed on Nov. 19, 2018.

(51) Int. Cl. C07H 19/01; A61P 31/04

(52) U.S. Cl. CPC C07H 19/01; A61P 31/04

(57) ABSTRACT

Provided are 13-membered macrolides for the treatment of infectious diseases. The 13-membered macrolides described herein are azaketolides. Also provided are methods for preparing the 13-membered macrolides, pharmaceutical compositions comprising the 13-membered macrolides, and methods of treating infectious diseases, and in particular, disease resulting from Gram negative bacteria using the disclosed macrolides.

### CROSS-REFERENCE TO RELATED APPLICATIONS

This application claims priority under 35 U.S.C. § 119(e) to U.S. Provisional Application 62/769,413 filed on Nov. 19, 2018. The disclosure of this prior application is considered part of the disclosure of this application and is hereby incorporated by reference in its entirety.

### BACKGROUND

Emerging resistance to existing antibiotics is rapidly developing as a crisis of global proportions, especially for infections originating from drug resistant Gram-negative bacteria. Pathogenic bacteria can transmit genes coding for antibiotic resistance both vertically (to their progeny) and horizontally (to neighboring bacteria of different lineages), and as a result antibiotic resistance can evolve quickly, particularly in nosocomial (hospital) settings. See, e.g., Wright, Chem. Commun. (2011) 47:4055-4061. More than 99,000 people die annually in the U.S. from healthcare-associated infections, more than all casualties from car accidents, HIV, and breast cancer combined, creating an estimated burden of up to $45 billion in U.S. healthcare costs. See, e.g., Klevens et al., Public Health Rep (2007) 122:160-166. The current crisis is exacerbated by decreased research in the development of new antibiotics by most major pharmaceutical companies. See, e.g., Projan, Curr. Opin. Microbiol. (2003) 6:427-430. The current rate of introduction of new antibiotics does not adequately address growing resistance, and with the ease of international travel and increasing population densities, the need for innovation in the field has never been higher.

The macrolides are one of the few major clinically important classes of antibiotics for which the only practical access has been through semi-synthesis, or chemical manipulation of structurally complex fermentation products, in routes as long as 16 steps. See, e.g., Paterson, Tetrahedron (1985) 41:3569-3624; Omura, Ed., Macrolide Antibiotics: Chemistry, Biology, and Practice, Second Edition; Academic Press, 2002. The macrolide class of antibiotics has proven safe and effective in the battle against pathogenic bacteria since the discovery of erythromycin over 60 years ago. See, e.g., Wu et al., Curr. Med. Chem. (2001) 8:1727-1758. Erythromycin displays a spectrum of antibacterial activity against Gram-positive bacteria similar to that of penicillin but has a lesser propensity to induce allergic interactions, and it has been routinely prescribed for upper and lower respiratory tract infections and urogenital infections. See, e.g., Washington et al., Mayo. Clin. Proc. (1985) 60:189-203; Washington et al., Mayo. Clin. Proc. (1985) 60:271-278. However, erythromycin is known to undergo acid-promoted internal ketalization (cyclization of the C6 and C12 hydroxyl groups onto the C9 ketone) in the gut, which leads to adverse gastrointestinal events. See, e.g., Kurath et al., Experientia (1971) 27:362. Second-generation macrolide antibiotics clarithromycin and azithromycin addressed issues of acid instability and were prepared semi-synthetically in 4-6 steps from erythromycin, which is readily available through large-scale fermentation. See, e.g., Ma et al., Curr. Med. Chem. (2011) 18:1993-2015; Wu et al., Curr. Pharm. Des. (2000) 6:181-223; Ma et al., Mini-Rev. Med. Chem. (2010) 10:272-286; Asaka et al., Curr. Top. Med. Chem. (Sharjah, United Arab Emirates) (2003) 3:961-989; Morimoto et al., J. Antibiot. (1990) 43:286-294; Morimoto et al., J. Antibiot. (1984) 37:187-189; Watanabe et al., J. Antibiot. (1993) 46: 1163-1167; Watanabe et al., J. Antibiot. (1993) 46:647-660; Bright et al., J. Antibiot. (1988) 41: 1029-1047; Djokic et al., J. Antibiot. (1987) 40:1006-1015; Mutak et al., J. Antibiot. (2007) 60: 85-122; and Retsema et al., Antimicrob. Agents Chemother. (1987) 31:1939-1947. Azithromycin has been shown to exhibit markedly improved efficacy against Gram negative organisms, and it has a longer half-life and higher tissue distribution than the other macrolide antibiotics, thought to correlate with its 15-membered ring containing a tertiary amine. See, e.g., Ferwerda et al., J. Antimicrob. Chemother. (2001) 47:441-446; Girard et al., Antimicrob. Agents Chemother. (1987) 31:1948-1954. The natural product tylosin, a 16-membered macrolide used in veterinary medicine, has been shown by X-ray crystallography to occupy the same binding pocket as erythromycin and azithromycin, suggesting that there is a high tolerance for variability in ring size and composition of the macrocycle.

The three primary causes of resistance to macrolides in bacterial organisms are: ribosome methylation encoded by erm genes, mutations in ribosomal RNA or peptides, and cell efflux mediated by mef and msr genes. See, e.g., Leclercq et al., Antimicrob. Agents Chemother. (1991) 35:1273-1276; Leclercq et al., Antimicrob. Agents Chemother. (1991) 35:1267-1272; Weisblum, Antimicrob. Agents Chemother. (1995) 39:577-585; Vester et al., Antimicrob. Agents Chemother. (2001) 45:1-12; Prunier et al., Antimicrob. Agents Chemother. (2002) 46:3054-3056; Li et al., J. Antimicrob. Chemother. (2011) 66:1983-1986; Sutcliffe et al., Antimicrob. Agents Chemother. (1996) 40:1817-1824; Wondrack et al., Antimicrob. Agents Chemother. (1996) 40: 992-998. Ketolides such as telithromycin and solithromycin defeat the efflux mechanism of resistance by replacement of the C3 cladinose sugar with a carbonyl group (hence the name “ketolides”) and are thought to exhibit greatly increased binding by virtue of favorable interactions between the novel aryl-alkyl sidechain and the ribosome. See, e.g., Ma et al., Curr. Med. Chem. (2011) 18:1993-2015; Ma et al., Mini-Rev. Med. Chem. (2010) 10:272-286. Despite greatly improved ribosomal binding, ketolides such as telithromycin and solithromycin have not addressed several of the newest forms of macrolide resistance that have evolved in nosocomial settings, especially ribosome methylation and RNA point mutations.

Accordingly, the discovery and development of new antibiotics effective against drug-resistant bacteria, especially Gram-negative bacteria, represents a currently unmet medical need.

### SUMMARY

Disclosed herein are compounds that are novel, synthetically accessible 13-membered macrolides. The disclosed compounds are novel antibiotics with unexpectedly potent antimicrobial activity.

In one aspect, the present disclosure provides compounds of Formula (I):

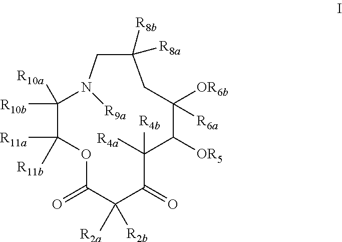

or a pharmaceutically acceptable salt thereof, wherein:

one of R₂ₐ and R_{2b} is selected from the group consisting of H, halo, optionally substituted C₁₋₁₀ alkyl, optionally substituted C₁₋₁₀ alkoxy, and optionally substituted C₁₋₁₀ alkenyl, wherein C₁₋₁₀ alkyl, C₁₋₁₀ alkoxy, and C₁₋₁₀ alkenyl are optionally substituted with one or more groups selected from the group consisting of halo, aryl, amino, alkyl, heteroalkyl, heteroalkenyl, heterocycloalkyl, and heteroaryl; and

the other of R₂ₐ and R_{2b} is selected from the group consisting of halo, optionally substituted C₁₋₁₀ alkyl, optionally substituted C₁₋₁₀ alkoxy, and optionally substituted C₁₋₁₀ alkenyl, wherein C₁₋₁₀ alkyl, C₁₋₁₀ alkoxy, and C₁₋₁₀ alkenyl are optionally substituted with one or more groups selected from the group consisting of halo, aryl, amino, alkyl, heteroalkyl, heteroalkenyl, heterocycloalkyl, and heteroaryl;

each of R₄ₐ and R_{4b} is independently selected from the group consisting of —H, and optionally substituted C₁₋₁₀alkyl;

R₅ is selected from the group consisting of —H, an oxygen protecting group, and

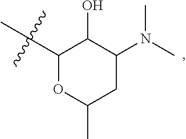 [SMILES: *C1OC(C)CC(N(C)C)C1O]

wherein “ [symbol] ” indicates appoint of attachment;

R₆ₐ is optionally substituted C₁₋₁₀ alkyl;

R_{6b} is —H, optionally substituted C₁₋₁₀ alkyl, optionally substituted C₁₋₁₀ hydroxyalkyl, and optionally substituted allyl;

R₈ₐ and R_{8b} are each independently selected from the group consisting of —H and optionally substituted C₁₋₁₀ alkyl;

R₉ₐ is selected from the group consisting of —H, —CO₂-alkylene-aryl, —C(═O)-alkyl, and optionally substituted C₁₋₁₀ alkyl;

one of R₁₀ₐ and R_{10b} is selected from the group consisting of —H, optionally substituted C₁₋₁₀ alkyl, —CO₂H, and —CO₂-alkyl; and

the other of R₁₀ₐ and R_{10b} is selected from the group consisting of optionally substituted saturated or partially unsaturated cycloalkyl containing at least one double bond, optionally substituted saturated or partially unsaturated heterocycloalkyl, optionally substituted aryl, and optionally substituted heteroaryl; and

R₁₁ₐ and R_{11b} are each independently selected from the group consisting of —H and optionally substituted C₁₋₁₀ alkyl.

The disclosed compounds have anti-microbial activity and may be used to treat and/or prevent infectious diseases. Pharmaceutical compositions of the compounds and methods of treatment and prevention using the compounds or compositions thereof are provided herein. Infectious diseases which may be treated with compounds of the invention include, but are not limited to, bacterial infections caused by Staphylococcus, Acinetobacter, Klebsiella, Escherichia, and Pseudomonas species.

Methods of preparing the compounds are also provided herein. The present disclosure also provides intermediates in the preparation of the compounds described herein.

The details of certain embodiments of the invention are set forth in the Detailed Description of Certain Embodiments, as described below. Other features, objects, and advantages of the invention will be apparent from the Definitions, Drawings, Examples, and Claims.

### DETAILED DESCRIPTION OF CERTAIN EMBODIMENTS

The compounds disclosed herein include 13-membered azaketolides. The disclosed compounds may have reduced structural complexity over known macrolides, providing compounds that may be accessed by less demanding synthetic routes over routes required for other macrolides. Despite their reduced structural complexity, the disclosed 13-membered azaketolides provide unexpected and potent activity against various microorganisms, including Gram negative bacteria. Also disclosed are methods for the preparation of the compounds, pharmaceutical compositions comprising the compounds, and methods of using the compounds (e.g., treatment of an infectious disease).

In certain embodiments, provided are compounds of formula I:

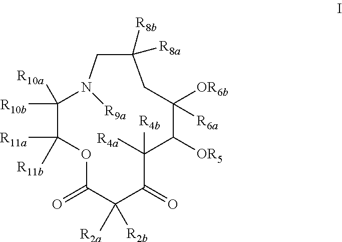

or a pharmaceutically acceptable salt thereof, wherein:

one of R₂ₐ and R_{2b} is selected from the group consisting of H, halo, optionally substituted C₁₋₁₀ alkyl, optionally substituted C₁₋₁₀ alkoxy, and optionally substituted C₁₋₁₀ alkenyl, wherein C₁₋₁₀ alkyl, C₁₋₁₀ alkoxy, and C₁₋₁₀ alkenyl are optionally substituted with one or more groups selected from the group consisting of halo, aryl, amino, alkyl, heteroalkyl, heteroalkenyl, heterocycloalkyl, and heteroaryl; and

the other of R₂ₐ and R_{2b} is selected from the group consisting of halo, optionally substituted C₁₋₁₀ alkyl, optionally substituted C₁₋₁₀ alkoxy, and optionally substituted C₁₋₁₀ alkenyl, wherein C₁₋₁₀ alkyl, C₁₋₁₀ alkoxy, and C₁₋₁₀ alkenyl are optionally substituted with one or more groups selected from the group consisting of halo, aryl, amino, alkyl, heteroalkyl, heteroalkenyl, heterocycloalkyl, and heteroaryl;

each of R₄ₐ and R_{4b} is independently selected from the group consisting of —H, and optionally substituted C₁₋₁₀ alkyl;

R₅ is selected from the group consisting of —H, an oxygen protecting group, and

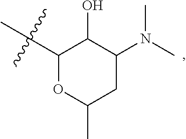 [SMILES: *C1OC(C)CC(N(C)C)C1O]

wherein “ [symbol] ” indicates appoint of attachment;

R₆ₐ is optionally substituted C₁₋₁₀ alkyl;

R_{6b} is —H, optionally substituted C₁₋₁₀ alkyl, optionally substituted C₁₋₁₀ hydroxyalkyl, and optionally substituted allyl;

R₈ₐ and R_{8b} are each independently selected from the group consisting of —H and optionally substituted C₁₋₁₀ alkyl;

R₉ₐ is selected from the group consisting of —H, —CO₂-alkylene-aryl, —C(═O)-alkyl, and optionally substituted C₁₋₁₀ alkyl;

one of R₁₀ₐ and R_{10b} is selected from the group consisting of —H, optionally substituted C₁₋₁₀ alkyl; —CO₂H, and —CO₂-alkyl; and

the other of R₁₀ₐ and R_{10b} is selected from the group consisting of optionally substituted saturated or partially unsaturated cycloalkyl containing at least one double bond, optionally substituted saturated or partially unsaturated heterocycloalkyl, optionally substituted aryl, and optionally substituted heteroaryl; and

R₁₁ₐ and R_{11b} are each independently selected from the group consisting of —H and optionally substituted C₁₋₁₀ alkyl.

In certain embodiments, provided are compounds of formula I:

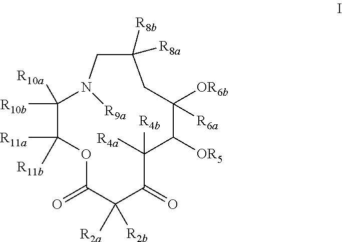

or a pharmaceutically acceptable salt thereof, wherein:

one of R₂ₐ and R_{2b} is selected from the group consisting of H, halo, optionally substituted C₁₋₁₀ alkyl, optionally substituted C₁₋₁₀ alkoxy, and optionally substituted C₁₋₁₀ alkenyl, wherein C₁₋₁₀ alkyl, C₁₋₁₀ alkoxy, and C₁₋₁₀ alkenyl are optionally substituted with one or more groups selected from the group consisting of halo, aryl, amino, alkyl, heteroalkyl, heteroalkenyl, heterocycloalkyl, and heteroaryl; and

the other of R₂ₐ and R_{2b} is selected from the group consisting of halo, optionally substituted C₁₋₁₀ alkyl, optionally substituted C₁₋₁₀ alkoxy, and optionally substituted C₁₋₁₀ alkenyl, wherein C₁₋₁₀ alkyl, C₁₋₁₀ alkoxy, and C₁₋₁₀ alkenyl are optionally substituted with one or more groups selected from the group consisting of halo, aryl, amino, alkyl, heteroalkyl, heteroalkenyl, heterocycloalkyl, and heteroaryl;

each of R₄ₐ and R_{4b} is independently selected from the group consisting of —H, and optionally substituted C₁₋₁₀ alkyl;

R₅ is selected from the group consisting of —H, an oxygen protecting group, and

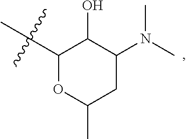 [SMILES: *C1OC(C)CC(N(C)C)C1O]

wherein “ [symbol] ” indicates appoint of attachment;

R₆ₐ is optionally substituted C₁₋₁₀ alkyl;

R_{6b} is —H, optionally substituted C₁₋₁₀ alkyl, optionally substituted C₁₋₁₀ hydroxyalkyl, and optionally substituted allyl;

R₈ₐ and R_{8b} are each independently selected from the group consisting of —H and optionally substituted C₁₋₁₀ alkyl;

R₉ₐ is selected from the group consisting of —H and optionally substituted C₁₋₁₀ alkyl;

one of R₁₀ₐ and R_{10b} is selected from the group consisting of —H, optionally substituted C₁₋₁₀ alkyl; —CO₂H, and —CO₂-alkyl; and

the other of R₁₀ₐ and R_{10b} is selected from the group consisting of optionally substituted saturated or partially unsaturated cycloalkyl containing at least one double bond, optionally substituted saturated or partially unsaturated heterocycloalkyl, optionally substituted aryl, and optionally substituted heteroaryl; and

R₁₁ₐ and R_{11b} are each independently selected from the group consisting of —H and optionally substituted C₁₋₁₀ alkyl.

One embodiment of a compound of formula I is a compound of formula IA:

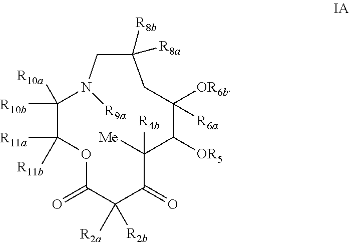

Another embodiment of a compound of formula I and IA is a compound of formula IB:

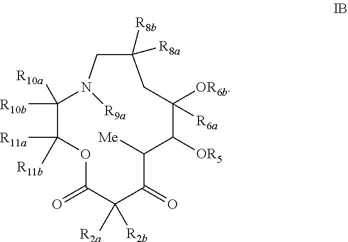

In certain embodiments of the compound of formula I, IA, and IB,

R₅ is

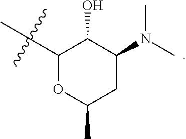 [SMILES: *C1O[C@H](C)C[C@H](N(C)C)[C@H]1O]

Another embodiment of a compound of formula I, IA, and IB is a compound of formula IC:

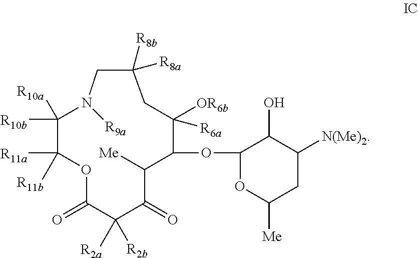

Another embodiment of a compound of formula I, IA, IB, and IC is a compound of formula ID:

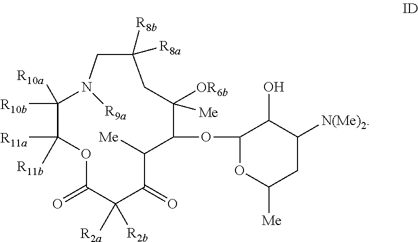

In another embodiment of a compound of formula I, IA, IB, IC, and ID, R_{6b} is selected from the group consisting of —H, optionally substituted C₁-C₁₀ alkyl, optionally substituted C₁-C₁₀ hydroxyalkyl, and allyl.

In another embodiment of a compound of formula I, IA, IB, IC, and ID, R_{6b} is selected from the group consisting of: methyl, hydroxymethyl, hydroxyethyl, hydroxypropyl, hydroxybutyl, hydroxypentyl, hydroxyhexyl, —CH₂CH(OH)CH₂OH, and allyl.

Another embodiment of a compound of formula I, IA, IB, IC, and ID is a compound of formula IE:

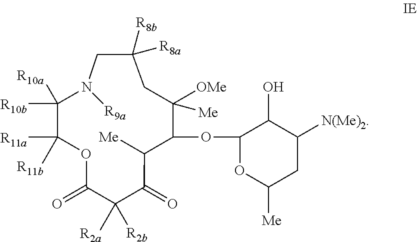

Another embodiment of a compound of formula I, IA, IB, IC, ID, and IE is a compound of formula IF:

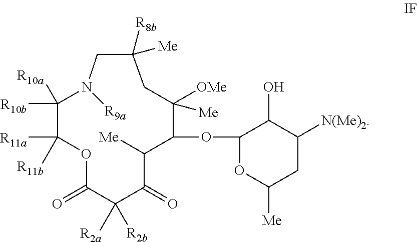

Another embodiment of a compound of formula I, IA, IB, IC, ID, IE, and IF is a compound of formula IG:

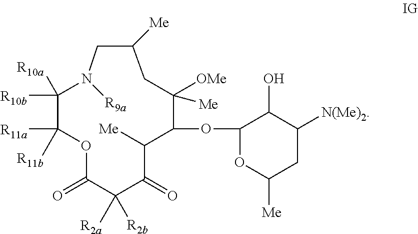

In another embodiment of a compound of formula I, IA, IB, IC, ID, IE, IF, and IG, R₉ₐ is —H or optionally substituted C₁₋₄ alkyl.

In another embodiment of a compound of formula I, IA, IB, IC, ID, IE, IF, and IG, R₉ₐ is —H, methyl, ethyl, propyl, isopropyl, butyl, or isobutyl. In another embodiment of a compound of formula I, IA, IB, IC, ID, IE, IF, and IG, R₉ₐ is —H, or methyl.

In another embodiment of a compound of formula I, IA, IB, IC, ID, IE, IF, and IG, R₁₁ₐ and R_{11b} are —H.

In another embodiment of a compound of formula I, IA, IB, IC, ID, IE, IF, and IG, one of R₁₁ₐ and R_{11b} is —H and the other is optionally substituted C₁₋₁₀ alkyl.

In another embodiment of a compound of formula I, IA, IB, IC, ID, IE, IF, and IG, one of R₁₁ₐ and R_{11b} is H and the other is methyl.

In another embodiment of a compound of formula I, IA, IB, IC, ID, IE, IF, and IG, R₁₁ₐ and R_{11b} are each independently optionally substituted C₁₋₁₀ alkyl.

In another embodiment of a compound of formula I, IA, IB, IC, ID, IE, IF, and IG, R₁₁ₐ and R_{11b} are each methyl.

In another embodiment of a compound of formula I, IA, IB, IC, ID, IE, IF, or IG, one of R₂ₐ and R_{2b} is optionally substituted C₁₋₁₀ alkyl.

In another embodiment of a compound of formula I, IA, IB, IC, ID, IE, IF, or IG, one of R₂ₐ and R_{2b} is optionally substituted C₁₋₁₀ alkyl and the other of R₂ₐ and R_{2b} is H.

In another embodiment of a compound of formula I, IA, IB, IC, ID, IE, IF, or IG, both of R₂ₐ and R_{2b} are optionally substituted C₁₋₁₀ alkyl.

In another embodiment of a compound of formula I, IA, IB, IC, ID, IE, IF, or IG, one of R₂ₐ and R_{2b} is methyl.

In another embodiment of a compound of formula I, IA, IB, IC, ID, IE, IF, or IG, one of R₂ₐ and R_{2b} is methyl and the other of R₂ₐ and R_{2b} is H.

In another embodiment of a compound of formula I, IA, IB, IC, ID, IE, IF, or IG, both of R₂ₐ and R_{2b} are methyl.

In another embodiment of a compound of formula I, IA, IB, IC, ID, IE, IF, or IG, one of R₂ₐ and R_{2b} is methyl and the other is halo. In a further embodiment, halo is selected from the group consisting of F and Cl.

In another embodiment of a compound of formula I, IA, IB, IC, ID, IE, IF, or IG, one of R₂ₐ and R_{2b} is methyl and the other is optionally substituted C₁₋₁₀ alkyl.

In another embodiment of a compound of formula I, IA, IB, IC, ID, IE, IF, or IG, one of R₂ₐ and R_{2b} is methyl and the other is selected from the group consisting of optionally substituted C₁₋₁₀ alkyl, optionally substituted C₁₋₁₀ alkoxy, and optionally substituted C₁₋₁₀ alkenyl, wherein C₁₋₁₀ alkyl, C₁₋₁₀ alkoxy, and C₁₋₁₀ alkenyl are optionally substituted with one or more groups selected from halo, aryl, and heteroaryl.

Another embodiment of a compound of formula I, IA, IB, IC, ID, IE, IF, or IG is a compound of formula IG-1.

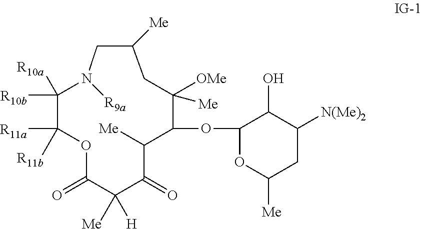

Another embodiment of a compound of formula I, IA, IB, IC, ID, IE, IF, and IG is a compound of formula IH:

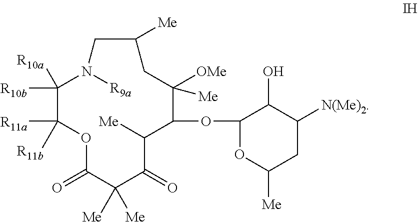

In another embodiment of a compound of formula I, IA, IB, IC, ID, IE, IF, IG, IG-1, or IH R₉ₐ is —H, methyl, ethyl, propyl, isopropyl, butyl, or isobutyl. In another embodiment of a compound of formula I, IA, IB, IC, ID, IE, IF, and IG, R₉ₐ is —H, or methyl.

In another embodiment of a compound of formula I, IA, IB, IC, ID, IE, IF, IG, IG-1, or IH, one of R₁₀ₐ and R_{10b} is H or optionally substituted C₁₋₁₀ alkyl.

In another embodiment of a compound of formula I, IA, IB, IC, ID, IE, IF, IG, IG-1, or IH, one of R₁₀ₐ and R_{10b} is H.

In another embodiment of a compound of formula I, IA, IB, IC, ID, IE, IF, IG, IG-1 or IH, one of R₁₀ₐ and R_{10b} is optionally substituted C₁₋₁₀ alkyl.

In another embodiment of a compound of formula I, IA, IB, IC, ID, IE, IF, IG, IG-1, or IH, one of R₁₀ₐ and R_{10b} is methyl.

Another embodiment of a compound of formula I, IA, IB, IC, ID, IE, IF, IG, and IH is a compound of formula IIA, IIB, IIC, or IID:

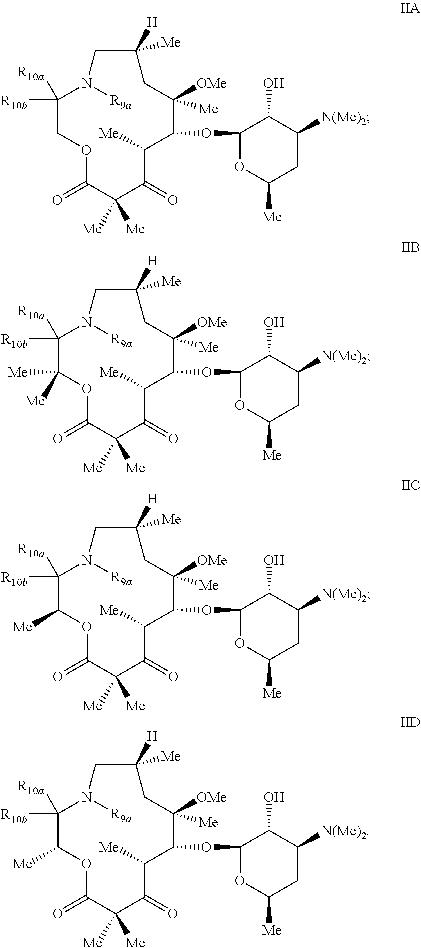

In one embodiment of a compound of formula IIA, IIB, IIC, and IID, R₉ₐ is —H, methyl, ethyl, propyl, isopropyl, butyl, or isobutyl. In another embodiment of a compound of formula IIA, IIB, IIC, and IID, R₉ₐ is —H, or methyl.

Another embodiment of a compound of formula IIA, IIB, IIC, and IID is a compound of formula IIA-1, IIA-2, IIB-1, IIB-2, IIC-1, IIC-2, IID-1, or IID-2:

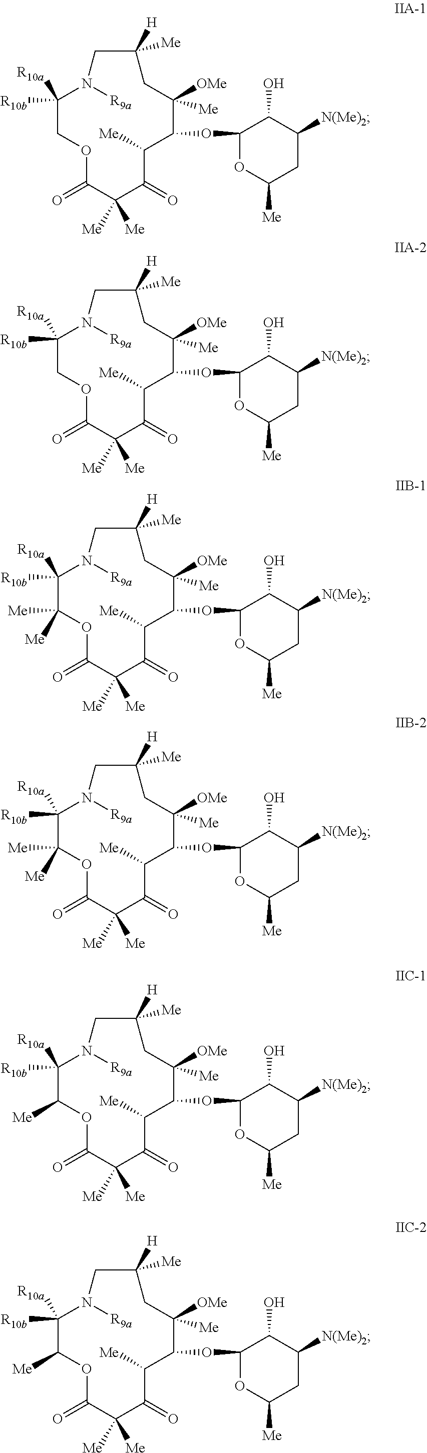

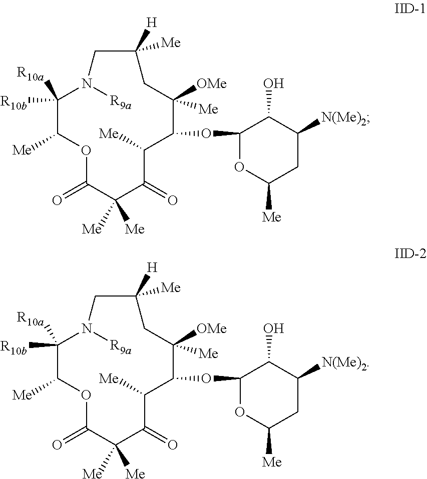

In one embodiment of a compound of formula IIA-1, IIA-2, IIB-1, IIB-2, IIC-1, IIC-2, IID-1, or IID-2, R₉ₐ is —H, methyl, ethyl, propyl, isopropyl, butyl, or isobutyl. In another embodiment of a compound of formula IIA-1, IIA-2, IIB-1, IIB-2, IIC-1, IIC-2, IID-1, or IID-2, R₉ₐ is —H, or methyl.

Another embodiment of a compound of formula IIA, IIB, IIC, and IID is a compound of formula IIA-1a, IIA-2a, IIB-1a, IIB-2a, IIC-1a, IIC-2a, IID-1a, or IID-2a:

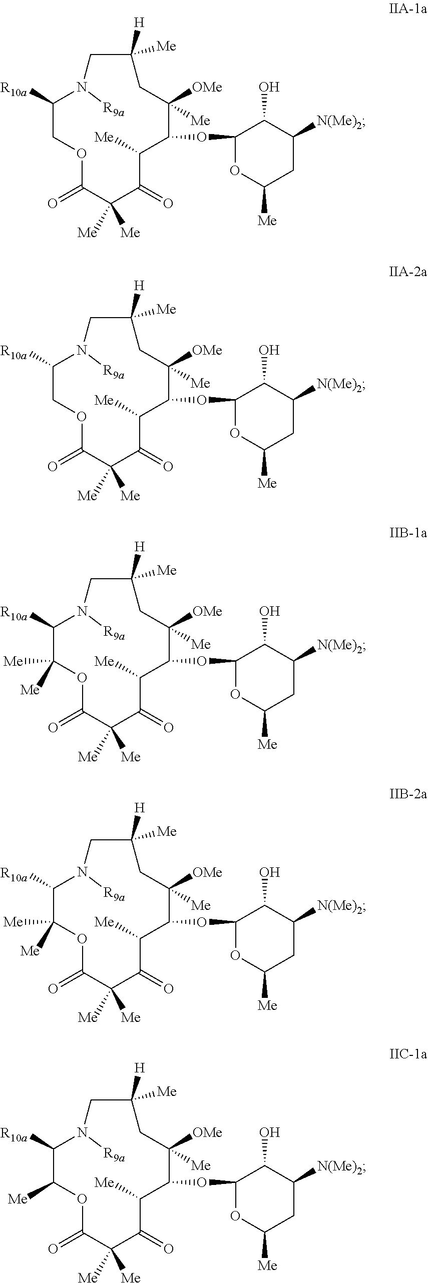

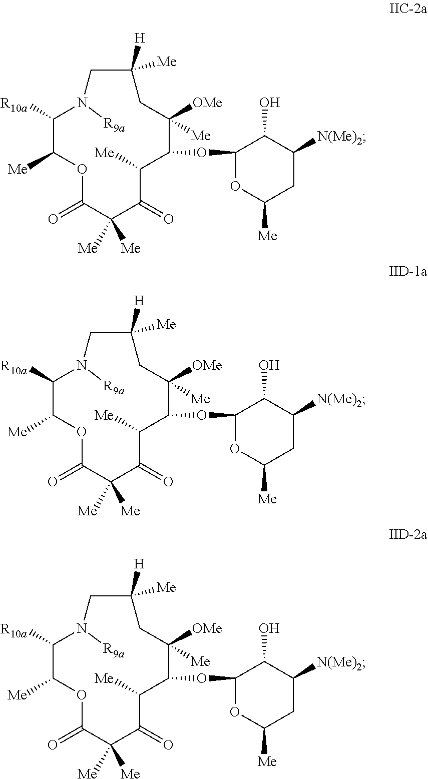

wherein R₁₀ₐ is selected from the group consisting of optionally substituted cycloalkyl, optionally substituted heterocycloalkyl, optionally substituted aryl, and an optionally substituted heteroaryl.

In one embodiment of a compound of formula IIA-1a, IIA-2a, IIB-1a, IIB-2a, IIC-1a, IIC-2a, IID-1a, or IID-2a, R₉ₐ is —H, methyl, ethyl, propyl, isopropyl, butyl, or isobutyl. In another embodiment of a compound of formula IIA-1a, IIA-2a, IIB-1a, IIB-2a, IIC-1a, IIC-2a, IID-1a, or IID-2a, R₉ₐ is —H, or methyl.

Another embodiment of a compound of formula IIA, IIB, IIC, and IID is a compound of formula IIA-1b, IIA-2b, IIB-1b, IIB-2b, IIC-1b, IIC-2b, IID-1b, or IID-2b:

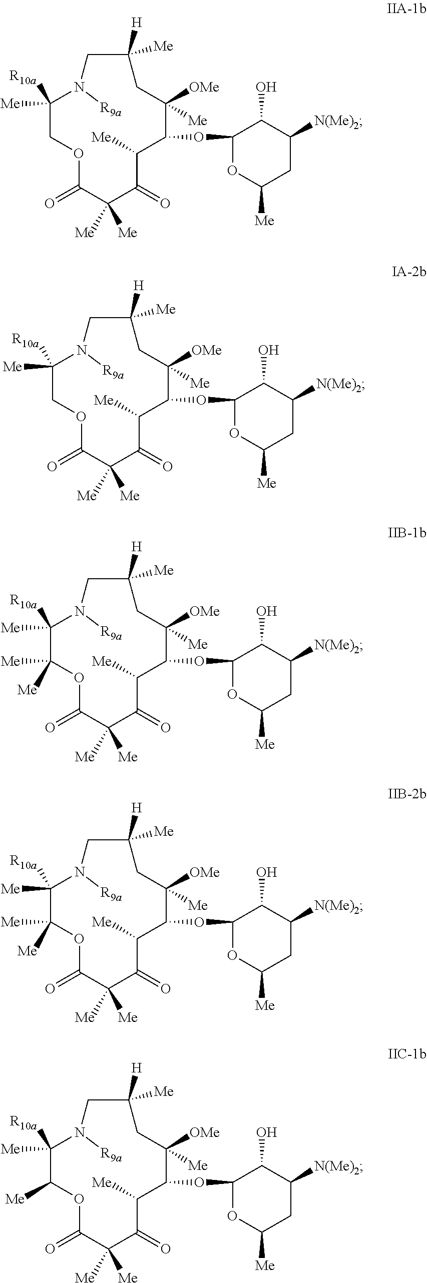

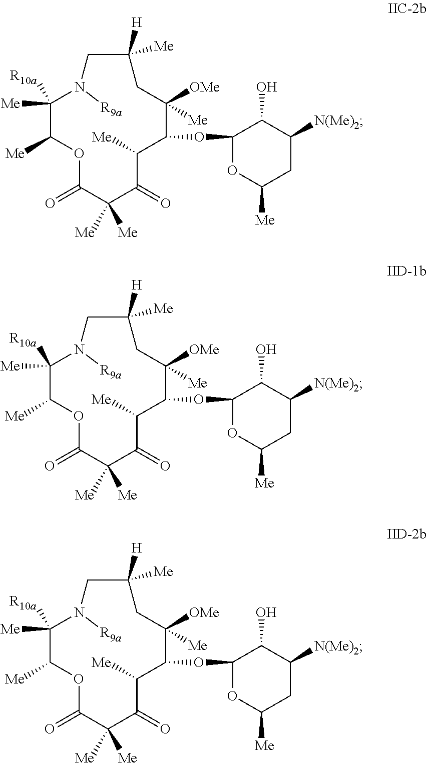

wherein R₁₀ₐ is selected from the group consisting of optionally substituted saturated or partially unsaturated cycloalkyl, optionally substituted saturated or partially unsaturated heterocycloalkyl, optionally substituted aryl, and an optionally substituted heteroaryl.

In one embodiment of a compound of formula IIA-1b, IIA-2b, IIB-1b, IIB-2b, IIC-1b, IIC-2b, IID-1b, or IID-2b, R₉ₐ is —H, methyl, ethyl, propyl, isopropyl, butyl, or isobutyl. In another embodiment of a compound of formula IIA-1b, IIA-2b, JIB-1b, IIB-2b, IIC-1b, IIC-2b, IID-1b, or IID-2b, R₉ₐ is —H, or methyl.

In another embodiment of formulas I, IA, IB, IC, ID, IE, IF, IG, IH, IIA, IIB, IIC, IID, IIA-1, IIA-2, IB-1, IIB-2, IIC-1, IIC-2, IID-1, and IID-2:

one of R₁₀ₐ and R_{10b} is selected from the group consisting of H and optionally substituted C₁₋₁₀ alkyl, —CO₂H, and —CO₂-alkyl; and

the other of R₁₀ₐ and R_{10b} is selected from the group consisting of optionally substituted saturated or partially unsaturated cycloalkyl, optionally substituted saturated or partially unsaturated heterocycloalkyl saturated or partially unsaturated heterocycloalkyl, optionally substituted aryl, and an optionally substituted heteroaryl.

In another embodiment of formulas I, IA, IB, IC, ID, IE, IF, IG, IH, IIA, IIB, IIC, IID, IIA-1, IIA-2, IIB-1, IIB-2, IIC-1, IIC-2, IID-1, IID-2, IIA-1a, IIA-2a, JIB-1a, IIB-2a, IIC-1a, IIC-2a, IID-1a, or IID-2a, IIA-1b, IIA-2b, JIB-1b, IIB-2b, IIC-1b, IIC-2b, IID-1b, and IID-2b:

R₁₀ₐ is optionally substituted -arylene-R₁₀₁; R₁₀₁ is selected from the group consisting of H, halo, —B(OH)₂, —B(O-alkyl)₂, optionally substituted aryl, and optionally substituted heteroaryl.

In another embodiment of formulas I, IA, IB, IC, ID, IE, IF, IG, IH, IIA, IIB, IIC, IID, IIA-1, IIA-2, IIB-1, IIB-2, IIC-1, IIC-2, IID-1, IID-2, IIA-1a, IIA-2a, IIB-1a, IIB-2a, IIC-1a, IIC-2a, IID-1a, or IID-2a, IIA-1b, IIA-2b, IIB-1b, IIB-2b, IIC-1b, IIC-2b, IID-1b, and IID-2b:

R₁₀ₐ is -phenylene-R₁₀₁ₐ; and

R₁₀₁ₐ is selected from the group consisting of —H, halo, —B(OH)₂, —B(O-alkyl)₂, optionally substituted phenyl, and optionally substituted heteroaryl.

In another embodiment of formulas I, IA, IB, IC, ID, IE, IF, IG, IH, IIA, IIB, IIC, IID, IIA-1, IIA-2, IIB-1, IIB-2, IIC-1, IIC-2, IID-1, IID-2, IIA-1a, IIA-2a, IIB-1a, IIB-2a, IIC-1a, IIC-2a, IID-1a, or IID-2a, IIA-1b, IIA-2b, IIB-1b, IIB-2b, IIC-1b, IIC-2b, IID-1b, and IID-2b:

R₁₀ₐ is selected from the group consisting of phenyl, bromophenyl, aminophenyl,

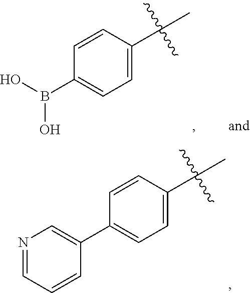 [SMILES: *c1ccc(B(O)O)cc1 ; *c1ccc(-c2cccnc2)cc1]

wherein “ [symbol] ” indicates a point of attachment.

In another embodiment of formulas I, IA, IB, IC, ID, IE, IF, IG, IH, IIA, IIB, IIC, IID, IIA-1, IIA-2, IIB-1, IIB-2, IIC-1, IIC-2, IID-1, IID-2, IIA-1a, IIA-2a, IIB-1a, IIB-2a, IIC-1a, IIC-2a, IID-1a, or IID-2a, IIA-1b, IIA-2b, IIB-1b, IIB-2b, IIC-1b, IIC-2b, IID-1b, and IID-2b:

R₁₀ₐ is optionally substituted -heteroarylene-R_{101b};

R_{101b} is selected from the group consisting of —H, halo, optionally substituted aryl, and optionally substituted heteroaryl.

In another embodiment of formulas I, IA, IB, IC, ID, IE, IF, IG, IH, IIA, IIB, IIC, IID, IIA-1, IIA-2, IIB-1, IIB-2, IIC-1, IIC-2, IID-1, IID-2, IIA-1a, IIA-2a, IIB-1a, IIB-2a, IIC-1a, IIC-2a, IID-1a, or IID-2a, IIA-1b, IIA-2b, IIB-1b, IIB-2b, IIC-1b, IIC-2b, IID-1b, and IID-2b:

R₁₀ₐ is optionally substituted pyridyl.

In another embodiment of formulas I, IA, IB, IC, ID, IE, IF, IG, IIA, IIB, IIC, IID, IIA-1, IIA-2, IIB-1, IIB-2, IIC-1, IIC-2, IID-1, IID-2, IIA-1a, IIA-2a, IIB-1a, IIB-2a, IIC-1a, IIC-2a, IID-1a, or IID-2a, IIA-1b, IIA-2b, IIB-1b, IIB-2b, IIC-1b, IIC-2b, IID-1b, and IID-2b:

R₁₀ₐ is optionally substituted pyridyl-R_{101b}, wherein R_{101b} is —H.

In another embodiment of formulas I, IA, IB, IC, ID, IE, IF, IG, IH, IIA, IIB, IIC, IID, IIA-1, IIA-2, IIB-1, IIB-2, IIC-1, IIC-2, IID-1, IID-2, IIA-1a, IIA-2a, IIB-1a, IIB-2a, IIC-1a, IIC-2a, IID-1a, or IID-2a, IIA-1b, IIA-2b, IIB-1b, IIB-2b, IIC-1b, IIC-2b, IID-1b, and IID-2b:

R₁₀ₐ is selected from the group consisting of optionally substituted saturated or partially unsaturated cycloalkyl selected from the group consisting of optionally substituted cyclopropyl, optionally substituted cyclobutyl, optionally substituted saturated or partially unsaturated cyclopentyl, optionally substituted saturated or partially unsaturated cyclohexyl, and optionally substituted saturated or partially unsaturated cycloheptyl.

In another embodiment of formulas I, IA, IB, IC, ID, IE, IF, IG, IIA, IIB, IIC, IID, IIA-1, IIA-2, IIB-1, IIB-2, IIC-1, IIC-2, IID-1, IID-2, IIA-1a, IIA-2a, IIB-1a, IIB-2a, IIC-1a, IIC-2a, IID-1a, or IID-2a, IIA-1b, IIA-2b, IIB-1b, IIB-2b, IIC-1b, IIC-2b, IID-1b, and IID-2b:

R₁₀ₐ is optionally substituted cyclopropyl.

In another embodiment of formulas I, IA, IB, IC, ID, IE, IF, IG, IIA, IIB, IIC, IID, IIA-1, IIA-2, IIB-1, IIB-2, IIC-1, IIC-2, IID-1, IID-2, IIA-1a, IIA-2a, IIB-1a, IIB-2a, IIC-1a, IIC-2a, IID-1a, or IID-2a, IIA-1b, IIA-2b, IIB-1b, IIB-2b, IIC-1b, IIC-2b, IID-1b, and IID-2b:

R₁₀ₐ is

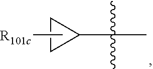

wherein “ [symbol] ” indicates a point of attachment;

R_{101c} is selected from the group consisting of —H, halo, —OH, alkoxy, —NRₓR_{x′}, and alkylene-R_{101c′}, wherein R_{101c′} is selected from the group consisting of —H, halo, —OH, alkoxy, and NRₓR_{x′}, wherein:

at each occurrence Rₓ and R_{x′} are each independently selected from the group consisting of —H, optionally substituted alkyl, —C(═O)-alkyl, and —C(═O)-alkylene-N(R_{y′})(R_{y″}); or

Rₓ and R_{x′} together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl; and wherein

R_{y′} and R_{y″} are each independently selected from the group consisting of —H and optionally substituted C₁₋₁₀ alkyl; or

R_{y′} and R_{y″}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

In another embodiment:

R₁₀ₐ is

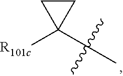

wherein “ [symbol] ” indicates a point of attachment;

R_{101c} is -alkylene-R_{101c′}, wherein R_{101c′} is selected from the group consisting of —H, halo, —OH, alkoxy, and —NRₓR_{x′}, wherein: at each occurrence Rₓ and R_{x′} are each independently selected from the group consisting of —H, optionally substituted alkyl, —C(═O)-alkyl, and —C(═O)-alkylene-N(R_{y′})(R_{y″}); or

Rₓ and R_{x′} together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl; and wherein R_{y′} and R_{y″} are each independently selected from the group consisting of —H and optionally substituted C₁₋₁₀ alkyl; or

R_{y′} and R_{y″}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

In another embodiment,

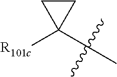

is selected from the group consisting of

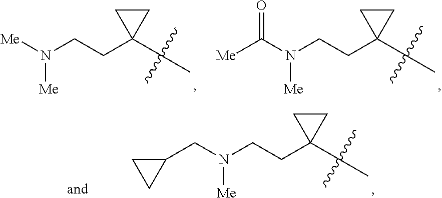

wherein “ [symbol] ” indicates a point of attachment.

In another embodiment, R₁₀ₐ is optionally substituted cyclobutyl.

In another embodiment:

R₁₀ₐ is

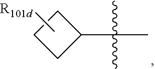

wherein “ [symbol] ” indicates a point of attachment;

R_{101d} is selected from the group consisting of —H, halo, —OH, alkoxy, −NRₓR_{x′}, and -alkylene-R_{101d′}, wherein R_{101d′} is selected the group consisting of —H, halo, —OH, alkoxy, and —NRₓR_{x′}, wherein:

at each occurrence Rₓ and R_{x′} are each independently selected from the group consisting of —H, optionally substituted alkyl, —C(═O)-alkyl, and —C(═O)-alkylene-N(R_{y′})(R_{y″}); or

Rₓ and R_{x′} together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl; and wherein

R_{y′} and R_{y″} are each independently selected from the group consisting of —H and optionally substituted C₁₋₁₀ alkyl; or

R_{y′} and R_{y″}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

In another embodiment:

R₁₀ₐ is

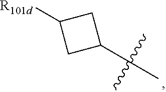

wherein “ [symbol] ” indicates a point of attachment.

In another embodiment, R_{101d} is selected from the group consisting of —NRₓR_{x′} and alkylene-R_{101d′}, wherein R_{101d′} is selected from the group consisting of —H, halo, —OH, alkoxy, and —NRₓR_{x′}, wherein:

at each occurrence Rₓ and R_{x′} are each independently selected from the group consisting of —H, optionally substituted alkyl, —C(═O)-alkyl, and —C(═O)-alkylene-N(R_{y′})(R_{y″}); or

Rₓ and R_{x′} together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl; and wherein

R_{y′} and R_{y″} are each independently selected from the group consisting of —H and optionally substituted C₁₋₁₀ alkyl; or

R_{y′} and R_{y″}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

In one embodiment,

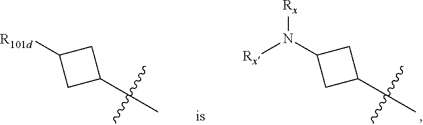

wherein one of Rₓ and R_{x′} is H or methyl and the other of Rₓ and R_{x′} is H, methyl, ethyl, propyl, isopropyl, butyl, isobutyl, —CH₂-cyclopropyl, wherein “ [symbol] ” indicates a point of attachment.

In another embodiment,

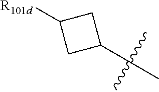

is selected from the group consisting of

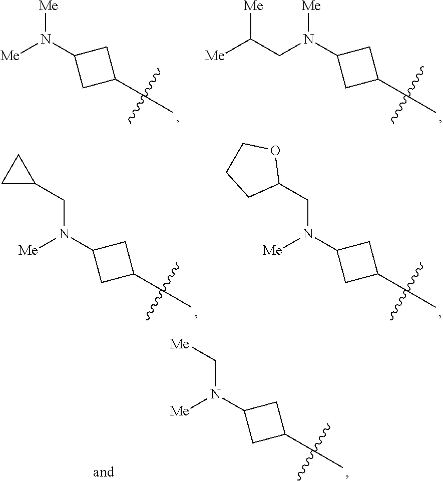 [SMILES: *C1CC(N(C)C)C1 ; *C1CC(N(C)CC(C)C)C1 ; *C1CC(N(C)CC2CC2)C1 ; *C1CC(N(C)CC2CCCO2)C1 ; *C1CC(N(C)CC)C1]

wherein “ [symbol] ” indicates a point of attachment.

In another embodiment:

R₁₀ₐ is optionally substituted saturated or partially unsaturated cyclopentyl.

In another embodiment:

R₁₀ₐ is optionally substituted saturated or partially unsaturated cyclohexyl.

In another embodiment:

R₁₀ₐ is

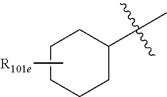

wherein “ [symbol] ” indicates a point of attachment;

R₁₀₁ₑ is selected from the group consisting of —H, halo, —OH, alkoxy, —NRₓR_{x′}, halo, —OH, alkoxy, —NR_{x′}R_{x′}, -alkylene-R_{101e′}, wherein R_{101e′} is selected from the group consisting of —H, halo, —OH, alkoxy, and —NR_{x′}R_{x′} wherein:

at each occurrence Rₓ and R_{x′} are each independently selected from the group consisting of —H, optionally substituted alkyl, —C(═O)-alkyl, and —C(═O)-alkylene-N(R_{y′})(R_{y″}); or

Rₓ and R_{x′} together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl; and wherein

R_{y′} and R_{y″} are each independently selected from the group consisting of —H and optionally substituted C₁₋₁₀ alkyl; or

R_{y′} and R_{y″}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

In another embodiment, R₁₀ₐ is

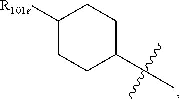

wherein “ [symbol] ” indicates a point of attachment; and wherein:

R₁₀₁ₑ is selected from the group consisting of —H, —NRₓR_{x′}, and alkylene-R_{101e′}, wherein R₁₀₁ₑ, is selected from the group consisting of —H, halo, —OH, alkoxy, —NRₓR_{x′}, cycloalkyl, and heterocycloalkyl, wherein:

at each occurrence Rₓ and R_{x′} are each independently selected from the group consisting of —H, optionally substituted alkyl, —C(═O)-alkyl, and —C(═O)-alkylene-N(R_{y′})(R_{y″}); or

Rₓ and R_{x′} together with the atom to which they are attached form a 3-, 4-, 5-, 6-, or 7-membered ring optionally containing an additional heteroatom selected from the group consisting of O, S, SO, SO₂, NR_{y}, and N—C₁-C₁₀ alkyl; and wherein

R_{y′} and R_{y″} are each independently selected from the group consisting of —H and optionally substituted C₁₋₁₀ alkyl; or

R_{y′} and R_{y″}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

In one embodiment of

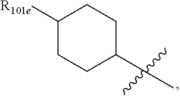

R₁₀₁ₑ is H or methyl.

In one embodiment of

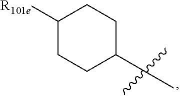

R₁₀₁ₑ is NRₓR_{x′}, wherein one of Rₓ and R_{x′} is H, methyl, or ethyl, and the other of Rₓ and R_{x′} is —C(═O)-alkylene-N(R_{y}j)(R_{y″}), wherein R_{y′} and R_{y″} are each independently H or optionally substituted C₁₋₁₀ alkyl. In another embodiment, one of Rₓ and R_{x′} is H, methyl, or ethyl, and the other of Rₓ and R_{x′} is —C(═O)—CH₂—N(R_{y′})(R_{y″}) wherein R_{y′} and R_{y″} are each independently H or methyl. In another embodiment, one of Rₓ and R_{x′} is H, methyl, or ethyl, and the other of Rₓ and R_{x′} is —C(═O)—CH₂—N(R_{y′})(R_{y″}) wherein one of R_{y′} and R_{y″} is H or methyl and the other of R_{y′} and R_{y″} is H, methyl, cyclopropyl, or —CH₂-cyclopropyl.

In another embodiment,

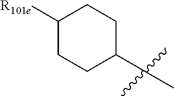

is selected from the group consisting of

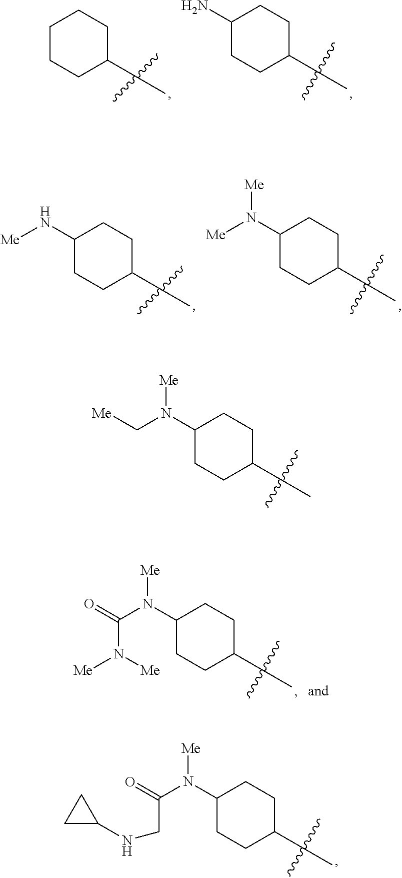

wherein “ [symbol] ” indicates a point of attachment.

In another embodiment:

R₁₀ₐ is optionally substituted saturated or partially unsaturated cycloheptyl.

In another embodiment:

R₁₀ₐ is optionally substituted heterocycloalkyl selected from the group consisting of optionally substituted aziridinyl, optionally substituted saturated or partially unsaturated pyrrolidinyl, and optionally substituted saturated or partially unsaturated piperidinyl.

In another embodiment:

R₁₀ₐ is optionally substituted aziridinyl.

In another embodiment:

R₁₀ₐ is optionally substituted azetidinyl.

In another embodiment:

R₁₀ₐ is azetidinyl optionally substituted with R_{101f}, wherein the point of attachment is the azetidinyl;

R_{101f} is selected from the group consisting of —H, halo, optionally substituted alkyl, —OH, —CO₂H, —CO₂-alkyl, alkoxy, optionally substituted cycloalkyl, optionally substituted heterocycloalkyl, NRₓR_{x′}, —C(═O)-alkyl, —C(═O)-optionally substituted heterocycloalkyl, alkenyl, —C(═O)-optionally substituted alkylene-R_{101f′}, -alkylene-C(═O)—R_{101f′} and -alkylene-R_{101f′}, wherein R_{101f′} is selected from the group consisting of —H, halo, —OH, alkoxy, —CO₂H, CO₂-optionally substituted alkyl, optionally substituted cycloalkyl, optionally substituted hetercycloalkyl, optionally substituted aryl, and optionally substituted heteroaryl, and —NRₓR_{x′}, wherein:

Rₓ and R_{x′} are each independently selected from the group consisting of —H, optionally substituted alkyl, —C(═O)-alkyl, and —C(═O)-alkylene-N(R_{y})₂; or

Rₓ and R_{x′} together with the atom to which they are attached form a 3-, 4-, 5-, 6-, 7-, optionally containing an additional heteroatom selected from the group consisting of O, S, SO, SO₂, NR_{y}, and N—C₁-C₁₀ alkyl; and wherein

each R_{y} is independently selected from the group consisting of —H and optionally substituted C₁₋₁₀ alkyl; or

each R_{y}, together with the atom to which they are attached form a 3-, 4-, 5-, 6-, 7, 8-, 9-, or 10-membered monocyclic or bicyclic ring optionally containing an additional heteroatom selected from the group consisting of O, S, SO, SO₂, NH, and N—C₁-C₁₀ alkyl.

In another embodiment, R₁₀ₐ is

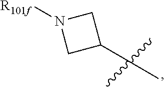

R_{101f} is H. In another embodiment, R_{101f} is -alkylene-R_{101f′}, wherein R_{101f′} is H. In some embodiments, -alkylene-R_{101f′} is selected from the group consisting of methyl, ethyl, propyl, isopropyl, butyl, sec-butyl, tert-butyl, pentyl, isopentyl, neopentyl, hexyl, and heptyl.

In another embodiment, R_{101f} is selected from the group consisting of —H, methyl, ethyl, propyl, isopropyl, butyl, isobutyl, pentyl, and neopentyl.

In another embodiment, R_{101f} is selected from the group consisting of —CH₂-cyclopropyl, —CH₂-cyclobutyl, —CH₂-cyclopentyl, and —CH₂-cyclohexyl.

In another embodiment, R_{101f} is —CH₂—CO₂H.

In another embodiment, R_{101f} is selected from the group consisting of —CHMe-CH₂—OMe.

In another embodiment, R_{101f} is selected from the group consisting of —CH₂—CH═C(Me)₂.

In another embodiment, R_{101f} is selected from the group consisting of —CH₂-oxiranyl, —CH₂-oxetanyl, —CH₂-tetrahydrofuranyl, —CH₂-aziridinyl, —CH₂-azetidinyl, —CH₂-pyrrolidinyl, and —CH₂-piperidinyl.

In another embodiment, R_{101f} is selected from the group consisting of —CH₂-phenyl, —CH₂-pyridyl, —CH₂-pyrazinyl, —CH₂-pyrazolyl, —CH₂-imidazolyl, and —CH₂-oxazolyl.

In another embodiment, R_{101f} is selected from the group consisting of —C(═O)—R_{101f′}, wherein R_{101f′} is selected from the group consisting of —CH₂-heterocycloalkyl, —CH₂—NRₓR_{x′}, and —C(Me)₂-NRₓR_{x′}.

In another embodiment, R_{101f} is selected from the group consisting of —C(═O)—R_{101f′}, wherein R_{101f′} is selected from the group consisting of —CH₂CH₃, —CH₂CH₂CH₃, —CH₂CH₂—NRₓR_{x′}, —CH₂CH₂CH₂—NRₓR_{x′}, —CH₂-heterocycloalkyl, —CHMe-NRₓR_{x′}, —CH₂—NRₓR_{x′}, and —CH₂—C(Me)₂-CH₂—NRₓR_{x′}.

In another embodiment, R_{101f} is selected from the group consisting of —CH₂—C(═O)—R_{101f′}, wherein R_{101f′} is selected from the group consisting of —NRₓR_{x′} and heterocycloalkyl.

In another embodiment,

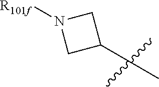

is selected from the group consisting of

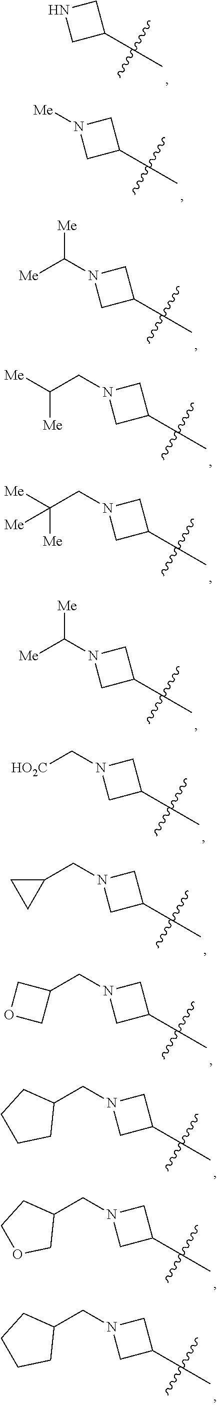

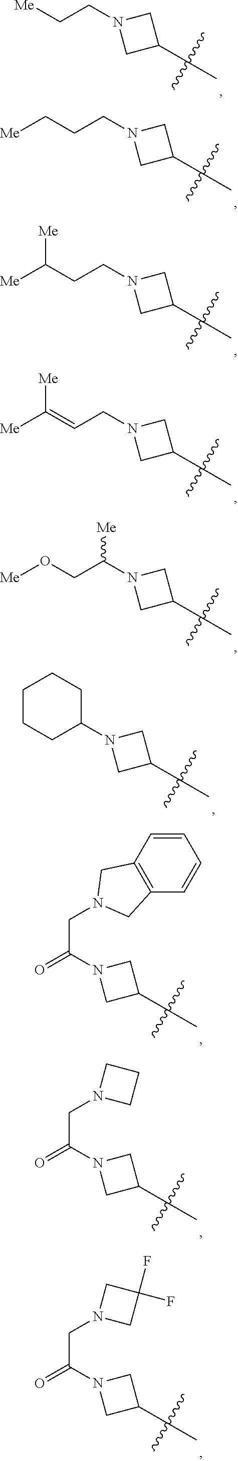

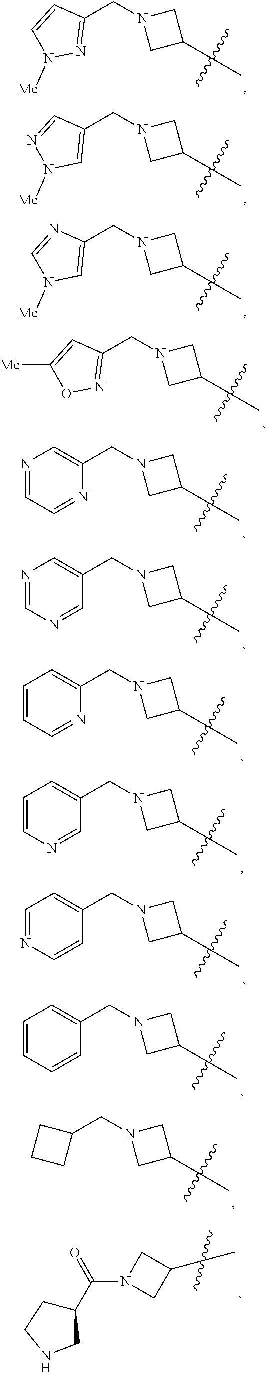

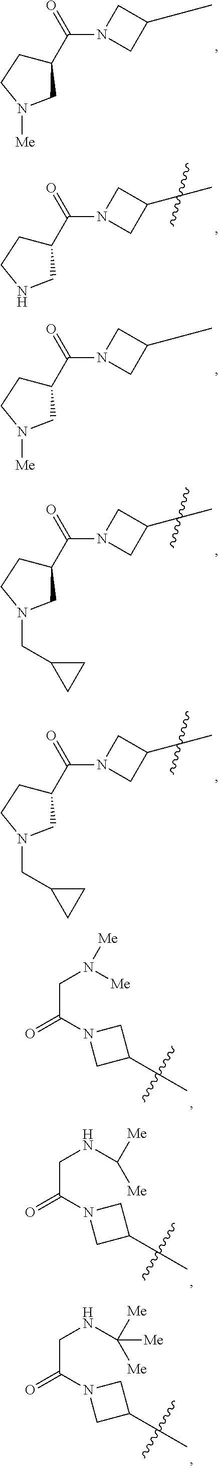

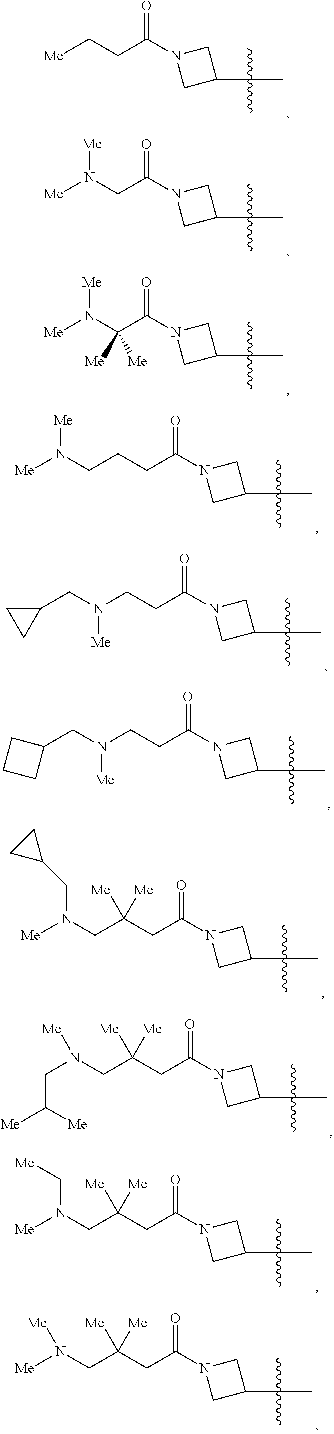

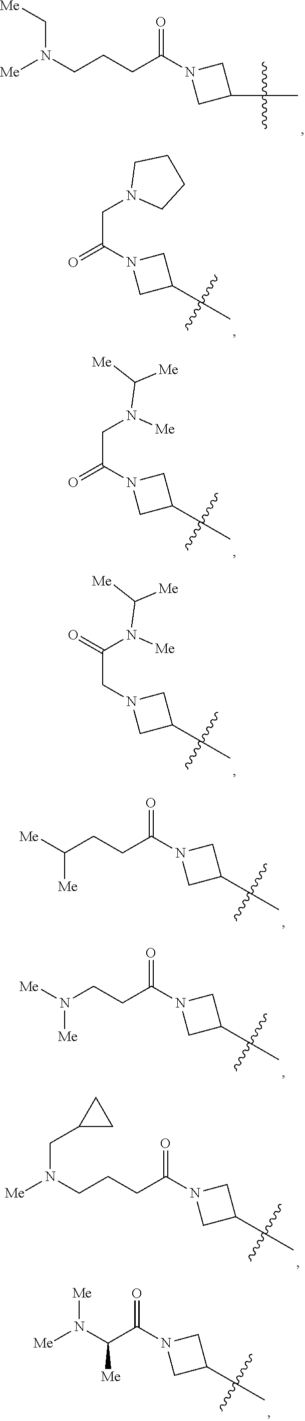

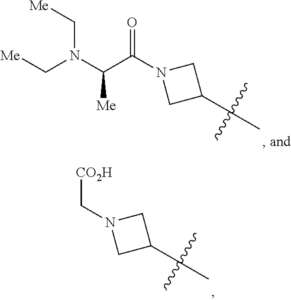 [SMILES: *C1CN(C(=O)[C@@H](C)N(CC)CC)C1 ; *C1CN(CC(=O)O)C1]

wherein “ [symbol] ” indicates a point of attachment.

In another embodiment:

R₁₀ₐ is optionally substituted saturated or partially unsaturated pyrrolidinyl. A pyrrolidine containing one double bonds is partially unsaturated and is known as dihydro pyrrole.

In another embodiment:

R₁₀ₐ is pyrrolidinyl-R_{101g}; wherein

R_{101g} is selected from the group consisting of —H, alkyl and —C(═O)-alkylene-NRₓR_{x′}; wherein:

R_{101g} is selected from the group consisting of —H, optionally substituted alkyl, —C(═O)-alkyl, and —C(═O)-alkylene-NRₓR_{x′}, wherein:

Rₓ and R_{x′} are each independently selected from the group consisting of —H, optionally substituted alkyl, —C(═O)-alkyl, —C(═O)-alkyl, and —C(═O)-alkylene-N(R_{y})₂; or

Rₓ and R_{x′} together with the atom to which they are attached form a 3-, 4-, 5-, 6-, or 7-membered ring optionally containing an additional heteroatom selected from the group consisting of O, S, SO, SO₂, NR_{y}, and N—C₁-C₁₀ alkyl; and wherein

each R_{y} is independently selected from the group consisting of —H and optionally substituted C₁₋₁₀ alkyl; or

each R_{y}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

In another embodiment: R₁₀ₐ is selected from the group consisting of

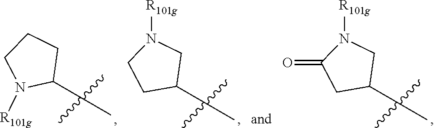

wherein “ [symbol] ” indicates a point of attachment.

In some embodiments of

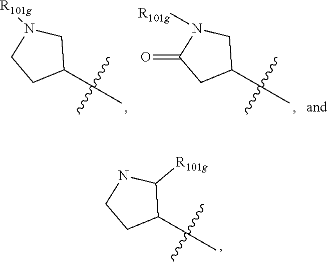

R_{101g} is H, methyl, ethyl, propyl, isopropyl, butyl, iso-butyl, tert-butyl, pentyl, isopentyl, neopentyl, hexyl, and heptyl, wherein “ [symbol] ” indicates a point of attachment.

In another embodiment: R_{101g} is selected from —C(═O)-methyl, —C(═O)—CH₂—N(Me)₂, and —C(═O)—CH₂—NHCH₂CH(Me)₂.

In some embodiments,

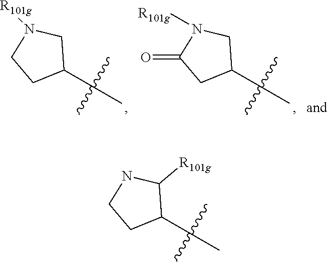

and are selected from the group consisting of

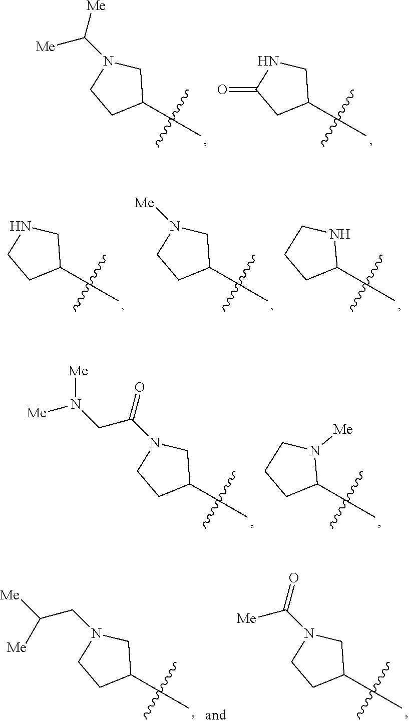

wherein “ [symbol] ” indicates a point of attachment

In another embodiment:

R₁₀ₐ is optionally substituted saturated or partially unsaturated piperindinyl. A piperidine with one double bond is partially unsaturated piperidine and is known as a tetrahydropyridine.

In another embodiment:

R₁₀ₐ is selected from the group consisting of

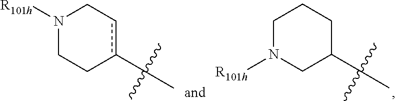

wherein “ [symbol] ” indicates a point of attachment; and R₁₀₁ₕ is selected from the group consisting of —H, optionally substituted alkyl, optionally substituted haloalkyl, optionally substituted alkoxy, optionally substituted hydroxyalkyl, optionally substituted alkenyl, optionally substituted -alkylene-cycloalkyl, optionally substituted -allylene-heterocycloalkyl, optionally substituted alkylene-aryl, optionally substituted alkylene-heteroaryl, —SO₂-optionally substituted alkyl, —C(═O)-optionally substituted alkyl, —C(═O)-optionally substituted alkylene-cycloalkyl, —C(═O)-optionally substituted alkylene-heterocycloalkyl, and —C(═O)-optionally substituted alkylene-NRₓR_{x′}; wherein

Rₓ and R_{x′} are each independently selected from the group consisting of —H, optionally substituted alkyl, —C(═O)-alkyl, —C(═O)-alkyl, and —C(═O)-alkylene-N(R_{y})₂; or

Rₓ and R_{x′} together with the atom to which they are attached form a 3-, 4-, 5-, 6-, or 7-membered ring optionally containing an additional heteroatom selected from the group consisting of O, S, SO, SO₂, NR_{y}, and N—C₁-C₁₀ alkyl; and wherein

each R_{y} is independently selected from the group consisting of —H and optionally substituted C₁₋₁₀ alkyl; or

each R_{y}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

In another embodiment: R₁₀₁ₕ is selected from the group consisting of —H, methyl, ethyl, propyl, isopropyl, butyl, isobutyl, pentyl, neopentyl, trifluoromethyl, CF₃—CH₂—, and CHF₂—CH₂—.

In another embodiment, R₁₀₁ₕ is selected from the group consisting of —CH₂— cyclopropyl, —CH₂-cyclobutyl, —CH₂-cyclopentyl, and —CH₂-cyclohexyl.

In another embodiment, R₁₀₁ₕ is selected from the group consisting of —CHMe-CH₂—OMe.

In another embodiment, R₁₀₁ₕ is selected from the group consisting of —CH₂—CH═C(Me)₂.

In another embodiment, R₁₀₁ₕ is selected from the group consisting of —CH₂-oxiranyl, —CH₂-oxetanyl, —CH₂-tetrahydrofuryl, aziridinyl, azetidinyl pyrrolidinyl, and piperidinyl.

In another embodiment R₁₀₁ₕ is selected from the group consisting of —CH₂-phenyl, —CH₂-pyridyl, —CH₂-pyrazinyl, —CH₂-pyrazolyl, —CH₂-imidazolyl and —CH₂-oxazolyl.

In another embodiment:

R₁₀₁ₕ is selected from the group consisting of —C(═O)—R_{101h′}, wherein R_{101h′} is selected from the group consisting of —CH₂-heterocycloalkyl, —CH₂—NRₓR_{x′}, and —C(Me)₂-NRₓR_{x′}.

In another embodiment:

R₁₀₁ₕ is selected from the group consisting of —C(═O)—R_{101h′}, wherein R_{101h′} is selected from the group consisting of —CH₂CH₃, —CH₂CH₂CH₃, —CH₂CH₂—NRₓR_{x′}, —CH₂CH₂CH₂—NRₓR_{x′}, —CH₂-heterocycloalkyl, —CHMe-NRₓR_{x′}, —CH₂—NRₓR_{x′}, and —CH₂—C(Me)₂-CH₂—NRₓR_{x′}.

In another embodiment: R₁₀₁ₕ is selected from the group consisting of —CH₂—C(═O)—R_{101h′}, wherein R_{101h′} is selected from the group consisting of —NRₓR_{x′} and heterocycloalkyl.

In another embodiment: R₁₀₁ₕ is selected from the group consisting of —SO₂-Me, —CH₂—CHOH—CH₂OH, —CH₂—CHNH₂—CH₂OH, and —CHMe-CH₂—OMe.

In another embodiment:

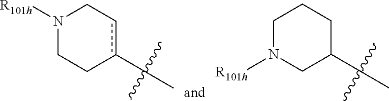

are selected from the group consisting of

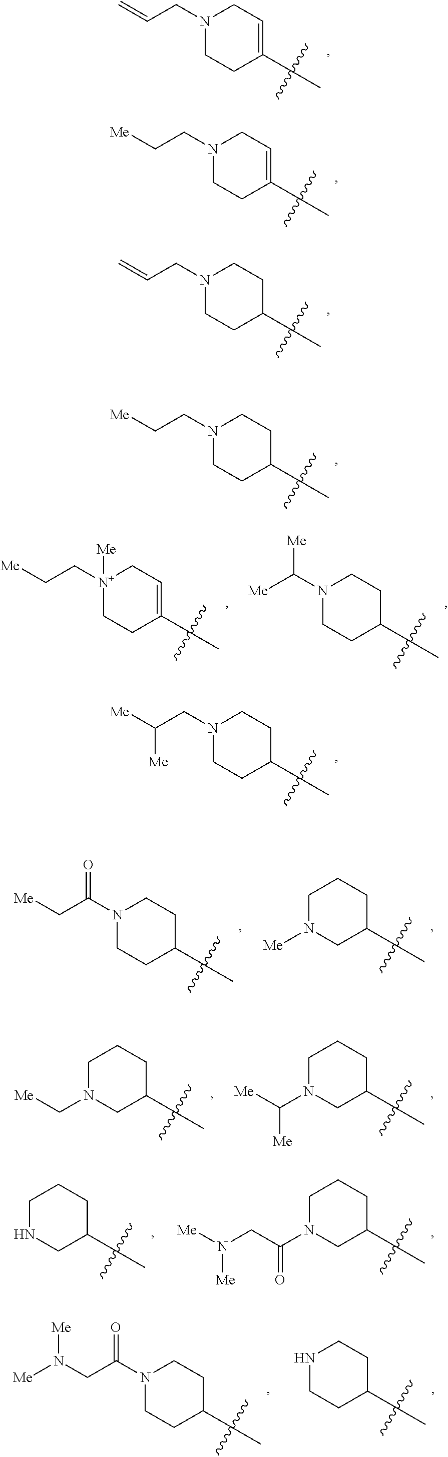

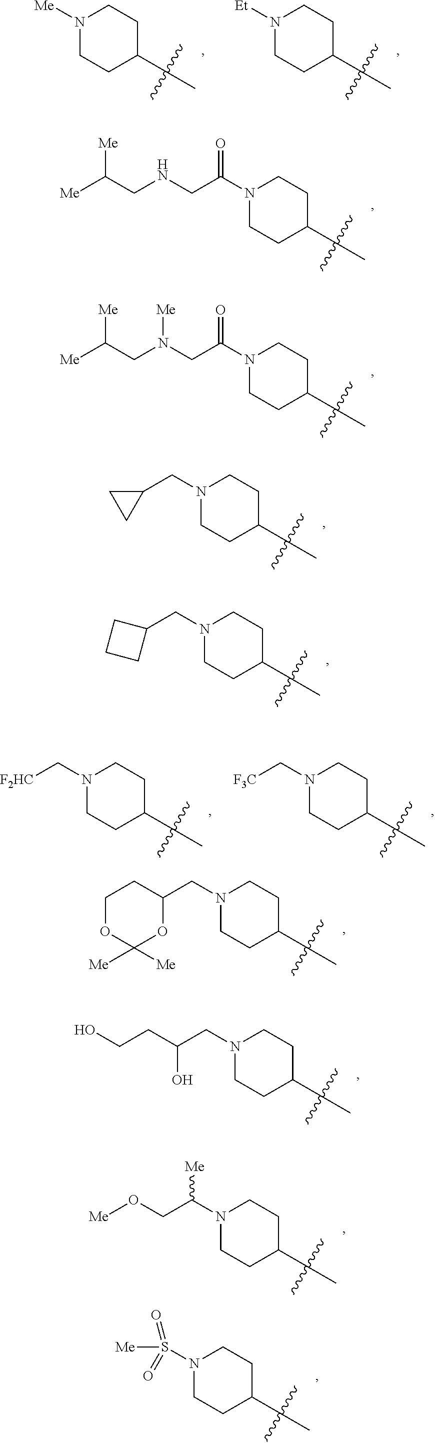

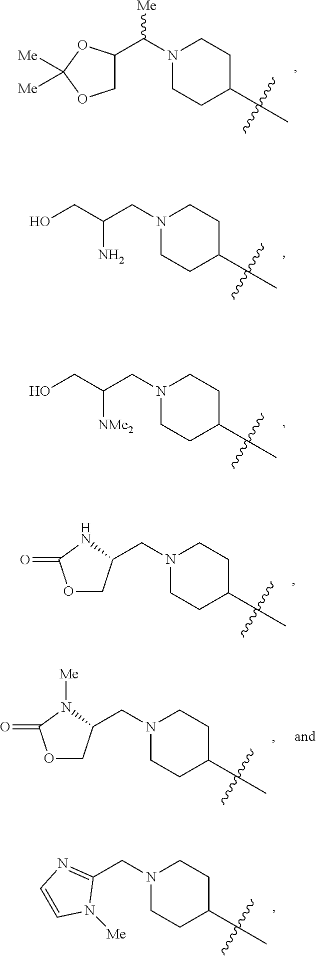 [SMILES: *C1CCN(C(C)C2COC(C)(C)O2)CC1 ; *C1CCN(CC(N)CO)CC1 ; *C1CCN(CC(CO)N(C)C)CC1 ; *C1CCN(C[C@@H]2COC(=O)N2)CC1 ; *C1CCN(C[C@@H]2COC(=O)N2C)CC1 ; *C1CCN(Cc2nccn2C)CC1]

wherein “ [symbol] ” indicates a point of attachment.

In another embodiment:

R₁₀ₐ is

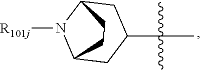

wherein “ [symbol] ” indicates a point of attachment; and

R₁₀₁ⱼ is selected from the group consisting of —H, optionally substituted alkyl, haloalkyl, alkoxy, hydroxyalkyl, optionally substituted alkenyl, -alkylene-optionally substituted cycloalkyl, -alkylene-optionally substituted heterocycloalkyl, alkylene-optionally substituted aryl, alkylene-optionally substituted heteroaryl, —SO₂-alkyl, —C(═O)-alkyl, —C(═O)-alkylene-optionally substituted cycloalkyl, —C(═O)-alkylene-heterocycloalkyl, and —C(═O)-alkylene-NRₓR_{x′}; wherein

Rₓ and R_{x′} are each independently selected from the group consisting of —H, optionally substituted alkyl, —C(═O)-alkyl, —C(═O)-alkyl, and —C(═O)-alkylene-N(R_{y})₂; or

Rₓ and R_{x′} together with the atom to which they are attached form a 3-, 4-, 5-, 6-, or 7-membered ring optionally containing an additional heteroatom selected from the group consisting of O, S, SO, SO₂, NR_{y}, and N—C₁-C₁₀ alkyl; and wherein

each R_{y} is independently selected from the group consisting of —H and optionally substituted C₁₋₁₀ alkyl; or

each R_{y}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

In another embodiment:

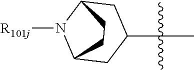

is selected from the group consisting of

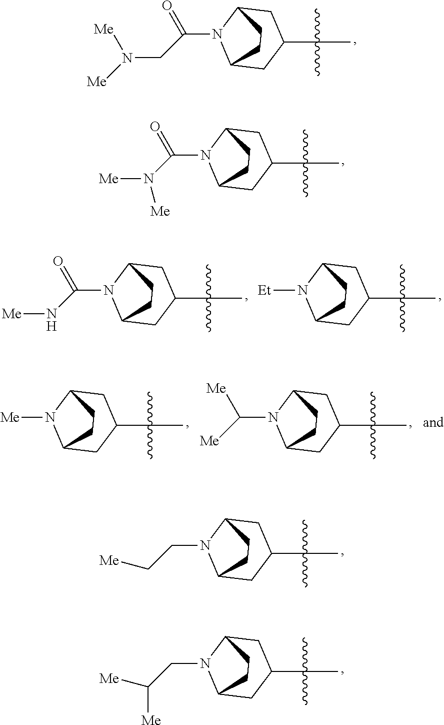

wherein “ [symbol] ” indicates a point of attachment.

Another embodiment of a compound of formula I and II is a compound of formula III:

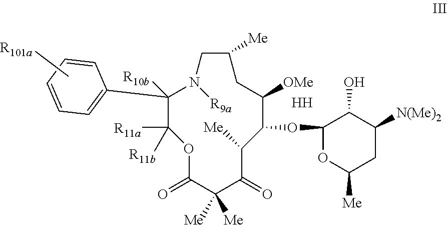

or a pharmaceutically acceptable salt thereof, wherein R₁₀₁ₐ is selected from the group consisting of —H, halo, optionally substituted aryl, and optionally substituted heteroaryl, wherein R₁₀₁ₐ is selected from the group consisting of —H, halo, —B(OH)₂, —B(O-alkyl)₂, optionally substituted phenyl, and optionally substituted heteroaryl.

In some embodiments of formula III, R₉ₐ is —H, methyl, ethyl, propyl, isopropyl, butyl, or isobutyl. In some embodiments of formula III, R₉ₐ is —H, or methyl R_{10b} is H or methyl. In some embodiments, R₁₁ₐ and R_{11b} are each independently H or methyl. In some embodiments, R_{10b} is H and R₁₁ₐ and R_{11b} are each independently H.

Another embodiment of a compound of formula I and II is a compound of formula IV:

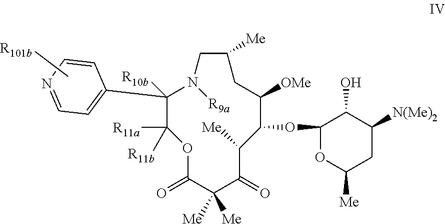

or a pharmaceutically acceptable salt thereof, wherein R_{10b} is selected from the group consisting of H, halo, optionally substituted aryl, and optionally substituted heteroaryl.

In some embodiments of formula IV, R₉ₐ is —H, methyl, ethyl, propyl, isopropyl, butyl, or isobutyl. In some embodiments of formula IV, R₉ₐ is —H, or methyl R_{10b} is H or methyl. In some embodiments, R₁₁ₐ and R_{11b} are each independently H or methyl. In some embodiments, R_{10b} is H and R₁₁ₐ and R_{11b} are each independently H.

Another embodiment of a compound of formula I and II is a compound of formula V:

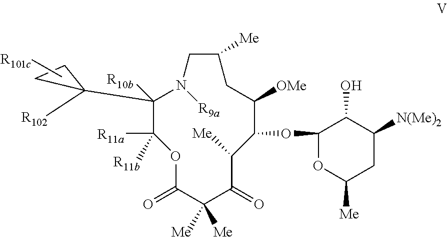

or a pharmaceutically acceptable salt thereof, wherein

R_{101c} is selected from the group consisting of —H, halo, —OH, alkoxy, —NRₓR_{x′}, and alkylene-R_{101d}, wherein R₁₀Id is selected from the group consisting of —H, halo, —OH, alkoxy, and —NRₓR_{x′}, wherein:

Rₓ and R_{x′} are each independently selected from the group consisting of —H, optionally substituted alkyl, —C(═O)-alkyl, and —C(═O)-alkylene-N(R_{y})₂; or

Rₓ and R_{x′} together with the atom to which they are attached form a 3-, 4-, 5-, 6-, or 7-membered ring optionally containing an additional heteroatom selected from the group consisting of O, S, SO, SO₂, NR_{y}, and N—C₁-C₁₀ alkyl; and wherein

each R_{y} is independently selected from the group consisting of —H and optionally substituted C₁₋₁₀ alkyl; or

each R_{y}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl; and R₁₀₂ is H or alkyl.

In some embodiments of formula V, R₉ₐ is —H, methyl, ethyl, propyl, isopropyl, butyl, or isobutyl. In some embodiments of formula V, R₉₀ is —H, or methyl R_{10b} is H or methyl. In some embodiments, R₁₁ₐ and R_{11b} are each independently H or methyl. In some embodiments, R_{10b} is H and R₁₁ₐ and R_{11b} are each independently H.

Another embodiment of a compound of formula I and II is a compound of formula VI:

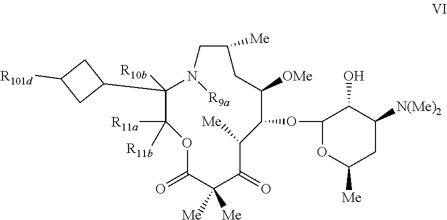

or a pharmaceutically acceptable salt thereof, wherein R_{101d} is selected from the group consisting of —H, halo, —OH, alkoxy, and —NRₓR_{x′}, wherein:

Rₓ and R_{x′} are each independently selected from the group consisting of —H, optionally substituted alkyl, —C(═O)-alkyl, and —C(═O)-alkylene-N(R_{y})₂; or

Rₓ and R_{x′} together with the atom to which they are attached form a 3-, 4-, 5-, 6-, or 7-membered ring optionally containing an additional heteroatom selected from the group consisting of O, S, SO, SO₂, NR_{y}, and N—C₁-C₁₀ alkyl; and wherein

each R_{y} is independently selected from the group consisting of —H and optionally substituted C₁₋₁₀ alkyl; or

each R_{y}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

In some embodiments of formula VI, R₉ₐ is —H, methyl, ethyl, propyl, isopropyl, butyl, or isobutyl. In some embodiments of formula VI, R₉ₐ is —H, or methyl R_{10b} is H or methyl. In some embodiments, R₁₁ₐ and R_{11b} are each independently H or methyl. In some embodiments, R_{10b} is H and R₁₁ₐ and R_{11b} are each independently H.

Another embodiment of a compound of formula I and II is a compound of formula VII:

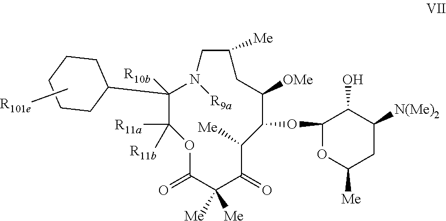

or a pharmaceutically acceptable salt thereof, wherein:

R₁₀₁ₑ is selected from the group consisting of —H, halo, —OH, alkoxy, —NRₓR_{x′}, and alkylene-R_{101e′}, wherein R_{101e′}, is selected from the group consisting of H, halo, —OH, alkoxy, and NRₓR_{x′}, wherein:

Rₓ and R_{x′} are each independently selected from the group consisting of H, optionally substituted alkyl, —C(═O)-alkyl, and —C(═O)-alkylene-N(R_{y})₂; or

Rₓ and R_{x′} together with the atom to which they are attached form a 3-, 4-, 5-, 6-, or 7-membered ring optionally containing an additional heteroatom selected from the group consisting of O, S, SO, SO₂, NR_{y}, and N—C₁-C₁₀ alkyl; and wherein

each R_{y} is independently selected from the group consisting of —H and optionally substituted C₁₋₁₀ alkyl; or

each R_{y}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

In some embodiments of formula VII, R₉ₐ is —H, methyl, ethyl, propyl, isopropyl, butyl, or isobutyl. In some embodiments of formula VII, R₉ₐ is —H, or methyl R_{10b} is H or methyl. In some embodiments, R₁₁ₐ and R_{11b} are each independently H or methyl. In some embodiments, R_{10b} is H and R₁₁ₐ and R_{11b} are each independently H.

Another embodiment of a compound of formula I and II is a compound of formula VIII:

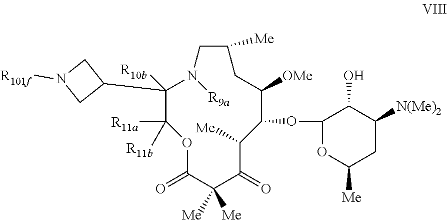

or a pharmaceutically acceptable salt thereof, wherein:

R_{101f} is selected from the group consisting of —H, halo, optionally substituted alkyl, —OH, —CO₂H, —CO₂-alkyl, alkoxy, optionally substituted cycloalkyl, optionally substituted heterocycloalkyl, NRₓR_{x′}, —C(═O)-alkyl, —C(═O)-optionally substituted heterocycloalkyl, alkenyl, —C(═O)-optionally substituted alkylene-R_{101f′}, -alkylene-C(═O)—R_{101f′} and -alkylene-R_{101f′}, wherein R_{101f′} is selected from the group consisting of —H, halo, —OH, alkoxy, —CO₂H, CO₂-optionally substituted alkyl, optionally substituted cycloalkyl, optionally substituted hetercycloalkyl, optionally substituted aryl, and optionally substituted heteroaryl, and —NRₓR_{x′}, wherein:

Rₓ and R_{x′} are each independently selected from the group consisting of —H, optionally substituted alkyl, —C(═O)-alkyl, and —C(═O)-alkylene-N(R_{y})₂; or

Rₓ and R_{x′} together with the atom to which they are attached form a 3-, 4-, 5-, 6-, 7-, optionally containing an additional heteroatom selected from the group consisting of O, S, SO, SO₂, NR_{y}, and N—C₁-C₁₀ alkyl; and wherein

each R_{y} is independently selected from the group consisting of —H and optionally substituted C₁₋₁₀ alkyl; or

each R_{y}, together with the atom to which they are attached form a 3-, 4-, 5-, 6-, 7, 8-, 9-, or 10-membered monocyclic or bicyclic ring optionally containing an additional heteroatom selected from the group consisting of O, S, SO, SO₂, NH, and N—C₁-C₁₀ alkyl.

In some embodiments of formula VIII, R₉ₐ is —H, methyl, ethyl, propyl, isopropyl, butyl, or isobutyl. In some embodiments VIII, R₉ₐ is —H, or methyl R_{10b} is H or methyl. In some embodiments, R₁₁ₐ and R_{11b} are each independently H or methyl. In some embodiments, R_{10b} is H and R₁₁ₐ and R_{11b} are each independently H.

In some embodiments of formula VIII,

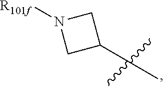

R_{101f} f is -alkylene-R_{101f′}, wherein R_{101f′} is H. In some embodiments, -alkylene-R_{101f′} is selected from the group consisting of methyl, ethyl, propyl, isopropyl, butyl, sec-butyl, tert-butyl, pentyl, isopentyl, neopentyl, hexyl, and heptyl.

In some embodiments of

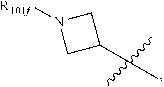

R_{101f} is -alkylene-R_{101f′}, wherein R_{101f′} is selected from the group consisting of cycloalkyl, hetercycloalkyl, aryl, and heteroaryl, wherein “ [symbol] ” indicates a point of attachment. In some embodiments, is

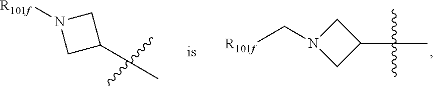

wherein R_{101f′} is selected from the group consisting of pyraozlyl, cyclobutyl, cyclopropyl, pyrazinyl, cyclohexyl, oxetanyl, phenyl, cyclopentyl, pyridinyl, tetrahydrofuranyl, isoxazolyl, imidazolyl, and pyrimidinyl, wherein “ [symbol] ” indicates a point of attachment.

In some embodiments of

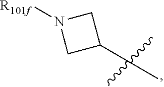

R_{101f} is -alkylene-R_{101f′}, wherein R_{101f′} is selected from alkoxy, —CO₂H, and CO₂-alkyl, wherein “ [symbol] ” indicates a point of attachment. In some embodiments,

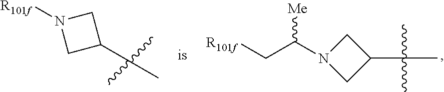

wherein R_{101f′} is methoxy “ [symbol] ” indicates a point of attachment. In some embodiments,

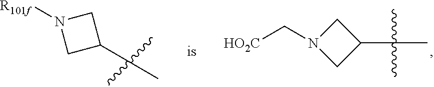

wherein “ [symbol] ” indicates a point of attachment.

In some embodiments of

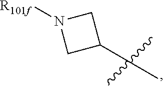

R_{101f} is alkenyl. In some embodiments, R_{101f} is

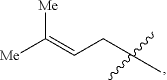 [SMILES: *CC=C(C)C]

wherein “ [symbol] ” indicates a point of attachment.

In some embodiments of

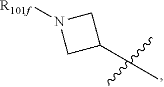

R_{101f} is —C(═O)-heterocycloalkyl, “ [symbol] ” indicates a point of attachment. In some embodiments,

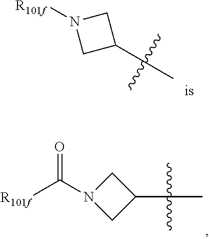

wherein R_{101f′} is optionally substituted pyrrolidinyl, wherein “ [symbol] ” indicates a point of attachment.

In some embodiments of

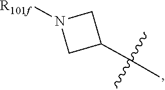

R_{101f} is C(═O)-alkylene-R_{101f′}, wherein R_{101f′} is selected from the group consisting of H, aryl, heteroaryl, cycloalkyl, heterocycloalkyl, and —NRₓR_{x′}, wherein “ [symbol] ” indicates a point of attachment. In some embodiments, R_{101f} is selected from the group consisting of —C(═O)—CH₂—R_{101f′}, —C(═O)—CH₂CH₂—R_{101f′}, —C(═O)—CH(Me)-R_{101f′}, —C(═O)—CH₂CH₂CH₂—R_{101f′}, —C(═O)—C(Me)₂-R_{101f′}, and —C(═O)—CH₂C(Me)₂CH₂—R_{101f′}. In some embodiments, R_{101f} is selected from the group consisting of H, —C(═O)—CH₂-heteroaryl, —C(═O)—CH₂-heterocyclo, and C(═O)—CH₂—NRₓR_{x′}. In some embodiments, R_{101f′} is selected from the group consisting of H, isoindolinyl, optionally substituted azetidinyl, and optionally substituted pyrrolidinyl. In some embodiments, R_{101f′} is NRₓR_{x′}, wherein one of Rₓ and R_{x′} is H, methyl, or ethyl, and the other of Rₓ and R_{x′}, is H, methyl, ethyl, isopropyl,

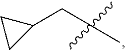 [SMILES: *CC1CC1]

butyl, isobutyl, tert-butyl, or

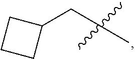 [SMILES: *CC1CCC1]

wherein “ [symbol] ” indicates a point of attachment.

In some embodiments of

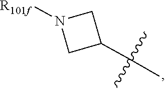

R_{101f} is —CH₂—C(═O)—NRₓR_{x′}. In some embodiments, one of Rₓ and R_{x′} is H or methyl and the other of Rₓ and R_{x′} is benzyl, isopropyl, or Rₓ and R_{x′} are joined together with the nitrogen to which they are attached to form a ring. In some embodiments, Rₓ and R_{x′} are joined together with the nitrogen to which they are attached to form a pyrrolidine or piperidine ring.

In some embodiments of

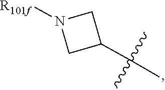

R_{101f} is —CH₂—C(═O)-alkylene-R_{101f′}, wherein R_{101f′} is selected from the group consisting of H, aryl, heteroaryl, cycloalkyl, heterocycloalkyl, and —NRₓR_{x′}, wherein “ [symbol] ” indicates a point of attachment. In some embodiments, R_{101f′} is selected from the group consisting of —CH₂—C(═O)—CH₂—R_{101f′}, —CH₂—C(═O)—CH₂CH₂—R_{101f′}, —CH₂—C(═O)—CH(Me)-R_{101f′}, —CH₂—C(═O)—CH₂CH₂CH₂—R_{101f′}, —CH₂—C(═O)—C(Me)₂-R_{101f′}, and —CH₂—C(═O)—CH₂C(Me)₂CH₂—R_{101f′}. In some embodiments, R_{101f′} is selected from the group consisting of H, —CH₂—C(═O)—CH₂-heteroaryl, —CH₂—C(═O)—CH₂-heterocyclo, and —CH₂—C(═O)—CH₂—NRₓR_{x′}. In some embodiments, R_{101f′} is selected from the group consisting of H, isoindolinyl, optionally substituted azetidinyl, and optionally substituted pyrrolidinyl. In some embodiments, R_{101f′} is NRₓR_{x′}, wherein one of Rₓ and R_{x′} is H, methyl, or ethyl, and the other of Rₓ and R_{x′} is H, methyl, ethyl, isopropyl,

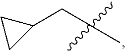 [SMILES: *CC1CC1]

butyl, isobutyl, tert-butyl,

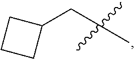 [SMILES: *CC1CCC1]

wherein “ [symbol] ” indicates a point of attachment.

In some embodiments, R_{10b} is H or Me and

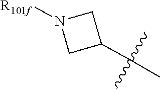

is selected from the group

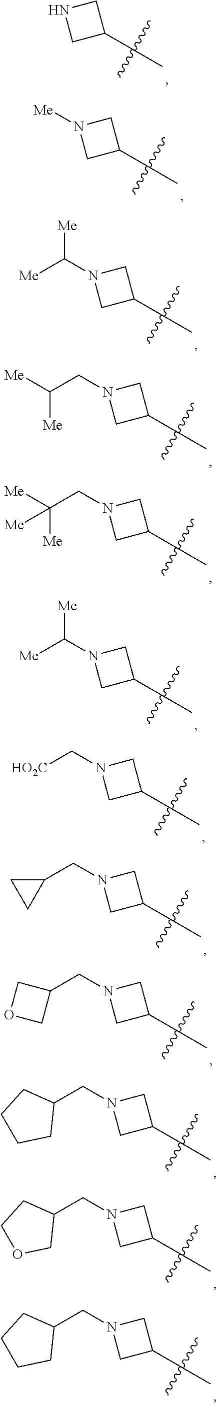

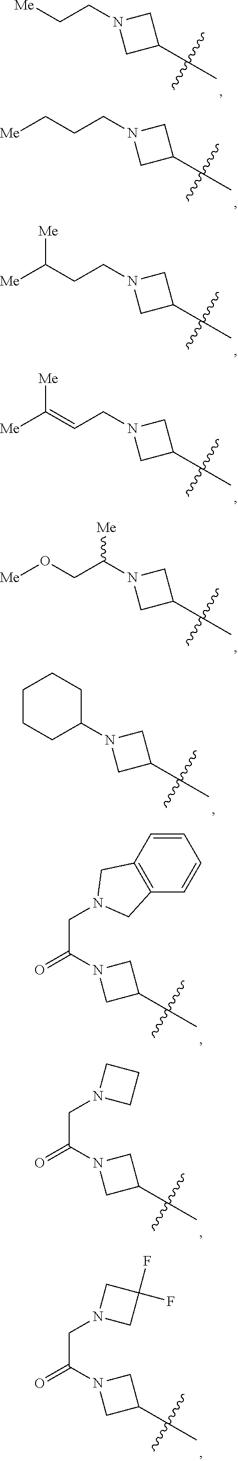

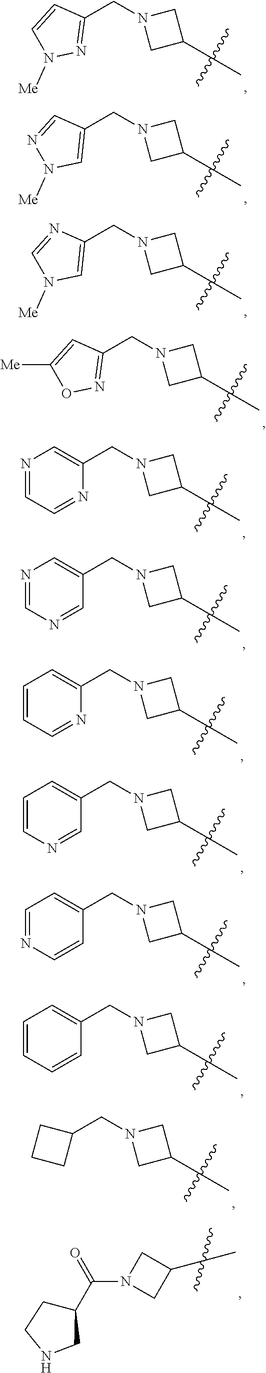

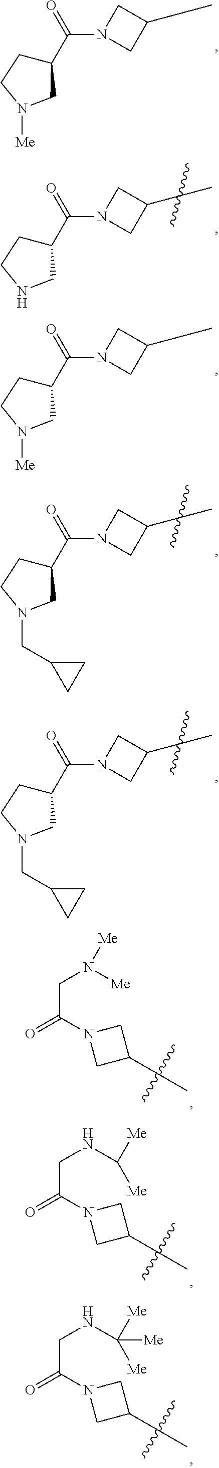

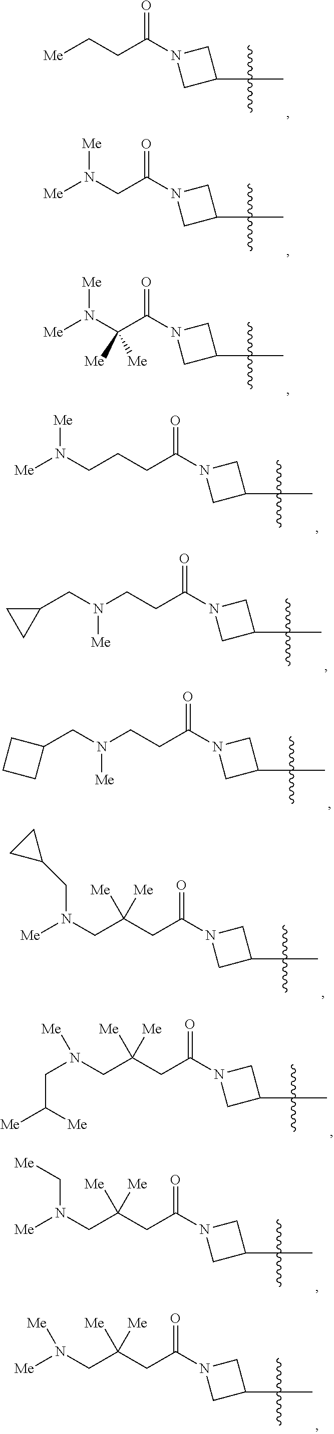

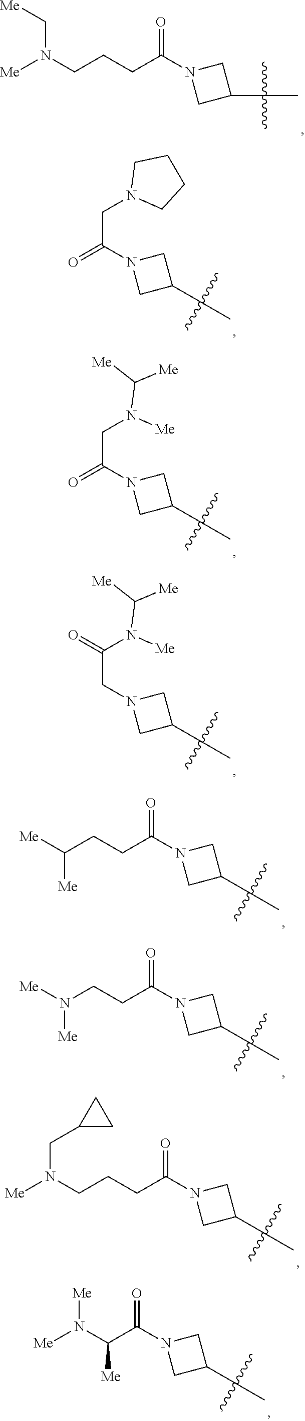

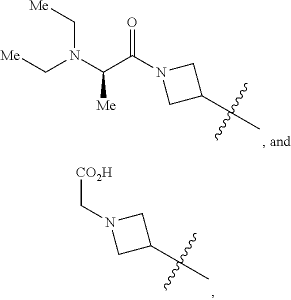 [SMILES: *C1CN(C(=O)[C@@H](C)N(CC)CC)C1 ; *C1CN(CC(=O)O)C1]

wherein “ [symbol] ” indicates a point of attachment.

Another embodiment of a compound of formula I and II is a compound of formula IXa, IXb, or IXc:

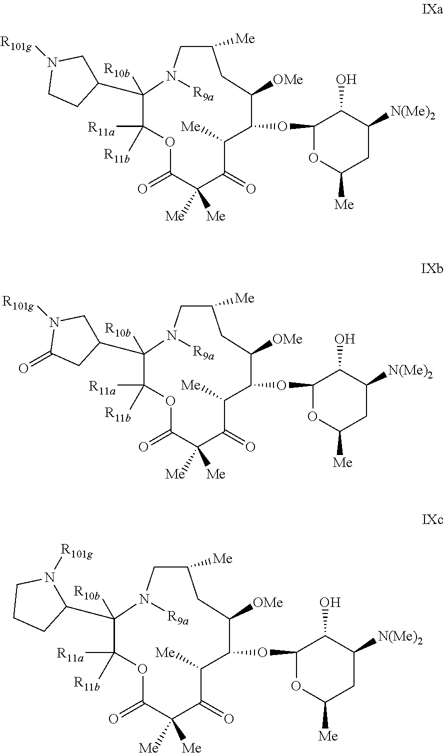

or a pharmaceutically acceptable salt thereof, wherein:

R_{101g} is selected from the group consisting of —H, optionally substituted alkyl, —C(═O)-alkyl, and —C(═O)-alkylene-NRₓR_{x′}, wherein:

Rₓ and R_{x′} are each independently selected from the group consisting of —H, optionally substituted alkyl, —C(═O)-alkyl, —C(═O)-alkyl, and —C(═O)-alkylene-N(R_{y})₂; or

Rₓ and R_{x′} together with the atom to which they are attached form a 3-, 4-, 5-, 6-, or 7-membered ring optionally containing an additional heteroatom selected from the group consisting of O, S, SO, SO₂, NR_{y}, and N—C₁-C₁₀ alkyl; and wherein

each R_{y} is independently selected from the group consisting of —H and optionally substituted C₁₋₁₀ alkyl; or

each R_{y}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

In some embodiments of formula IXA, IXB, and IXC, R₉ₐ is —H, methyl, ethyl, propyl, isopropyl, butyl, or isobutyl. In some embodiments of formula XI, IXB, and IXC, R₉ₐ is —H, or methyl R_{10b} is H or methyl. In some embodiments, R₁₁ₐ and R_{11b} are each independently H or methyl. In some embodiments, R_{10b} is H and R₁₁ₐ and R_{11b} are each independently H.

In some embodiments of formula IXa, IXb, and IXc,

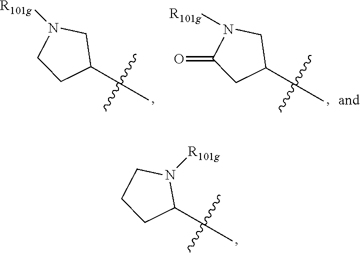

R_{101g} is H, methyl, ethyl, propyl, isopropyl, but V sec-butyl, tert-butyl, pentyl, isopentyl, neopentyl, hexyl, and heptyl, wherein “ [symbol] ” indicates a point of attachment.

In some embodiments of

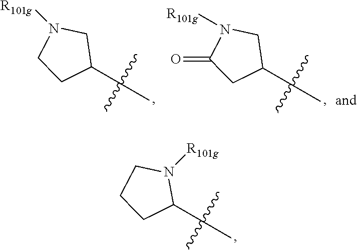

R_{101g} is (C═O)-alkyl, wherein “ [symbol] ” indicates a point of attachment. In some embodiments, R_{101g} is —(C═O)-methyl, —(CO)-ethyl, or —(C═O)-propyl.

In some embodiments of

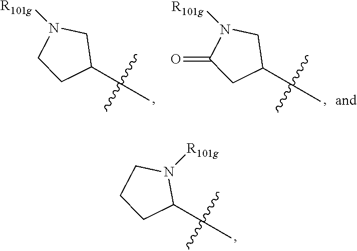

R_{101g} is —(C═O)-alkylene-NRₓR_{x′}, wherein “ [symbol] ” indicates a point of attachment. In some embodiments, R_{101g} is —(C═O)—CH₂—NRₓR_{x′}, wherein one of Rₓ and R_{x′} is H or methyl and the other of Rₓ and R_{x′} is and the other of Rₓ and R_{x′} is H, methyl, ethyl, isopropyl,

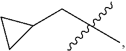 [SMILES: *CC1CC1]

butyl, isobutyl, tert-butyl,

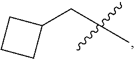 [SMILES: *CC1CCC1]

wherein “ [symbol] ” indicates a point of attachment.

In some embodiments, R_{10b} is H and

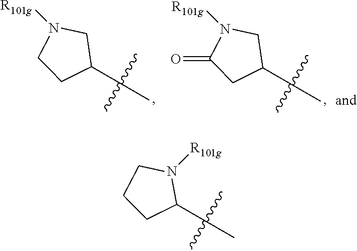

are selected from the group consisting of

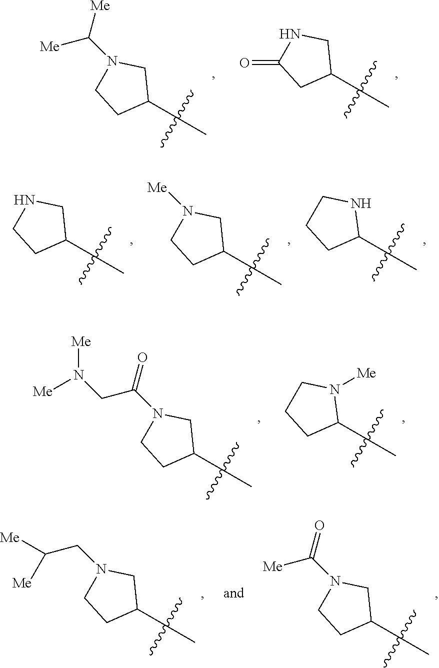

wherein “ [symbol] ” indicates a point of attachment.

Another embodiment of a compound of formula I and II is a compound of formula Xa or Xb:

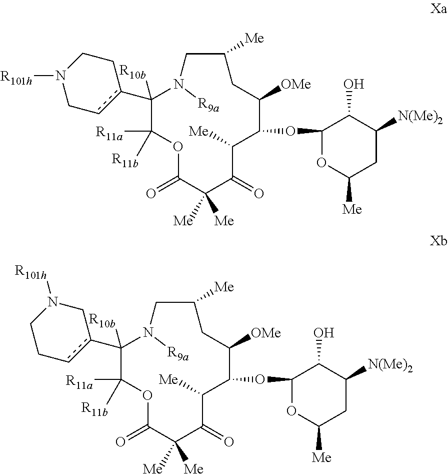

or a pharmaceutically acceptable salt thereof, wherein:

R₁₀₁ₕ is selected from the group consisting of —H, optionally substituted alkyl, optionally substituted haloalkyl, optionally substituted alkoxy, optionally substituted hydroxyalkyl, optionally substituted alkenyl, optionally substituted -alkylene-cycloalkyl, optionally substituted -allylene-heterocycloalkyl, optionally substituted alkylene-aryl, optionally substituted alkylene-heteroaryl, —SO₂-optionally substituted alkyl, —C(═O)-optionally substituted alkyl, —C(═O)-optionally substituted alkylene-cycloalkyl, —C(═O)-optionally substituted alkylene-heterocycloalkyl, and —C(═O)-optionally substituted alkylene-NRₓR_{x′}; wherein

Rₓ and R_{x′} are each independently selected from the group consisting of —H, optionally substituted alkyl, —C(═O)-alkyl, —C(═O)-alkyl, and —C(═O)-alkylene-N(R_{y})₂; or

Rₓ and R_{x′} together with the atom to which they are attached form a 3-, 4-, 5-, 6-, or 7-membered ring optionally containing an additional heteroatom selected from the group consisting of O, S, SO, SO₂, NR_{y}, and N—C₁-C₁₀ alkyl; and wherein

each R_{y} is independently selected from the group consisting of —H and optionally substituted C₁₋₁₀ alkyl; or

each R_{y}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

In some embodiments of formula XA or XB, R₉ₐ is —H, methyl, ethyl, propyl, isopropyl, butyl, or isobutyl. In some embodiments of formula XA or XB, R₉ₐ is —H, or methyl R_{10b} is H or methyl. In some embodiments, R₁₁ₐ and R_{11b} are each independently H or methyl. In some embodiments, R_{10b} is H; and R₁₁ₐ and R_{11b} are each independently H.

In some embodiments, R_{10b} is H or methyl.

In some embodiments of

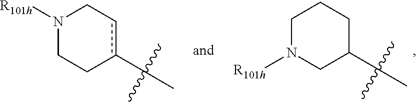

R₁₀₁ₕ is H, methyl, ethyl, propyl, isopropyl, butyl, sec-butyl, tert-butyl, pentyl, isopentyl, neopentyl, hexyl, and heptyl, wherein “ [symbol] ” indicates a point of attachment.

In some embodiments of

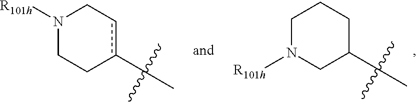

R₁₀₁ₕ is optionally substituted alkyl selected from the group consisting of —CH₂—CHOH—CH₂OH, —CH₂—CHNH₂—CH₂OH, and —CH₂—CHN(Me)₂-CH₂OH,

In some embodiments the nitrogen atom of

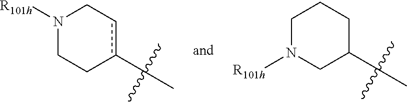

can be quarternized with R_{q} to form a quartenary ammonium ion, wherein R_{q} is methyl, ethyl, CF₂H—CH₂—, propyl, isopropyl, butyl, sec-butyl, tert-butyl, pentyl, isopentyl, neopentyl, hexyl, and heptyl, wherein “ [symbol] ” indicates a point of attachment.

In some embodiments of

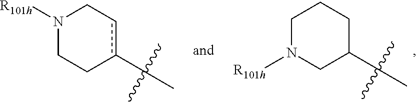

R₁₀₁ₕ is -alkylene-cycloalkyl, -alkylene-heterocycloalkyl, -alkylene-aryl, or -alkylene-heteroaryl wherein indicates a point of attachment. In some embodiments, R₁₀₁ₕ is —CH₂-imidazolyl

In some embodiments of

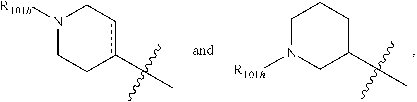

R₁₀₁ₕ is -alkylene-alkoxy, wherein “ [symbol] ” indicates a point of attachment. In some embodiments, R₁₀₁ₕ is —CHMe-CH₂—OMe.

In some embodiments of

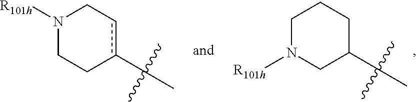

R₁₀₁ₕ is alkenyl, wherein “ [symbol] ” indicates a point of attachment. In some embodiments, R₁₀₁ₕ is propenyl.

In some embodiments of

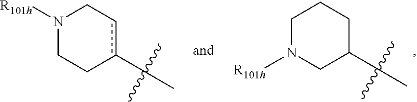

R₁₀₁ₕ is —SO₂-alkyl, wherein “ [symbol] ” indicates a point of attachment. In some embodiments, R₁₀₁ₕ is —SO₂-methyl.

In some embodiments of

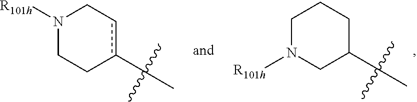

R₁₀₁ₕ is —(C═O)-alkyl, wherein “ [symbol] ” indicates a point of attachment. In some embodiments, R₁₀₁ₕ is —(C═O)-ethyl.

In some embodiments of

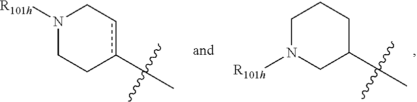

R₁₀₁ₕ is —(C═O)— alkylene-R_{101h′} wherein R_{101h′} is cycloalkyl, heterocycloalkyl, or NRₓR_{x′}, wherein “ [symbol] ” indicates a point of attachment. In some embodiments, R₁₀₁ₕ is selected from the group consisting of —C(═O)—CH₂—R_{101h′}, —C(═O)—CH₂CH₂—R_{101h′}, —C(═O)—CH(Me)-R_{101h′}, —C(═O)—CH₂CH₂CH₂—R_{101h′}, —C(═O)—C(Me)₂-R_{101h′}, and —C(═O)—CH₂C(Me)₂CH₂—R_{101h′}. In some embodiments, R₁₀₁ₕ is selected from the group consisting of —C(═O)—CH₂-heteroaryl, —C(═O)—CH₂-cycloalkyl, —C(═O)—CH₂-heterocyclo, and C(═O)—CH₂—NRₓR_{x′}, In some embodiments, R_{101h′} is selected from the group consisting of H, isoindolinyl, imidazolyl, cyclobutyl, pyrrolidinyl,

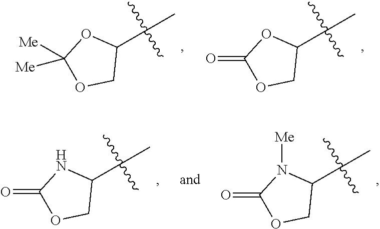 [SMILES: *C1COC(C)(C)O1 ; *C1COC(=O)O1 ; *C1COC(=O)N1 ; *C1COC(=O)N1C]

wherein “ [symbol] ” indicates a point of attachment. In some embodiments, R_{101h′} is NRₓR_{x′}, wherein one of Rₓ and R_{x′} is H, methyl, or ethyl, and the other of Rₓ and R_{x′} is H, methyl, ethyl, isopropyl,

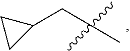 [SMILES: *CC1CC1]

butyl, isobutyl, tert-butyl, or

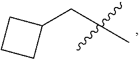 [SMILES: *CC1CCC1]

wherein “ [symbol] ” indicates a point of attachment.

In some embodiments, R_{10b} is H and

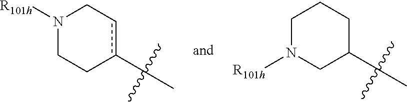

are selected from the group consisting of:

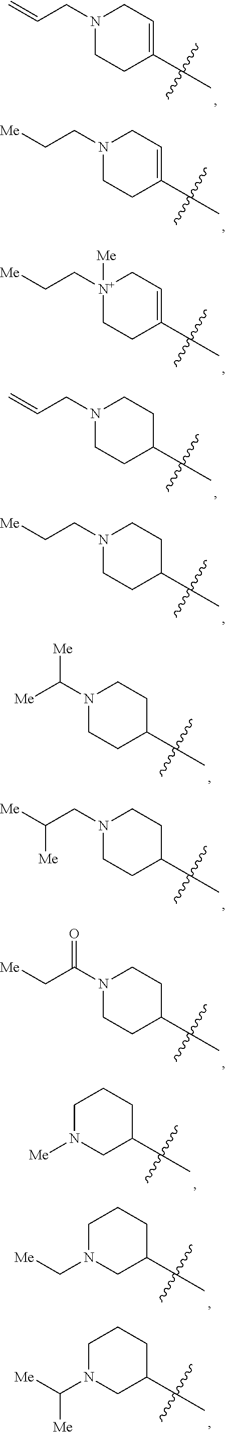

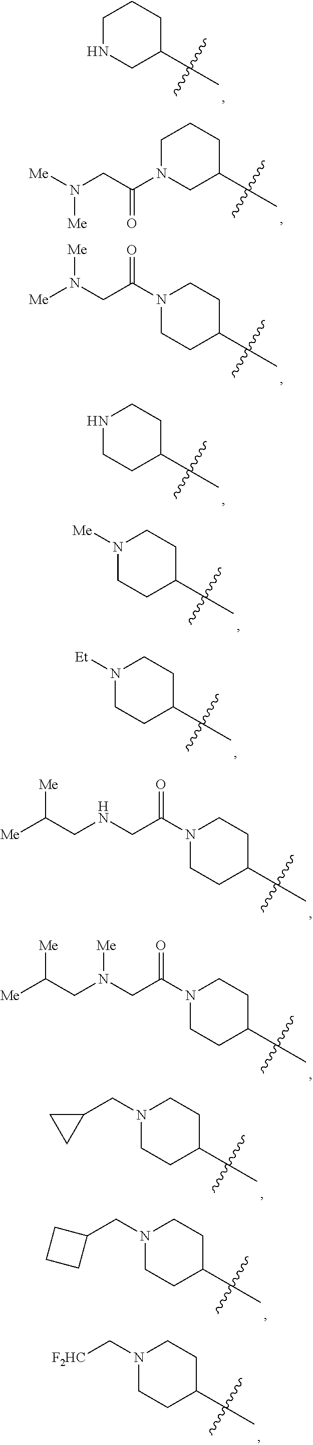

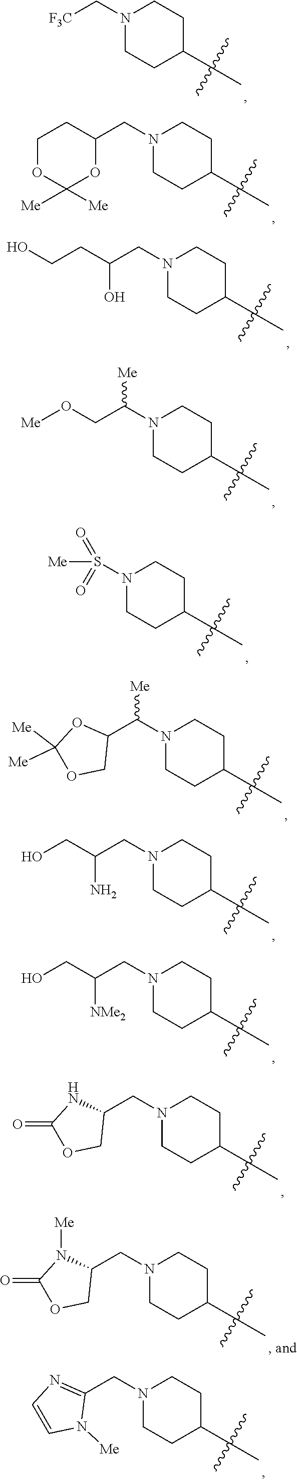

wherein “ [symbol] ” indicates a point of attachment.

Another embodiment of a compound of formula I and II is a compound of formula XI:

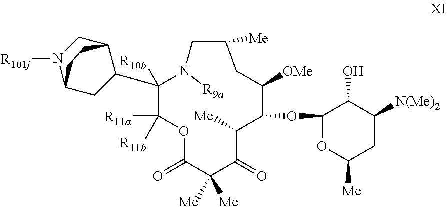

or a pharmaceutically acceptable salt thereof, wherein:

R₁₀₁ⱼ is selected from the group consisting of —H, optionally substituted alkyl, haloalkyl, alkoxy, hydroxyalkyl, optionally substituted alkenyl, -alkylene-optionally substituted cycloalkyl, -alkylene-optionally substituted heterocycloalkyl, alkylene-optionally substituted aryl, alkylene-optionally substituted heteroaryl, —SO₂-alkyl, —C(═O)-alkyl, —C(═O)-alkylene-optionally substituted cycloalkyl, —C(═O)-alkylene-heterocycloalkyl, and —C(═O)-alkylene-NRₓR_{x′}; wherein

Rₓ and R_{x′} are each independently selected from the group consisting of —H, optionally substituted alkyl, —C(═O)-alkyl, —C(═O)-alkyl, and —C(═O)-alkylene-N(R_{y})₂; or

Rₓ and R_{x′} together with the atom to which they are attached form a 3-, 4-, 5-, 6-, or 7-membered ring optionally containing an additional heteroatom selected from the group consisting of O, S, SO, SO₂, NR_{y}, and N—C₁-C₁₀ alkyl; and wherein

each R_{y} is independently selected from the group consisting of —H and optionally substituted C₁₋₁₀ alkyl; or

each R_{y}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

In some embodiments of formula XI, R_{10b} is —H, methyl, ethyl, propyl, isopropyl, butyl, or isobutyl. In some embodiments of formula XI, R₉ₐ is —H, or methyl R_{10b} is H or methyl. In some embodiments, R₁₁ₐ and R_{11b} are each independently H or methyl. In some embodiments, R_{10b} is H; and R₁₁ₐ and R_{11b} are each independently H.

In some embodiments, R_{10b} is H or methyl.

In some embodiments of

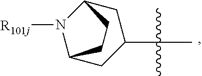

R₁₀₁ⱼ is H, methyl, ethyl, propyl, isopropyl, butyl, sec-butyl, tert-butyl, pentyl, isopentyl, neopentyl, hexyl, and heptyl, wherein “ [symbol] ” indicates a point of attachment.

In some embodiments of

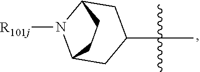

R₁₀₁ⱼ is —C(═O)-alkylene-R_{101j′}, wherein R_{101j′} is selected from cycloalkyl, heterocycloalkyl, and NRₓR_{x′}, wherein “ [symbol] ” indicates a point of attachment. In some embodiments, R₁₀₁ⱼ is —C(═O)—CH₂—R_{101j′}. In some embodiments, R₁₀₁ⱼ, is NRₓR_{x′}, wherein one of Rₓ and R_{x′} is H, methyl, or ethyl, and the other of Rₓ and R_{x′} is H, methyl, ethyl, isopropyl,

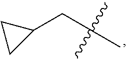 [SMILES: *CC1CC1]

butyl, isobutyl, tert-butyl, or

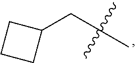 [SMILES: *CC1CCC1]

wherein “ [symbol] ” indicates a point of attachment

R_{10b} is H and

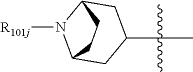

is selected from the group consisting of

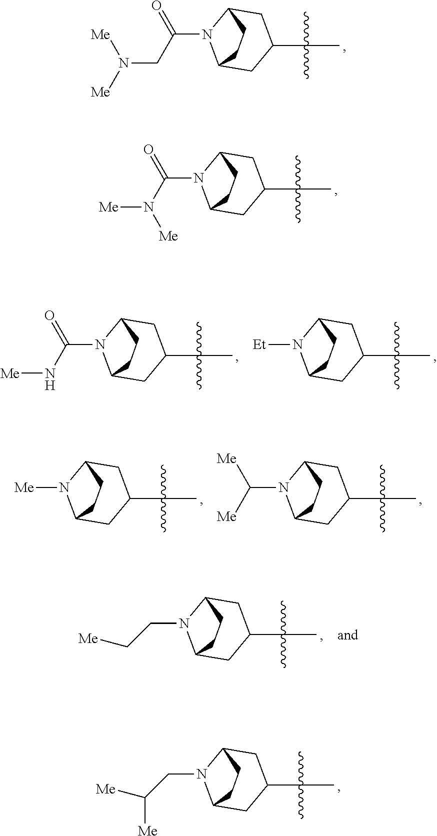

wherein “ [symbol] ” indicates a point of attachment.

Another embodiment of a compound of formula I and II is a compound depicted in w or a pharmaceutically acceptable salt thereof.

TABLE A

| Compound # | Structure |
|---|---|
| 1 | 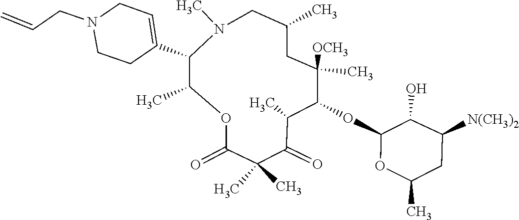 |
| 2 | 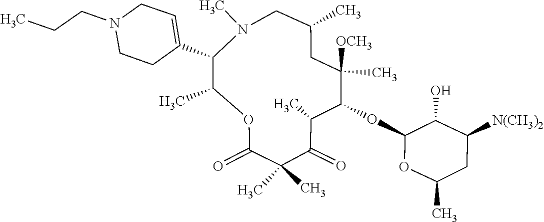 |
| 4 | 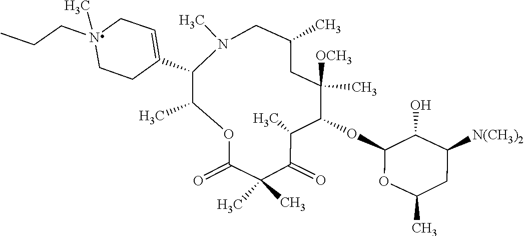 |
| 5 | 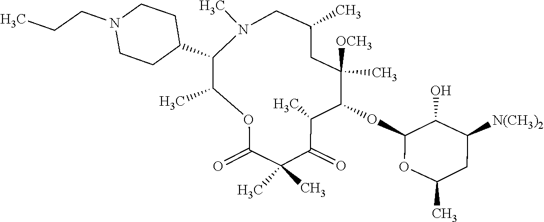 |
| 6 | 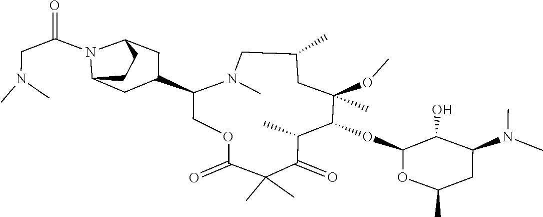 |
| 7 | 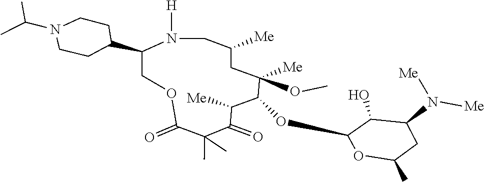 [SMILES: CO[C@]1(C)C[C@@H](C)CN[C@H](C2CCN(C(C)C)CC2)COC(=O)C(C)(C)C(=O)[C@H](C)[C@H]1O[C@@H]1O[C@H](C)C[C@H](N(C)C)[C@H]1O] |
| 8 | 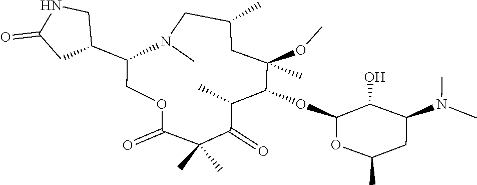 |
| 9 | 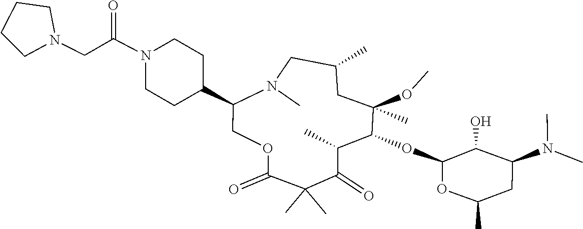 [SMILES: CO[C@]1(C)C[C@@H](C)CN(C)[C@H](C2CCN(C(=O)CN3CCCC3)CC2)COC(=O)C(C)(C)C(=O)[C@H](C)[C@H]1O[C@@H]1O[C@H](C)C[C@H](N(C)C)[C@H]1O] |
| 10 | 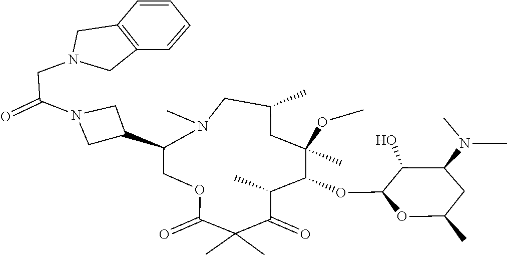 [SMILES: CO[C@]1(C)C[C@@H](C)CN(C)[C@H](C2CN(C(=O)CN3Cc4ccccc4C3)C2)COC(=O)C(C)(C)C(=O)[C@H](C)[C@H]1O[C@@H]1O[C@H](C)C[C@H](N(C)C)[C@H]1O] |
| 11 | 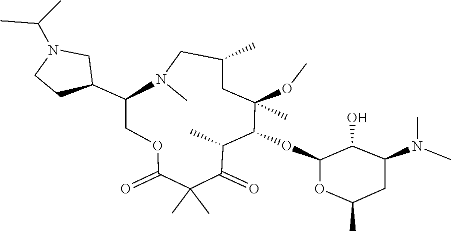 [SMILES: CO[C@]1(C)C[C@@H](C)CN(C)[C@H]([C@H]2CCN(C(C)C)C2)COC(=O)C(C)(C)C(=O)[C@H](C)[C@H]1O[C@@H]1O[C@H](C)C[C@H](N(C)C)[C@H]1O] |
| 12 | 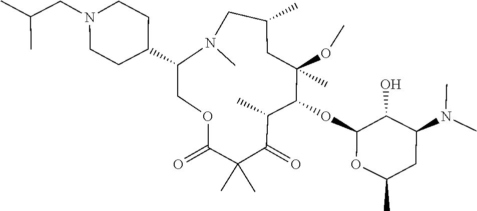 [SMILES: CO[C@]1(C)C[C@@H](C)CN(C)[C@@H](C2CCN(CC(C)C)CC2)COC(=O)C(C)(C)C(=O)[C@H](C)[C@H]1O[C@@H]1O[C@H](C)C[C@H](N(C)C)[C@H]1O] |
| 13 | 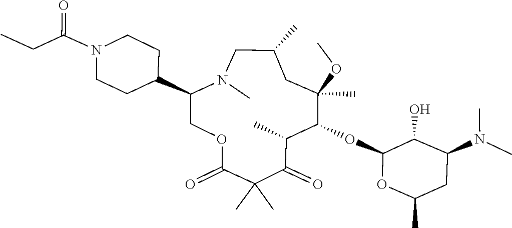 [SMILES: CCC(=O)N1CCC([C@@H]2COC(=O)C(C)(C)C(=O)[C@H](C)[C@@H](O[C@@H]3O[C@H](C)C[C@H](N(C)C)[C@H]3O)[C@](C)(OC)C[C@@H](C)CN2C)CC1] |
| 14 | 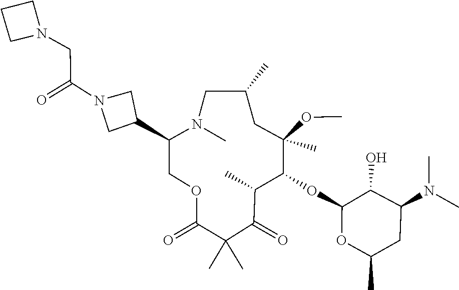 [SMILES: CO[C@]1(C)C[C@@H](C)CN(C)[C@H](C2CN(C(=O)CN3CCC3)C2)COC(=O)C(C)(C)C(=O)[C@H](C)[C@H]1O[C@@H]1O[C@H](C)C[C@H](N(C)C)[C@H]1O] |
| 15 | 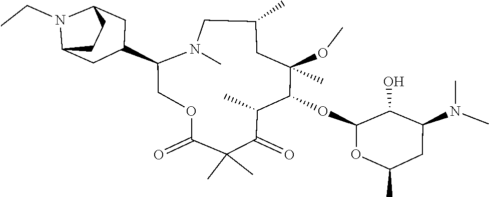 [SMILES: CCN1[C@@H]2CC[C@H]1CC([C@@H]1COC(=O)C(C)(C)C(=O)[C@H](C)[C@@H](O[C@@H]3O[C@H](C)C[C@H](N(C)C)[C@H]3O)[C@](C)(OC)C[C@@H](C)CN1C)C2] |
| 16 | 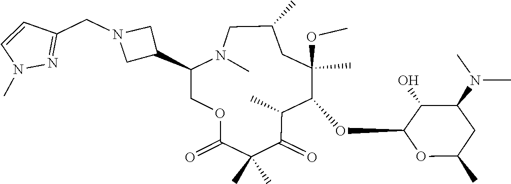 |
| 17 | 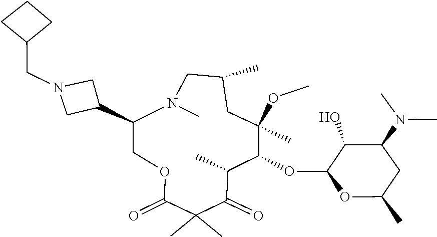 [SMILES: CO[C@]1(C)C[C@@H](C)CN(C)[C@H](C2CN(CC3CCC3)C2)COC(=O)C(C)(C)C(=O)[C@H](C)[C@H]1O[C@@H]1O[C@H](C)C[C@H](N(C)C)[C@H]1O] |
| 18 | 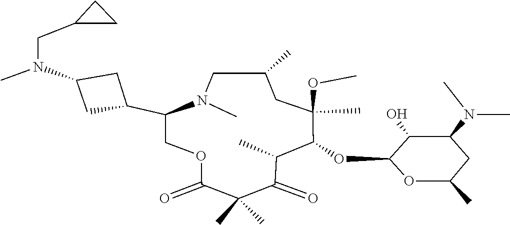 |
| 19 | 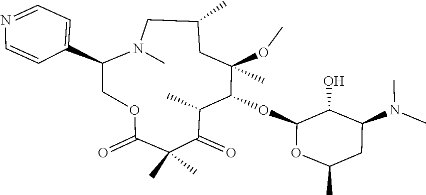 |
| 20 | 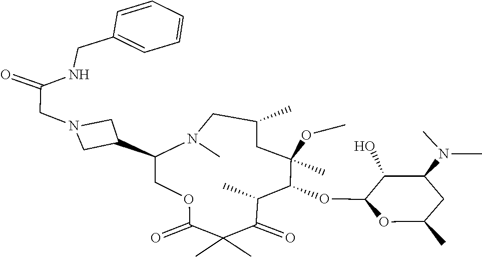 [SMILES: CO[C@]1(C)C[C@@H](C)CN(C)[C@H](C2CN(CC(=O)NCc3ccccc3)C2)COC(=O)C(C)(C)C(=O)[C@H](C)[C@H]1O[C@@H]1O[C@H](C)C[C@H](N(C)C)[C@H]1O] |
| 21 | 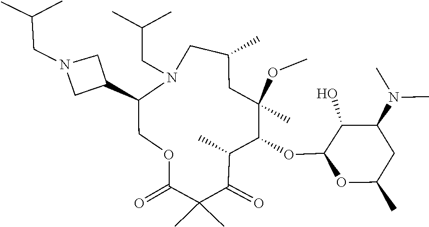 [SMILES: CO[C@]1(C)C[C@@H](C)CN(CC(C)C)[C@H](C2CN(CC(C)C)C2)COC(=O)C(C)(C)C(=O)[C@H](C)[C@H]1O[C@@H]1O[C@H](C)C[C@H](N(C)C)[C@H]1O] |
| 22 | 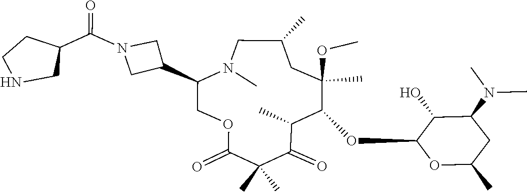 |
| 23 | 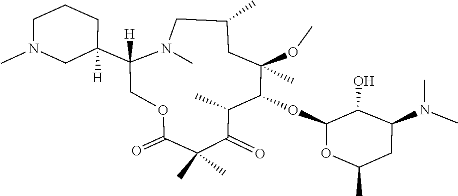 |
| 24 | 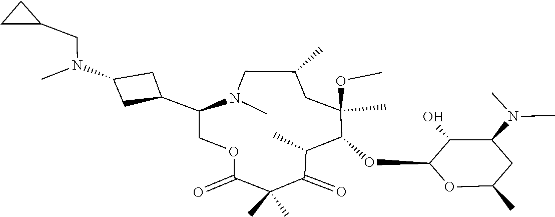 |
| 25 | 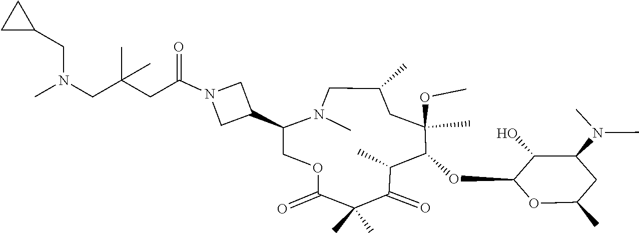 |
| 26 | 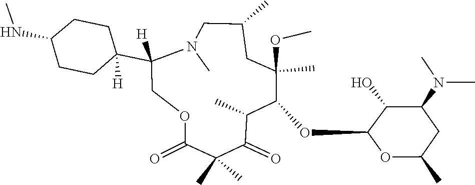 |
| 27 | 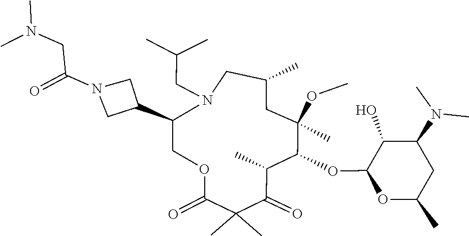 |
| 28 | 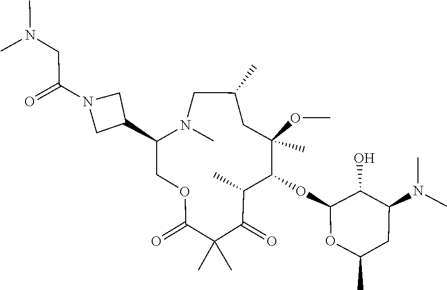 |
| 29 | 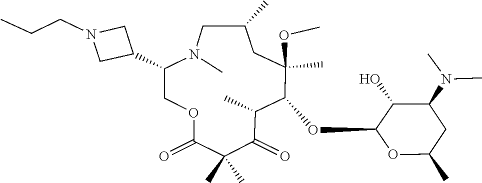 |
| 30 | 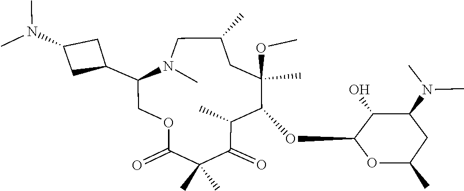 |
| 31 | 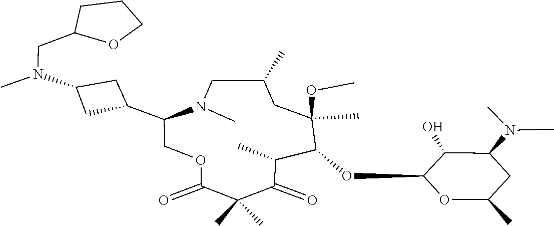 |
| 32 | 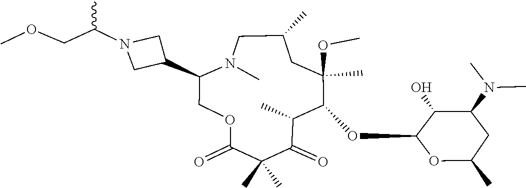 |
| 33 | 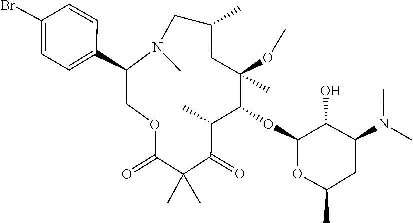 [SMILES: CO[C@]1(C)C[C@@H](C)CN(C)[C@H](c2ccc(Br)cc2)COC(=O)C(C)(C)C(=O)[C@H](C)[C@H]1O[C@@H]1O[C@H](C)C[C@H](N(C)C)[C@H]1O] |
| 34 | 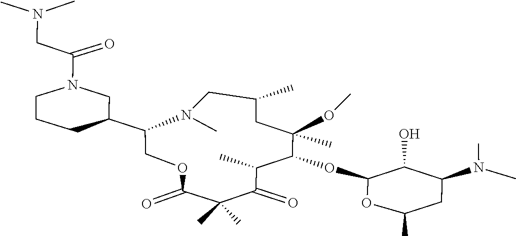 |
| 35 | 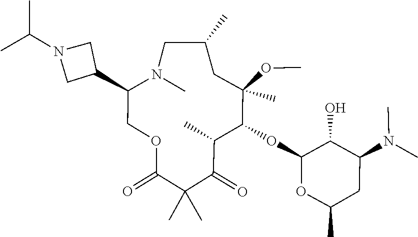 [SMILES: CO[C@]1(C)C[C@@H](C)CN(C)[C@H](C2CN(C(C)C)C2)COC(=O)C(C)(C)C(=O)[C@H](C)[C@H]1O[C@@H]1O[C@H](C)C[C@H](N(C)C)[C@H]1O] |
| 36 | 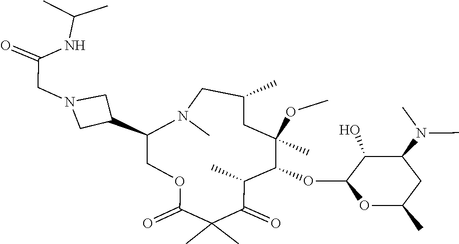 [SMILES: CO[C@]1(C)C[C@@H](C)CN(C)[C@H](C2CN(CC(=O)NC(C)C)C2)COC(=O)C(C)(C)C(=O)[C@H](C)[C@H]1O[C@@H]1O[C@H](C)C[C@H](N(C)C)[C@H]1O] |
| 37 | 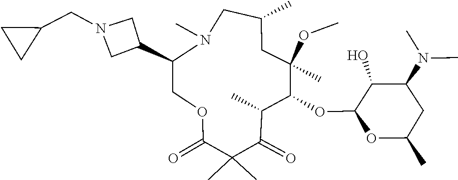 [SMILES: CO[C@]1(C)C[C@@H](C)CN(C)[C@H](C2CN(CC3CC3)C2)COC(=O)C(C)(C)C(=O)[C@H](C)[C@H]1O[C@@H]1O[C@H](C)C[C@H](N(C)C)[C@H]1O] |
| 38 | 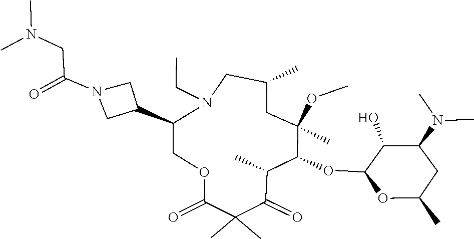 |
| 39 | 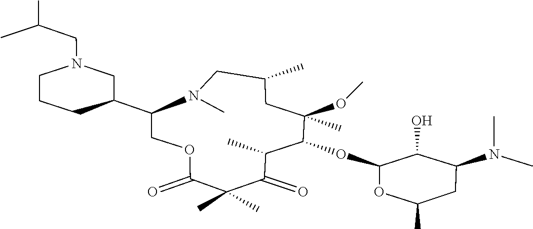 |
| 40 | 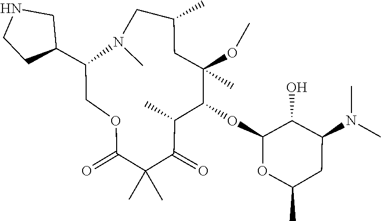 [SMILES: CO[C@]1(C)C[C@@H](C)CN(C)[C@@H]([C@H]2CCNC2)COC(=O)C(C)(C)C(=O)[C@H](C)[C@H]1O[C@@H]1O[C@H](C)C[C@H](N(C)C)[C@H]1O] |
| 41 | 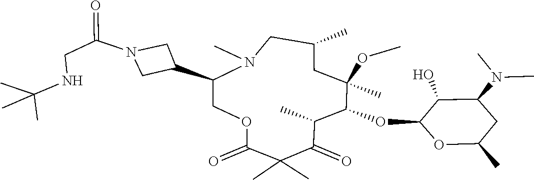 [SMILES: CO[C@]1(C)C[C@@H](C)CN(C)[C@H](C2CN(C(=O)CNC(C)(C)C)C2)COC(=O)C(C)(C)C(=O)[C@H](C)[C@H]1O[C@@H]1O[C@H](C)C[C@H](N(C)C)[C@H]1O] |
| 42 | 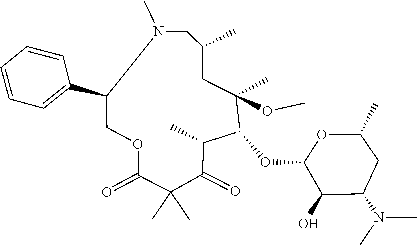 |
| 43 | 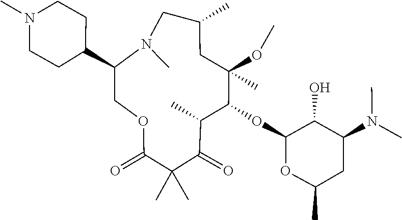 |
| 44 | 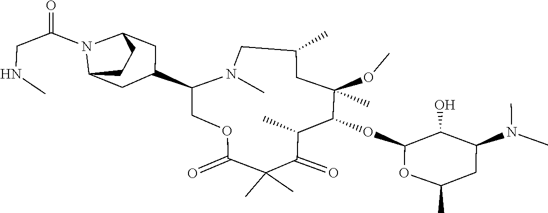 [SMILES: CNCC(=O)N1[C@@H]2CC[C@H]1CC([C@@H]1COC(=O)C(C)(C)C(=O)[C@H](C)[C@@H](O[C@@H]3O[C@H](C)C[C@H](N(C)C)[C@H]3O)[C@](C)(OC)C[C@@H](C)CN1C)C2] |
| 45 | 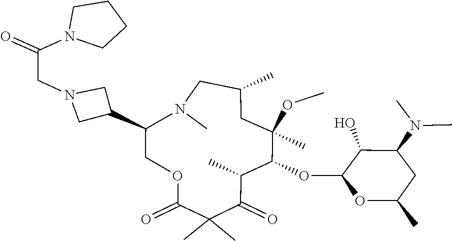 [SMILES: CO[C@]1(C)C[C@@H](C)CN(C)[C@H](C2CN(CC(=O)N3CCCC3)C2)COC(=O)C(C)(C)C(=O)[C@H](C)[C@H]1O[C@@H]1O[C@H](C)C[C@H](N(C)C)[C@H]1O] |
| 46 | 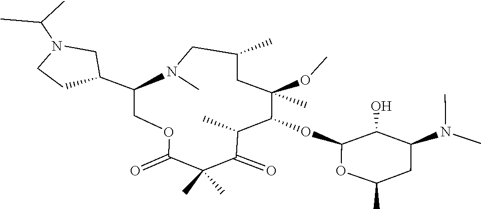 |
| 47 | 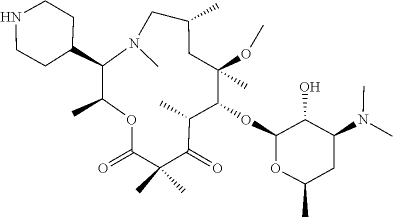 |
| 48 | 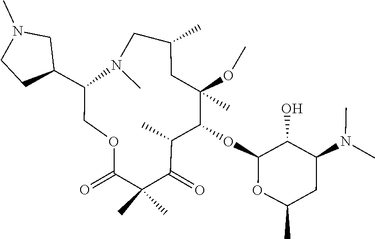 |
| 49 | 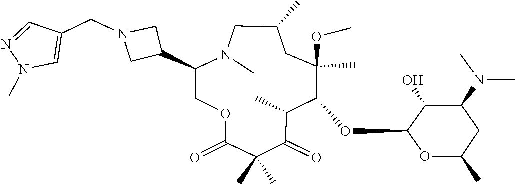 |
| 50 | 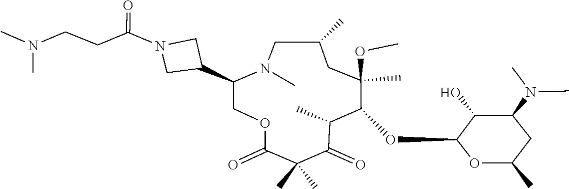 |
| 51 | 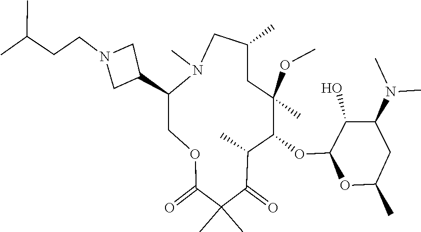 [SMILES: CO[C@]1(C)C[C@@H](C)CN(C)[C@H](C2CN(CCC(C)C)C2)COC(=O)C(C)(C)C(=O)[C@H](C)[C@H]1O[C@@H]1O[C@H](C)C[C@H](N(C)C)[C@H]1O] |
| 52 | 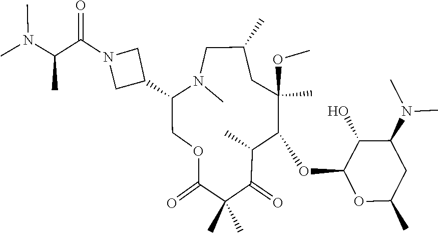 |
| 53 | 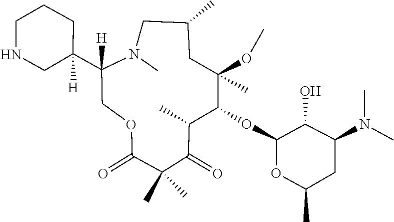 |
| 54 | 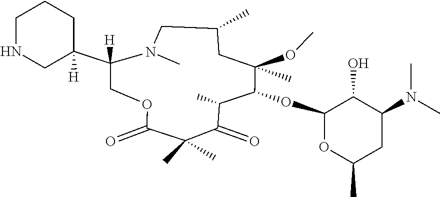 |
| 55 | 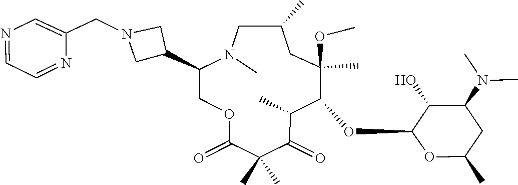 |
| 56 | 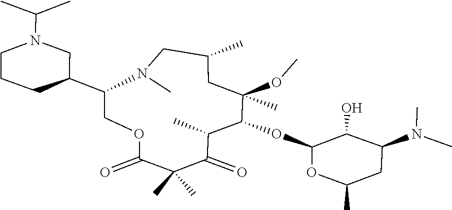 |
| 57 | 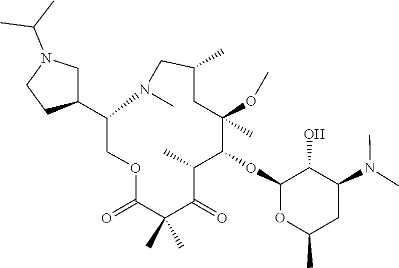 |
| 60 | 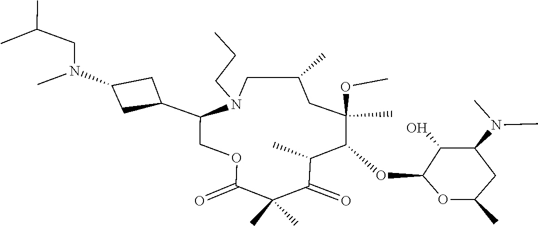 |
| 61 | 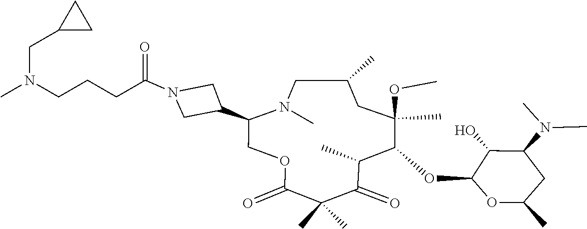 |
| 62 | 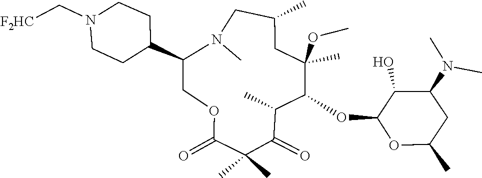 |
| 63 | 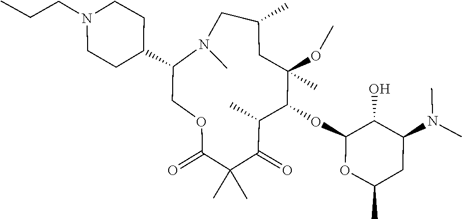 [SMILES: CCCN1CCC([C@H]2COC(=O)C(C)(C)C(=O)[C@H](C)[C@@H](O[C@@H]3O[C@H](C)C[C@H](N(C)C)[C@H]3O)[C@](C)(OC)C[C@@H](C)CN2C)CC1] |
| 64 | 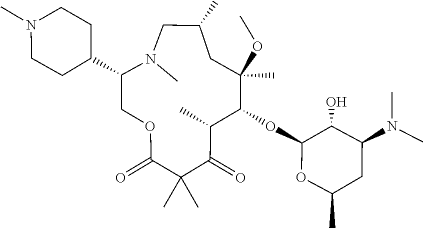 |
| 65 | 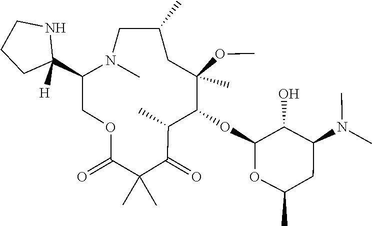 [SMILES: CO[C@]1(C)C[C@@H](C)CN(C)[C@H]([C@@H]2CCCN2)COC(=O)C(C)(C)C(=O)[C@H](C)[C@H]1O[C@@H]1O[C@H](C)C[C@H](N(C)C)[C@H]1O] |
| 66 | 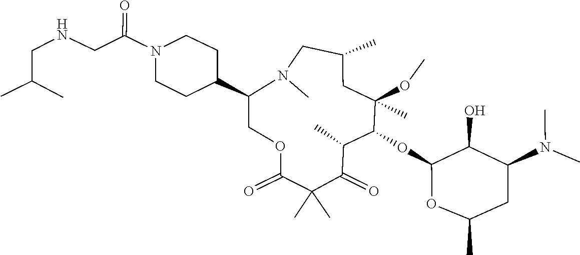 [SMILES: CO[C@]1(C)C[C@@H](C)CN(C)[C@H](C2CCN(C(=O)CNCC(C)C)CC2)COC(=O)C(C)(C)C(=O)[C@H](C)[C@H]1O[C@@H]1O[C@H](C)C[C@H](N(C)C)[C@@H]1O] |
| 67 | 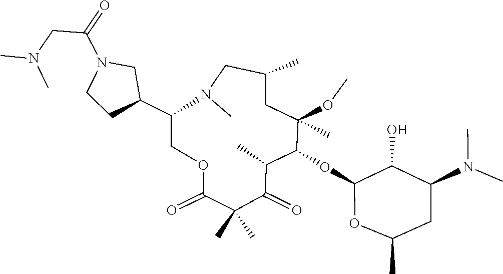 |
| 68 | 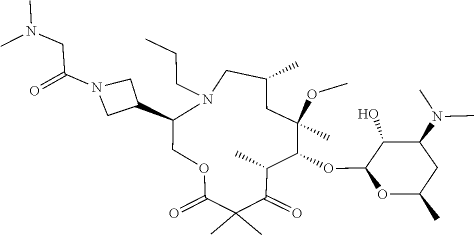 |
| 69 | 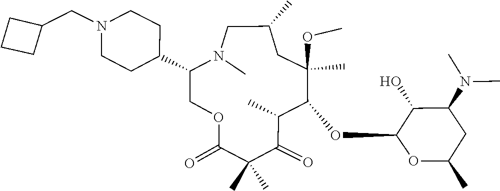 |
| 70 | 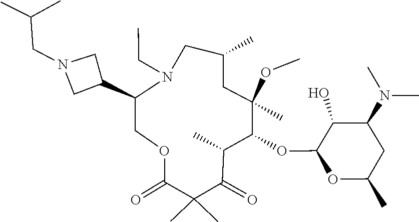 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@H]1C1CN(CC(C)C)C1] |
| 71 | 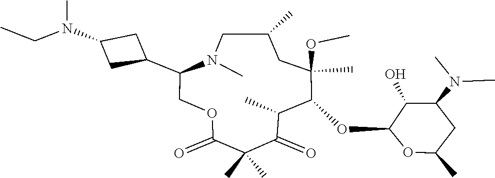 |
| 72 | 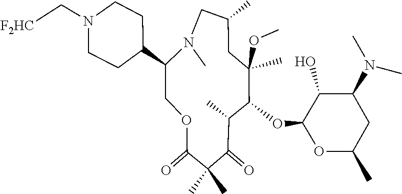 |
| 73 | 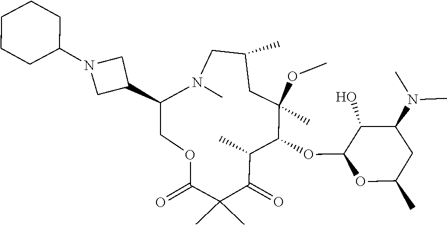 [SMILES: CO[C@]1(C)C[C@@H](C)CN(C)[C@H](C2CN(C3CCCCC3)C2)COC(=O)C(C)(C)C(=O)[C@H](C)[C@H]1O[C@@H]1O[C@H](C)C[C@H](N(C)C)[C@H]1O] |
| 74 | 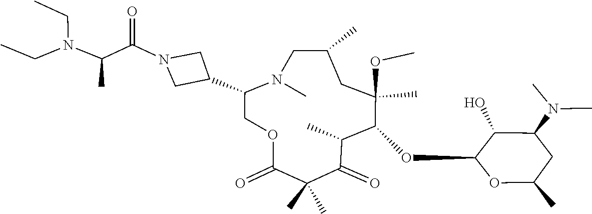 |
| 76 | 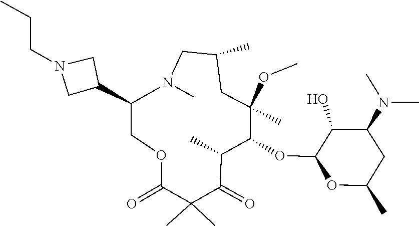 [SMILES: CCCN1CC([C@@H]2COC(=O)C(C)(C)C(=O)[C@H](C)[C@@H](O[C@@H]3O[C@H](C)C[C@H](N(C)C)[C@H]3O)[C@](C)(OC)C[C@@H](C)CN2C)C1] |
| 77 | 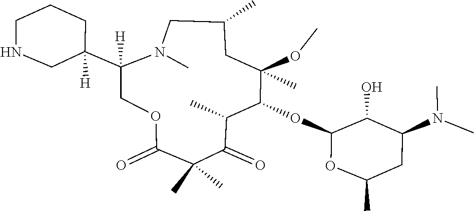 |
| 78 | 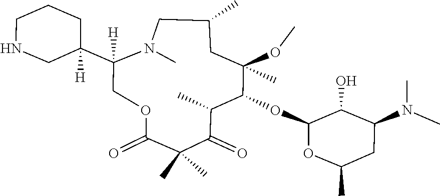 |
| 79 | 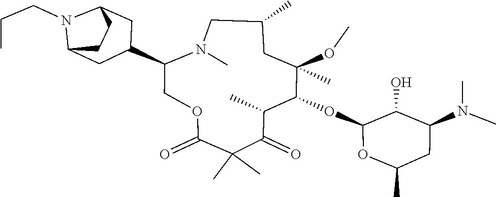 [SMILES: CCCN1[C@@H]2CC[C@H]1CC([C@@H]1COC(=O)C(C)(C)C(=O)[C@H](C)[C@@H](O[C@@H]3O[C@H](C)C[C@H](N(C)C)[C@H]3O)[C@](C)(OC)C[C@@H](C)CN1C)C2] |
| 80 | 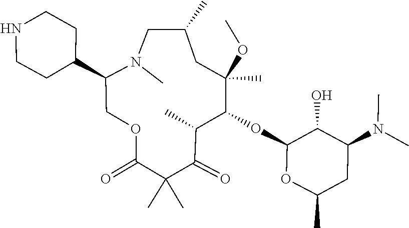 [SMILES: CO[C@]1(C)C[C@@H](C)CN(C)[C@H](C2CCNCC2)COC(=O)C(C)(C)C(=O)[C@H](C)[C@H]1O[C@@H]1O[C@H](C)C[C@H](N(C)C)[C@H]1O] |
| 81 | 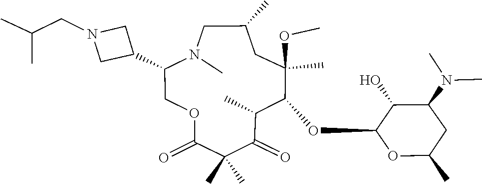 |
| 82 | 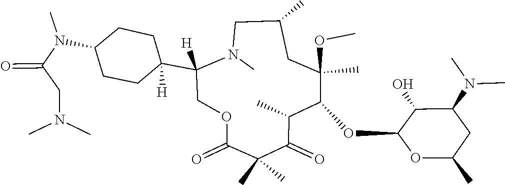 |
| 84 | 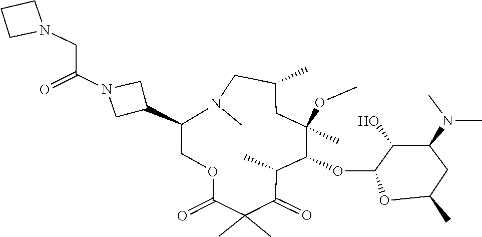 [SMILES: CO[C@]1(C)C[C@@H](C)CN(C)[C@H](C2CN(C(=O)CN3CCC3)C2)COC(=O)C(C)(C)C(=O)[C@H](C)[C@H]1O[C@H]1O[C@H](C)C[C@H](N(C)C)[C@H]1O] |
| 85 | 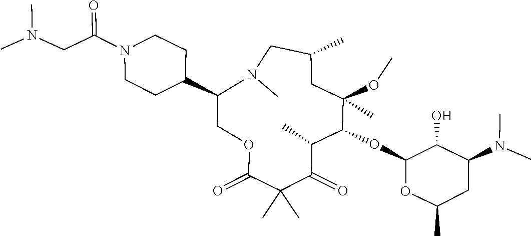 |
| 86 | 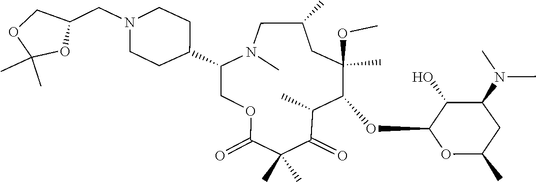 |
| 87 | 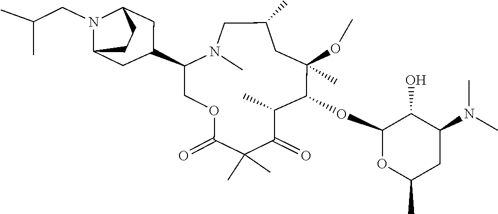 [SMILES: CO[C@]1(C)C[C@@H](C)CN(C)[C@H](C2C[C@H]3CC[C@@H](C2)N3CC(C)C)COC(=O)C(C)(C)C(=O)[C@H](C)[C@H]1O[C@@H]1O[C@H](C)C[C@H](N(C)C)[C@H]1O] |
| 88 | 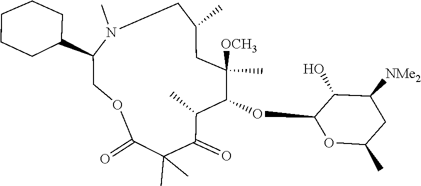 [SMILES: CO[C@]1(C)C[C@@H](C)CN(C)[C@H](C2CCCCC2)COC(=O)C(C)(C)C(=O)[C@H](C)[C@H]1O[C@@H]1O[C@H](C)C[C@H](N(C)C)[C@H]1O] |
| 89 | 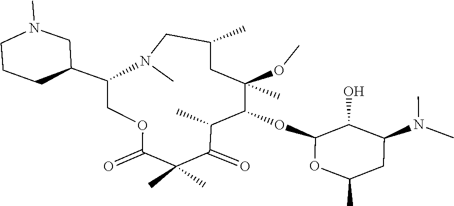 |
| 90 | 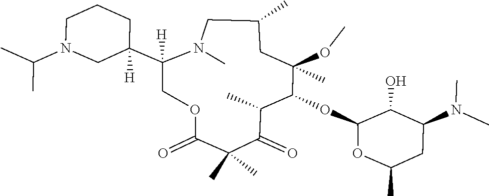 |
| 91 | 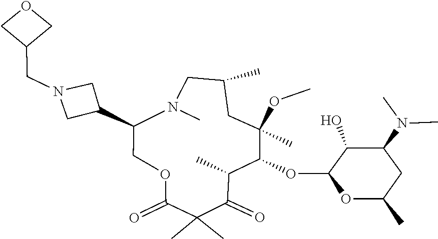 [SMILES: CO[C@]1(C)C[C@@H](C)CN(C)[C@H](C2CN(CC3COC3)C2)COC(=O)C(C)(C)C(=O)[C@H](C)[C@H]1O[C@@H]1O[C@H](C)C[C@H](N(C)C)[C@H]1O] |
| 92 | 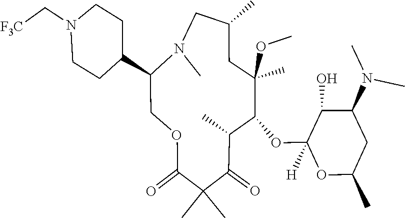 [SMILES: CO[C@]1(C)C[C@@H](C)CN(C)[C@H](C2CCN(CC(F)(F)F)CC2)COC(=O)C(C)(C)C(=O)[C@H](C)[C@H]1O[C@@H]1O[C@H](C)C[C@H](N(C)C)[C@H]1O] |
| 93 | 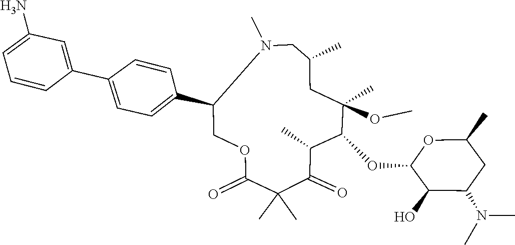 |
| 94 | 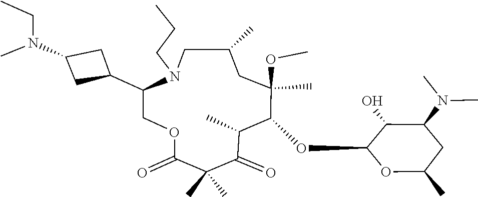 |
| 95 | 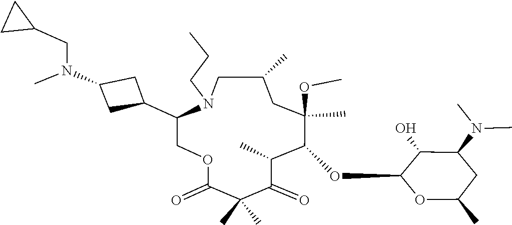 |
| 96 | 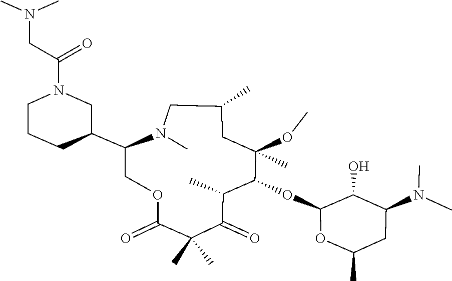 |
| 97 | 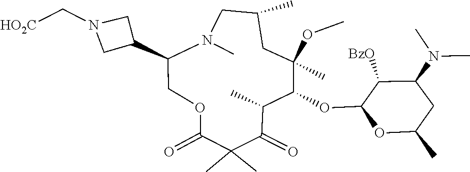 |
| 98 | 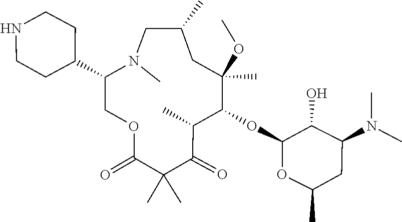 [SMILES: CO[C@]1(C)C[C@@H](C)CN(C)[C@@H](C2CCNCC2)COC(=O)C(C)(C)C(=O)[C@H](C)[C@H]1O[C@@H]1O[C@H](C)C[C@H](N(C)C)[C@H]1O] |
| 99 | 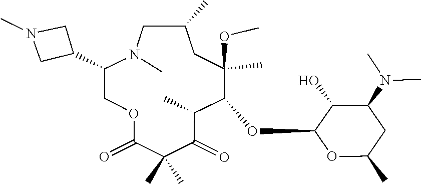 |
| 100 | 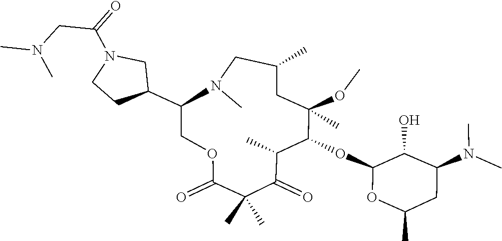 |
| 101 | 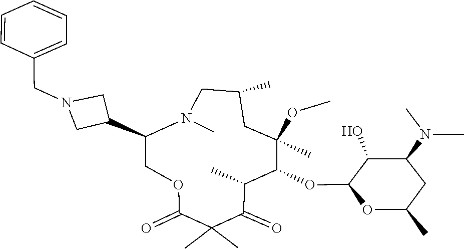 [SMILES: CO[C@]1(C)C[C@@H](C)CN(C)[C@H](C2CN(Cc3ccccc3)C2)COC(=O)C(C)(C)C(=O)[C@H](C)[C@H]1O[C@@H]1O[C@H](C)C[C@H](N(C)C)[C@H]1O] |
| 102 | 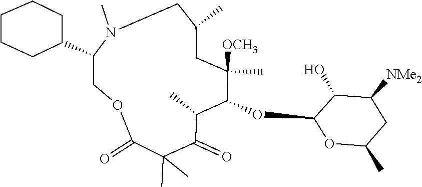 [SMILES: CO[C@]1(C)C[C@@H](C)CN(C)[C@@H](C2CCCCC2)COC(=O)C(C)(C)C(=O)[C@H](C)[C@H]1O[C@@H]1O[C@H](C)C[C@H](N(C)C)[C@H]1O] |
| 103 | 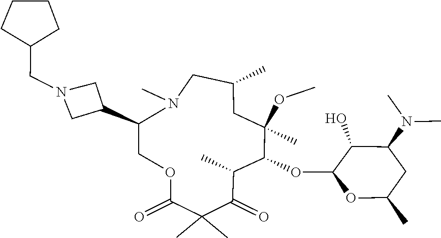 [SMILES: CO[C@]1(C)C[C@@H](C)CN(C)[C@H](C2CN(CC3CCCC3)C2)COC(=O)C(C)(C)C(=O)[C@H](C)[C@H]1O[C@@H]1O[C@H](C)C[C@H](N(C)C)[C@H]1O] |
| 104 | 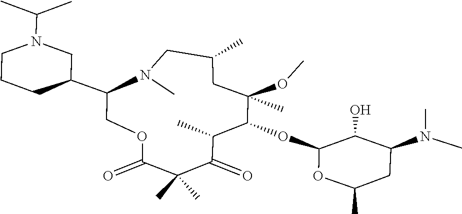 |
| 105 | 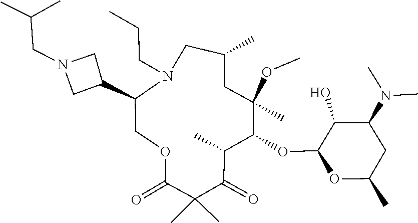 [SMILES: CCCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@H]1C1CN(CC(C)C)C1] |
| 106 | 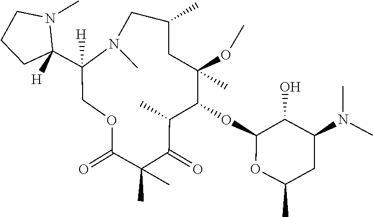 |
| 107 | 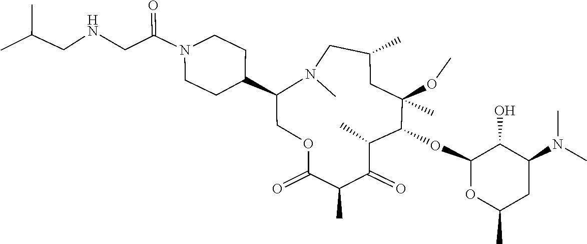 [SMILES: CO[C@]1(C)C[C@@H](C)CN(C)[C@H](C2CCN(C(=O)CNCC(C)C)CC2)COC(=O)[C@H](C)C(=O)[C@H](C)[C@H]1O[C@@H]1O[C@H](C)C[C@H](N(C)C)[C@H]1O] |
| 108 | 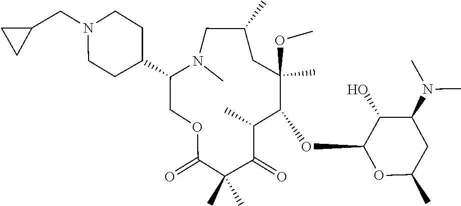 |
| 109 | 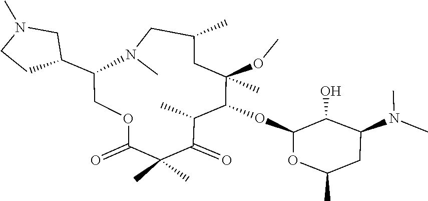 |
| 110 | 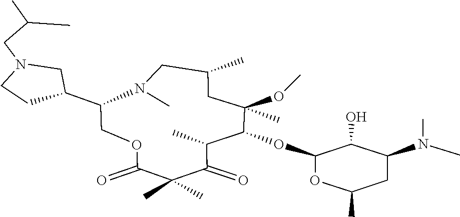 |
| 111 | 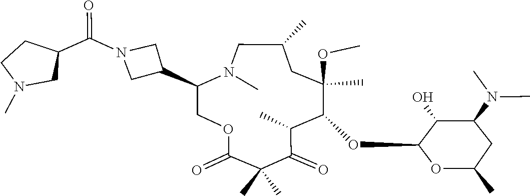 |
| 112 | 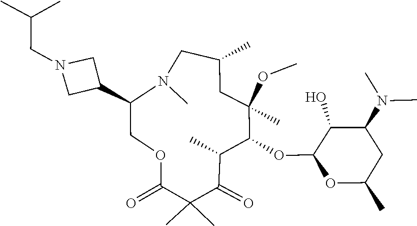 [SMILES: CO[C@]1(C)C[C@@H](C)CN(C)[C@H](C2CN(CC(C)C)C2)COC(=O)C(C)(C)C(=O)[C@H](C)[C@H]1O[C@@H]1O[C@H](C)C[C@H](N(C)C)[C@H]1O] |
| 113 | 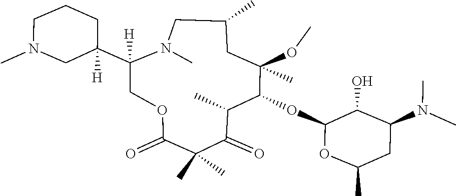 |
| 114 | 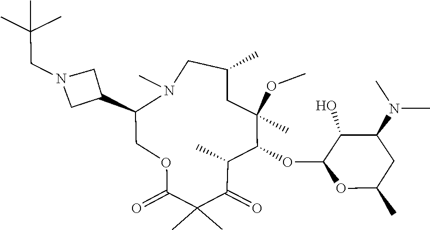 [SMILES: CO[C@]1(C)C[C@@H](C)CN(C)[C@H](C2CN(CC(C)(C)C)C2)COC(=O)C(C)(C)C(=O)[C@H](C)[C@H]1O[C@@H]1O[C@H](C)C[C@H](N(C)C)[C@H]1O] |
| 115 | 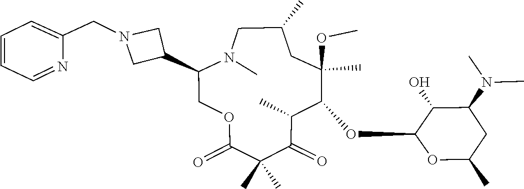 |
| 116 | 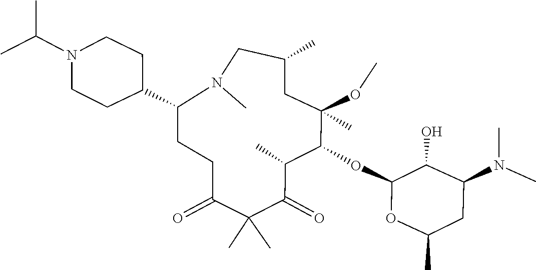 [SMILES: CO[C@]1(C)C[C@@H](C)CN(C)[C@@H](C2CCN(C(C)C)CC2)CCC(=O)C(C)(C)C(=O)[C@H](C)[C@H]1O[C@@H]1O[C@H](C)C[C@H](N(C)C)[C@H]1O] |
| 117 | 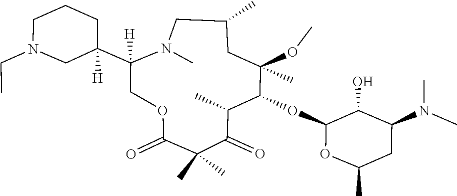 |
| 118 | 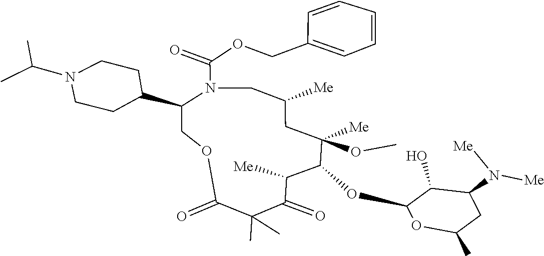 [SMILES: CO[C@]1(C)C[C@@H](C)CN(C(=O)OCc2ccccc2)[C@H](C2CCN(C(C)C)CC2)COC(=O)C(C)(C)C(=O)[C@H](C)[C@H]1O[C@@H]1O[C@H](C)C[C@H](N(C)C)[C@H]1O] |
| 119 | 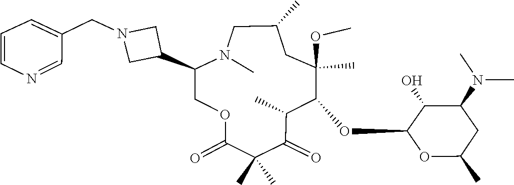 |
| 120 | 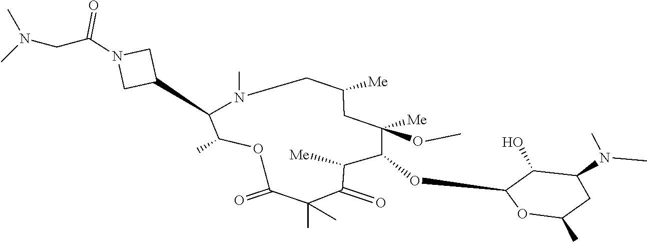 |
| 121 | 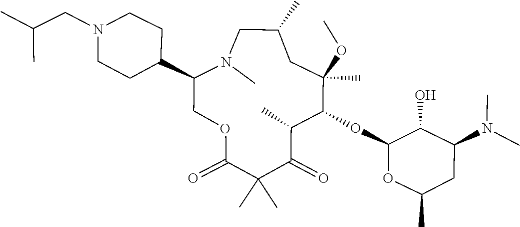 [SMILES: CO[C@]1(C)C[C@@H](C)CN(C)[C@H](C2CCN(CC(C)C)CC2)COC(=O)C(C)(C)C(=O)[C@H](C)[C@H]1O[C@@H]1O[C@H](C)C[C@H](N(C)C)[C@H]1O] |
| 122 | 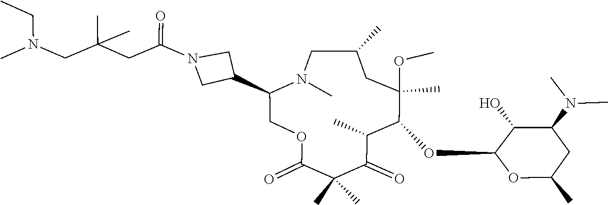 |
| 123 | 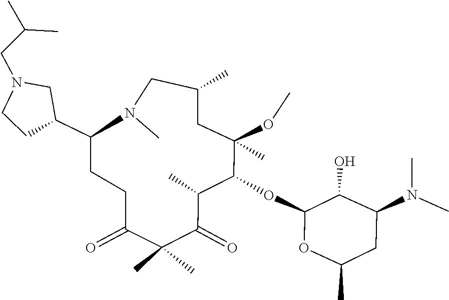 |
| 124 | 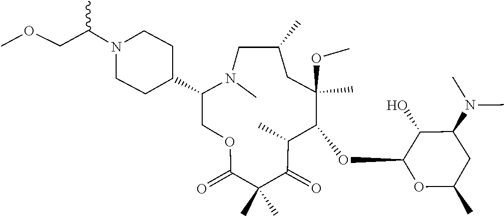 |
| 125 | 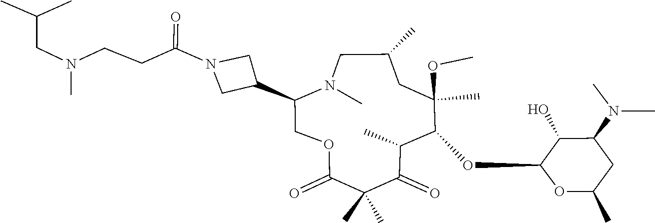 |
| 126 | 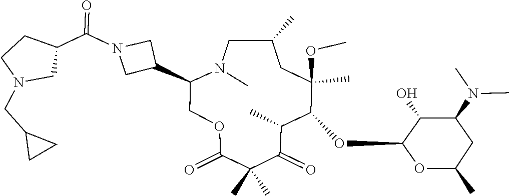 |
| 127 | 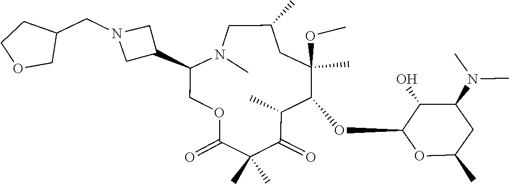 |
| 128 | 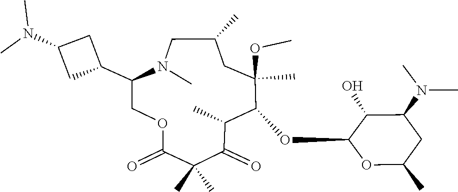 |
| 129 | 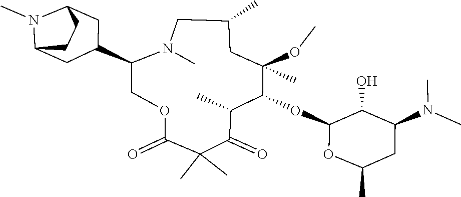 |
| 130 | 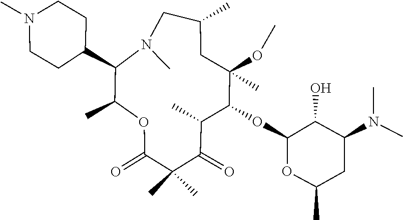 |
| 131 | 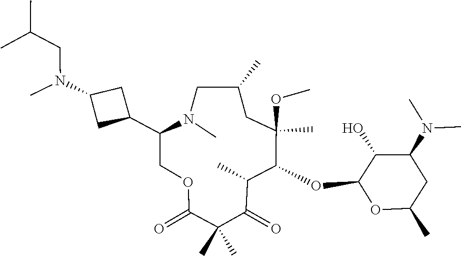 |
| 132 | 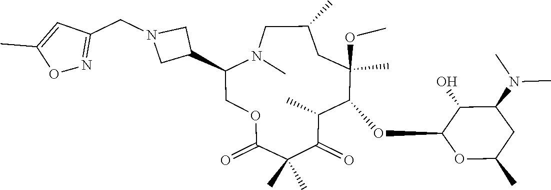 |
| 133 | 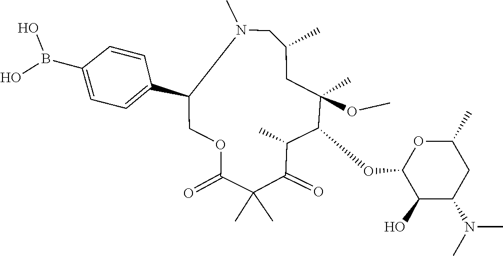 |
| 134 | 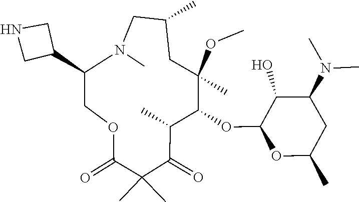 [SMILES: CO[C@]1(C)C[C@@H](C)CN(C)[C@H](C2CNC2)COC(=O)C(C)(C)C(=O)[C@H](C)[C@H]1O[C@@H]1O[C@H](C)C[C@H](N(C)C)[C@H]1O] |
| 135 | 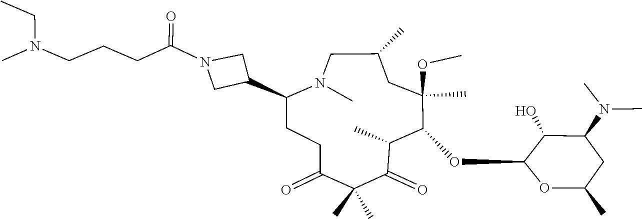 |
| 136 | 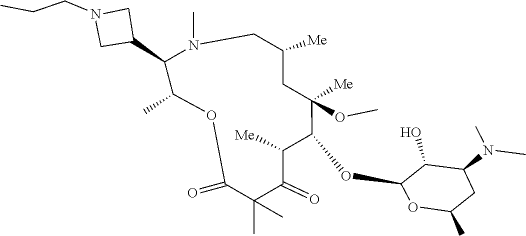 |
| 137 | 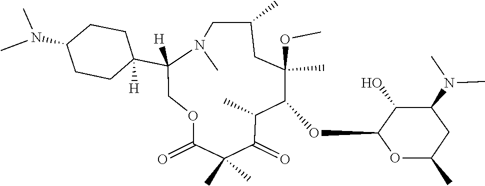 |
| 138 | 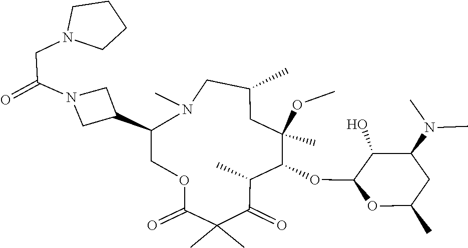 [SMILES: CO[C@]1(C)C[C@@H](C)CN(C)[C@H](C2CN(C(=O)CN3CCCC3)C2)COC(=O)C(C)(C)C(=O)[C@H](C)[C@H]1O[C@@H]1O[C@H](C)C[C@H](N(C)C)[C@H]1O] |
| 139 | 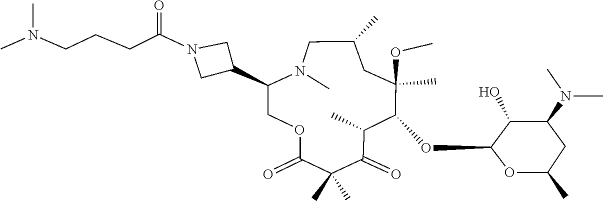 |
| 140 | 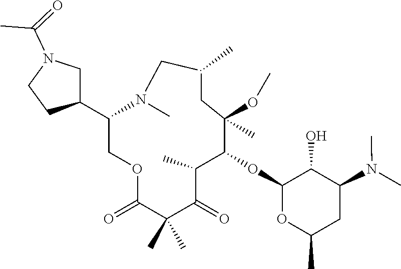 |
| 142 | 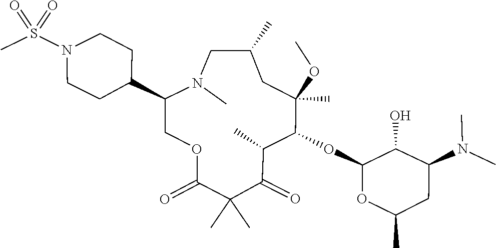 |
| 143 | 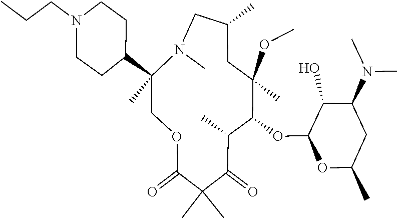 [SMILES: CCCN1CCC([C@]2(C)COC(=O)C(C)(C)C(=O)[C@H](C)[C@@H](O[C@@H]3O[C@H](C)C[C@H](N(C)C)[C@H]3O)[C@](C)(OC)C[C@@H](C)CN2C)CC1] |
| 144 | 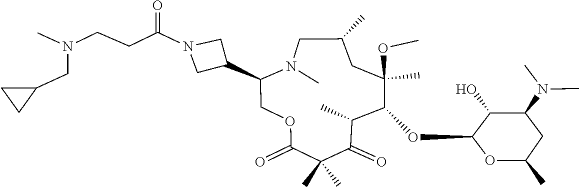 |
| 145 | 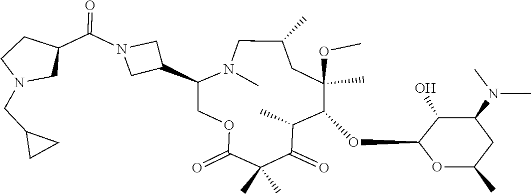 |
| 146 | 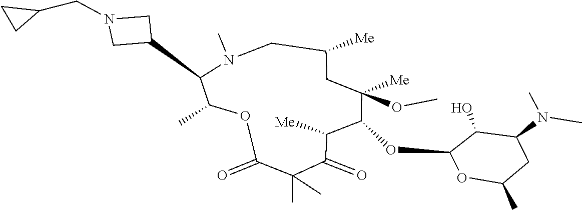 |
| 147 | 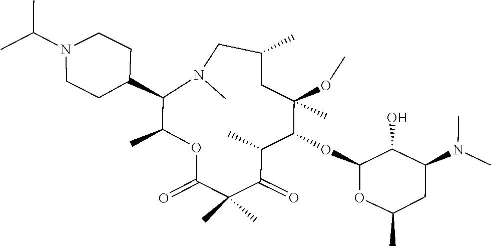 |
| 148 | 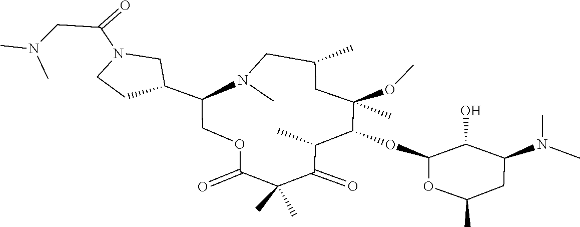 |
| 149 | 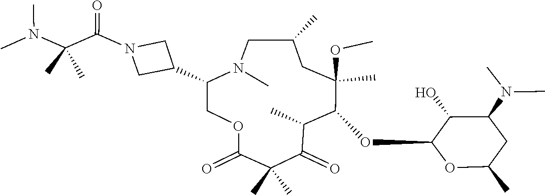 |
| 150 | 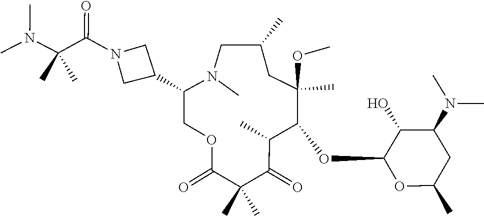 |
| 151 | 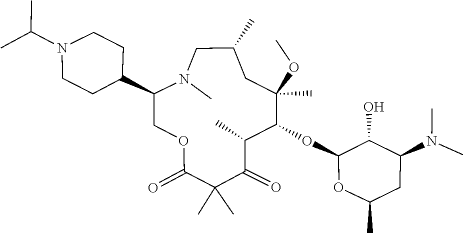 [SMILES: CO[C@]1(C)C[C@@H](C)CN(C)[C@H](C2CCN(C(C)C)CC2)COC(=O)C(C)(C)C(=O)[C@H](C)[C@H]1O[C@@H]1O[C@H](C)C[C@H](N(C)C)[C@H]1O] |
| 154 | 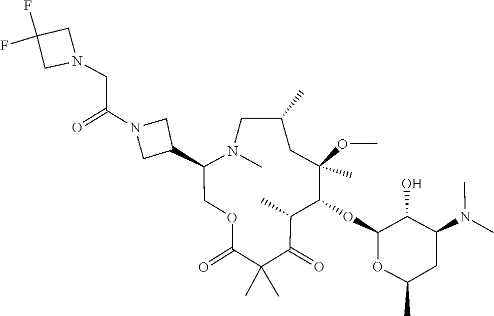 [SMILES: CO[C@]1(C)C[C@@H](C)CN(C)[C@H](C2CN(C(=O)CN3CC(F)(F)C3)C2)COC(=O)C(C)(C)C(=O)[C@H](C)[C@H]1O[C@@H]1O[C@H](C)C[C@H](N(C)C)[C@H]1O] |
| 155 | 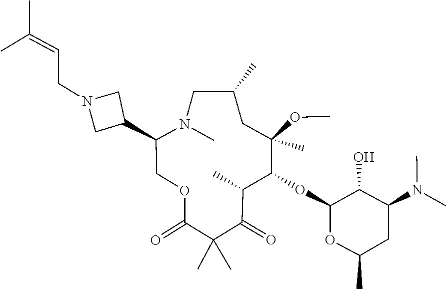 [SMILES: CO[C@]1(C)C[C@@H](C)CN(C)[C@H](C2CN(CC=C(C)C)C2)COC(=O)C(C)(C)C(=O)[C@H](C)[C@H]1O[C@@H]1O[C@H](C)C[C@H](N(C)C)[C@H]1O] |
| 156 | 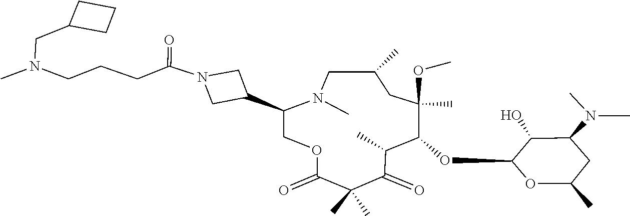 |
| 157 | 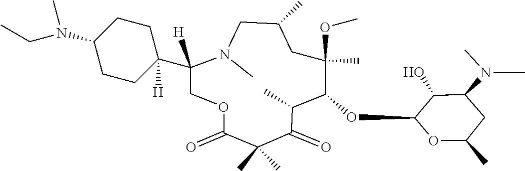 |
| 158 | 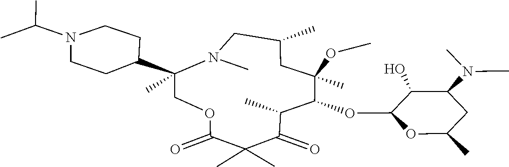 [SMILES: CO[C@]1(C)C[C@@H](C)CN(C)[C@](C)(C2CCN(C(C)C)CC2)COC(=O)C(C)(C)C(=O)[C@H](C)[C@H]1O[C@@H]1O[C@H](C)C[C@H](N(C)C)[C@H]1O] |
| 159 | 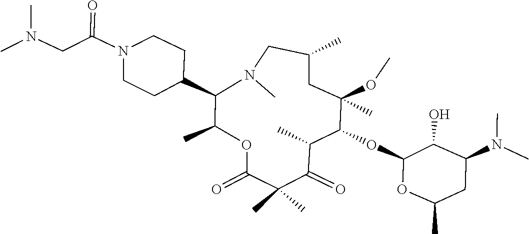 |
| 160 | 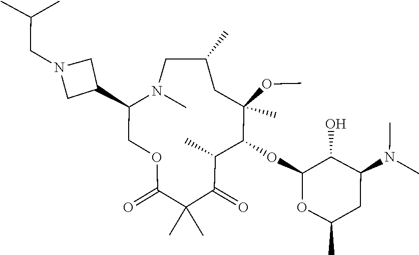 [SMILES: CO[C@]1(C)C[C@@H](C)CN(C)[C@H](C2CN(CC(C)C)C2)COC(=O)C(C)(C)C(=O)[C@H](C)[C@H]1O[C@@H]1O[C@H](C)C[C@H](N(C)C)[C@H]1O] |
| 161 | 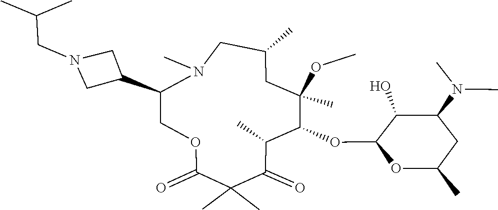 [SMILES: CO[C@]1(C)C[C@@H](C)CN(C)[C@H](C2CN(CC(C)C)C2)COC(=O)C(C)(C)C(=O)[C@H](C)[C@H]1O[C@@H]1O[C@H](C)C[C@H](N(C)C)[C@H]1O] |
| 164 | 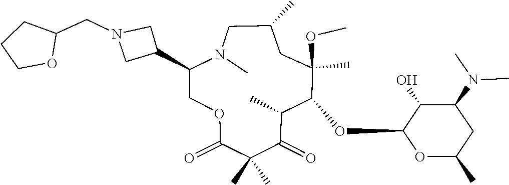 |
| 165 | 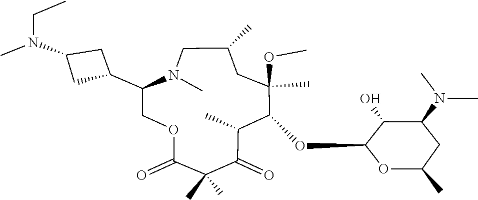 |
| 166 | 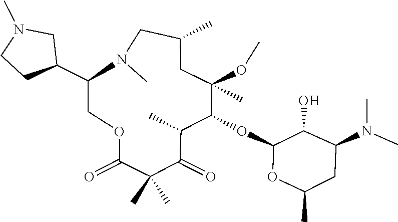 |
| 167 | 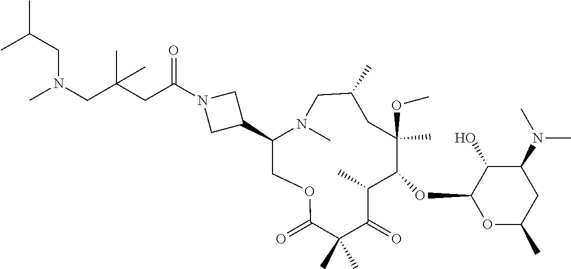 |
| 168 | 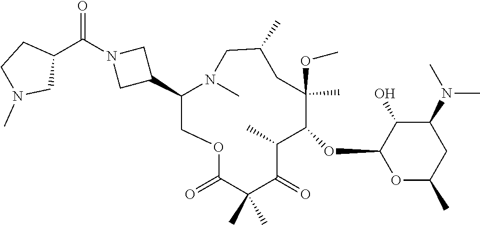 |
| 169 | 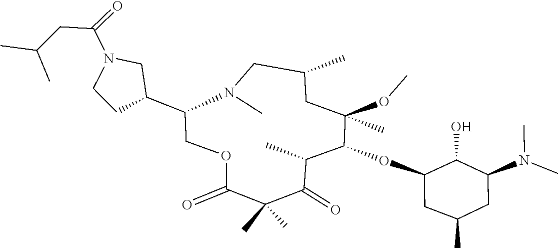 |
| 170 | 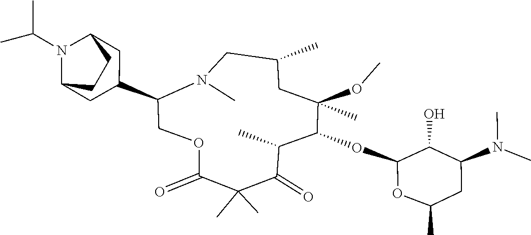 [SMILES: CO[C@]1(C)C[C@@H](C)CN(C)[C@H](C2C[C@H]3CC[C@@H](C2)N3C(C)C)COC(=O)C(C)(C)C(=O)[C@H](C)[C@H]1O[C@@H]1O[C@H](C)C[C@H](N(C)C)[C@H]1O] |
| 171 | 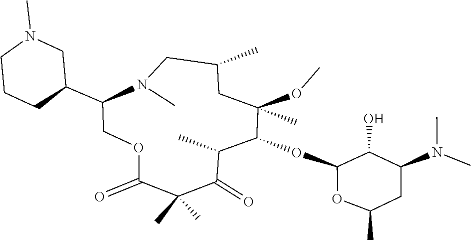 |
| 172 | 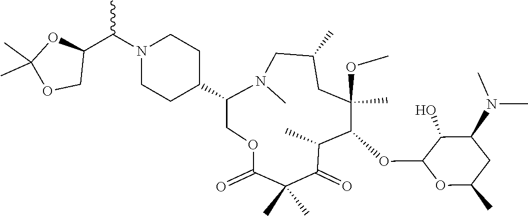 |
| 173 | 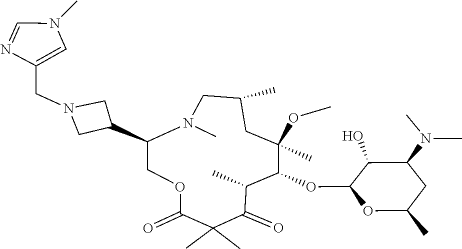 [SMILES: CO[C@]1(C)C[C@@H](C)CN(C)[C@H](C2CN(Cc3cn(C)cn3)C2)COC(=O)C(C)(C)C(=O)[C@H](C)[C@H]1O[C@@H]1O[C@H](C)C[C@H](N(C)C)[C@H]1O] |
| 174 | 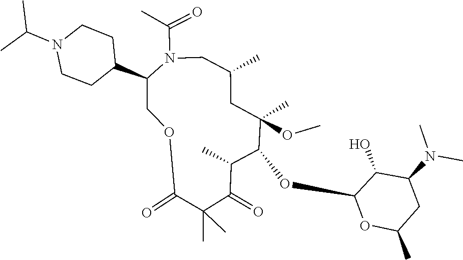 [SMILES: CO[C@]1(C)C[C@@H](C)CN(C(C)=O)[C@H](C2CCN(C(C)C)CC2)COC(=O)C(C)(C)C(=O)[C@H](C)[C@H]1O[C@@H]1O[C@H](C)C[C@H](N(C)C)[C@H]1O] |
| 175 | 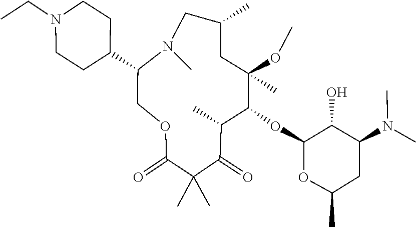 [SMILES: CCN1CCC([C@H]2COC(=O)C(C)(C)C(=O)[C@H](C)[C@@H](O[C@@H]3O[C@H](C)C[C@H](N(C)C)[C@H]3O)[C@](C)(OC)C[C@@H](C)CN2C)CC1] |
| 176 | 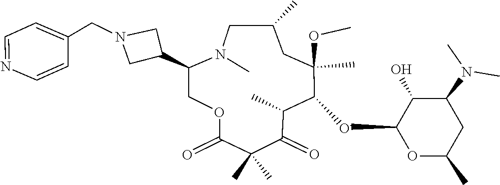 |
| 177 | 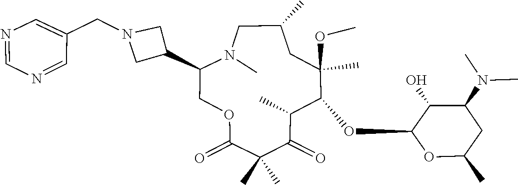 |
| 178 | 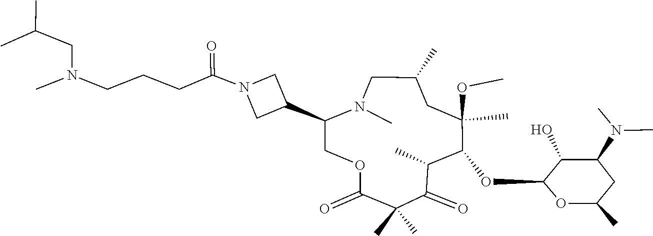 |
| 179 | 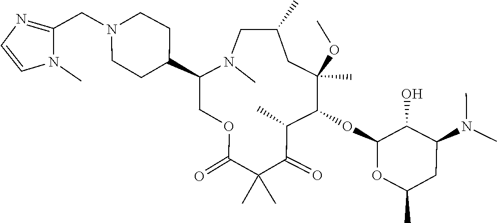 [SMILES: CO[C@]1(C)C[C@@H](C)CN(C)[C@H](C2CCN(Cc3nccn3C)CC2)COC(=O)C(C)(C)C(=O)[C@H](C)[C@H]1O[C@@H]1O[C@H](C)C[C@H](N(C)C)[C@H]1O] |
| 180 | 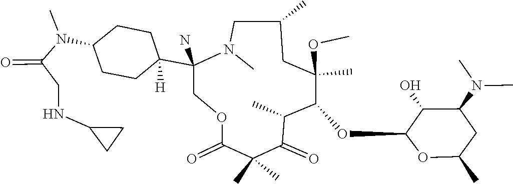 |
| 181 | 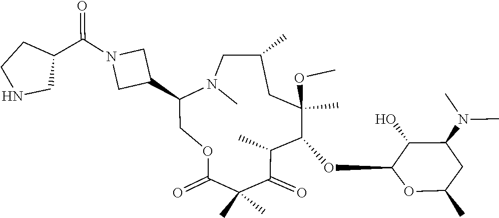 |
| 182 | 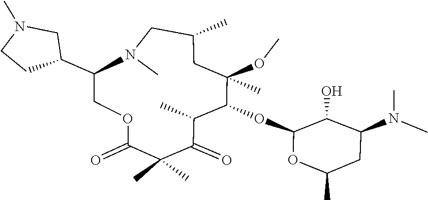 |
| 183 | 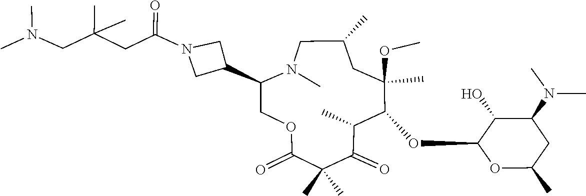 |
| 184 | 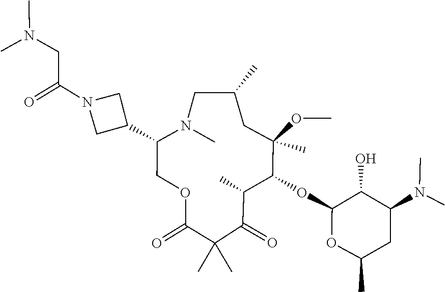 |
| 185 | 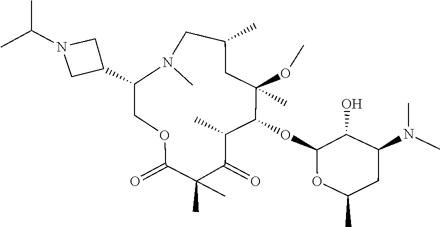 |
| 186 | 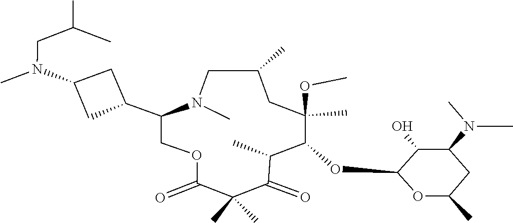 |
| 187 | 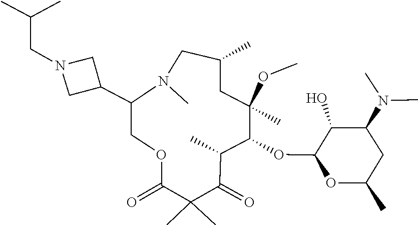 [SMILES: CO[C@]1(C)C[C@@H](C)CN(C)C(C2CN(CC(C)C)C2)COC(=O)C(C)(C)C(=O)[C@H](C)[C@H]1O[C@@H]1O[C@H](C)C[C@H](N(C)C)[C@H]1O] |
| 188 | 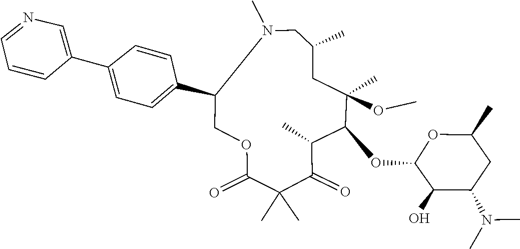 [SMILES: CO[C@]1(C)C[C@@H](C)CN(C)[C@H](c2ccc(-c3cccnc3)cc2)COC(=O)C(C)(C)C(=O)[C@H](C)[C@@H]1O[C@@H]1O[C@@H](C)C[C@H](N(C)C)[C@H]1O] |
| 189 | 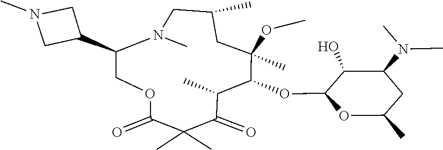 |
| 190 | 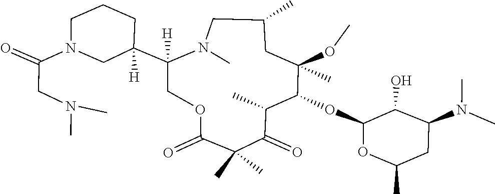 |
| 191 | 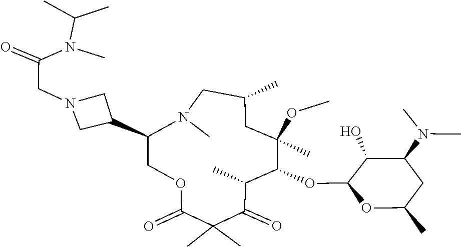 [SMILES: CO[C@]1(C)C[C@@H](C)CN(C)[C@H](C2CN(CC(=O)N(C)C(C)C)C2)COC(=O)C(C)(C)C(=O)[C@H](C)[C@H]1O[C@@H]1O[C@H](C)C[C@H](N(C)C)[C@H]1O] |
| 192 | 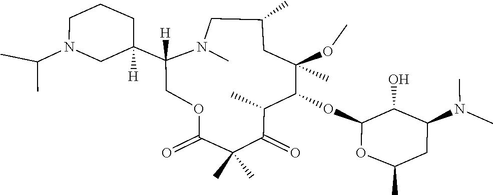 |
| 193 | 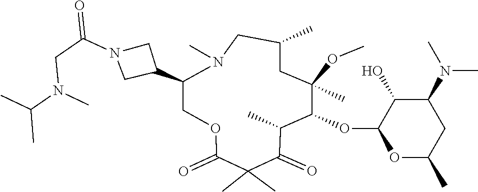 [SMILES: CO[C@]1(C)C[C@@H](C)CN(C)[C@H](C2CN(C(=O)CN(C)C(C)C)C2)COC(=O)C(C)(C)C(=O)[C@H](C)[C@H]1O[C@@H]1O[C@H](C)C[C@H](N(C)C)[C@H]1O] |
| 194 | 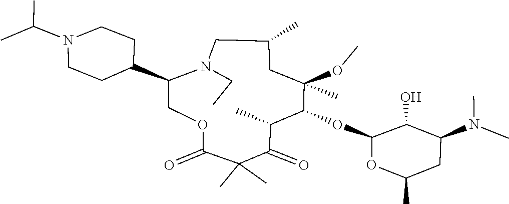 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@H]1C1CCN(C(C)C)CC1] |
| 195 | 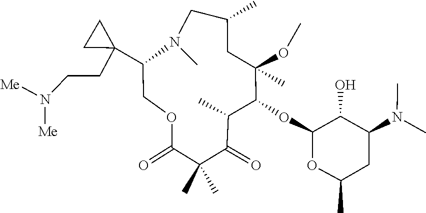 |
| 196 | 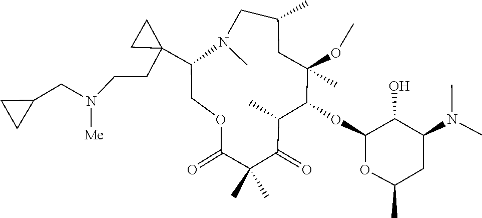 |
| 197 | 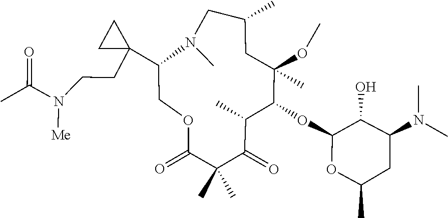 |
| 198 | 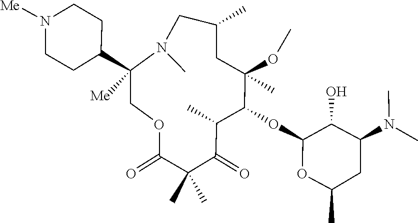 |
| 199 | 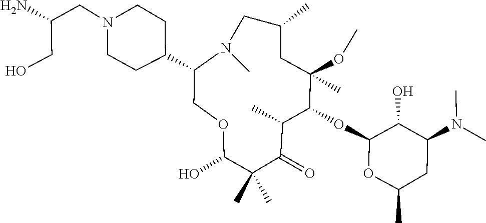 |
| 200 | 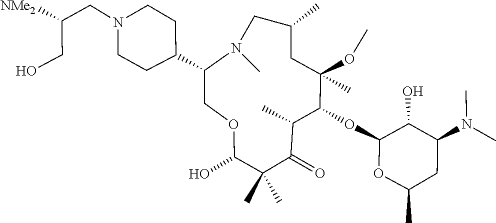 |
| 201 | 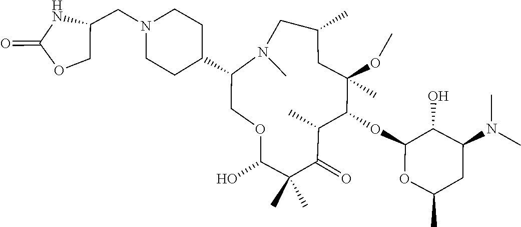 |
| 202 | 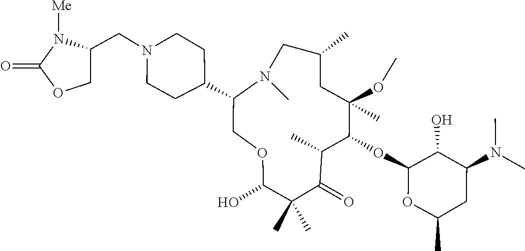 |
| 203 | 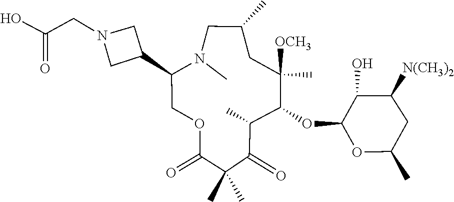 |
| 204 | 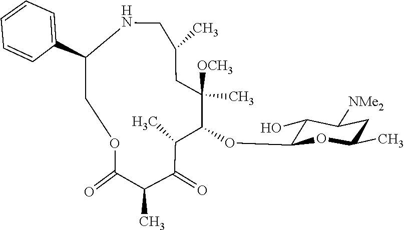 |
| 205 | 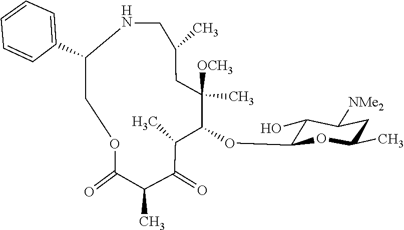 |

Unless otherwise stated, any formulae described herein are also meant to include salts, solvates, hydrates, polymorphs, co-crystals, tautomers, stereoisomers, and isotopically labeled derivatives thereof. In certain embodiments, the provided compound is a salt of any of the formulae described herein. In certain embodiments, the provided compound is a pharmaceutically acceptable salt of any of the formulae described herein. In certain embodiments, the provided compound is a solvate of any of the formulae described herein. In certain embodiments, the provided compound is a hydrate of any of the formulae described herein. In certain embodiments, the provided compound is a polymorph of any of the formulae described herein. In certain embodiments, the provided compound is a co-crystal of any of the formulae described herein. In certain embodiments, the provided compound is a tautomer of any of the formulae described herein. In certain embodiments, the provided compound is a stereoisomer of any of the formulae described herein. In certain embodiments, the provided compound is of an isotopically labeled form of any of the formulae described herein. For example, compounds having the present structures except for the replacement of hydrogen by deuterium or tritium, replacement of ¹⁹F with ¹⁸F, or the replacement of a ¹²C by a ¹³C or ¹⁴C are within the scope of the disclosure. In certain embodiments, the provided compound is a deuterated form of any of the formulae or compounds described herein.

### Additional Formulae

Provided herein are certain intermediates that may be prepared during the preparation of a macrolide described herein. Such intermediates include the eastern half of a macrolide prior to coupling and uncyclized precursors prior to macrolactonization.

In one aspect, the present disclosure provides a macrolide eastern half intermediate of Formula (M):

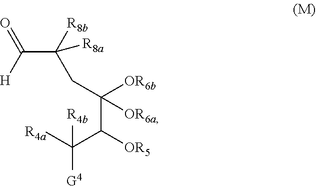

or salt thereof, wherein:

R³, R^{4a}, R^{4b}, R⁵, R^{6a}, R^{6b}, R^{8a}, and R^{8b} are as defined herein; and

G⁴ is of formula:

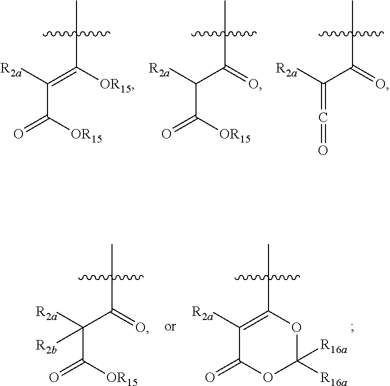

each instance of R¹⁵ is independently silyl, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R¹⁵ groups are joined to form an optionally substituted heterocyclyl or heteroaryl ring; and

each instance of R^{16a} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl.

In another aspect, the present disclosure provides an uncyclized macrolide intermediate of Formula (N):

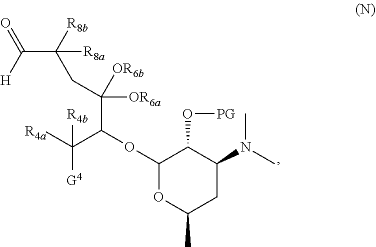

or salt thereof, wherein:
PG is a protecting group;
R₄ₐ, R_{4b}, R₅, R₆ₐ, R_{6b}, R₈ₐ, and R_{8b} are as defined herein;
G⁴ is of formula:

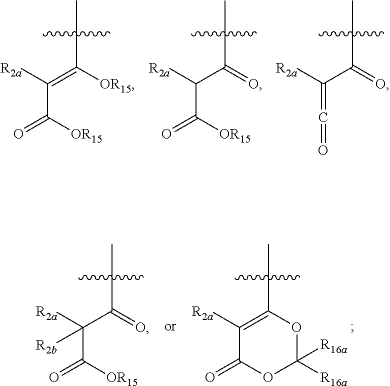

each instance of R¹⁵ is independently silyl, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R¹⁵ groups are joined to form an optionally substituted heterocyclyl or heteroaryl ring; and each instance of R^{16a} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl.

In some embodiments, -OPG is -OBz.

In certain embodiments, the compound of Formula (N) is a compound of Formula (N-a):

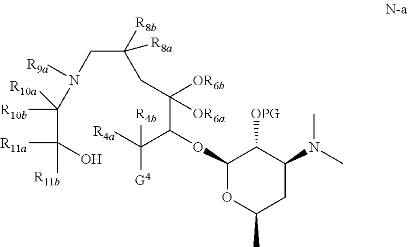

or salt thereof, wherein the variables are as defined herein.

### Preparation by Coupling and Macrolactonization

In certain embodiments, macrolides of the present disclosure are prepared by coupling a compound of Formula (N-2) (the eastern half) wherein Rₛ is a sugar residue

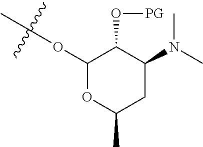

wherein PG is a protecting group and “ [symbol] ” indicates a point of attachment, and a compound of Formula (N-1) (the western half) to provide an uncyclized macrolide precursor of Formula (N-a) as depicted in Scheme 1.

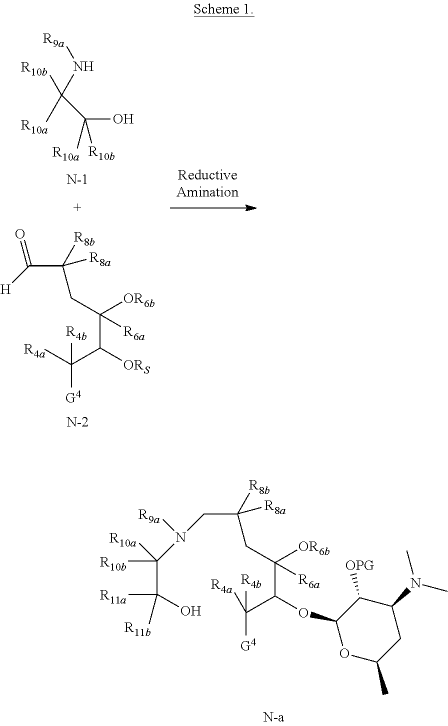

Formula (N-a) is cyclized to give, after deprotection of the sugar residue

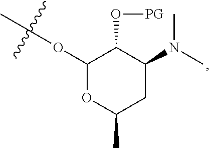

a macrolide of Formula (I) as depicted in Scheme 2.

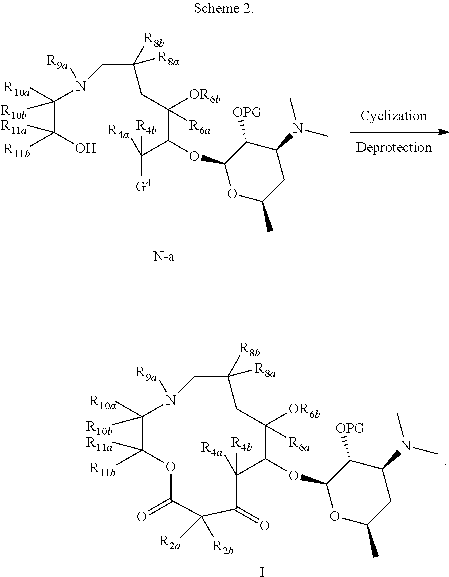

Alternatively, the macrolide precursor of Formula (N-a) is cyclized to provide a macrolide of Formula (P) (i.e., a compound of Formula (I), wherein R₉ₐ is hydrogen), which can undergo reductive amination to provide a compound of Formula (I) as shown in Scheme 3.

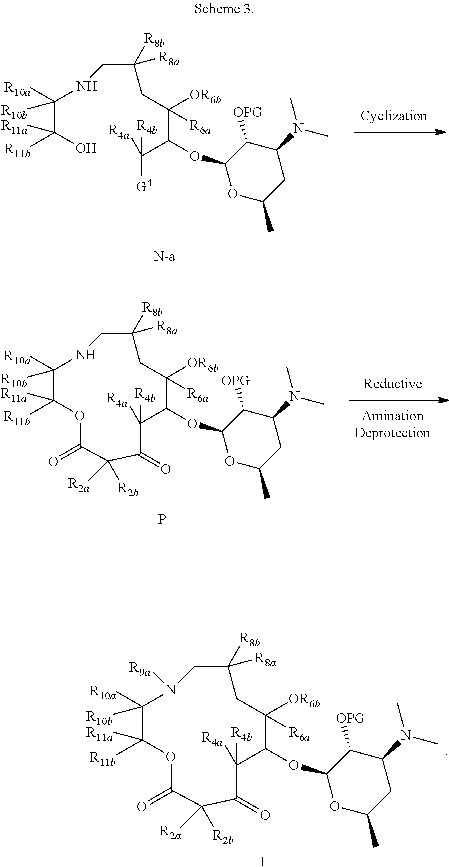

Late-stage installment of the R_{2b} group can be achieved via treatment of a compound of Formula (A) prepared as provide above with a base and a suitable electrophile group (e.g., halogenating agent or R₂-LG, wherein LG is a leaving group) as depicted in Scheme 4. The compound of Formula (A) may be prepared in the same manner as the compound of Formula (I) as depicted in Schemes 2 and 3 with the exception that one of R₂ₐ or R_{2b} is hydrogen.

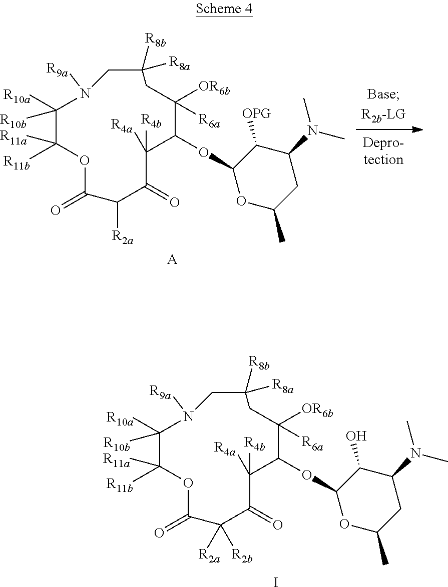

For all intermediates, the variables are as defined herein for a compound of Formula (I).

Other variables depicted for intermediates and precursors are defined as follows:

R₂ₐ is optionally substituted alkyl, optionally substituted alkenyl, optionally substituted alkynyl, optionally substituted carbocyclyl, or optionally substituted heterocyclyl;

LG is a leaving group;

G⁴ is of formula:

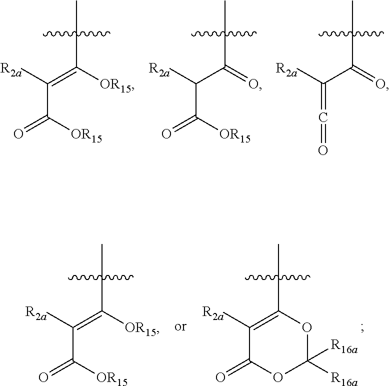

each instance of R¹⁵ is independently silyl, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R¹⁵ groups are joined to form an optionally substituted heterocyclyl or heteroaryl ring; and

each instance of R^{16a} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl.

In some embodiments, Rₛ is the sugar moiety

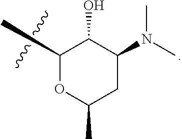

The sugar moiety is typically attached to the macrolide framework during synthesis of the eastern half, but may also be attached at other stages of the preparation. The sugar moiety may be attached by a chemical or enzymatic glycosylation reaction between the hydroxyl group at the C5 position and a glycosyl donor. In certain embodiments, the sugar moiety is attached to the macrolide framework as a thioglycoside. In certain embodiments, substituents of the sugar moiety are modified after the glycosylation of the macrolide or macrolide precursor (e.g., eastern half).

### Pharmaceutical Compositions and Administration

The present disclosure provides pharmaceutical compositions comprising a macrolide as described herein, or a pharmaceutically acceptable salt thereof, and a pharmaceutically acceptable excipient.

Pharmaceutically acceptable excipients include any and all solvents, diluents, or other liquid vehicles, dispersions, suspension aids, surface active agents, isotonic agents, thickening or emulsifying agents, preservatives, solid binders, lubricants and the like, as suited to the particular dosage form desired. General considerations in formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions described herein can be prepared by any method known in the art of pharmacology. In general, such preparatory methods include the steps of bringing the macrolide of the present invention into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the macrolide of the present invention. The amount of the macrolide is generally equal to the dosage of the macrolide which would be administered to a subject and/or a convenient fraction of such a dosage such as, for example, one-half or one-third of such a dosage.

Relative amounts of the macrolide, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) macrolide.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Liquid dosage forms for oral and parenteral administration include pharmaceutically acceptable emulsions, microemulsions, solutions, suspensions, syrups and elixirs. In addition to the macrolides, the liquid dosage forms may comprise inert diluents commonly used in the art such as, for example, water or other solvents, solubilizing agents, and emulsifiers, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium.

Solid dosage forms for oral administration include capsules, tablets, pills, powders, and granules. In such solid dosage forms, the macrolide is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Dosage forms for topical and/or transdermal administration of a macrolide of this invention may include ointments, pastes, creams, lotions, gels, powders, solutions, sprays, inhalants and/or patches. Generally, the macrolide is admixed under sterile conditions with a pharmaceutically acceptable carrier and/or any needed preservatives and/or buffers as can be required.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Macrolides provided herein are typically formulated in dosage unit form for ease of administration and uniformity of dosage. It will be understood, however, that the total daily amount of the macrolide will be decided by the attending physician within the scope of sound medical judgment. The specific therapeutically effective dose level for any particular subject will depend upon a variety of factors including the disease, disorder, or condition being treated and the severity of the disorder; the activity of the specific macrolide employed; the specific composition employed; the age, body weight, general health, sex and diet of the subject; the time of administration, route of administration, and rate of excretion of the specific macrolide employed; the duration of the treatment; drugs used in combination or coincidental with the specific macrolide employed; and like factors well known in the medical arts.

The macrolides and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. In general, the most appropriate route of administration will depend upon a variety of factors including the nature of the agent, the therapeutic regimen, and/or the condition of the subject. Oral administration is the preferred mode of administration. However, in certain embodiments, the subject may not be in a condition to tolerate oral administration, and thus intravenous, intramuscular, and/or rectal administration are also preferred alternative modes of administration.

An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein.

It will be also appreciated that a macrolide or composition, as described herein, can be administered in combination with one or more additional therapeutically active agents. The macrolide or composition can be administered concurrently with, prior to, or subsequent to, one or more additional therapeutically active agents. In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of the inventive macrolide with the additional therapeutically active agent and/or the desired therapeutic effect to be achieved. In general, it is expected that additional therapeutically active agents utilized in combination be utilized at levels that do not exceed the levels at which they are utilized individually. In certain embodiments, the levels utilized in combination will be lower than those utilized individually.

Exemplary additional therapeutically active agents include, but are not limited to, antibiotics, anti-viral agents, anesthetics, anti-coagulants, inhibitors of an enzyme, steroidal agents, steroidal or non-steroidal anti-inflammatory agents, antihistamine, immunosuppressant agents, antigens, vaccines, antibodies, decongestant, sedatives, opioids, pain-relieving agents, analgesics, anti-pyretics, hormones, and prostaglandins. Therapeutically active agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

In certain embodiments, the additional therapeutically active agent is an antibiotic. Exemplary antibiotics include, but are not limited to, penicillins (e.g., penicillin, amoxicillin), cephalosporins (e.g., cephalexin), macrolides (e.g., erythromycin, clarithormycin, azithromycin, troleandomycin), fluoroquinolones (e.g., ciprofloxacin, levofloxacin, ofloxacin), sulfonamides (e.g., co-trimoxazole, trimethoprim), tetracyclines (e.g., tetracycline, chlortetracycline, oxytetracycline, demeclocycline, methacycline, sancycline, doxycline, aureomycin, terramycin, minocycline, 6-deoxytetracycline, lymecycline, meclocycline, methacycline, rolitetracycline, and glycylcycline antibiotics (e.g., tigecycline)), aminoglycosides (e.g., gentamicin, tobramycin, paromomycin), aminocyclitol (e.g., spectinomycin), chloramphenicol, sparsomycin, and quinupristin/dalfoprisin (Syndercid™).

Also encompassed by the invention are kits (e.g., pharmaceutical packs). The kits provided may comprise an inventive pharmaceutical composition or macrolide and a container (e.g., a vial, ampule, bottle, syringe, and/or dispenser package, or other suitable container). In certain embodiments, provided kits may optionally further include a second container comprising a pharmaceutical excipient for dilution or suspension of an inventive pharmaceutical composition or macrolide. In certain embodiments, the inventive pharmaceutical composition or macrolide provided in the container and the second container are combined to form one unit dosage form.

### Methods of Treatment and Uses

The present disclosure contemplates using macrolides of the present invention for the treatment of infectious diseases, for example, fungal, bacterial, viral, or parasitic infections, and for the treatment of inflammatory conditions. Ketolides are known to exhibit anti-bacterial activity as well as anti-parasitic activity. See, for example, Clark et al., Bioorganic & Medicinal Chemistry Letters (2000) 10:815-819 (anti-bacterial activity); and Lee et al., J Med. Chem. (2011) 54:2792-2804 (anti-bacterial and anti-parasitic activity). Ketolides are also known to exhibit an anti-inflammatory effect. See, for example, Amsden, Journal of Antimicrobial Chemotherapy (2005) 55:10-21 (chronic pulmonary inflammatory syndromes).

Thus, as generally described herein, provided is a method of treating an infectious disease comprising administering an effective amount of a macrolide of the present disclosure, or a pharmaceutically acceptable salt thereof, to a subject in need thereof. Such a method can be conducted in vivo (i.e., by administration to a subject) or in vitro (e.g., upon contact with the pathogen, tissue, or cell culture). Treating, as used herein, encompasses therapeutic treatment and prophylactic treatment.

In certain embodiments, the effective amount is a therapeutically effective amount. For example, in certain embodiments, the method slows the progress of an infectious disease in the subject. In certain embodiments, the method improves the condition of the subject suffering from an infectious disease. In certain embodiments, the subject has a suspected or confirmed infectious disease.

In certain embodiments, the effective amount is a prophylactically effective amount. For example, in certain embodiments, the method prevents or reduces the likelihood of an infectious disease, e.g., in certain embodiments, the method comprises administering a macrolide of the present invention to a subject in need thereof in an amount sufficient to prevent or reduce the likelihood of an infectious disease. In certain embodiments, the subject is at risk of an infectious disease (e.g., has been exposed to another subject who has a suspected or confirmed infectious disease or has been exposed or thought to be exposed to a pathogen).

In another aspect, provided is an in vitro method of inhibiting pathogenic growth comprising contacting an effective amount of the macrolide of the present invention with a pathogen (e.g., a bacteria, virus, fungus, or parasite) in a cell culture.

As used herein, “infectious disease” and “microbial infection” are used interchangeably, and refer to an infection with a pathogen, such as a fungus, bacteria, virus, or a parasite. In certain embodiments, the infectious disease is caused by a pathogen resistant to other treatments. In certain embodiments, the infectious disease is caused by a pathogen that is multidrug tolerant or resistant, e.g., the infectious disease is caused by a pathogen that neither grows nor dies in the presence of or as a result of other treatments.

In certain embodiments, the infectious disease is a bacterial infection. For example, in certain embodiments, provided is a method of treating a bacterial infection comprising administering an effective amount of a macrolide of the present invention, or a pharmaceutically acceptable salt thereof, to a subject in need thereof.

In certain embodiments, the macrolide has a mean inhibitory concentration (MIC), with respect to a particular bacterial isolate, of less than 50 μg/mL, less than 25 μg/mL, less than 20 μg/mL, less than 10 μg/mL, less than 5 μg/mL, or less than 1 μg/mL.

In certain embodiments, the bacterial isolate is susceptible (e.g., responds to) or resistant to known commercial macrolides, such as azithromycin, clindamycin, telithromycin, erythromycin, spiramycin, and the like. In certain embodiments, the bacterial isolate is resistant to a known macrolide. For example, in certain embodiments, the bacterium is erythromycin resistant (ER). In certain other embodiments, the bacterium is azithromycin resistant (AR).

In certain embodiments, the bacterial infection is resistant to other antibiotics (e.g., non-macrolide) therapy. For example, in certain embodiments, the pathogen is vancomycin resistant (VR). In certain embodiments, the pathogen is methicillin-resistant (MR), e.g., in certain embodiments, the bacterial infection is a methicillin-resistant S. aureus infection (a MRSA infection). In certain embodiments, the pathogen is quinolone resistant (QR). In certain embodiments, the pathogen is fluoroquinolone resistant (FR).

In certain embodiments, the bacterial isolates have an efflux (e.g., mef, msr) genotype. In certain embodiments, the bacteria have a methylase (e.g., erm) genotype. In certain embodiments, the bacterial isolates have a constitutive genotype. In certain embodiments, the bacterial isolates have an inducible genotype.

Exemplary bacterial infections include, but are not limited to, infections with a Gram positive bacteria (e.g., of the phylum Actinobacteria, phylum Firmicutes, or phylum Tenericutes); Gram negative bacteria (e.g., of the phylum Aquificae, phylum Deinococcus-Thermus, phylum Fibrobacteres/Chlorobi/Bacteroidetes (FCB), phylum Fusobacteria, phylum Gemmatimonadest, phylum Ntrospirae, phylum Planctomycetes/Verrucomicrobia/Chlamydiae (PVC), phylum Proteobacteria, phylum Spirochaetes, or phylum Synergistetes); or other bacteria (e.g., of the phylum Acidobacteria, phylum Chlroflexi, phylum Chrystiogenetes, phylum Cyanobacteria, phylum Deferrubacteres, phylum Dictyoglomi, phylum Thermodesulfobacteria, or phylum Thermotogae).

In certain embodiments, the bacterial infection is an infection with a Gram positive bacterium.

In certain embodiments, the Gram positive bacterium is a bacterium of the phylum Firmicutes.

In certain embodiments, the bacteria are members of the phylum Firmicutes and the genus Enterococcus, i.e., the bacterial infection is an Enterococcus infection. Exemplary Enterococci bacteria include, but are not limited to, E. avium, E. durans, E. faecalis, E. faecium, E. gallinarum, E. solitarius, E. casseliflavus, and E. raffinosus.

In certain embodiments, the bacteria are members of the phylum Firmicutes and the genus Staphylococcus, i.e., the bacterial infection is a Staphylococcus infection. Exemplary Staphylococci bacteria include, but are not limited to, S. arlettae, S. aureus, S. auricularis, S. capitis, S. caprae, S. carnous, S. chromogenes, S. cohii, S. condimenti, S. croceolyticus, S. delphini, S. devriesei, S. epidermis, S. equorum, S. felis, S. fluroettii, S. gallinarum, S. haemolyticus, S. hominis, S. hyicus, S. intermedius, S. kloosii, S. leei, S. lenus, S. lugdunesis, S. lutrae, S. lyticans, S. massiliensis, S. microti, S. muscae, S. nepalensis, S. pasteuri, S. penttenkoferi, S. piscifermentans, S. psuedointermedius, S. psudolugdensis, S. pulvereri, S. rostri, S. saccharolyticus, S. saprophyticus, S. schleiferi, S. sciuri, S. simiae, S. simulans, S. stepanovicii, S. succinus, S. vitulinus, S. warneri, and S. xylosus. In certain embodiments, the Staphylococcus infection is an S. aureus infection. In certain embodiments, the S. aureus has an efflux (e.g., mef, msr) genotype. In certain embodiments, the S. aureus has a methylase (e.g., erm) genotype.

In certain embodiments, the bacteria are members of the phylum Firmicutes and the genus Bacillus, i.e., the bacterial infection is a Bacillus infection. Exemplary Bacillus bacteria include, but are not limited to, B. alcalophilus, B. alvei, B. aminovorans, B. amyloliquefaciens, B. aneurinolyticus, B. anthracis, B. aquaemaris, B. atrophaeus, B. boroniphilus, B. brevis, B. caldolyticus, B. centrosporus, B. cereus, B. circulans, B. coagulans, B. firmus, B. flavothermus, B. fusiformis, B. globigii, B. infernus, B. larvae, B. laterosporus, B. lentus, B. licheniformis, B. megaterium, B. mesentericus, B. mucilaginosus, B. mycoides, B. natto, B. pantothenticus, B. polymyxa, B. pseudoanthracis, B. pumilus, B. schlegelii, B. sphaericus, B. sporothermodurans, B. stearothermophilus, B. subtilis, B. thermoglucosidasius, B. thuringiensis, B. vulgatis, and B. weihenstephanensis. In certain embodiments, the Bacillus infection is a B. subtilis infection. In certain embodiments, the B. subtilis has an efflux (e.g., mef, msr) genotype. In certain embodiments, the B. subtilis has a methylase (e.g., erm) genotype.

In certain embodiments, the bacteria are members of the phylum Firmicutes and the genus Streptococcus, i.e., the bacterial infection is a Streptococcus infection. Exemplary Streptococcus bacteria include, but are not limited to, S. agalactiae, S. anginosus, S. bovis, S. canis, S. constellatus, S. dysgalactiae, S. equinus, S. iniae, S. intermedius, S mitis, S. mutans, S. oralis, S. parasanguinis, S peroris, S pneumoniae, S. pyogenes, S. ratti, S. salivarius, S. thermophilus, S. sanguinis, S. sobrinus, S suis, S uberis, S. vestibularis, S. viridans, and S. zooepidemicus. In certain embodiments, the Streptococcus infection is an S. pyogenes infection. In certain embodiments, the Streptococcus infection is an S. pneumoniae infection. In certain embodiments, the S. pneumoniae has an efflux (e.g., mef, msr) genotype. In certain embodiments, the S. pneumoniae has a methylase (e.g., erm) genotype.

In certain embodiments, the bacteria are members of the phylum Actinobacteria and the genus Mycobacterium, i.e., the bacterial infection is a Mycobacterium infection. Exemplary Mycobacteriaceae bacteria include, but are not limited to, M. tuberculosis, M. avium, M. gordonae, M. kansasi, M. nonchromogenicum, M. terrae, M. ulcerans, M. simiae, M. leprae, M. abscessus, M. chelonae, M. fortuitum, M. mucogenicum, M. parafortuitum, and M. vaccae.

In certain embodiments, the bacterial infection is an infection with a Gram negative bacteria.

In certain embodiments, the Gram negative bacteria are bacteria of the phylum Proteobacteria and the genus Escherichia. i.e., the bacterial infection is an Escherichia infection. Exemplary Escherichia bacteria include, but are not limited to, E. albertii, E. blattae, E. coli, E. fergusonii, E. hermannii, and E. vulneris. In certain embodiments, the Escherichia infection is an E. coli infection.

In certain embodiments, the Gram negative bacteria are bacteria of the phylum Proteobacteria and the genus Haemophilus. i.e., the bacterial infection is an Haemophilus infection. Exemplary Haemophilus bacteria include, but are not limited to, H. aegyptius, H. aphrophilus, H. avium, H. ducreyi, H. felis, H. haemolyticus, H. influenzae, H. parainfluenzae, H. paracuniculus, H. parahaemolyticus, H. pittmaniae, Haemophilus segnis, and H. somnus. In certain embodiments, the Haemophilus infection is an H. influenzae infection.

In certain embodiments, the Gram negative bacteria are bacteria of the phylum Proteobacteria and the genus Acinetobacter. i.e., the bacterial infection is an Acinetobacter infection. Exemplary Acinetobacter bacteria include, but are not limited to, A. baumanii, A. haemolyticus, and A. lwoffri. In certain embodiments, the Acinetobacter infection is an A. baumanii infection.

In certain embodiments, the Gram negative bacteria are bacteria of the phylum Proteobacteria and the genus Klebsiella. i.e., the bacterial infection is a Klebsiella infection. Exemplary Klebsiella bacteria include, but are not limited to, K. granulomatis, K. oxytoca, K. michiganensis, K. pneumoniae, K. quasipneumoniae, and K. variicola. In certain embodiments, the Klebsiella infection is a K. pneumoniae infection.

In certain embodiments, the Gram negative bacteria are bacteria of the phylum Proteobacteria and the genus Pseudomonas. i.e., the bacterial infection is a Pseudomonas infection. Exemplary Pseudomonas bacteria include, but are not limited to, P. aeruginosa, P. oryzihabitans, P. plecoglissicida, P. syringae, P. putida, and P. fluoroscens. In certain embodiments, the Pseudomonas infection is a P. aeruginosa infection.

In certain embodiments, the bacterium is an atypical bacteria, i.e., are neither Gram positive nor Gram negative.

In certain embodiments, the infectious disease is an infection with a parasitic infection. Thus, in certain embodiments, provided is a method of treating a parasitic infection comprising administering an effective amount of a macrolide of the present invention, or a pharmaceutically acceptable salt thereof, to a subject in need thereof.

In certain embodiments, the macrolide has an IC₅₀ (uM) with respect to a particular parasite, of less than 50 uM, less than 25 uM, less than 20 uM, less than 10 uM, less than 5 uM, or less than 1 uM.

Exemplary parasites include, but are not limited to, Trypanosoma spp. (e.g., Trypanosoma cruzi, Trypansosoma brucei), Leishmania spp., Giardia spp., Trichomonas spp., Entamoeba spp., Naegleria spp., Acanthamoeba spp., Schistosoma spp., Plasmodium spp. (e.g., P. flaciparum), Crytosporidium spp., Isospora spp., Balantidium spp., Loa Loa, Ascaris lumbricoides, Dirofilaria immitis, and Toxoplasma ssp. (e.g. T. gondii).

As generally described herein, the present disclosure further provides a method of treating an inflammatory condition comprising administering an effective amount of a macrolide of the present disclosure, or a pharmaceutically acceptable salt thereof, to a subject in need thereof. Such a method can be conducted in vivo (i.e., by administration to a subject) or in vitro (e.g., upon contact with the pathogen, tissue, or cell culture). Treating, as used herein, encompasses therapeutic treatment and prophylactic treatment.

In certain embodiments, the effective amount is a therapeutically effective amount. For example, in certain embodiments, the method slows the progress of an inflammatory condition in the subject. In certain embodiments, the method improves the condition of the subject suffering from an inflammatory condition. In certain embodiments, the subject has a suspected or confirmed inflammatory condition.

In certain embodiments, the effective amount is a prophylatically effective amount. For example, in certain embodiments, the method prevents or reduces the likelihood of an inflammatory condition, e.g., in certain embodiments, the method comprises administering a macrolide of the present invention to a subject in need thereof in an amount sufficient to prevent or reduce the likelihood of an inflammatory condition. In certain embodiments, the subject is at risk to an inflammatory condition.

In another aspect, provided is an in vitro method of treating an inflammatory condition comprising contacting an effective amount of the macrolide of the present invention with an inflammatory cell culture.

The term “inflammatory condition” refers to those diseases, disorders, or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent). Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation.

Exemplary inflammatory conditions include, but are not limited to, chronic pulmonary inflammatory syndromes (e.g., diffuse panbronchiolitis, cystic fibrosis, asthma, bronchiectasis, and chronic obstructive pulmonary disease).

In certain embodiments, the inflammatory condition is an acute inflammatory condition (e.g., for example, inflammation resulting from an infection). In certain embodiments, the inflammatory condition is a chronic inflammatory condition. In certain embodiments, the inflammatory condition is inflammation associated with cancer.

### Definitions

### Chemical Terms

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H. Tables of Resolving Agents and Optical Resolutions p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

In a formula, [symbol] is a single bond where the stereochemistry of the moieties immediately attached thereto is not specified, [symbol] is absent or a single bond, and [symbol] or [symbol] is a single or double bond. When a variable is defined generically, with a number of possible substituents, each individual radical can be defined with our without the bond. For example, if Rzz can be hydrogen, this can be indicated as “—H” or “H” in the definition of Rzz.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₁₀ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl. The ranges can be written as, for example, C₁₋₁₀ or as C₁-C₁₀.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In certain embodiments, an alkyl group has 1 to 9 carbon atoms (“C₁₋₉ alkyl”). In certain embodiments, an alkyl group has 1 to 8 carbon atoms (“C₁₋₈ alkyl”). In certain embodiments, an alkyl group has 1 to 7 carbon atoms (“C₁₋₇ alkyl”). In certain embodiments, an alkyl group has 1 to 6 carbon atoms (“C₁₋₆ alkyl”). In certain embodiments, an alkyl group has 1 to 5 carbon atoms (“C₁₋₅ alkyl”). In certain embodiments, an alkyl group has 1 to 4 carbon atoms (“C₁₋₄ alkyl”). In certain embodiments, an alkyl group has 1 to 3 carbon atoms (“C₁₋₃ alkyl”). In certain embodiments, an alkyl group has 1 to 2 carbon atoms (“C₁₋₂ alkyl”). In certain embodiments, an alkyl group has 1 carbon atom (“C₁ alkyl”). In certain embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In certain embodiments, the haloalkyl moiety has 1 to 8 carbon atoms (“C₁₋₈ haloalkyl”). In certain embodiments, the haloalkyl moiety has 1 to 6 carbon atoms (“C₁₋₆ haloalkyl”). In certain embodiments, the haloalkyl moiety has 1 to 4 carbon atoms (“C₁₋₄ haloalkyl”). In certain embodiments, the haloalkyl moiety has 1 to 3 carbon atoms (“C₁₋₃ haloalkyl”). In certain embodiments, the haloalkyl moiety has 1 to 2 carbon atoms (“C₁₋₂ haloalkyl”). Examples of haloalkyl groups include —CF₃, —CF₂CF₃, —CF₂CF₂CF₃, —CCl₃, —CFCl₂, —CF₂Cl, and the like.

The term “alkoxy” refers to a moiety of the formula —OR′, wherein R′ is an (C₁-C₆)alkyl moiety as defined herein. The term “Cₙ₋ₘ alkoxy” or (Cₙ-Cₘ) alkoxy refers to an alkoxy group, the alkyl group of which has n to m carbons. Examples of alkoxy moieties include, but are not limited to, methoxy, ethoxy, isopropoxy, and the like.

The term “hydroxyalkyl” refers to a moiety of the formula HOR′, wherein R′ is an (C₁-C₆)alkyl moiety as defined herein. The term “Cₙ₋ₘ alkoxy” or (Cₙ-Cₘ) alkoxy refers to an alkoxy group, the alkyl group of which has n to m carbons. Examples of alkoxy moieties include, but are not limited to, methoxy, ethoxy, isopropoxy, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In certain embodiments, a heteroalkyl group is a saturated group having 1 to 9 carbon atoms and 1 or more heteroatoms within the parent chain (“heteroC₁₋₉ alkyl”). In certain embodiments, a heteroalkyl group is a saturated group having 1 to 8 carbon atoms and 1 or more heteroatoms within the parent chain (“heteroC₁₋₈ alkyl”). In certain embodiments, a heteroalkyl group is a saturated group having 1 to 7 carbon atoms and 1 or more heteroatoms within the parent chain (“heteroC₁₋₇ alkyl”). In certain embodiments, a heteroalkyl group is a saturated group having 1 to 6 carbon atoms and 1 or more heteroatoms within the parent chain (“heteroC₁₋₆ alkyl”). In certain embodiments, a heteroalkyl group is a saturated group having 1 to 5 carbon atoms and 1 or 2 heteroatoms within the parent chain (“heteroC₁₋₅ alkyl”). In certain embodiments, a heteroalkyl group is a saturated group having 1 to 4 carbon atoms and 1 or 2 heteroatoms within the parent chain (“heteroC₁₋₄ alkyl”). In certain embodiments, a heteroalkyl group is a saturated group having 1 to 3 carbon atoms and 1 heteroatom within the parent chain (“heteroC₁₋₃ alkyl”). In certain embodiments, a heteroalkyl group is a saturated group having 1 to 2 carbon atoms and 1 heteroatom within the parent chain (“heteroC₁₋₂ alkyl”). In certain embodiments, a heteroalkyl group is a saturated group having 1 carbon atom and 1 heteroatom (“heteroC₁ alkyl”). In certain embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In certain embodiments, an alkenyl group has 2 to 9 carbon atoms (“C₂₋₉ alkenyl”). In certain embodiments, an alkenyl group has 2 to 8 carbon atoms (“C₂₋₈ alkenyl”). In certain embodiments, an alkenyl group has 2 to 7 carbon atoms (“C₂₋₇ alkenyl”). In certain embodiments, an alkenyl group has 2 to 6 carbon atoms (“C₂₋₆ alkenyl”). In certain embodiments, an alkenyl group has 2 to 5 carbon atoms (“C₂₋₅ alkenyl”). In certain embodiments, an alkenyl group has 2 to 4 carbon atoms (“C₂₋₄ alkenyl”). In certain embodiments, an alkenyl group has 2 to 3 carbon atoms (“C₂₋₃ alkenyl”). In certain embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

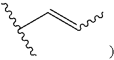

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In certain embodiments, a heteroalkenyl group has 2 to 9 carbon atoms at least one double bond, and 1 or more heteroatoms within the parent chain (“heteroC₂₋₉ alkenyl”). In certain embodiments, a heteroalkenyl group has 2 to 8 carbon atoms, at least one double bond, and 1 or more heteroatoms within the parent chain (“heteroC₂₋₈ alkenyl”). In certain embodiments, a heteroalkenyl group has 2 to 7 carbon atoms, at least one double bond, and 1 or more heteroatoms within the parent chain (“heteroC₂₋₇ alkenyl”). In certain embodiments, a heteroalkenyl group has 2 to 6 carbon atoms, at least one double bond, and 1 or more heteroatoms within the parent chain (“heteroC₂₋₆ alkenyl”). In certain embodiments, a heteroalkenyl group has 2 to 5 carbon atoms, at least one double bond, and 1 or 2 heteroatoms within the parent chain (“heteroC₂₋₈ alkenyl”). In certain embodiments, a heteroalkenyl group has 2 to 4 carbon atoms, at least one double bond, and 1 or 2 heteroatoms within the parent chain (“heteroC₂₋₄ alkenyl”). In certain embodiments, a heteroalkenyl group has 2 to 3 carbon atoms, at least one double bond, and 1 heteroatom within the parent chain (“heteroC₂₋₃ alkenyl”). In certain embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In certain embodiments, an alkynyl group has 2 to 9 carbon atoms (“C₂₋₉ alkynyl”). In certain embodiments, an alkynyl group has 2 to 8 carbon atoms (“C₂₋₈ alkynyl”). In certain embodiments, an alkynyl group has 2 to 7 carbon atoms (“C₂₋₇ alkynyl”). In certain embodiments, an alkynyl group has 2 to 6 carbon atoms (“C₂₋₆ alkynyl”). In certain embodiments, an alkynyl group has 2 to 5 carbon atoms (“C₂₋₅ alkynyl”). In certain embodiments, an alkynyl group has 2 to 4 carbon atoms (“C₂₋₄ alkynyl”). In certain embodiments, an alkynyl group has 2 to 3 carbon atoms (“C₂₋₃ alkynyl”). In certain embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In certain embodiments, a heteroalkynyl group has 2 to 9 carbon atoms, at least one triple bond, and 1 or more heteroatoms within the parent chain (“heteroC₂₋₉ alkynyl”). In certain embodiments, a heteroalkynyl group has 2 to 8 carbon atoms, at least one triple bond, and 1 or more heteroatoms within the parent chain (“heteroC₂₋₈ alkynyl”). In certain embodiments, a heteroalkynyl group has 2 to 7 carbon atoms, at least one triple bond, and 1 or more heteroatoms within the parent chain (“heteroC₂₋₇ alkynyl”). In certain embodiments, a heteroalkynyl group has 2 to 6 carbon atoms, at least one triple bond, and 1 or more heteroatoms within the parent chain (“heteroC₂₋₆ alkynyl”). In certain embodiments, a heteroalkynyl group has 2 to 5 carbon atoms, at least one triple bond, and 1 or 2 heteroatoms within the parent chain (“heteroC₂₋₅ alkynyl”). In certain embodiments, a heteroalkynyl group has 2 to 4 carbon atoms, at least one triple bond, and 1 or 2 heteroatoms within the parent chain (“heteroC₂₋₄ alkynyl”). In certain embodiments, a heteroalkynyl group has 2 to 3 carbon atoms, at least one triple bond, and 1 heteroatom within the parent chain (“heteroC₂₋₃ alkynyl”). In certain embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In certain embodiments, a carbocyclyl group has 3 to 10 ring carbon atoms (“C₃₋₁₀ carbocyclyl”). In certain embodiments, a carbocyclyl group has 3 to 8 ring carbon atoms (“C₃₋₈ carbocyclyl”). In certain embodiments, a carbocyclyl group has 3 to 7 ring carbon atoms (“C₃₋₇ carbocyclyl”). In certain embodiments, a carbocyclyl group has 3 to 6 ring carbon atoms (“C₃₋₆ carbocyclyl”). In certain embodiments, a carbocyclyl group has 4 to 6 ring carbon atoms (“C₄₋₆ carbocyclyl”). In certain embodiments, a carbocyclyl group has 5 to 6 ring carbon atoms (“C₅₋₆ carbocyclyl”). In certain embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In certain embodiments, “carbocyclyl” is a monocyclic, saturated carbocyclyl group having from 3 to 14 ring carbon atoms (“C₃₋₁₄ cycloalkyl”). In certain embodiments, a cycloalkyl group has 3 to 10 ring carbon atoms (“C₃₋₁₀ cycloalkyl”). In certain embodiments, a cycloalkyl group has 3 to 8 ring carbon atoms (“C₃₋₈ cycloalkyl”). In certain embodiments, a cycloalkyl group has 3 to 6 ring carbon atoms (“C₃₋₆ cycloalkyl”). In certain embodiments, a cycloalkyl group has 4 to 6 ring carbon atoms (“C₄₋₆ cycloalkyl”). In certain embodiments, a cycloalkyl group has 5 to 6 ring carbon atoms (“C₅₋₆ cycloalkyl”). In certain embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

A cycloalkyl group can be partially unsaturated. “Partially unsaturated” means that at least one of the single bonds of the cycloalkyl group can be replaced by a double bond.

The term “heterocycloalkyl” or heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In certain embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In certain embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In certain embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In certain embodiments, the 5-6 membered heterocyclyl has 1-3 ring heteroatoms selected from nitrogen, oxygen, and sulfur. In certain embodiments, the 5-6 membered heterocyclyl has 1-2 ring heteroatoms selected from nitrogen, oxygen, and sulfur. In certain embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetra-hydro-benzo-thienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetra-hydro-pyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

A heterocycloalkyl group can be partially unsaturated. “Partially unsaturated” means that at least one of the single bonds of the heterocycloalkyl group can be replaced by a double bond.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In certain embodiments, an aryl group has 6 ring carbon atoms (“C₆ aryl”; e.g., phenyl). In certain embodiments, an aryl group has 10 ring carbon atoms (“C₁₀ aryl”; e.g., naphthyl such as 1-naphthyl and 2-naphthyl). In certain embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In certain embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In certain embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In certain embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In certain embodiments, the 5-6 membered heteroaryl has 1-3 ring heteroatoms selected from nitrogen, oxygen, and sulfur. In certain embodiments, the 5-6 membered heteroaryl has 1-2 ring heteroatoms selected from nitrogen, oxygen, and sulfur. In certain embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Heteroaralkyl” is a subset of “alkyl” and refers to an alkyl group substituted by a heteroaryl group, wherein the point of attachment is on the alkyl moiety.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen (halo), —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂₀R^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR′, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{aa}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{aa}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R is, independently, selected from hydrogen, C₁₋₁₀ alkyl, C₁₋₁₀ perhaloalkyl, C₂₋₁₀ alkenyl, C₂-10 alkynyl, heteroC₁₋₂₀alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{aa})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NRf)N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R⁹⁹ substituents can be joined to form ═O or ═S; wherein X⁻ is a counterion.

The term “halo” or “halogen” refers to fluorine (fluoro, —F), chlorine (chloro, —C₁), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{aa})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(NR^{bb})₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(NR^{bb})₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “silyl” refers to the group —Si(R^{aa})₃, wherein R^{aa} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{aa})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{aa}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{CC} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

As used herein, a “leaving group” (LG) is an art-understood term referring to a molecular fragment that departs with a pair of electrons in heterolytic bond cleavage, wherein the molecular fragment is an anion or neutral molecule. As used herein, a leaving group can be an atom or a group capable of being displaced by a nucleophile. See, for example, Smith, March Advanced Organic Chemistry 6th ed. (501-502). Exemplary leaving groups include, but are not limited to, halo (e.g., chloro, bromo, iodo), —OR^{aa} (when the 0 atom is attached to a carbonyl group, wherein R^{aa} is as defined herein), —O(C═O)R^{LG}, or —O(SO)₂R^{LG} (e.g., tosyl, mesyl, besyl), wherein R^{LG} is optionally substituted alkyl, optionally substituted aryl, or optionally substituted heteroaryl. In certain embodiments, the leaving group is a halogen. In certain embodiments, the leaving group is 1.

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

The term “carbohydrate” or “saccharide” refers to an aldehydic or ketonic derivative of polyhydric alcohols. Carbohydrates include compounds with relatively small molecules (e.g., sugars) as well as macromolecular or polymeric substances (e.g., starch, glycogen, and cellulose polysaccharides). The term “sugar” refers to monosaccharides, disaccharides, or polysaccharides. Monosaccharides are the simplest carbohydrates in that they cannot be hydrolyzed to smaller carbohydrates. Most monosaccharides can be represented by the general formula C_{y}H_{2y}O_{y} (e.g., C₆H₁₂O₆ (a hexose such as glucose)), wherein y is an integer equal to or greater than 3. Certain polyhydric alcohols not represented by the general formula described above may also be considered monosaccharides. For example, deoxyribose is of the formula C₅H1004 and is a monosaccharide. Monosaccharides usually consist of five or six carbon atoms and are referred to as pentoses and hexoses, receptively. If the monosaccharide contains an aldehyde it is referred to as an aldose; and if it contains a ketone, it is referred to as a ketose. Monosaccharides may also consist of three, four, or seven carbon atoms in an aldose or ketose form and are referred to as trioses, tetroses, and heptoses, respectively. Glyceraldehyde and dihydroxyacetone are considered to be aldotriose and ketotriose sugars, respectively. Examples of aldotetrose sugars include erythrose and threose; and ketotetrose sugars include erythrulose. Aldopentose sugars include ribose, arabinose, xylose, and lyxose; and ketopentose sugars include ribulose, arabulose, xylulose, and lyxulose. Examples of aldohexose sugars include glucose (for example, dextrose), mannose, galactose, allose, altrose, talose, gulose, and idose; and ketohexose sugars include fructose, psicose, sorbose, and tagatose. Ketoheptose sugars include sedoheptulose. Each carbon atom of a monosaccharide bearing a hydroxyl group (—OH), with the exception of the first and last carbons, is asymmetric, making the carbon atom a stereocenter with two possible configurations (R or S). Because of this asymmetry, a number of isomers may exist for any given monosaccharide formula. The aldohexose D-glucose, for example, has the formula C₆H₁₂O₆, of which all but two of its six carbons atoms are stereogenic, making D-glucose one of the 16 (i.e., 24) possible stereoisomers. The assignment of D or L is made according to the orientation of the asymmetric carbon furthest from the carbonyl group: in a standard Fischer projection if the hydroxyl group is on the right the molecule is a D sugar, otherwise it is an L sugar. The aldehyde or ketone group of a straight-chain monosaccharide will react reversibly with a hydroxyl group on a different carbon atom to form a hemiacetal or hemiketal, forming a heterocyclic ring with an oxygen bridge between two carbon atoms. Rings with five and six atoms are called furanose and pyranose forms, respectively, and exist in equilibrium with the straight-chain form. During the conversion from the straight-chain form to the cyclic form, the carbon atom containing the carbonyl oxygen, called the anomeric carbon, becomes a stereogenic center with two possible configurations: the oxygen atom may take a position either above or below the plane of the ring. The resulting possible pair of stereoisomers is called anomers. In an a anomer, the —OH substituent on the anomeric carbon rests on the opposite side (trans) of the ring from the —CH₂OH side branch. The alternative form, in which the —CH₂OH substituent and the anomeric hydroxyl are on the same side (cis) of the plane of the ring, is called a p anomer. A carbohydrate including two or more joined monosaccharide units is called a disaccharide or polysaccharide (e.g., a trisaccharide), respectively. The two or more monosaccharide units bound together by a covalent bond known as a glycosidic linkage formed via a dehydration reaction, resulting in the loss of a hydrogen atom from one monosaccharide and a hydroxyl group from another. Exemplary disaccharides include sucrose, lactulose, lactose, maltose, isomaltose, trehalose, cellobiose, xylobiose, laminaribiose, gentiobiose, mannobiose, melibiose, nigerose, or rutinose. Exemplary trisaccharides include, but are not limited to, isomaltotriose, nigerotriose, maltotriose, melezitose, maltotriulose, raffinose, and kestose. The term carbohydrate also includes other natural or synthetic stereoisomers of the carbohydrates described herein.

These and other exemplary substituents are described in more detail in the Detailed Description, Examples, and claims. The invention is not intended to be limited in any manner by the above exemplary listing of substituents.

### Other Definitions

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N+(C₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R.0.5 H₂O)), and polyhydrates (x is a number greater than 1, e.g., dihydrates (R.2 H₂O) and hexahydrates (R.6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁₋₈ alkyl, C₂₋₈ alkenyl, C₂₋₈ alkynyl, aryl, C₇₋₁₂ substituted aryl, and C₇-C₁₂ arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal “Disease,” “disorder,” and “condition” are used interchangeably herein.

The term “administer,” “administering,” or “administration” refers to implanting, absorbing, ingesting, injecting, inhaling, or otherwise introducing a compound described herein, or a composition thereof, in or on a subject.

As used herein, and unless otherwise specified, the terms “treat,” “treating” and “treatment” contemplate an action that occurs while a subject is suffering from the specified infectious disease or inflammatory condition, which reduces the severity of the infectious disease or inflammatory condition, or retards or slows the progression of the infectious disease or inflammatory condition (“therapeutic treatment”), and also contemplates an action that occurs before a subject begins to suffer from the specified infectious disease or inflammatory condition (“prophylactic treatment”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of an infectious disease or inflammatory condition, or to delay or minimize one or more symptoms associated with the infectious disease or inflammatory condition. A therapeutically effective amount of a compound means an amount of therapeutic agent, alone or in combination with other therapies, which provides a therapeutic benefit in the treatment of the infectious disease or inflammatory condition. The term “therapeutically effective amount” can encompass an amount that improves overall therapy, reduces or avoids symptoms or causes of infectious disease or inflammatory condition, or enhances the therapeutic efficacy of another therapeutic agent.

As used herein, and unless otherwise specified, a “prophylactically effective amount” of a compound is an amount sufficient to prevent an infectious disease or inflammatory condition, or one or more symptoms associated with the infectious disease or inflammatory condition, or prevent its recurrence. A prophylactically effective amount of a compound means an amount of a therapeutic agent, alone or in combination with other agents, which provides a prophylactic benefit in the prevention of the infectious disease or inflammatory condition. The term “prophylactically effective amount” can encompass an amount that improves overall prophylaxis or enhances the prophylactic efficacy of another prophylactic agent.

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

### Examples

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

Table 1 provides a list of commercially available aminoalcohol intermediates that were used to prepare various compounds.

TABLE 1
List of Commercially Available Aminoalcohol Intermediates.

| Aminoalcohol Number | Structure |
|---|---|
| I1 | 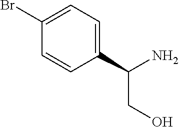 [SMILES: N[C@@H](CO)c1ccc(Br)cc1] |
| I2 | 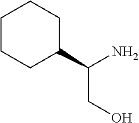 |
| I3 | 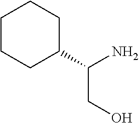 |
| I4 | 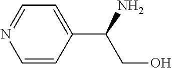 |

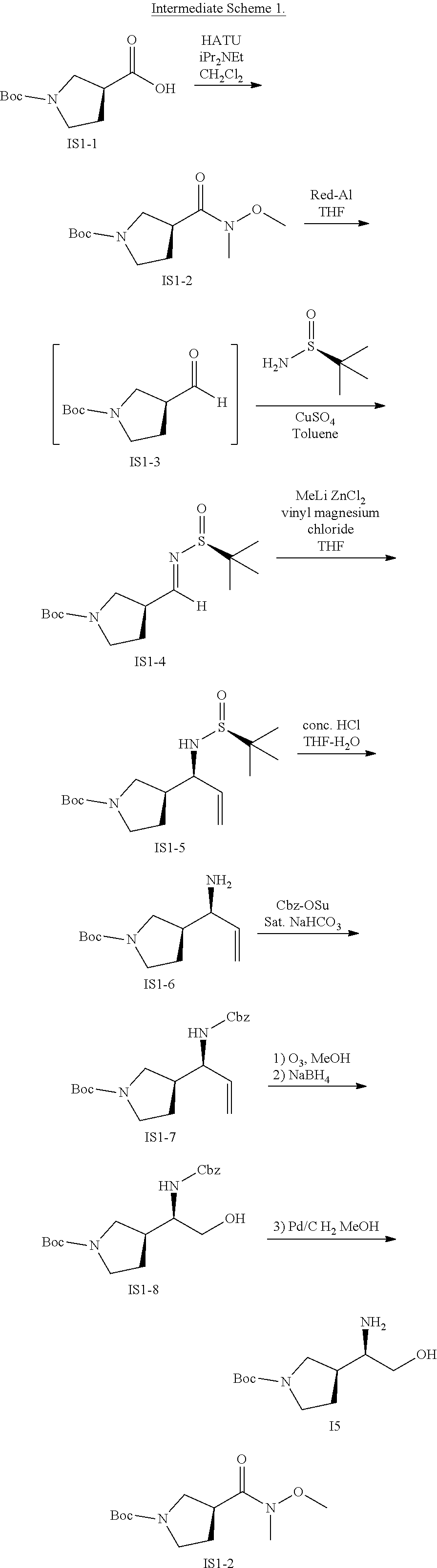

### tert-Butyl (S)-3-(methoxy(methyl)carbamoyl)pyrrolidine-1-carboxylate (IS1-2)

To a solution of (S)-1-(tert-butoxycarbonyl)pyrrolidine-3-carboxylic acid (5.03 g, 23.3 mmol) in dichloromethane (93.2 mL) was added methyoxyl(methyl)amine hydrochloride (3.4 g, 34.9 mmol), N,N-diisopropylethylamine (12.1 mL, 69.9 mmol), and 1-[Bis(dimethylamino) methylene]-1H-1,2,3-triazolo[4,5-b]pyridinium 3-oxid hexafluorophosphate (HATU) (13.2 g, 34.9 mmol). The reaction mixture was stirred at room temperature for 16 hours (h). The reaction mixture was poured into 1 M NaOH and stirred vigorously for 10 minutes (min), the organic layer was separated, and further washed with 2N HCl (2 times), water (1 time), and brine (1 time). The washed solution was dried over sodium sulfate and concentrated in vacuo. The crude material was purified by column chromatography (80 g silica gel column, 0-50% EtOAC/Hex) to give IS1-2 as a white powder (6.01 g, 23.2 mmol, 100%). MS (ESI+) m/z: 281.12 [M+Na]⁺; ¹H NMR (400 MHz, Chloroform-d) δ 3.71 (s, 3H), 3.57 (d, 2H), 3.45 (t, 1H), 3.41-3.23 (m, 2H), 3.20 (s, 3H), 2.22-2.04 (m, 2H), 1.45 (s, 9H).

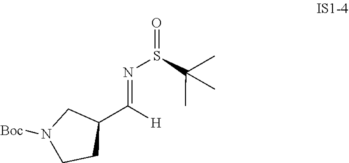 [SMILES: CC(C)(C)OC(=O)N1CC[C@H](/C=N/[S@@](=O)C(C)(C)C)C1]

### tert-Butyl (S)-3-((E)-(((S)-tert-butylsulfinyl)imino)methyl)pyrrolidine-1-carboxylate (IS1-4)

The Weinreb amide IS1-2 (6.01 g, 23.2 mmol) was dissolved in THF (93 mL) and the resulting mixture was cooled to −40° C. Sodium bis(2-methoxyethoxy)aluminum dihydride (Red-Al® (8.5 mL, 70 wt % in toluene, 30.1 mmol) was then added. The reaction mixture was allowed to warm to room temperature and stirred for 16 h. Ethyl acetate and saturated (sat.) aqueous (aq.) potassium sodium tartrate tetrahydrate (Rochelle salt) was added and the mixture was stirred vigorously for 2 h. The organic layer was separated and washed with brine (1 time), dried over sodium sulfate, filtered and concentrated in vacuo to give aldehyde IS1-3 as a clear oil. Aldehyde IS1-3 (2.3 g, 11.5 mmol) was dissolved in toluene (19.1 mL), and (S)-2-methylpropane-2-sulfinamide (1.8 g, 14.9 mmol) followed by copper (II) sulfate (9.16 g, 57.4 mmol) was added. The reaction mixture was stirred at room temperature for 18 h and then filtered through diatomaceous earth (Celite®) with ethyl acetate, and the filtrate was concentrated in vacuo. The crude material was purified by silica gel column chromatography (40 g, 0-70% EtOAc/Hex) to give the product as a white solid (2.01 g, 6.64 mmol, 58%). ¹H NMR (400 MHz, Chloroform-d) δ 8.03 (q, 1H), 3.68-3.28 (m, 4H), 3.23 (s, 1H), 2.25-1.93 (m, 2H), 1.45 (s, 9H), 1.18 (s, 9H).

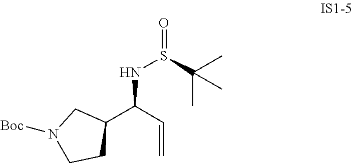 [SMILES: C=C[C@H](N[S@@](=O)C(C)(C)C)[C@H]1CCN(C(=O)OC(C)(C)C)C1]

### tert-Butyl (S)-3-((((S)-tert-butylsulfinyl)amino)methyl)pyrrolidine-1-carboxylate (IS1-5)

A solution of ZnCl₂ (6.94 mL, 1.9 M in Me-THF, 13.2 mmol) was added to dry THF (6.5 mL) and cooled to −78° C. A solution of methyl lithium (8.54 mL, 3.1M in DME, 26.5 mmol) was added slowly, keeping an internal reaction below −65° C. The mixture was stirred for 10 min and a solution of vinylmagnesium chloride (8.24 mL, 1.9 M in THF, 13.2 mmol) was added slowly, keeping the reaction temperature below −65° C. and the mixture was stirred for 5 min. A solution of IS1-4 (2.01 g, 6.64 mmol) in THF (1 M) was added dropwise and the reaction mixture was stirred for 30 min. Acetic acid (1 mL) was added slowly, the bath was removed, and the reaction mixture was allowed to warm to room temperature (rt) over 20 min. Half sat. aq NH₄Cl was added followed by MTBE. The layers were separated, the aqueous layer was extracted with MBTE (2 times), the combined organic extracts were dried over Na₂SO₄, filtered and concentrated. The crude material was used in the next step without further purification. ¹H NMR (400 MHz, Chloroform-d) δ 5.65 (dd, 1H), 5.32-5.19 (m, 2H), 3.72 (td, 1H), 3.47 (m, 2H), 3.26 (m, 2H), 3.06 (m, 1H), 2.43-2.22 (m, 1H), 1.45 (s, 9H), 1.21 (s, 9H).

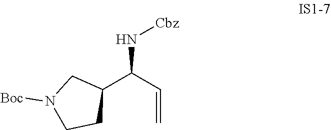 [SMILES: C=C[C@H](NC(=O)OCc1ccccc1)[C@H]1CCN(C(=O)OC(C)(C)C)C1]

### tert-Butyl (S)-3-((S)-1-(((benzyloxy)carbonyl)amino)allyl)pyrrolidine-1-carboxylate (IS1-7)

A solution of IS1-5 (2.15 g, 6.5 mmol) in THF/water (5:2, 14.5 mL) was added with concentrated (conc.) HCl (0.56 mL, 6.82 mmol), the reaction mixture was stirred at room temperature for 18 h. Sat. aq. NaHCO₃ (10 mL) was added followed by N-(benzyloxycarbonyloxy)succinimide (1.69 g, 6.82 mmol). The reaction mixture was stirred at room temperature for 1 h and extracted with EtOAc (2 times). The combined organic extracts were washed with brine, dried over sodium sulfate, filtered and concentrated in vacuo. The crude material was purified silica gel column chromatography (40 g, 0-70% EtOAc/Hex) to give IS1-7 as a white powder (1.7 g, 4.71 mmol, 73%). MS (ESI+) m/z: 383.21 [M+Na]⁺; ¹H NMR (400 MHz, Chloroform-d) δ 7.43-7.29 (m, 5H), 5.80-5.66 (m, 1H), 5.19 (dd, 2H), 5.11 (s, 2H), 4.74 (d, 1H), 4.24-4.05 (m, 1H), 3.57-3.37 (m, 2H), 3.37-3.16 (m, 1H), 3.03 (dt, 1H), 2.27 (s, 1H), 1.96 (s, 1H), 1.45 (s, 9H).

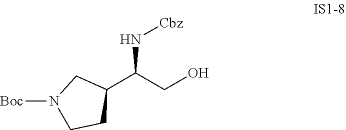 [SMILES: CC(C)(C)OC(=O)N1CC[C@H]([C@H](CO)NC(=O)OCc2ccccc2)C1]

### tert-Butyl (S)-3-((R)-1-(((benzyloxy)carbonyl)amino)-2-hydroxyethyl)pyrrolidine-1-carboxylate (IS1-8)

IS1-7 (1.7 g, 4.71 mmol) was dissolved in methanol (94 mL) and cooled to −78° C. A stream of ozone (7 PSI, 2 liters per minute (LPM)) was bubbled through the reaction mixture for 8 min, and slight blue coloration was observed. The ozone stream was removed, and nitrogen was then bubbled through the solution for 5 min (blue color disappeared). Sodium borohydride (442 mg, 11.7 mmol) was added, and the reaction mixture was removed from the bath and allowed to warm to room temperature for 30 mins. The reaction mixture was quenched with sat. aq NH₄Cl and extracted with dichloromethane (3 times). The combined organic extracts were dried over Na₂SO₄, filtered, and concentrated in vacuo to give the product as a white foam (1.49 g, 4.08 mmol, 87%). The material was used in the next step without further purification. MS (ESI+) m/z: 387.19 [M+Na]⁺; ¹H NMR (400 MHz, Chloroform-d) δ 7.41-7.28 (m, 5H), 5.10 (s, 2H), 3.79-3.35 (m, 5H), 3.22 (s, 1H), 3.00 (s, 1H), 2.38 (s, 1H), 1.97 (d, 1H), 1.60 (s, 1H), 1.44 (d, 9H).

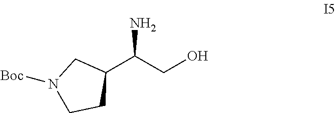 [SMILES: CC(C)(C)OC(=O)N1CC[C@H]([C@@H](N)CO)C1]

### tert-Butyl (S)-3-((R)-1-amino-2-hydroxyethyl)pyrrolidine-1-carboxylate (I5)

A solution of Cbz-amino alcohol IS1-8 (600 mg, 1.64 mmol) was dissolved in methanol (8.2 mL) and Pd/C was added (174 mg, 5 wt % on charcoal, 0.5 mol %). A balloon of hydrogen was bubbled through the reaction mixture for 1 h. The reaction mixture was filtered through Celite® with methanol and the filtrate was concentrated in vacuo to give 15 as a clear oil (377 mg, 1.645 mmol, 100%). MS (ESI+) m/z: 253.18 [M+Na]⁺; ¹H NMR (400 MHz, Chloroform-d) δ 3.60 (dd, 1H), 3.56-3.48 (m, 1H), 3.44 (d, 1H), 3.31 (dd, 1H), 3.26-3.16 (m, 1H), 2.94 (q, 1H), 2.73 (td, 1H), 2.06 (d, 2H), 1.92 (dt, 1H).

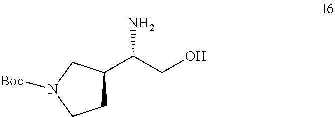 [SMILES: CC(C)(C)OC(=O)N1CC[C@H]([C@H](N)CO)C1]

### tert-Butyl (S)-3-((S)-1-amino-2-hydroxyethyl)pyrrolidine-1-carboxylate (I6)

Prepared according to the methods of I5, substituting (R)-2-methylpropane-2-sulfinamide gave I6 as a clear oil. MS (ESI+) m/z: 253.18 [M+Na]⁺.

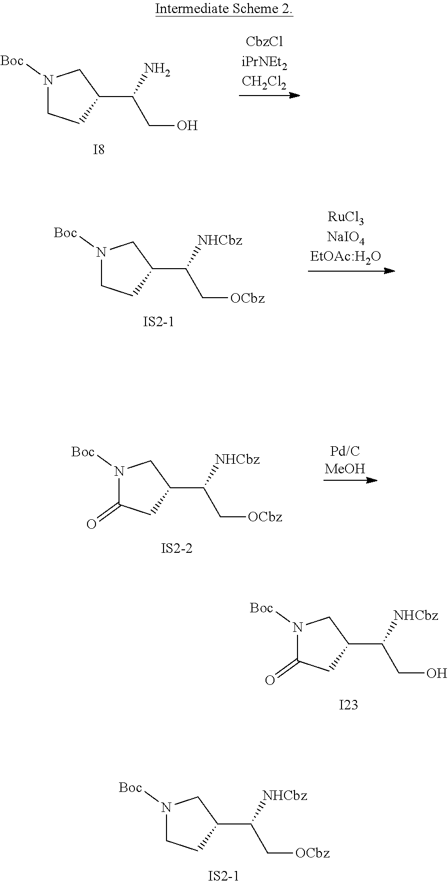

### tert-Butyl (R)-3-((S)-3,8-dioxo-1,10-diphenyl-2,7,9-trioxa-4-azadecan-5-yl)pyrrolidine-1-carboxylate (IS2-1)

To a solution of amino alcohol 18 (0.6 g, 2.61 mmol) in dichloromethane (8.7 mL) was added N, N-diisopropylethylamine (2.7 mL, 15.6 mmol) followed by benzyl chloroformate (1.84 mL, 13 mmol). The reaction mixture was diluted with dichloromethane and washed with sat. NH₄Cl. The washed solution was dried over Na₂SO₄, filtered and concentrated in vacuo. The crude residue was purified silica gel column chromatography (24 g, 0-40% EtOAc/hexane) to give IS2-1 as a white solid (0.45 g, 0.9 mmol, 35%). MS (ESI+) m/z: 521.10 [M+Na]⁺; ¹H NMR (400 MHz, Chloroform-d) δ 7.35 (dd, 10H), 5.15 (s, 2H), 5.09 (s, 2H), 5.01 (d, 1H), 4.28-4.17 (m, 1H), 4.14 (dd, 1H), 3.89 (s, 1H), 3.50 (dt, 2H), 3.21 (d, 1H), 3.00 (dt, 1H), 2.35 (s, 1H), 1.95 (d, 1H), 1.79-1.63 (m, 1H), 1.45 (d, 9H).

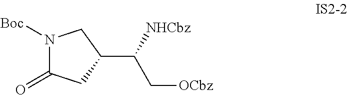

### tert-Butyl (R)-4-((S)-3,8-dioxo-1,10-diphenyl-2,7,9-trioxa-4-azadecan-5-yl)-2-oxopyrrolidine-1-carboxylate (IS2-2)

To a solution of IS2-1 (0.45 g, 0.9 mmol) in ethyl acetate (10 mL) was added a solution of sodium periodate (0.88 g, 4.11 mmol) in water (10 mL). Ruthenium chloride (18.9 mg, 0.01 mmol) was added in one portion and the reaction mixture was stirred at room temperature for 1.5 hr. It was poured into a separating funnel, the organic layer was separated and the aqueous layer was extracted with ethyl acetate (2 times). The combined extracts were washed with brine, dried over Na₂SO₄, filtered and concentrated in vacuo. The crude residue was purified by silica gel column chromatography (24 g, 0-60% EtOAc/Hexane) to give IS2-2 as a white solid (220 mg, 0.43 mmol, 47%). MS (ESI+) m/z: 535.23 [M+Na]⁺; ¹H NMR (400 MHz, Chloroform-d) δ 7.39-7.33 (m, 9H), 5.15 (s, 2H), 5.09 (s, 2H), 4.20 (t, 2H), 3.98 (d, 1H), 3.92-3.79 (m, 1H), 3.44 (dd, 1H), 2.58-2.49 (m, 2H), 1.52 (s, 9H).

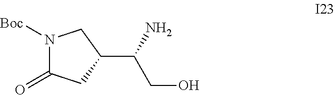 [SMILES: CC(C)(C)OC(=O)N1C[C@H]([C@H](N)CO)CC1=O]

### tert-Butyl (R)-4-((S)-1-amino-2-hydroxyethyl)-2-oxopyrrolidine-1-carboxylate (I23)

To A solution of Cbz-amino alcohol IS2-2 (220 mg, 0.43 mmol) in methanol (2 mL) and Pd/C (45.3 mg, 5 wt %, 5 mol %), the reaction mixture was bubbled with hydrogen for 15 mins and stirred under an atmosphere of hydrogen for 1 h. Upon completion, the mixture was filtered through Celite® with ethyl acetate and the filtrate was concentrated in vacuo to give amino alcohol I23 (104 mg, 0.43 mmol, 100%) as a clear oil. The crude material was used in the next step without further purification. MS (ESI+) m/z: 267.11 [M+Na]⁺; ¹H NMR (400 MHz, Chloroform-d) δ 3.88 (dd, 1H), 3.69 (td, 1H), 3.07-2.98 (m, 2H), 2.98-2.85 (m, 2H), 2.69 (dd, 1H), 2.58 (d, 1H), 2.54-2.47 (m, 1H), 1.51 (s, 9H).

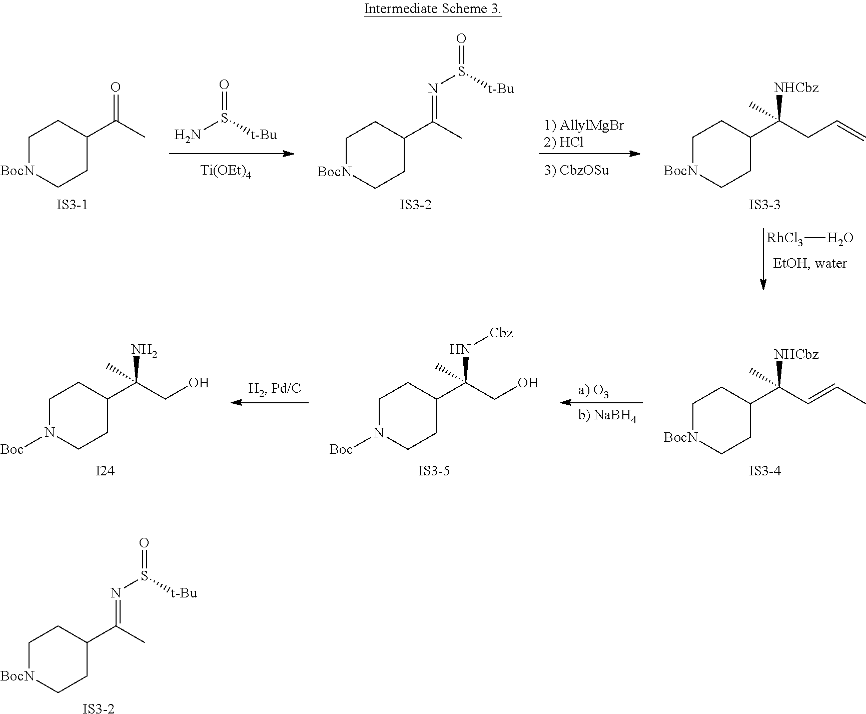

### tert-Butyl (R,E)-4-(1-((tert-butylsulfinyl)imino)ethyl)piperidine-1-carboxylate (IS3-2)

To tert-butyl 4-acetylpiperidine-1-carboxylate (4.7 g, 20.6 mmol) and (R)-2-methylpropane-2-sulfinamide (4.99 g, 41.2 mmol) in THF (25 mL) was added tetraethoxytitanium (9 mL, 41.2 mmol), and the reaction mixture was stirred at 70° C. for 24 h. N,N,N′,N′-tetrakis(2-hydroxyethyl)ethylenediamine (15 g, 64 mmol) was added, and the mixture was allowed to cool to 20° C. The reaction mixture was split between 1 N ammonium hydroxide (150 mL) and ethyl acetate (150 mL). Solids were removed by filtration through a small pad of Celite®. The filtrate was concentrated in vacuo. The material was purified by silica gel column chromatography (0-70% EtOAc/hexanes gradient) to give 4.2 g (62%) of the title compound. ¹H NMR (400 MHz, Chloroform-d) δ 4.12 (m, 2H), 2.74 (m, 2H), 1.83 (m, 2H), 2.36 (m, 1H), 2.34 (s, 2H), 1.51 (m, 2H), 1.44 (s, 9H), 1.22 (s, 9H).

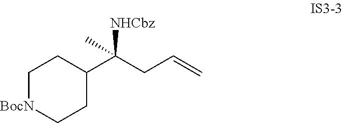

### tert-Butyl (S)-4-(2-(((benzyloxy)carbonyl)amino)pent-4-en-2-yl)piperidine-1-carboxylate (IS3-3)

To IS3-2 (4.2 g, 12.7 mmol) in dichoromethane (40 mL) was added allyl magnesium bromide in ether (26 mL) slowly at −20° C., at a rate such that precipitation of salts did not prevent stirring. The mixture was warmed to 0° C. and stirred for 1 h and was then quenched with saturated aqueous ammonium chloride (50 mL). The organics were separated and concentrated. The residue was purified by silica gel column chromatography to give 4.1 g of the sulfonamide intermediate. The material was dissolved in THF (16 mL), and water (3 mL) and 37% HCl (2 mL) were added. After 2.5 h, saturated, aqueous sodium bicarbonate (40 mL) and EtOAc (20 mL) were added, followed by N-(benzyloxycarbonyloxy)succinimide (3.5 g, 14 mmol). After stirring overnight, the organics were separated and concentrated, and the resulting residue was purified by chromatography to give 4.1 g (80%) of the title compound. ¹H NMR (400 MHz, Chloroform-d) δ 7.34 (m, 5H), 5.75 (m, 1H) 5.14-4.94 (m, 4H), 0.97 (s, 1H), 4.15 (m, 2H), 2.63 (m, 2H), 2.24 (m, 1H), 2.14 (m, 1H), 1.60 (m, 2H), 1.44 (s, 9H), 1.20 (m, 2H) 1.14 (s, 3H).

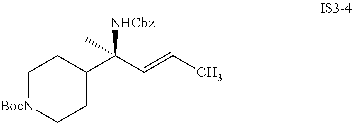

### tert-Butyl (R,E)-4-(2-(((benzyloxy)carbonyl)amino)pent-3-en-2-yl)piperidine-1-carboxylate (IS3-4)

IS3-3 (2 g, 5 mmol) was dissolved in EtOH (18 mL) and water (2 mL). RhCl₃-hydrate (300 mg, 1.2 mmol) was added, and the mixture was heated to 50° C. for 2.5 h. The reaction was concentrated, and the residue was purified by silica gel column chromatography to give 2.1 g of an approximately 90:10 ratio of IS3-4:IS3-3.

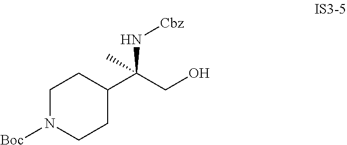 [SMILES: CC(C)(C)OC(=O)N1CCC([C@](C)(CO)NC(=O)OCc2ccccc2)CC1]

### tert-Butyl (R)-4-(2-(((benzyloxy)carbonyl)amino)-1-hydroxypropan-2-yl)piperidine-1-carboxylate (IS3-5)

IS3-4 (1.2 g, 2.99 mmol) was dissolved in methanol (94 mL) and cooled to −78° C. A stream of ozone (7 PSI, 2 LPM) was bubbled through the reaction mixture for 8 mins, and a slight blue coloration was observed. The ozone stream was removed, and nitrogen was then bubbled through the solution for 5 min (blue color disappeared). Sodium borohydride (225 mg, 5.96 mmol) was added, and the reaction mixture was removed from the bath and allowed to warm to room temperature for 30 mins. The reaction mixture was quenched with sat. aq NH₄C₁ and extracted with dichloromethane (3 times). The combined organic extracts were dried over Na₂SO₄, filtered, and concentrated in vacuo. The crude residue was purified by silica gel column chromatography (24 g, 0-60% EtOAc/Hexane) to give the title compound as a white foam (0.85 g, 2.16 mmol, 73%). MS (ESI+) m/z: 414.79 [M+Na]⁺; ¹H NMR (400 MHz, Chloroform-d) δ 7.42-7.28 (m, 5H), 5.12-5.01 (m, 2H), 4.85 (s, 1H), 4.18 (s, 3H), 3.79-3.63 (m, 2H), 2.66 (d, 2H), 2.20 (t, 1H), 1.75 (d, 1H), 1.45 (s, 9H), 1.31-1.08 (m, 3H), 1.02 (s, 3H).

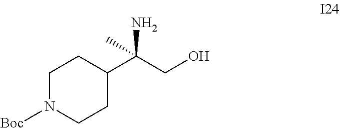 [SMILES: CC(C)(C)OC(=O)N1CCC([C@@](C)(N)CO)CC1]

### tert-Butyl (R)-4-(2-amino-1-hydroxypropan-2-yl)piperidine-1-carboxylate (I24)

A solution of IS3-5 (850 mg, 2.16 mmol) was dissolved in methanol (5 mL) and Pd/C was added (114 mg, 10 wt % on charcoal, 0.5 mol %). A balloon of hydrogen was bubbled through the reaction mixture for 1 hr. The reaction mixture was filtered through Celite® with methanol and the filtrate was concentrated in vacuo to give the title compound as a clear oil (552 mg, 2.13 mmol, 99%). MS (ESI+) m/z: 258.98 [M+H]⁺.

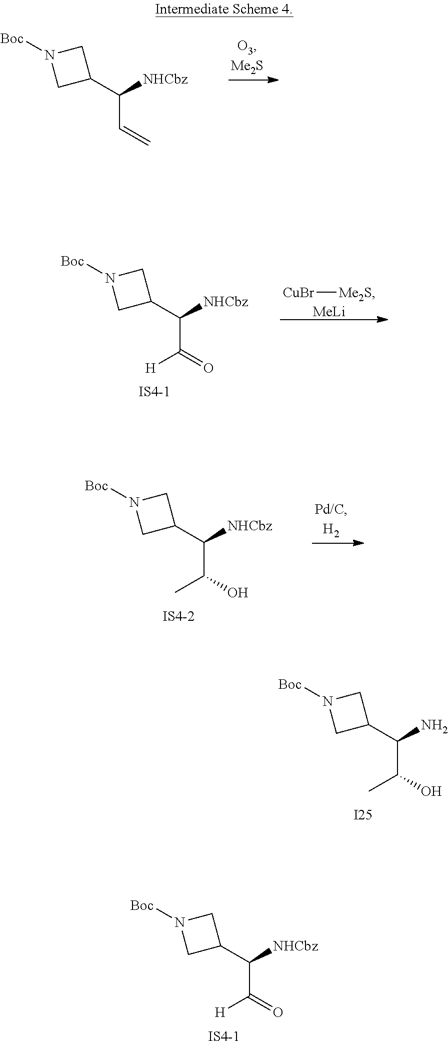

### tert-Butyl (R)-3-(1-(((benzyloxy)carbonyl)amino)-2-oxoethyl)azetidine-1-carboxylate (IS4-1)

tert-Butyl (S)-3-(1-(((benzyloxy)carbonyl)amino)allyl)azetidine-1-carboxylate (5 g, 14.4 mmol, prepared from 1-(tert-butoxycarbonyl)azetidine-3-carboxylic acid as described for IS1-7) was dissolved in dichloromethane (50 mL, 10 vol.) and was cooled to −78° C. A stream of ozone (7 PSI, 2 LPM) was bubbled through the reaction mixture until a blue color persisted (˜3-10 min). The ozone stream was removed, and nitrogen was then bubbled through the solution for 5 min (until blue color disappeared). Dimethyl sulfide (5-10 equiv.) was added, and the reaction mixture was removed from the bath and was allowed to warm to room temperature overnight. Formation of the desired aldehyde (5 g, 14.3 mmol) was confirmed by ¹H NMR. After concentration, the crude material was used without further purification. ¹H NMR (400 MHz, Chloroform-d) δ 9.70 (s, 1H), 7.42-7.28 (m, 5H), 5.16-5.07 (m, 2H), 3.94-3.64 (m, 5H), 2.80 (m, 1H), 1.43 (s, 9H).

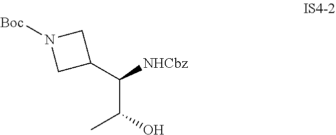 [SMILES: C[C@@H](O)[C@H](NC(=O)OCc1ccccc1)C1CN(C(=O)OC(C)(C)C)C1]

### tert-Butyl 3-((1R,2R)-1-(((benzyloxy)carbonyl)amino)-2-hydroxypropyl)azetidine-1-carboxylate (IS4-2)

To a slurry of copper (I) bromide-dimethyl sulfide (6.45 g, 31.4 mmol, 2.2 equiv.) in diethyl ether (20 mL) at approximately −70 to −78° C. was added methyl lithium (20.2 mL, 62.9 mmol, 4.4 equiv., 3.1 M solution in THF) slowly, maintaining the batch temperature below −70° C. Once the addition was complete, the batch was warmed up to rt to dissolve all the salts. The batch was then cooled to −78° C. and IS4-1 (5 g, 14.3 mmol) in diethyl ether/THF (10 mL, 2 vol.) was added slowly, maintaining an internal temp approximately −65 to −78° C. After approximately 2 h, the batch was quenched with aqueous sat. NH₄Cl solution (20 mL, 4 vol.), and MTBE (25 mL, 5 vol.) was added. The organic layer was separated, was washed with aqueous sat. NaHCO₃ solution and aqueous sat. NaCl solution, and was dried over sodium sulfate. Concentration of the organic layer provided the crude product. Following flash chromatography, the product was isolated as a mixture of diastereomers (>8:1) in 66% yield (3.5 g); ¹H NMR (400 MHz, Chloroform-d) δ 7.42-7.28 (m, 5H), 5.16-5.07 (m, 2H), 3.94-3.64 (m, 6H), 2.80 (m, 1H), 1.43 (s, 9H), 1.22 (d, 3H).

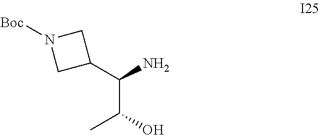 [SMILES: C[C@@H](O)[C@H](N)C1CN(C(=O)OC(C)(C)C)C1]

### tert-Butyl 3-((1R,2R)-1-amino-2-hydroxypropyl)azetidine-1-carboxylate (I25)

In a 50 mL vial was added a solution of Cbz amine in 5 mL of MeOH. The mixture was stirred at rt under nitrogen. 5% Pd/C was added and hydrogen was bubbled through with vigorous stirring for 90 min. The reaction mixture was filtered through a plug of Celite® and concentrated to give I25 (333 mg, 95%). MS (ESI+) m/z: 175.2 [M−C₄H₈+H]⁺, 231.2 [M+H]⁺.

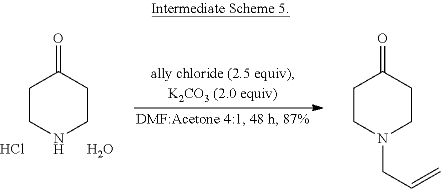

### 1-Allylpiperidin-4-one

To a stirred solution of piperidin-4-one hydrochloride hydrate (8.0 g, 52.1 mmol) in DMF:Acetone (4:1) (40 mL:10 mL) at 0° C., K₂C₀₃ was added to the reaction mixture and stirred for 15 mins. Allyl chloride was added to the reaction mixture and stirred for 48 hr at room temperature. After 48 hr the reaction was neutralized with cold water (25 mL) and ethyl acetate (50 mL) was added to the reaction. The organic layer was separated and the aqueous layer was washed ethyl acetate (50 mL×3). The combined organic layers were washed with brine and the organic layer was dried over anhydrous Na₂SO₄, concentrated under vacuum, and purified by flash column chromatography to give 1-allylpiperidin-4-one (6.31 g, 87%).

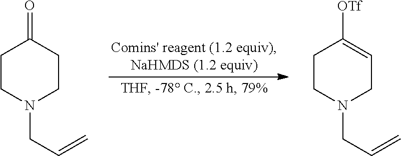

### 1-Allyl-1,2,3,6-tetrahydropyridin-4-yl trifluoromethanesulfonate

To a stirred solution of 1-allylpiperidin-4-one (6.31 g, 43.3 mmol) in THF at −78° C. under argon, sodium bis(trimethylsilyl)amide (54.4 ml, 1M, 54.4 mmol) was added slowly to the reaction mixture under argon for 1 h, then 2-(N,N-bis(trifluoromethylsulfonyl)amino)-5-chloropyridine (21.36 g, 54.4 mmol) was added and stirred at −78° C. under argon for another 1.5 h. Reaction was monitored by TLC (30% acetone in hexanes, with 1% TEA, KMnO₄), which indicated complete consumption of starting material to give vinyl triflate product. Reaction was quenched with 20 mL sat. aq. NH₄C₁, and 20 ml of cold water stirred while warming to rt. Organic layer was separated, transferred to a separatory funnel, diluted with EtOAc and water. The layers were separated. Extracted with 3×30 mL EtOAc. Combined organic phase washed with brine, dried over Na₂SO₄, filtered, and concentrated under vacuum, purified with flash column chromatography to give 1-allyl-1,2,3,6-tetrahydropyridin-4-yl trifluoromethanesulfonate (5.61 g, 79%).

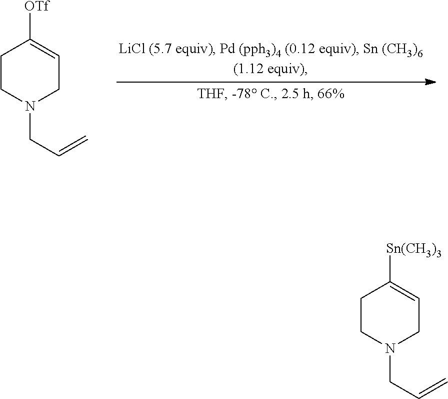

### 1-Allyl-4-(trimethylstannyl)-1,2,3,6-tetrahydropyridine

LiCi (5.61 g, 132 mmol) was flame dried under vacuum, added to a dried 250 mL round bottom flask, cooled to room temperature under argon, and 100 ml of THF was added. Pd(PPh₃)₄ (3.22 g, 2.79 mmol) was charged in a separate 25 mL pear-shaped vial with THF (15 ml×2). Another 25 mL round bottom flask was charged with vinyl triflate (6.3 g, 23.23 mmol) and THF (15 ml×2), and cannulated to the 250 ml reaction flash having anhydrous LiCi. Hexamethyltin (8.52 g, 26.0 mmol) was added to the reaction mixture at room temperature. The reaction was heated to reflux for 1.5 h. The reaction was cooled to 23° C. then hexanes was added to the reaction mixture followed by cold water (60 ml). The organic layer was separated and the aqueous layer was washed with ethyl acetate (50 mL×3). The combined organic layer was washed with brine and organic layer was dried over anhydrous Na₂SO₄, concentrated under vacuum, and purified with flash column chromatography to give 1-allyl-4-(trimethylstannyl)-1,2,3,6-tetrahydropyridine (4.43 g, 66%).

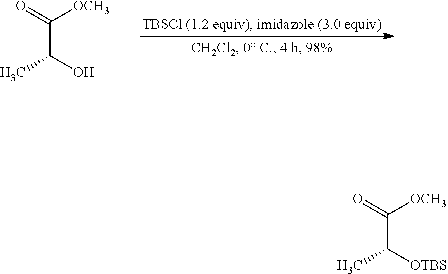

### Methyl (R)-2-((tert-butyldimethylsilyl)oxy)propanoate

To a stirred solution of methyl (R)-2-hydroxypropanoate (8.0 g, 77 mmol) in CH₂Cl₂ (150 mL) at 0° C., imidazole (10.46 g, 154 mmol) was added to the reaction mixture and stirred for 5 mins. TBDMSCl (13.90 g, 92 mmol) was added to the reaction mixture at 0° C. and the reaction was allowed to stir for 4 h at room temperature. Reaction was neutralized with cold water (25 mL) and CH₂Cl₂ (50 mL) was added to the reaction. Organic layer was separated, and aqueous layer was washed CH₂Cl₂ (50 mL×3). The combined organic layers were washed with brine, dried over anhydrous Na₂SO₄, concentrated under vacuum, purified with flash column chromatography to give methyl (R)-2-((tert-butyldimethylsilyl)oxy)propanoate (16.78 g, 98%) as colorless oil.

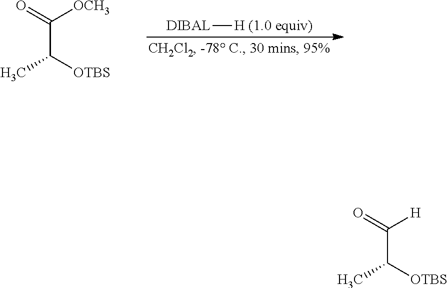

### (R)-2-((tert-Butyldimethylsilyl)oxy)propanal

To a stirred solution of methyl (R)-2-((tert-butyldimethylsilyl)oxy)propanoate (16 g, 73.3 mmol) in CH₂Cl₂ (200 mL) at −78° C. was added DIBAL-H (74 ml, 74.0 mmol) slowly to the reaction mixture and stirred for 30 mins. Reaction was monitored by TLC which shows complete reduction of ester into aldehyde. Reaction was neutralized with saturated solution of sodium potassium tartatrate (50 ml) at same temperature. CH₂Cl₂ (100 ml) was added and allowed to stir reaction until layer separation at 23° C. Organic layer was separated and aqueous layer was washed with CH₂Cl₂ (50 mL×3). The combined organic layers were washed with brine, dried over anhydrous Na₂SO₄, concentrated under vacuum, purified with flash column chromatography to give (R)-2-((tert-butyldimethylsilyl)oxy)propanal (13.1 g, 95%) as colorless oil.

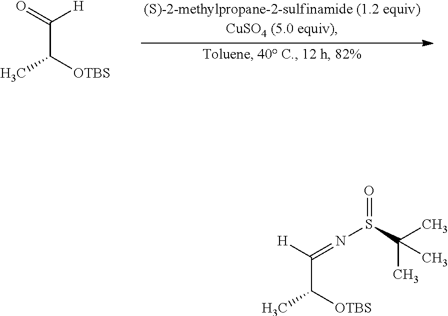 [SMILES: C[C@H](C=O)O[Si](C)(C)C(C)(C)C ; C[C@H](C=N[S@@](=O)C(C)(C)C)O[Si](C)(C)C(C)(C)C]

### (S)—N—((R)-2-((tert-butyldimethylsilyl)oxy)propylidene)-2-methylpropane-2-sulfinamide

(R)-2-((tert-Butyldimethylsilyl)oxy)propanal (13.0 g, 69.0 mmol), (S)-2-methylpropane-2-sulfinamide (1.2 equiv), and flame dried anhydrous CuSO₄ were added in 250 mL round bottom flask with 120 mL of anhydrous toluene and heated to 40° C. for 12 h. After completion of reaction it was cooled to 23° C. and filtered through small pad of celite. The cake was washed with CH₂Cl₂ (50 mL×4). The organic layer was dried over anhydrous Na₂SO₄, concentrated under vacuum, purified with flash column chromatography to provide (S)—N—((R)-2-((tert-butyldimethylsilyl)oxy)propylidene)-2-methylpropane-2-sulfinamide (16.4 g, 82%) as a colourless oil.

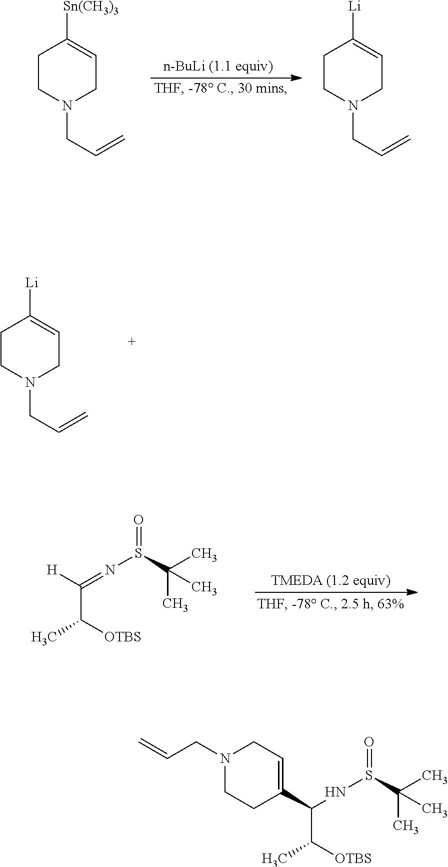

### (S)—N-((1R,2R)-1-(1-allyl-1,2,3,6-tetrahydropyridin-4-yl)-2-((tert-butyldimethylsilyl)oxy) propyl)-2-methylpropane-2-sulfinamide

To a stirred solution of 1-allyl-4-(trimethylstannyl)-1,2,3,6-tetrahydropyridine (4.43 g, 15.49 mmol) in an oven dried 250 mL round bottom in THF (20 mL) at −78° C. was added n-butyllithium (6.20 mL, 2.5 M, 15.49 mmol) to the reaction mixture and stirred for 1 h at the same temperature to give the lithiation adduct (1-allyl-1,2,3,6-tetrahydropyridin-4-yl)lithium. TMEDA (6.47 mL, 42.9 mmol) was added to the lithiation adduct and stirred for 15 mins. (S)—N—((R)-2-((tert-butyldimethylsilyl)oxy)propylidene)-2-methylpropane-2-sulfinamide (2.5 g, 8.58 mmol) was added to the reaction mixture diluting with 20 mL of THF and the reaction was stirred for another 1.5 h. A saturated solution of NH₄Cl (10 mL) and 10 mL of cold water followed by 30 ml EtOAc were added. The organic layer was separated, and the aqueous layer was washed EtOAc (50 mL×3). The combined organic layers were washed with brine, dried over anhydrous Na₂SO₄, concentrated under vacuum, purified with flash column chromatography to give (S)—N-((1R,2R)-1-(1-allyl-1,2,3,6-tetrahydropyridin-4-yl)-2-((tert-butyldimethylsilyl)oxy) propyl)-2-methylpropane-2-sulfinamide (2.16 g, 61%) as a thick liquid.

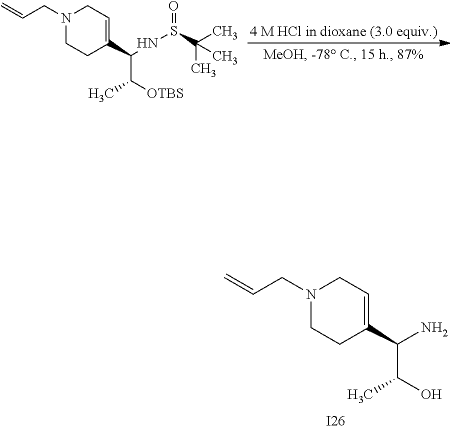 [SMILES: C=CCN1CC=C([C@@H](N[S@@](=O)C(C)(C)C)[C@@H](C)O[Si](C)(C)C(C)(C)C)CC1 ; "I26" ; C=CCN1CC=C([C@@H](N)[C@@H](C)O)CC1]

### (1R,2R)-1-(1-allyl-1,2,3,6-tetrahydropyridin-4-yl)-1-aminopropan-2-ol (I26)

To a stirred solution of (S)—N-((1R,2R)-1-(1-allyl-1,2,3,6-tetrahydropyridin-4-yl)-2-((tert-butyldimethylsilyl)oxy)propyl)-2-methylpropane-2-sulfinamide (2.16 g, 52.1 mmol) in methanol at 0° C., HCl in dioxane (4 mL, 4 M, 15.62 mmol) was added to the reaction mixture and stirred for 15 h at 23° C. After 15 h methanol was removed under vacuum and the reaction mixture was diluted with CH₂Cl₂ (15 mL) and neutralized with saturated solution of NaHCO₃. The organic layer was separated and the aqueous layer was washed with CH₂Cl₂ (20 mL×3). Combined organic layer was washed with brine and dried over anhydrous Na₂SO₄, concentrated under vacuum, purified with flash column chromatography to give (1R,2R)-1-(1-allyl-1,2,3,6-tetrahydropyridin-4-yl)-1-aminopropan-2-ol (I26) (890 mg, 87%) as a thick liquid.

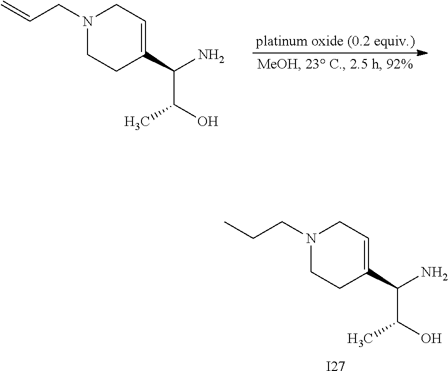

### (1R,2R)-1-amino-1-(1-propyl-1,2,3,6-tetrahydropyridin-4-yl)propan-2-ol (I27)

To a stirred solution of (2R)-1-(1-allyl-1,2,3,6-tetrahydropyridin-4-yl)-1-aminopropan-2-ol (2 mg, 52.1 mmol) in MeOH (3 mL) at 23° C., platinum oxide (46 mg, 0.204 mmol) was added to the reaction mixture. 1 Atmosphere pressure of hydrogen gas was applied into the reaction mixture with double balloon and stirred for 2.5 h. The mixture was filtered through small pad of celite and washed the cake many times with MeOH to ensure the complete recovery of product. Concentrated under vacuum, to give (1R,2R)-1-amino-1-(1-propyl-1,2,3,6-tetrahydropyridin-4-yl)propan-2-ol (I27) (187 mg, 92%).

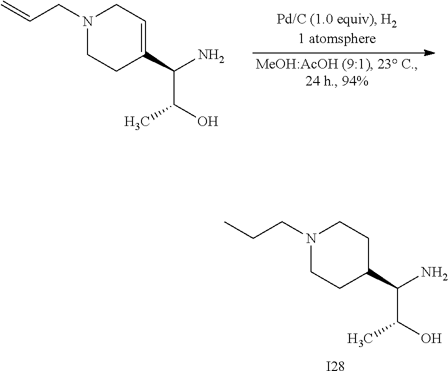

### (1R,2R)-1-Amino-1-(1-propyl-1,2,3,6-tetrahydropyridin-4-yl)propan-2-ol (I28)

To a stirred solution of (2R)-1-(1-allyl-1,2,3,6-tetrahydropyridin-4-yl)-1-aminopropan-2-ol (2 mg, 52.1 mmol) in MeOH (3 mL) at 23° C., Pd/C (130 mg, 0.204 mmol) was added to the reaction mixture. One atmosphere pressure of hydrogen gas was applied into the reaction mixture with double balloon and stirred for 2.5 h. The mixture was filtered through a small pad of celite and washed the cake many times with MeOH to ensure the complete recovery of product. Concentrated under vacuum, to give (1R,2R)-1-amino-1-(1-propyl-1,2,3,6-tetrahydropyridin-4-yl)propan-2-ol (191 mg, 94%).

TABLE 2
List of Synthetic Aminoalcohol Intermediates.

| Amino- | | |
|---|---|---|
| alcohol | | |
| Num- | | |
| ber | Structure | Source |
| I5 | 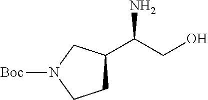 [SMILES: CC(C)(C)OC(=O)N1CC[C@H]([C@@H](N)CO)C1] | Methods of Intermediate Scheme 1 |
| I6 | 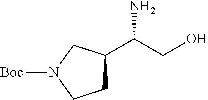 [SMILES: CC(C)(C)OC(=O)N1CC[C@H]([C@H](N)CO)C1] | Methods of Intermediate Scheme 1 |
| I7 | 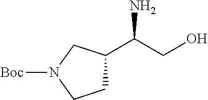 [SMILES: CC(C)(C)OC(=O)N1CC[C@@H]([C@@H](N)CO)C1] | Methods of I5 |
| I8 | 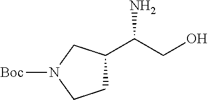 [SMILES: CC(C)(C)OC(=O)N1CC[C@@H]([C@H](N)CO)C1] | Methods of I6 |
| I9 | 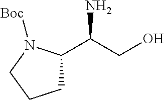 [SMILES: CC(C)(C)OC(=O)N1CCC[C@H]1[C@@H](N)CO] | Methods of I5 |
| I10 | 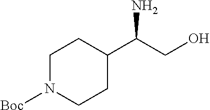 [SMILES: CC(C)(C)OC(=O)N1CCC([C@@H](N)CO)CC1] | Methods of I5 |
| I11 | 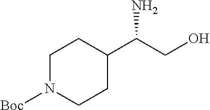 [SMILES: CC(C)(C)OC(=O)N1CCC([C@H](N)CO)CC1] | Methods of I6 |
| I12 | 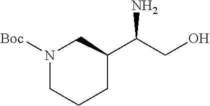 [SMILES: CC(C)(C)OC(=O)N1CCC[C@H]([C@@H](N)CO)C1] | Methods of I5 |
| I13 | 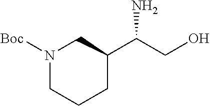 [SMILES: CC(C)(C)OC(=O)N1CCC[C@H]([C@H](N)CO)C1] | Methods of I6 |
| I14 | 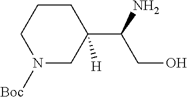 [SMILES: CC(C)(C)OC(=O)N1CCC[C@@H]([C@@H](N)CO)C1] | Methods of I5 |
| I15 | 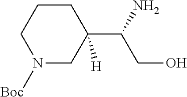 [SMILES: CC(C)(C)OC(=O)N1CCC[C@@H]([C@H](N)CO)C1] | Methods of I6 |
| I16 | 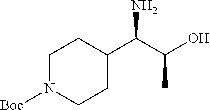 [SMILES: C[C@H](O)[C@H](N)C1CCN(C(=O)OC(C)(C)C)CC1] | Methods of I5 |
| I17 | 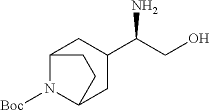 [SMILES: CC(C)(C)OC(=O)N1C2CCC1CC([C@@H](N)CO)C2] | Methods of I5 |
| I18 | 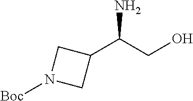 [SMILES: CC(C)(C)OC(=O)N1CC([C@@H](N)CO)C1] | Methods of I5 |
| I19 | 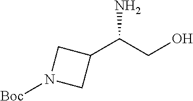 [SMILES: CC(C)(C)OC(=O)N1CC([C@H](N)CO)C1] | Methods of I6 |
| I20 | 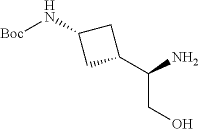 [SMILES: CC(C)(C)OC(=O)N[C@H]1C[C@@H]([C@@H](N)CO)C1] | Methods of I5 |
| I21 | 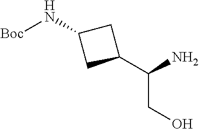 [SMILES: CC(C)(C)OC(=O)N[C@H]1C[C@H]([C@@H](N)CO)C1] | Methods of I5 |
| I22 | 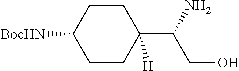 [SMILES: CC(C)(C)OC(=O)N[C@H]1CC[C@H]([C@H](N)CO)CC1] | Methods of I6 |
| I23 | 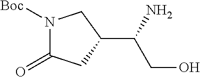 [SMILES: CC(C)(C)OC(=O)N1C[C@H]([C@H](N)CO)CC1=O] | Methods of Intermediate Scheme 2 |
| I24 | 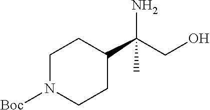 [SMILES: CC(C)(C)OC(=O)N1CCC([C@@](C)(N)CO)CC1] | Methods of Intermediate Scheme 3 |
| I25 | 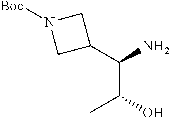 [SMILES: C[C@@H](O)[C@H](N)C1CN(C(=O)OC(C)(C)C)C1] | Methods of Intermediate Scheme 4 |
| I26 | 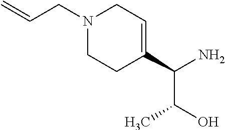 | Methods of Intermediate Scheme 5 |
| I27 | 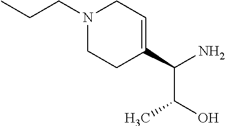 | Methods of Intermediate Scheme 5 |
| I28 | 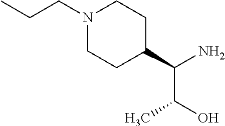 | Methods of Intermediate Scheme 5 |

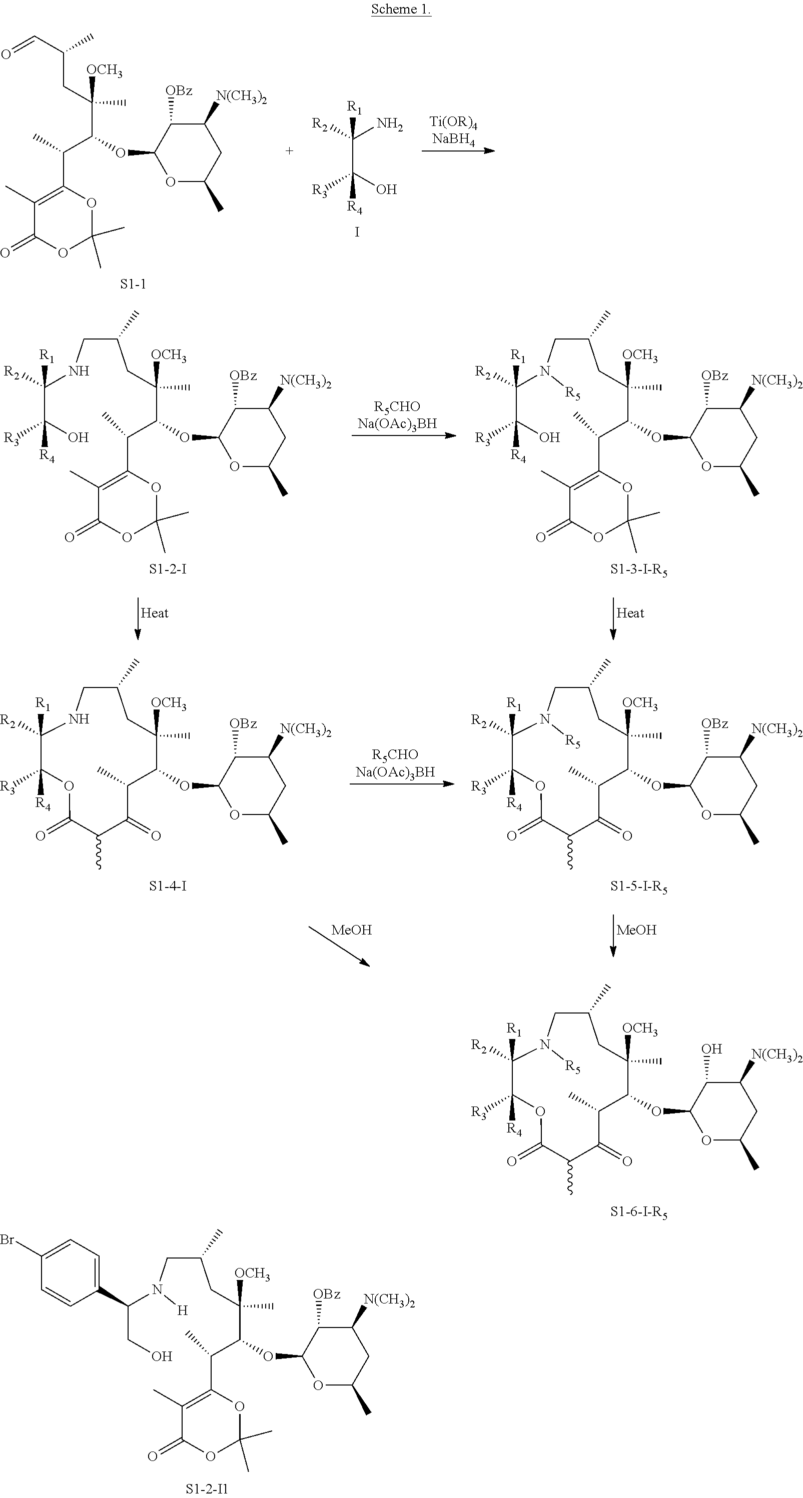

### (2S,3R,4S,6R)-2-(((2R,3R,4R,6R)-7-(((R)-1-(4-Bromophenyl)-2-hydroxyethyl)amino)-4-methoxy-4,6-dimethyl-2-(2,2,5-trimethyl-4-oxo-4H-1,3-dioxin-6-yl)heptan-3-yl)oxy)-4-(dimethylamino)-6-methyltetrahydro-2H-pyran-3-yl benzoate (S1-2-I1)

In a 40 mL vial S1-1 (490 mg, 0.83 mmol) was dissolved in EtOH (4 mL) and (R)-2-amino-2-(4-bromophenyl)ethan-1-ol (I1) (215 mg, 1.00 mmol) was added to give a solution which was stirred at rt. Ti(OEt)₄ (0.38 mL, 1.66 mmol) was added over 30 seconds and stirred for 2 h. A small aliquot was added to a suspension of a small amount of NaBH₄ in MeOH and was analysed by LC/MS and showed complete conversion. The reaction mixture was cooled in an ice bath for 10 minutes, then NaBH₄ (47 mg, 1.24 mmol) was added in one portion. When gas evolution ceased, 30% aqueous NH₄OH (5 mL) was added and stirred for 5 mins, then the mixture was filtered through a pad of Celite® with the aid of EtOAc. The filtrate was washed with brine, dried over MgSO₄, filtered and concentrated. The residue was used in the next step without further purification.

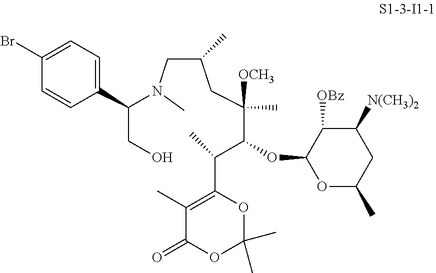

### (2S,3R,4S,6R)-2-(((2R,3R,4R,6R)-7-(((R)-1-(4-Bromophenyl)-2-hydroxyethyl)(methyl)amino)-4-methoxy-4,6-dimethyl-2-(2,2,5-trimethyl-4-oxo-4H-1,3-dioxin-6-yl)heptan-3-yl)oxy)-4-(dimethylamino)-6-methyltetrahydro-2H-pyran-3-yl benzoate (S1-3-I1-1)

S1-2-I1 (670 mg, 0.85 mmol) was dissolved in dry dichloromethane (5 mL) and formaldehyde (0.69 mL, 8.5 mmol) was added. Then NaBH(OAc)₃ (358 mg, 1.69 mmol) was added to the reaction mixture in one portion. The reaction was allowed to stir at rt for 10 min. LC/MS showed full conversion. The reaction was quenched by adding saturated NaHCO₃ (5 mL) and the aqueous layer was extracted with dichloromethane three times (10 mL). The combined organic layers were dried over MgSO₄, filtered and concentrated. The residue was purified on 24 g of silica gel (elution with 0-10% MeOH-dichloromethane+0.5% of 30% aq NH₄OH) to give the title compound as a white solid (420 mg, 62% in two steps). MS (ESI+) m/z: 402.2 [M+2H]²⁺, 803.3 [M+H]⁺.

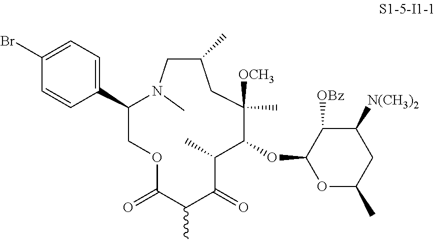

### (2S,3R,4S,6R)-2-(((3R,6R,8R,9R,10R)-3-(4-Bromophenyl)-8-methoxy-4,6,8,10,12-pentamethyl-11,13-dioxo-1-oxa-4-azacyclotridecan-9-yl)oxy)-4-(dimethylamino)-6-methyltetrahydro-2H-pyran-3-yl benzoate (S1-5-I1-1)

S1-3-I1-1 (420 mg, 0.52 mmol) was concentrated twice from toluene in a 250 mL flask. The flask was fitted with a reflux condenser and the condenser was flame dried under vacuum, allowed to cool and backfilled with nitrogen. Chlorobenzene (130 mL) was added via cannula and the flask was placed under mild vacuum and sonicated for 2 mins, then backfilled with nitrogen. The degassing procedure was repeated, then the mixture was heated at a bath temperature of 155° C. for 16 h and then at a bath temperature of 165° C. for 4 h. The reaction was allowed to cool to rt and was concentrated. The residue was purified on 24 g of silica gel (elution with 0-10% MeOH-dichloromethane+0.5% of 30% aq NH₄₀H) to give the title compound as a white solid (411 mg, 99%). MS (ESI+) m/z: 373.2 [M+2H]²⁺, 745.3 [M+H]⁺.

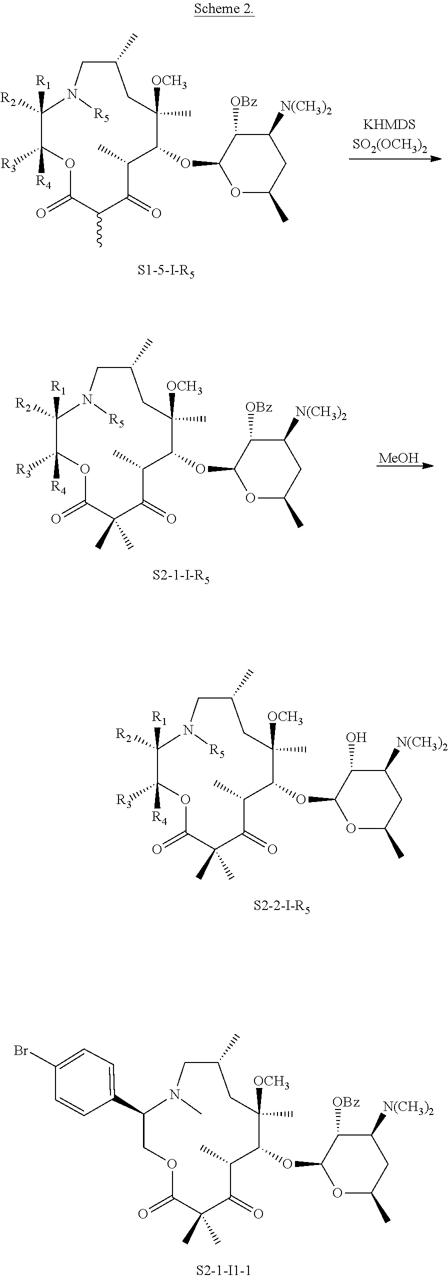

### (2S,3R,4S,6R)-2-(((3R,6R,8R,9R,10R)-3-(4-Bromophenyl)-8-methoxy-4,6,8,10,12,12-hexamethyl-11,13-dioxo-1-oxa-4-azacyclotridecan-9-yl)oxy)-4-(dimethylamino)-6-methyltetrahydro-2H-pyran-3-yl benzoate (S2-1-I1-1)

In a 20 mL vial was a solution of S1-5-I1-1 (411 mg, 0.55 mmol) in 1,2-dimethoxyethane (10 mL) precooled at −60° C. Potassium bis(trimethylsilyl)amide (KHMDS) (0.83 mL, 0.83 mmol) was added dropwise. The reaction mixture was stirred at −60° C. for 20 min. Then Me₂SO₄ (0.10 μL, 1.1 mmol) was added. The reaction mixture was allowed to warm to −15° C. LC/MS showed full conversion. The reaction was quenched by adding triethylamine (1 mL) and the resulting mixture was diluted with dichloromethane and saturated NaHCO₃ was added. The aqueous layer was extracted with dichloromethane and the combined organic layers were dried over MgSO₄, filtered and concentrated. The residue was purified on 24 g of silica gel (elution with 0-10% MeOH-dichloromethane+0.5% of 30% aq NH₄₀H) to give the title compound as a white solid (287 mg, 69%). MS (ESI+) m/z: 380.2 [M+2H]²⁺, 759.3 [M+H]⁺.

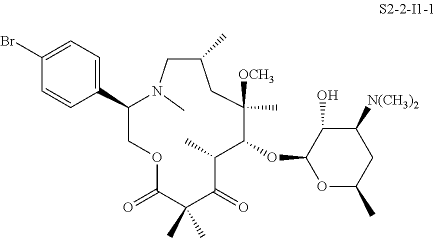

### (3R,6R,8R,9R,10R)-3-(4-bromophenyl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S2-2-I1-1) (Compound 33)

S2-1-I1-1 (20 mg, 0.026 mmol) was dissolved in MeOH (0.5 mL) and heated at 60° C. until LC/MS indicated complete consumption of starting material (16 hours). The reaction mixture was filtered through a syringe filter with the aid of methanol and concentrated. The residue was purified by HPLC (MeCN-water-0.1% HCO₂H) to yield the title compound as a formate salt (6.66 mg). MS (ESI+) m/z: 222.4 [M+3H]³⁺, 333.2 [M+2H]²⁺, 665.3 [M+H]⁺; ¹H NMR (400 MHz, Methanol-d₄) δ 8.56 (s, 2H), 7.53 (d, 3H), 7.21 (d, 3H), 4.47 (dd, 3H), 4.32 (s, 1H), 4.08 (d, 2H), 3.78 (s, 1H), 3.64 (ddd, 2H), 3.40-3.28 (m, 3H), 2.92 (s, 6H), 2.52 (s, 9H), 2.30 (s, 4H), 2.19-2.10 (m, 3H), 2.06 (s, 1H), 1.86 (ddd, 3H), 1.55 (s, 4H), 1.43-1.25 (m, 23H), 0.90 (d, 1H), 0.84 (d, 4H).

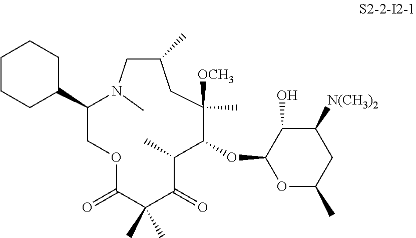

### (3R,6R,8R,9R,10R)-3-cyclohexyl-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S2-2-I2-1) (Compound 88)

Prepared according to the methods of S2-2-I1-1, substituting 12, to provide the 18.1 mg of the title compound as a formate salt. MS (ESI+) m/z: 292.33 [M+2H]²⁺, 583.4 [M+H]⁺. ¹H NMR (400 MHz, Methanol-d₄) δ 8.54 (s, 1H), 4.40 (d, 1H), 4.28 (d, 1H), 4.18 (s, 1H), 3.72-3.59 (m, 1H), 3.43-3.32 (m, 2H), 3.18-2.75 (m, 7H), 2.59 (s, 6H), 1.97-1.62 (m, 7H), 1.49 (s, 3H), 1.46-1.25 (m, 15H), 1.23-1.10 (m, 3H), 1.09-0.79 (m, 3H).

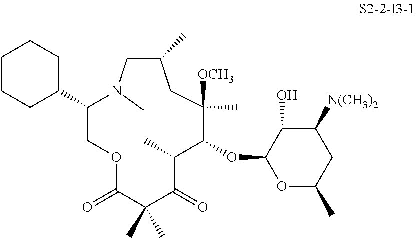

### (3S,6R,8R,9R,10R)-3-cyclohexyl-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S2-2-I3-1) (Compound 102)

Prepared according to the methods of S2-2-I1-1, substituting 13 to give 59 mg of the title compound as a formate salt. MS (ESI+) m/z: 292.3 [M+2H]²⁺, 583.4 [M+H]⁺. ¹H NMR (400 MHz, Methanol-d₄) δ 8.54 (s, 2H), 4.77 (s, 1H), 4.44 (d, 1H), 4.35 (s, 1H), 4.28-4.21 (m, 1H), 3.70 (ddt, 1H), 3.42 (dd, 2H), 3.26 (t, 1H), 3.01 (s, 3H), 2.95-2.80 (m, 2H), 2.74 (s, 6H), 2.20 (s, 1H), 2.05-1.94 (m, 2H), 1.89-1.67 (m, 7H), 1.62 (d, 1H), 1.55 (s, 3H), 1.48 (q, 3H), 1.40-1.35 (m, 7H), 1.32 (m, 8H), 1.29-1.18 (m, 3H), 1.03 (d, 3H).

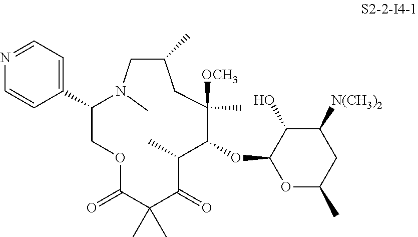 [SMILES: CO[C@]1(C)C[C@@H](C)CN(C)[C@@H](c2ccncc2)COC(=O)C(C)(C)C(=O)[C@H](C)[C@H]1O[C@@H]1O[C@H](C)C[C@H](N(C)C)[C@H]1O]

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(pyridin-4-yl)-1-oxa-4-azacyclotridecane-11,13-dione (S2-2-I4-1) (Compound 19)

Prepared according to the methods of S2-2-I1-1, substituting 14, giving the title compound as a formate salt. MS (ESI+) m/z: 578.29 [M+H]⁺; ¹H NMR (400 MHz, Chloroform-d) δ 8.70-8.51 (m, 2H), 7.22-7.06 (m, 2H), 4.51 (d, 1H), 4.39-4.33 (m, 1H), 4.04 (dd, 1H), 3.84-3.73 (m, 1H), 3.61 (dddd, 2H), 3.47 (dd, 1H), 3.22-2.98 (m, 2H), 2.94 (s, 3H), 2.89-2.83 (m, 1H), 2.82-2.56 (m, 7H), 2.31 (s, 2H), 2.29-2.17 (m, 1H), 2.16-1.97 (m, 2H), 1.94-1.81 (m, 1H), 1.71 (s, 1H), 1.55 (m, 1H), 1.50-1.35 (m, 4H), 1.36-1.15 (m, 11H), 1.16-0.96 (m, 1H), 0.88 (d, 3H).

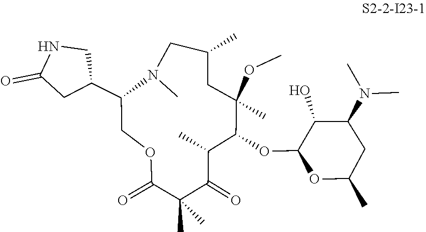

### (3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((R)-5-oxopyrrolidin-3-yl)-1-oxa-4-azacyclotridecane-11,13-dione (S2-2-I23-1) (Compound 8)

Prepared according to the methods of S2-2-I1-1, substituting I23 to give 1.63 mg of the title compound as a formate salt. MS (ESI+) m/z: 292.84 [M+2H]²⁺, 584.37 [M+H]⁺; ¹H NMR (400 MHz, Methanol-d₄) δ 8.50 (s, 2.5H), 4.46 (d, 1H), 4.23-3.96 (m, 3H), 3.71 (t, 1H), 3.53 (s, 1H), 3.49-3.41 (m, 2H), 3.35 (d, 1H), 3.22 (d, 3H), 2.88 (d, 3H), 2.79 (s, 6H), 2.58-2.24 (m, 6H), 2.01 (d, 1H), 1.95-1.60 (m, 3H), 1.47 (s, 4H), 1.40-1.10 (m, 12H), 0.92 (d, 3H).

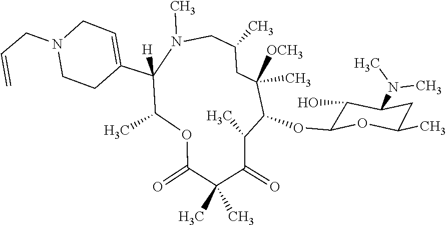

### (2R,3S,6R,8R,9R,10R)-3-(1-Allyl-1,2,3,6-tetrahydropyridin-4-yl)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-1-oxa-4-azacyclotridecane-11,13-dione (Compound 1)

Prepared according to the methods of S2-2-I1-1, substituting I26 to give 10 mg of the title compound. ¹H NMR (600 MHz, Methanol-d₄) δ 8.43 (s, 3H), 5.98 (tt, 1H), 5.47 (d, 2H), 5.32 (d, 1H), 4.44 (d, 1H), 4.10-3.93 (m, 1H), 3.74-3.61 (m, 3H), 3.56 (s, 3H), 3.49-3.36 (m, 4H), 2.92 (t, 3H), 2.83 (s, 6H), 2.67 (d, 4H), 2.45 (s, 2H), 2.01 (dt, 2H), 1.56 (d, 3H), 1.52 (dd, 3H), 1.35 (s, 6H), 1.32 (s, 3H), 1.31 (s, 3H), 1.27 (d, 3H), 1.21 (d, 3H).

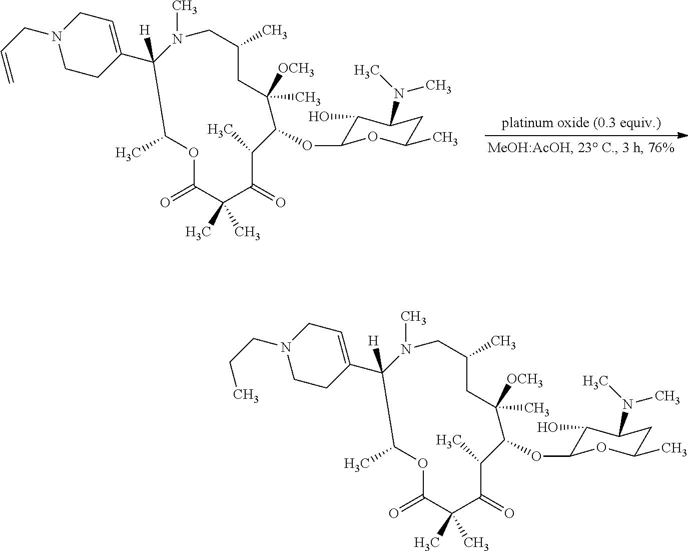

### (2R,3S,6R,8R,9R,10R)-3-(1-Allyl-1,2,3,6-tetrahydropyridin-4-yl)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-1-oxa-4-azacyclotridecane-11,13-dione (Compound 2)

To a stirred solution of (2R,6R,8R,9R,10R)-3-(1-allyl-1,2,3,6-tetrahydropyridin-4-yl)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-1-oxa-4-azacyclotridecane-11,13-dione (20.0 mg, 0.031 mmol) in methanol:AcOH (9:1) (2 mL) was added platinum(IV) oxide (3.57 mg, 0,016 mmol). Argon gas was bubbled through the mixture for 5 mins, and was then purged with hydrogen gas and allowed to stir for 3 h. The reaction mixture was filtered over small pad of celite, methanol was concentrated under vacuum, and the compound was purified with HPLC to give the formate salt of the title compound as a white solid. ¹H NMR (600 MHz, Methanol-d₄) δ 8.45 (s, 3H), 5.81 (s, 11H), 5.23 (d, 1H), 4.44 (d, 1H), 3.96 (d, 1H), 3.81 (d, 11H), 3.68 (dd, 2H), 3.47 (dd, 2H), 3.44-3.37 (m, 11H), 3.21-3.13 (m, 1H), 3.10-2.99 (m, 2H), 2.95 (s, 3H), 2.83 (s, 6H), 2.80-2.71 (m, 1H), 2.56 (dd, 2H), 2.33 (s, 3H), 2.05-1.97 (m, 2H), 1.77 (dt, 3H), 1.59-1.48 (m, 3H), 1.33 (s, 6H), 1.31 (s, 6H), 1.30 (d, 3H), 1.27 (d, 3H), 1.19 (d, 3H), 1.02 (t, 3H), 0.92 (d, 3H).

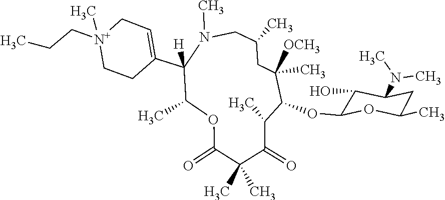

### 4-((2R,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-11,13-dioxo-1-oxa-4-azacyclotridecan-3-yl)-1-methyl-1-propyl-1,2,3,6-tetrahydropyridin-1-ium (Compound 4)

Prepared according to the methods of S2-2-I1-1, substituting I27 to give the title compound. ¹H NMR (600 MHz, Methanol-d₄) δ 8.45 (s, 3H), 5.23 (d, 1H), 4.44 (d, 1H), 3.96 (d, 1H), 3.81 (d, 1H), 3.68 (dd, 2H), 3.47 (dd, 2H), 3.44-3.37 (m, 11H), 3.21-3.13 (m, 11H), 3.10-2.99 (m, 2H), 2.95 (s, 3H), 2.83 (s, 6H), 2.80-2.71 (m, 1H), 2.56 (dd, 2H), 2.33 (s, 3H), 2.05-1.97 (m, 2H), 1.77 (dt, 3H), 1.59-1.48 (m, 3K), 1.33 (s, 6H), 1.31 (s, 6H), 1.30 (d, 3H), 1.27 (d, 3H), 1.19 (d, 3H), 1.02 (t, 3H), 0.92 (d, 3H).

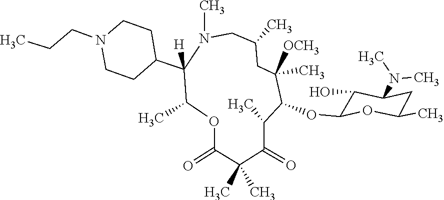

### (2R,3S,6R,8R,9R,10R)-3-(1-allyl-1,2,3,6-tetrahydropyridin-4-yl)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-1-oxa-4-azacyclotridecane-11,13-dione (Compound 5)

Prepared according to the methods of S2-2-I1-1, substituting I28 to give the title compound. ¹H NMR (600 MHz, Methanol-d₄) δ 8.50 (s, 3H), 4.97 (s, 1H), 4.45 (d, 1H), 4.10 (d, 1H), 3.70 (dd, 1H), 3.54 (s, 3H), 3.45 (dt, 1H), 3.40-3.34 (m, 1H), 2.99 (d, 2H), 2.87 (s, 3H), 2.79 (s, 6H), 2.62 (d, 2H), 2.52 (s, 3H), 2.01 (t, 3H), 1.74 (td, 2H), 1.65-1.45 (m, 5H), 1.40-1.36 (m, 6H), 1.36 (s, 3H), 1.31 (d, 3H), 1.26-1.22 (m, 6H), 1.02 (t, 3H), 0.94 (d, 3H).

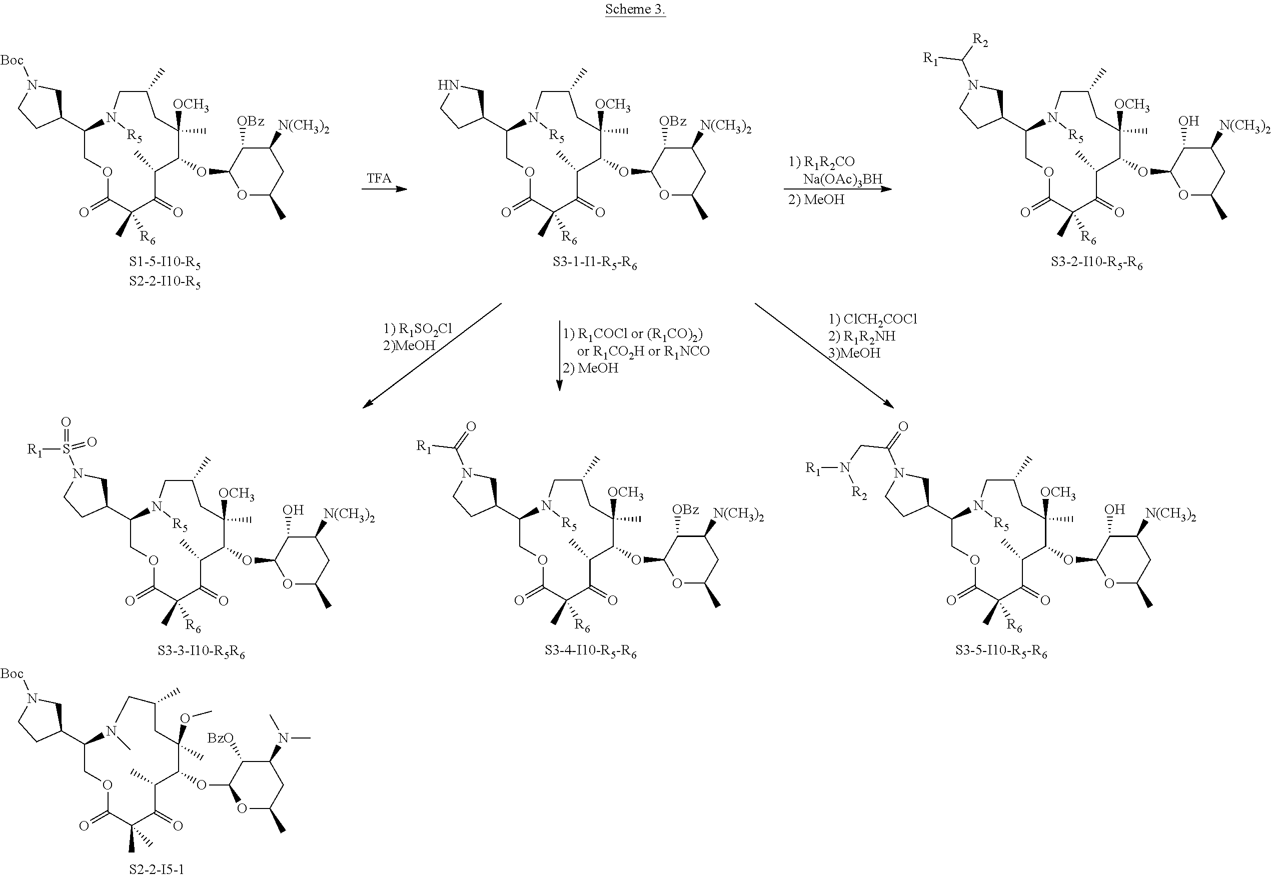

### tert-Butyl (S)-3-((3R,6R,8R,9R,10R)-9-(((2S,3R,4S,6R)-3-(benzoyloxy)-4-(dimethylamino)-6-methyltetrahydro-2H-pyran-2-yl)oxy)-8-methoxy-4,6,8,10,12,12-hexamethyl-11,13-dioxo-1-oxa-4-azacyclotridecan-3-yl)pyrrolidine-1-carboxylate (S2-2-I5-1)

Prepared according to the methods of S2-1-I1-1 from I5 to give 176 mg of the title compound. MS (ESI+) m/z: 387.91 [M+2H]²⁺, 774.37 [M+H]⁺; ¹H NMR (400 MHz, Chloroform-d) δ 8.10-7.91 (m, 2H), 7.59-7.48 (m, 1H), 7.48-7.35 (m, 2H), 5.06 (ddd, 1H), 4.59 (d, 1H), 4.12-3.93 (m, 1H), 3.93-3.77 (m, 1H), 3.54 (s, 4H), 3.39 (s, 6H), 3.25-3.05 (m, 1H), 2.96-2.76 (m, 4H), 2.33-2.20 (m, 7H), 2.20-2.05 (m, 2H), 2.02 (d, 1H), 1.77 (d, 2H), 1.50-1.42 (m, 8H), 1.39 (s, 2H), 1.35-1.29 (m, 3H), 1.27 (dd, 3H), 1.22 (s, 3H), 1.03 (dd, 3H), 0.83 (d, 3H).

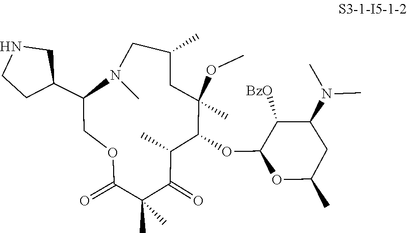

### (2S,3R,4S,6R)-4-(Dimethylamino)-2-(((3R,6R,8R,9R,10R)-8-methoxy-4,6,8,10,12,12-hexamethyl-11,13-dioxo-3-((S)-pyrrolidin-3-yl)-1-oxa-4-azacyclotridecan-9-yl)oxy)-6-methyltetrahydro-2H-pyran-3-yl benzoate (S3-1-I5-1-2)

S2-2-I5-1 (176 mg, 0.227 mmol) was dissolved in dichloromethane (1 mL) and trifluoroacetic acid (0.25 mL) was added. The reaction mixture was stirred at room temperature for 2 h and was concentrated. The residue was suspended in ethyl acetate and washed with sat. aq. NaHCO₃ (2 times). The washed solution was dried over sodium sulfate, filtered, and concentrated to give the amine intermediate (150 mg, 99%). MS (ESI+) m/z: 225.66 [M+3H]³⁺, 337.83 [M+2H]²⁺, 674.40 [M+H]⁺.

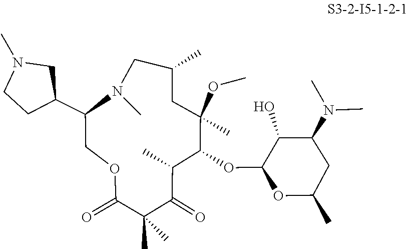

### (3R,6R,8R,9R,10R)-9-(((2S,3R,4S,6R)-4-(Dimethylamino)-3-hydroxy-6-methyltetrahydro-2H-pyran-2-yl)oxy)-8-methoxy-4,6,8,10,12,12-hexamethyl-3-((S)-1-methylpyrrolidin-3-yl)-1-oxa-4-azacyclotridecane-11,13-dione (S3-2-I5-1-2-1) (Compound 166)

S3-1-I5-1-2 (47 mg, 0.0697 mmol) was dissolved in dichloromethane (0.5 mL) and Na(OAc)₃BH (22 mg; 0.104 mmol) was added. Formaldehyde (37 wt % solution in water, 0.047 mL, 0.070 mmol) was added. After 10 min., the reaction mixture was quenched by the addition of NaHCO₃ (sat., aq. solution). The layers were separated, and the aqueous layer was extracted with dichloromethane (1 time). The combined dichloromethane extracts were dried over Na₂SO₄, were filtered, and were concentrated. The crude material was dissolved in methanol and the reaction mixture was heated to 45° C. for 16 h. The solvent was removed and the crude material was purified by HPLC (MeCN-water-0.1% HCO₂H) to yield 11.42 mg of the title compound as a formate salt. MS (ESI+) m/z: 195.53 [M+3H]³⁺, 292.78 [M+2H]²⁺, 584.37 [M+H]⁺; ¹H NMR (400 MHz, Methanol-d₄) δ 8.45 (d, 2.7H), 4.46 (t, 1H), 4.11 (dt, 2H), 3.69 (p, 1H), 3.61-3.49 (m, 1H), 3.46-3.20 (m, 6H), 2.99 (d, 1H), 2.94-2.85 (m, 3H), 2.79 (d, 10H), 2.73-2.58 (m, 2H), 2.43 (d, 3H), 2.27 (d, 1H), 1.99 (p, 2H), 1.84 (d, 2H), 1.50 (d, 4H), 1.37-1.21 (m, 12H), 0.89 (t, 3H).

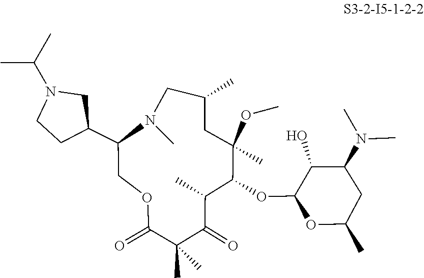

### (3R,6R,8R,9R,10R)-9-(((2S,3R,4S,6R)-4-(Dimethylamino)-3-hydroxy-6-methyltetrahydro-2H-pyran-2-yl)oxy)-3-((S)-1-isopropylpyrrolidin-3-yl)-8-methoxy-4,6,8,10,12,12-hexamethyl-1-oxa-4-azacyclotridecane-11,13-dione (S3-2-I5-1-2-2) (Compound 11)

Prepared according to the methods of S3-2-10-1-2-1 from S3-1-I5-1-2 and acetone to provide 8.8 mg of the title compounds as a formate salt. MS (ESI+) m/z: 204.92 [M+3H]³⁺, 306.83 [M+2H]²⁺, 612.43 [M+H]⁺; ¹H NMR (400 MHz, Methanol-d₄) δ 8.46 (s, 2.7H), 4.50 (d, 1H), 4.20 (d, 1H), 4.11 (d, 1H), 3.77-3.67 (m, 1H), 3.59 (q, 1H), 3.52-3.33 (m, 6H), 3.05 (q, 1H), 2.93 (s, 3H), 2.82 (s, 7H), 2.68-2.58 (m, 1H), 2.44 (s, 3H), 2.37-2.19 (d, 1H), 2.08-1.93 (m, 2H), 1.93-1.75 (m, 2H), 1.60-1.47 (m, 4H), 1.40-1.34 (m, 9H), 1.34-1.28 (m, 9H), 0.91 (d, 3H).

The following examples were prepared according to the methods of S3-2-I5-1-2-1, substituting the appropriate intermediate (Table 2) in Scheme 1 and aldehyde or ketone in Scheme 3.

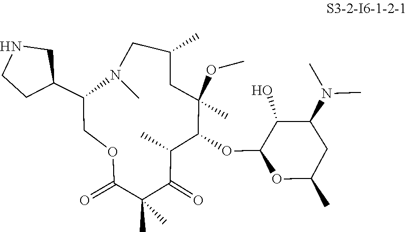

### (3S,6R,8R,9R,10R)-9-(((2S,3R,4S,6R)-4-(Dimethylamino)-3-hydroxy-6-methyltetrahydro-2H-pyran-2-yl)oxy)-8-methoxy-4,6,8,10,12,12-hexamethyl-3-((S)-pyrrolidin-3-yl)-1-oxa-4-azacyclotridecane-11,13-dione (S3-2-I6-1-2-1) (Compound 40)

Prepared according to the methods of S3-2-I5-1-2-1 from I6 and deprotection prior to reductive alkylation to provide 9.29 mg of the title compound as a formate salt. MS (ESI+) m/z: 285.83 [M+2H]²⁺, 570.29 [M+H]⁺; ¹H NMR (400 MHz, Methanol-d₄) δ 8.45 (s, 3H), 4.47 (d, 1H), 4.24 (d, 1H), 4.09 (d, 1H), 3.72 (dtd, 1H), 3.58-3.34 (m, 5H), 3.23 (q, 1H), 3.06 (t, 1H), 2.89 (d, 3H), 2.83 (d, 7H), 2.67-2.35 (m, 5H), 2.22-2.11 (m, 1H), 2.03 (ddd, 1H), 1.94-1.63 (m, 3H), 1.57-1.45 (m, 4H), 1.36 (s, 3H), 1.34-1.21 (m, 10H), 0.96 (dd, 3H).

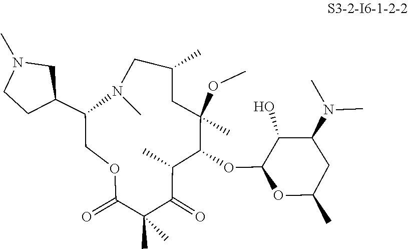

### (3S,6R,8R,9R,10R)-9-(((2S,3R,4S,6R)-4-(Dimethylamino)-3-hydroxy-6-methyltetrahydro-2H-pyran-2-yl)oxy)-8-methoxy-4,6,8,10,12,12-hexamethyl-3-((S)-1-methylpyrrolidin-3-yl)-1-oxa-4-azacyclotridecane-11,13-dione (S3-2-I6-1-2-2) (Compound 48)

Prepared according to the methods of S3-2-I5-1-2-1 from I6 and formaldehyde to proved 13.7 mg of the title compound as a formate salt. MS (ESI+) m/z: 195.54 [M+3H]³⁺, 292.74 [M+2H]²⁺, 584.37 [M+H]⁺; ¹H NMR (400 MHz, Methanol-d₄) δ 8.48 (s, 2.6H), 4.46 (d, 1H), 4.24 (d, 1H), 4.09 (d, 1H), 3.72 (dqd, 1H), 3.54 (t, 1H), 3.49-3.32 (m, 4H), 3.28-3.20 (m, 1H), 3.13-3.05 (m, 1H), 2.91 (s, 3H), 2.86 (s, 2H), 2.82 (s, 7H), 2.77-2.64 (m, 2H), 2.59-2.38 (m, 2H), 2.18 (tt, 1H), 2.07-1.99 (m, 1H), 1.96-1.77 (m, 2H), 1.59-1.45 (m, 4H), 1.36 (s, 3H), 1.31 (dd, 10H), 0.97 (d, 3H).

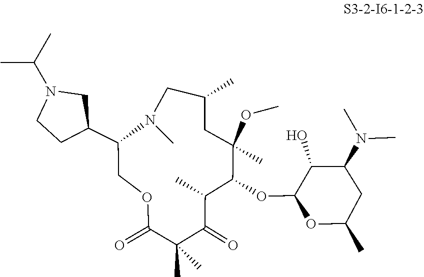

### (3S,6R,8R,9R,10R)-9-(((2S,3R,4S,6R)-4-(Dimethylamino)-3-hydroxy-6-methyltetrahydro-2H-pyran-2-yl)oxy)-3-((S)-1-isopropylpyrrolidin-3-yl)-8-methoxy-4,6,8,10,12,12-hexamethyl-1-oxa-4-azacyclotridecane-11,13-dione (S3-2-I6-1-2-3) (Compound 57)

Prepared according to the methods of S3-2-I5-1-2-1 from I6 and acetone to provide 13.1 mg of the title compound as a formate salt. MS (ESI+) m/z: 204.94 [M+3H]³⁺, 306.87 [M+2H]²⁺, 612.38 [M+H]⁺; ¹H NMR (400 MHz, Methanol-d₄) δ 8.47 (s, 2.6H), 4.46 (d, 1H), 4.25 (d, 1H), 4.17-3.97 (m, 1H), 3.76-3.66 (m, 1H), 3.65-3.56 (M, 1H), 3.52-3.33 (m, 6H), 3.15 (t, 1H), 2.93 (s, 3H), 2.83 (d, 7H), 2.76-2.34 (m, 5H), 2.18 (dq, 1H), 2.03 (ddd, 1H), 1.97-1.66 (m, 3H), 1.52 (s, 4H), 1.40-1.34 (m, 9H), 1.34-1.25 (m, 9H), 1.09-0.86 (m, 3H).

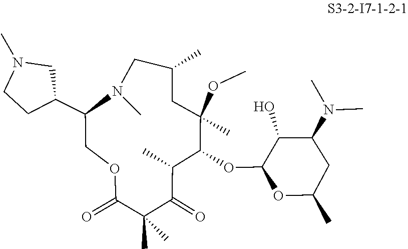

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((R)-1-methylpyrrolidin-3-yl)-1-oxa-4-azacyclotridecane-11,13-dione (S3-2-I7-1-2-1) (Compound 182)

Prepared according to the methods of S3-2-I5-1-2-1 from I7 and formaldehyde to provide 18.1 mg of the title compound as a formate salt. MS (ESI+) m/z: 292.82 [M+2H]²⁺, 584.38 [M+H]⁺; ¹H NMR (400 MHz, Methanol-d₄) δ 8.45 (s, 3H), 4.51-4.44 (m, 1H), 4.21 (d, 2H), 4.11 (d, 1H), 3.70 (ddt, 1H), 3.54 (dt, 2H), 3.48-3.33 (m, 3H), 3.33-3.21 (m, 3H), 3.17 (t, 1H), 2.93 (d, 3H), 2.86 (d, 3H), 2.81 (d, 6H), 2.71 (t, 2H), 2.46 (s, 3H), 2.14 (tt, 1H), 2.01 (ddd, 1H), 1.95-1.74 (m, 3H), 1.51 (dd, 4H), 1.34 (d, 4H), 1.32-1.21 (m, 9H), 0.91 (d, 3H).

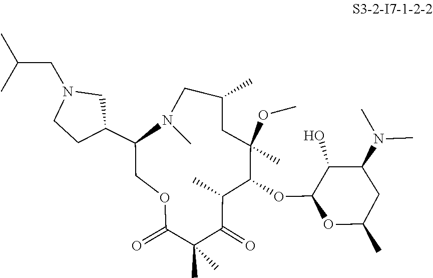

### (3R,6R,8R,9R,10R)-9-(((2S,3R,4S,6R)-4-(dimethylamino)-3-hydroxy-6-methyltetrahydro-2H-pyran-2-yl)oxy)-3-((R)-1-isobutylpyrrolidin-3-yl)-8-methoxy-4,6,8,10,12,12-hexamethyl-1-oxa-4-azacyclotridecane-11,13-dione (S3-2-I7-1-2-2) (Compound 123)

Prepared according to the methods of S3-2-I5-1-2-1 from I7 and isobutyraldehyde to provide 5.47 mg of the title compound as a formate salt. MS (ESI+) m/z: 209.66 [M+3H]³⁺, 313.84 [M+2H]²⁺, 626.46 [M+H]⁺; ¹H NMR (400 MHz, Methanol-d₄) δ 8.53 (s, 2H), 4.50 (d, 1H), 4.21 (s, 2H), 4.11 (d, 1H), 3.71 (ddd, 1H), 3.66-3.53 (m, 1H), 3.46 (dd, 2H), 3.41-3.34 (m, 1H), 3.28-3.16 (m, 2H), 3.08 (s, 1H), 2.94 (s, 5H), 2.79 (s, 6H), 2.71-2.54 (m, 2H), 2.42 (s, 4H), 2.18-2.04 (m, 1H), 2.04-1.96 (m, 2H), 1.92-1.75 (m, 3H), 1.58-1.45 (m, 4H), 1.36 (s, 4H), 1.33-1.21 (m, 9H), 1.03 (d, 6H), 0.91 (d, 3H).

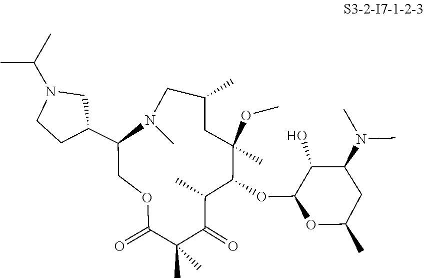

### (3R,6R,8R,9R,10R)-9-(((2S,3R,4S,6R)-4-(Dimethylamino)-3-hydroxy-6-methyltetrahydro-2H-pyran-2-yl)oxy)-3-((R)-1-isopropylpyrrolidin-3-yl)-8-methoxy-4,6,8,10,12,12-hexamethyl-1-oxa-4-azacyclotridecane-11,13-dione (S3-2-I7-1-2-3) (Compound 46)

Prepared according to the methods of S3-2-I5-1-2-1 from I7 and acetone to provide 12.1 mg of the title compound as a formate salt. MS (ESI+) m/z: 204.99 [M+3H]³⁺, 306.84 [M+2H]²⁺, 612.44 [M+H]⁺, H NMR (400 MHz, Methanol-d₄) δ 8.53 (s, 2H), 4.51 (d, 1H), 4.18 (s, 2H), 4.09 (d, 1H), 3.76-3.66 (m, 1H), 3.61 (t, 1H), 3.56-3.42 (m, 2H), 3.42-3.32 (m, 3H), 3.14 (s, 2H), 2.92 (s, 3H), 2.81 (s, 6H), 2.70-2.49 (m, 2H), 2.39 (s, 4H), 2.18-2.04 (m, 11H), 2.01 (ddd, 1H), 1.90-1.71 (m, 3H), 1.59-1.48 (m, 4H), 1.36 (d, 9H), 1.33-1.26 (m, 10H), 0.90 (d, 3H).

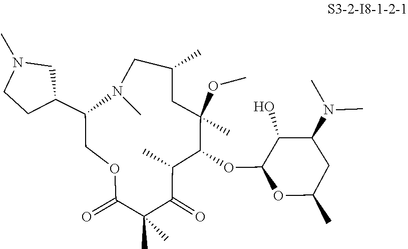

### (3S,6R,8R,9R,10R)-9-(((2S,3R,4S,6R)-4-(Dimethylamino)-3-hydroxy-6-methyltetrahydro-2H-pyran-2-yl)oxy)-8-methoxy-4,6,8,10,12,12-hexamethyl-3-((R)-1-methylpyrrolidin-3-yl)-1-oxa-4-azacyclotridecane-11,13-dione (S3-2-I8-1-2-1) (Compound 109)

Prepared according to the methods of S3-2-I5-1-2-1 from I8 and formaldehyde to provide 15.8 mg of the title compound as a formate salt. MS (ESI+) m/z: 292.81 [M+2H]²⁺, 584.37 [M+H]⁺; ¹H NMR (400 MHz, Methanol-d₄) δ 8.46 (s, 2.5H), 4.44 (d, 1H), 4.19 (d, 1H), 4.11-3.99 (m, 1H), 3.76-3.65 (m, 1H), 3.55-3.31 (m, 5H), 3.31-3.16 (m, 2H), 3.05 (t, 1H), 2.93-2.83 (m, 3H), 2.84-2.76 (m, 9H), 2.72-2.41 (m, 4H), 2.41-2.12 (m, 2H), 2.08-1.94 (m, 2H), 1.92-1.61 (m, 2H), 1.51 (s, 4H), 1.34 (s, 3H), 1.32-1.16 (m, 9H), 0.94 (dd, 3H).

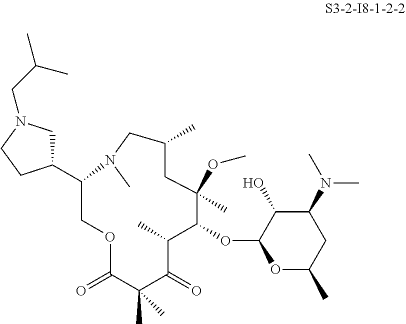

### (3S,6R,8R,9R,10R)-9-(((2S,3R,4S,6R)-4-(dimethylamino)-3-hydroxy-6-methyltetrahydro-2H-pyran-2-yl)oxy)-3-((R)-1-isobutylpyrrolidin-3-yl)-8-methoxy-4,6,8,10,12,12-hexamethyl-1-oxa-4-azacyclotridecane-11,13-dione (S3-2-I8-1-2-2) (Compound 110)

Prepared according to the methods of S3-2-I5-1-2-1 from I8 and isobutyraldehyde to provide 11.99 mg of the title compound as a formate salt. MS (ESI+) m/z: 209.65 [M+3H]³⁺, 313.87 [M+2H]²⁺, 626.49 [M+H]⁺; ¹H NMR (400 MHz, Methanol-d₄) δ 8.46 (s, 2.6H), 4.46 (d, 1H), 4.20 (d, 1H), 4.11 (s, 1H), 3.79-3.66 (m, 1H), 3.60-3.34 (m, 5H), 3.13-2.97 (m, 1H), 2.97-2.86 (m, 2H), 2.96-2.88 (m, 4H), 2.82 (s, 7H), 2.78-2.36 (m, 5H), 2.29 (s, 1H), 2.11-1.96 (m, 3H), 1.95-1.62 (m, 2H), 1.53 (s, 4H), 1.37 (s, 3H), 1.31 (dd, 9H), 1.03 (d, 6H), 1.00-0.88 (m, 3H).

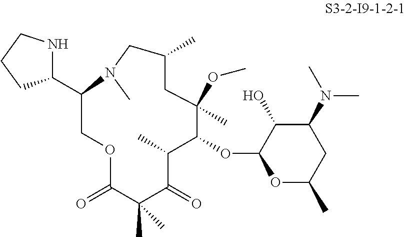

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((S)-pyrrolidin-2-yl)-1-oxa-4-azacyclotridecane-11,13-dione (S3-2-I9-1-2-1) (Compound 65)

Prepared according to the methods of S3-2-I5-1-2-1 from I9 and no reductive alkylation to provide the title compound as a formate salt. MS (ESI+) m/z: 570.30 [M+H]⁺. ¹H NMR (400 MHz, Methanol-d) δ 8.21 (s, 4H), 4.43 (d, 1H), 4.19 (d, 1H), 4.06 (d, 1H), 3.74-3.56 (m, 2H), 3.49-3.34 (m, 4H), 3.29-3.16 (m, 3H), 2.87 (s, 3H), 2.83-2.77 (m, 6H), 2.64-2.51 (m, 4H), 2.31-2.17 (m, 1H), 2.09-1.97 (m, 7H), 1.97-1.70 (m, 2H), 1.55-1.44 (m, 4H), 1.35 (d, 3H), 1.32-1.23 (m, 10H), 0.92 (d, 3H).

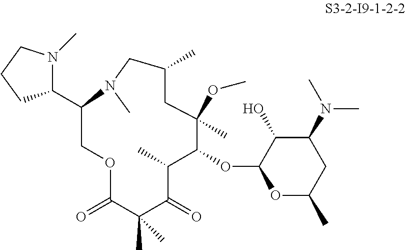

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((S)-1-methylpyrrolidin-2-yl)-1-oxa-4-azacyclotridecane-11,13-dione (S3-2-I9-1-2-2) (Compound 106)

Prepared according to the methods of S3-2-I5-1-2-1 from I9 and formaldehyde to provide the title compound as a formate salt. MS (ESI+) m/z: 584.36 [M+H]⁺. ¹H NMR (400 MHz, Methanol-d) δ 8.48 (s, 2H), 4.46 (d, 1H), 4.30 (d, 1H), 4.22-3.98 (m, 1H), 3.72 (ddd, 1H), 3.50-3.35 (m, 4H), 3.31 (s, 7H), 3.02-2.85 (m, 4H), 2.81 (s, 6H), 2.76-2.35 (m, 5H), 2.27 (q, 1H), 2.08-1.86 (m, 4H), 1.57-1.46 (m, 4H), 1.39 (s, 3H), 1.37-1.20 (m, 10H), 1.08-0.94 (m, 4H).

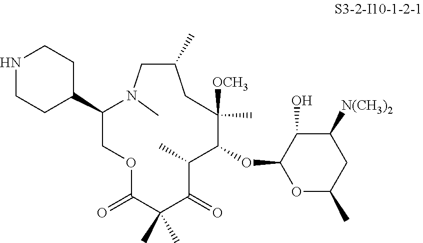

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(piperidin-4-yl)-1-oxa-4-azacyclotridecane-11,13-dione (S3-2-I10-1-2-1) (Compound 80)

Prepared according to the methods of S3-2-I5-1-2-1 from I10 and deprotection prior to reductive alkylation to provide 10.2 mg of the title compound as a formate salt. MS (ESI+) m/z: 584.43 [M+H]⁺; ¹H NMR (400 MHz, Methanol-d₄) δ 8.49 (br s, 3H), 4.79 (d, 1H), 4.47 (d, 1H), 4.40 (dd, 1H), 4.27 (d, 1H), 3.80-3.70 (m, 1H), 3.70-3.58 (m, 1H), 3.55-3.36 (m, 5H), 3.26-3.17 (m, 1H), 3.04 (s, 6H), 2.94 (s, 3H), 2.84 (s, 6H), 2.49-2.34 (m, 1H), 2.30-2.16 (m, 1H), 2.11-2.00 (m, 2H), 2.00-1.91 (m, 1H), 1.91-1.69 (m, 3H), 1.69-1.47 (m, 5H), 1.47-1.23 (m, 13H), 1.06 (d, 3H).

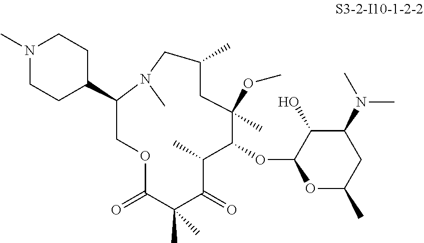

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-methylpiperidin-4-yl)-1-oxa-4-azacyclotridecane-11,13-dione (S3-2-I10-1-2-2) (Compound 43)

Prepared according to the methods of S3-2-I5-1-2-1 from I10 and formaldehyde to provide 7.1 mg of the title compound as a formate salt. MS (ESI+) m/z: 299.78 [M+2H]²⁺, 598.39 [M+H]⁺; ¹H NMR (400 MHz, Methanol-d₄) δ 8.48 (s, 2.7H), 4.49 (d, 1H), 4.31-4.08 (m, 2H), 3.72 (dtd, 1H), 3.52 (s, 1H), 3.49-3.36 (m, 4H), 2.96 (s, 3H), 2.82 (s, 9H), 2.74 (s, 3H), 2.68-2.46 (m, 3H), 2.15-1.93 (m, 4H), 1.89 (d, 1H), 1.81-1.58 (m, 3H), 1.57-1.49 (m, 4H), 1.37 (s, 4H), 1.33-1.22 (m, 9H), 0.95 (d, 3H).

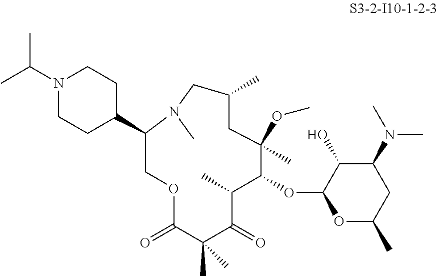

### (3R,6R,8R,9R,10R)-9-(((2S,3R,4S,6R)-4-(dimethylamino)-3-hydroxy-6-methyltetrahydro-2H-pyran-2-yl)oxy)-3-(1-isopropylpiperidin-4-yl)-8-methoxy-4,6,8,10,12,12-hexamethyl-1-oxa-4-azacyclotridecane-11,13-dione (S3-2-I10-1-2-3)

Prepared according to the methods of S3-2-I5-1-2-1 from I10 and acetone to provide the title compound as a formate salt. MS (ESI+) m/z: 626.31 [M+H]⁺, ¹H NMR (400 MHz, Methanol-d) 1H NMR (400 MHz, Chloroform-d) δ 8.46 (s, 3H), 4.71-4.52 (m, 1H), 4.48 (d, 1H), 4.37-4.24 (m, 1H), 4.21 (d, 1H), 3.80-3.69 (m, 1H), 3.56-3.37 (m, 7H), 3.10-2.92 (m, 6H), 2.90-2.67 (m, 10H), 2.32-2.15 (m, 1H), 2.15-1.93 (m, 4H), 1.93-1.63 (m, 3H), 1.63-1.47 (m, 5H), 1.44-1.26 (m, 18H), 1.00 (d, 3H).

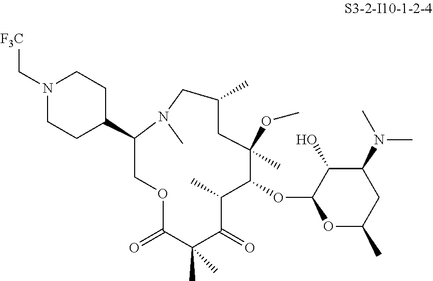

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-(2,2,2-trifluoroethyl)piperidin-4-yl)-1-oxa-4-azacyclotridecane-11,13-dione (S3-2-I10-1-2-4) (Compound 92)

Prepared according to the methods of S3-2-I5-1-2-1 from I10 and trifluoroacetaldehyde to provide the title compound as a formate salt. MS (ESI+) m/z: 666.34 [M+H]⁺. ¹H NMR (400 MHz, Methanol-d) δ 8.55 (s, 2H), 4.78 (d, 1H), 4.46 (d, 1H), 4.39 (dd, 1H), 4.28 (d, 1H), 3.74 (ddd, 1H), 3.66-3.54 (m, 1H), 3.50-3.37 (m, 3H), 3.28-3.17 (m, 1H), 3.12-3.00 (m, 8H), 2.92 (s, 3H), 2.82 (s, 6H), 2.44 (ddd, 2H), 2.30-2.16 (m, 1H), 2.09-1.98 (m, 1H), 1.75 (dd, 3H), 1.66-1.46 (m, 6H), 1.45-1.37 (m, 6H), 1.34 (dd, 7H), 1.06 (d, 3H), 0.95 (q, 2H).

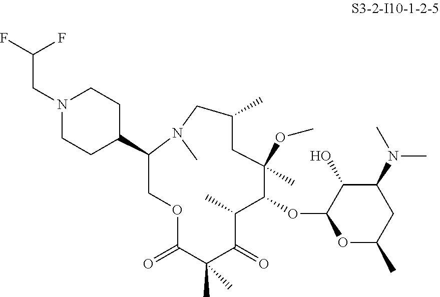

### (3R,6R,8R,9R,10R)-3-(1-(2,2-difluoroethyl)piperidin-4-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-2-I10-1-2-5) (Compound 62)

Prepared according to the methods of S3-2-I5-1-2-1 from I10 and difluoroacetaldehyde to provide the title compound as a formate salt. MS (ESI+) m/z: 648.35 [M+H]⁺. ¹H NMR (400 MHz, Methanol-d) δ 8.48 (s, 3H), 5.97 (tt, 1H), 4.79-4.64 (m, 0H), 4.47 (d, 1H), 4.40-4.30 (m, 1H), 4.29-4.19 (m, 1H), 3.79-3.68 (m, 1H), 3.61 (t, 1H), 3.50-3.33 (m, 4H), 3.12-2.86 (m, 7H), 2.83-2.70 (m, 11H), 2.36-2.19 (m, 2H), 2.08-1.94 (m, 2H), 1.85-1.76 (m, 2H), 1.74-1.47 (m, 7H), 1.42-1.27 (m, 15H), 1.03 (s, 3H), 0.93 (s, 1H).

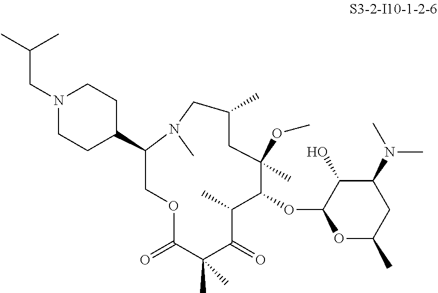

### (3R,6R,8R,9R,10R)-9-(((2S,3R,4S,6R)-4-(dimethylamino)-3-hydroxy-6-methyltetrahydro-2H-pyran-2-yl)oxy)-3-(1-isobutylpiperidin-4-yl)-8-methoxy-4,6,8,10,12,12-hexamethyl-1-oxa-4-azacyclotridecane-11,13-dione (S3-2-I10-1-2-6) (Compound 121)

Prepared according to the methods of S3-2-I5-1-2-1 from I10 and isobutyraldehyde to provide the title compound as a formate salt. MS (ESI+) m/z: 640.48 [M+H]⁺. ¹H NMR (400 MHz, Methanol-d) δ 8.40 (s, 3H), 4.74-4.60 (m, 1H), 4.48 (dd, 1H), 4.40-4.28 (m, 1H), 4.23 (d, 1H), 3.74 (ddd, 1H), 3.65-3.51 (m, 2H), 3.51-3.37 (m, 3H), 3.16-3.03 (m, 1H), 3.01 (s, 3H), 2.97-2.70 (m, 13H), 2.23 (d, 1H), 2.20-2.08 (m, 2H), 2.08-1.98 (m, 3H), 1.98-1.78 (m, 3H), 1.75-1.60 (m, 2H), 1.60-1.46 (m, 4H), 1.44-1.26 (m, 12H), 1.04 (d, 9H).

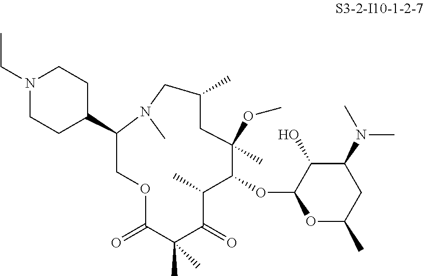

### (3R,6R,8R,9R,10R)-9-(((2S,3R,4S,6R)-4-(dimethylamino)-3-hydroxy-6-methyltetrahydro-2H-pyran-2-yl)oxy)-3-(1-ethylpiperidin-4-yl)-8-methoxy-4,6,8,10,12,12-hexamethyl-1-oxa-4-azacyclotridecane-11,13-dione (S3-2-I10-1-2-7)

Prepared according to the methods of S3-2-I5-1-2-1 from I10 and acetaldehyde to provide 2.5 mg of the title compound as a formate salt. MS (ESI+) m/z: 612.42 [M+H]⁺. ¹H NMR (400 MHz, Methanol-d) δ 8.45 (s, 3H), 4.49 (d, 1H), 4.30-4.18 (m, 1H), 4.18-4.06 (m, 1H), 3.79-3.65 (m, 2H), 3.59-3.37 (m, 6H), 3.14-3.02 (m, 2H), 3.02-2.91 (m, 4H), 2.91-2.75 (m, 9H), 2.72-2.37 (m, 3H), 2.07-1.99 (m, 1H), 1.98-1.85 (m, 2H), 1.85-1.60 (m, 3H), 1.60-1.45 (m, 5H), 1.41-1.21 (m, 17H), 0.94 (s, 3H).

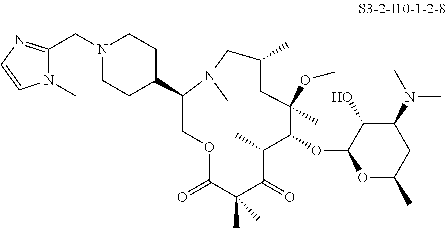

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-((1-methyl-1H-imidazol-2-yl)methyl)piperidin-4-yl)-1-oxa-4-azacyclotridecane-11,13-dione (S3-2-I10-1-2-8) (Compound 179)

Prepared according to the methods of S3-2-I5-1-2-1 from I10 and 1-methyl-1H-imidazole-2-carbaldehyde to provide 9.5 mg of the title compound as a formate salt. MS (ESI+) m/z: 678.42 [M+H]⁺. ¹H NMR (400 MHz, Methanol-d₄) δ 8.31 (s, 3H), 7.30 (s, 1H), 7.17 (s, 1H), 4.83-4.74 (m, 1H), 4.46 (d, 1H), 4.40 (dd, 1H), 4.29 (d, 1H), 3.86-3.69 (m, 6H), 3.69-3.61 (m, 1H), 3.52-3.34 (m, 3H), 3.29-3.22 (m, 1H), 3.15-3.06 (m, 1H), 3.06-2.90 (m, 8H), 2.83 (s, 6H), 2.38-2.19 (m, 3H), 2.16-2.00 (m, 2H), 1.88-1.67 (m, 4H), 1.65-1.47 (m, 6H), 1.40 (d, 6H), 1.34 (dd, 6H), 1.07 (d, 3H).

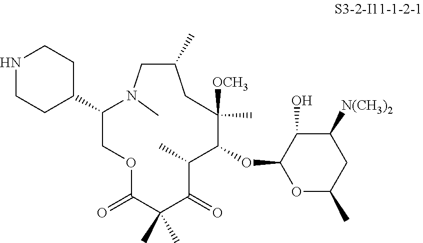

### (3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(piperidin-4-yl)-1-oxa-4-azacyclotridecane-11,13-dione (S3-2-I11-1-2-1) (Compound 98)

Prepared according to the methods of S3-2-I5-1-2-1 from I11 and no reductive alkylation to provide the title compound as a formate salt. MS (ESI+) m/z: 584.43 [M+H]⁺; ¹H NMR (400 MHz, Methanol-d₄) δ 4.46 (d, 1H), 4.30 (d, 1H), 4.21 (d, 1H), 3.81-3.60 (m, 2H), 3.52-3.37 (m, 5H), 3.20-2.94 (m, 9H), 2.83 (s, 6H), 2.52-2.33 (m, 1H), 2.27-2.09 (m, 1H), 2.09-1.93 (m, 4H), 1.93-1.78 (m, 1H), 1.78-1.62 (m, 2H), 1.62-1.46 (m, 5H), 1.40 (s, 6H), 1.37-1.26 (m, 7H), 1.08 (d, 3H).

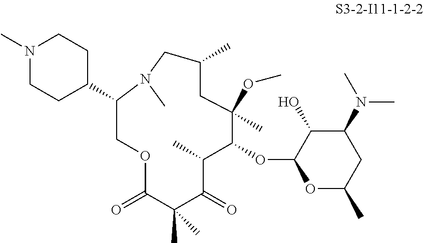

### (3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-methylpiperidin-4-yl)-1-oxa-4-azacyclotridecane-11,13-dione (S3-2-I11-1-2-2) (Compound 64)

Prepared according to the methods of S3-2-I5-1-2-1 from I11 and formaldehyde to provide 14.4 mg of the title compound as a formate salt. MS (ESI+) m/z: 598.33 [M+H]⁺; ¹H NMR (400 MHz, Methanol-d₄) δ 4.46 (d, 1H), 4.29 (d, 1H), 4.18 (d, 1H), 3.80-3.67 (m, 1H), 3.55-3.38 (m, 6H), 3.15-2.86 (m, 9H), 2.83 (s, 6H), 2.79 (s, 3H), 2.38-2.08 (m, 2H), 2.08-1.94 (m, 3H), 1.94-1.81 (m, 1H), 1.81-1.65 (m, 2H), 1.65-1.45 (m, 6H), 1.44-1.27 (m, 13H), 1.05 (d, 3H).

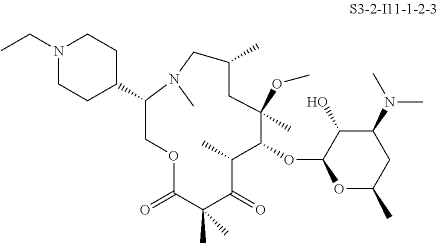

### (3S,6R,8R,9R,10R)-9-(((2S,3R,4S,6R)-4-(dimethylamino)-3-hydroxy-6-methyltetrahydro-2H-pyran-2-yl)oxy)-3-(1-ethylpiperidin-4-yl)-8-methoxy-4,6,8,10,12,12-hexamethyl-1-oxa-4-azacyclotridecane-11,13-dione (S3-2-I11-1-2-3) (Compound 175)

Prepared according to the methods of S3-2-I5-1-2-1 from I11 and acetaldehyde to provide 2.7 mg of the title compound as a formate salt. MS (ESI+) m/z: 612.37 [M+H]⁺; ¹H NMR (400 MHz, Methanol-d₄) δ 4.46 (d, 1H), 4.27 (d, 1H), 4.21-3.99 (m, 1H), 3.81-3.64 (m, 2H), 3.58-3.33 (m, 5H), 3.19-2.84 (m, 8H), 2.84-2.75 (m, 7H), 2.73-2.30 (m, 4H), 2.10-1.93 (m, 4H), 1.87-1.58 (m, 4H), 1.58-1.45 (m, 5H), 1.42-1.20 (m, 15H), 1.09-0.85 (m, 3H).

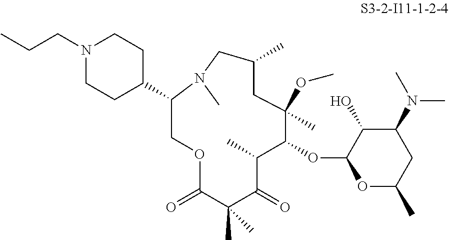

### (3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-propylpiperidin-4-yl)-1-oxa-4-azacyclotridecane-11,13-dione (S3-2-I11-1-2-4) (Compound 63)

Prepared according to the methods of S3-2-I5-1-2-1 from I11 and propionaldehyde to provide the title compound as a formate salt. MS (ESI+) m/z: 626.47 [M+H]⁺; ¹H NMR (400 MHz, Methanol-d₄) δ 4.46 (d, 1H), 4.28 (d, 1H), 4.19 (d, 1H), 3.79-3.68 (m, 1H), 3.66-3.51 (m, 3H), 3.51-3.34 (m, 3H), 3.19-2.87 (m, 11H), 2.87-2.77 (m, 6H), 2.49-2.24 (m, 1H), 2.24-2.10 (m, 1H), 2.10-1.96 (m, 4H), 1.96-1.85 (m, 1H), 1.85-1.65 (m, 4H), 1.65-1.45 (m, 6H), 1.39 (s, 6H), 1.36-1.26 (m, 6H), 1.14-0.96 (m, 6H).

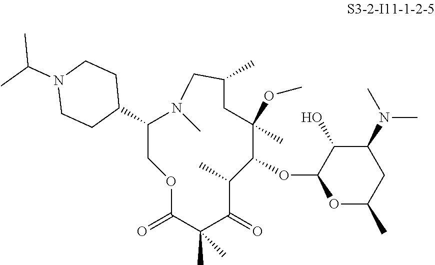

### (3S,6R,8R,9R,10R)-9-(((2S,3R,4S,6R)-4-(dimethylamino)-3-hydroxy-6-methyltetrahydro-2H-pyran-2-yl)oxy)-3-(1-isopropylpiperidin-4-yl)-8-methoxy-4,6,8,10,12,12-hexamethyl-1-oxa-4-azacyclotridecane-11,13-dione (S3-2-I11-1-2-5) (Compound 116)

Prepared according to the methods of S3-2-I5-1-2-1 from I11 and acetone to provide the title compound as a formate salt. MS (ESI+) m/z: 626.47 [M+H]⁺; ¹H NMR (400 MHz, Methanol-d₄) δ 8.44 (s, 3H), 4.46 (d, 1H), 4.29 (d, 1H), 4.20 (d, 1H), 3.79-3.62 (m, 2H), 3.56-3.34 (m, 6H), 3.20-2.94 (m, 10H), 2.83 (s, 6H), 2.51-2.34 (m, 1H), 2.25-2.11 (m, 1H), 2.11-1.91 (m, 5H), 1.91-1.61 (m, 2H), 1.61-1.46 (m, 5H), 1.44-1.27 (m, 18H), 1.08 (d, 3H).

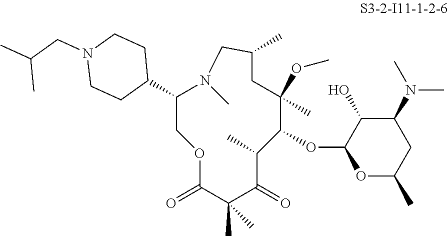

### (3S,6R,8R,9R,10R)-9-(((2S,3R,4S,6R)-4-(dimethylamino)-3-hydroxy-6-methyltetrahydro-2H-pyran-2-yl)oxy)-3-(1-isobutylpiperidin-4-yl)-8-methoxy-4,6,8,10,12,12-hexamethyl-1-oxa-4-azacyclotridecane-11,13-dione (S3-2-I11-1-2-6) (Compound 12)

Prepared according to the methods of S3-2-I5-1-2-1 from I11 and isobutyraldehyde to provide 11.1 mg of the title compound as a formate salt. MS (ESI+) m/z: 640.47 [M+H]⁺; ¹H NMR (400 MHz, Methanol-d₄) δ 4.46 (d, 1H), 4.28 (d, 1H), 4.20 (d, 1H), 3.79-3.67 (m, 1H), 3.64-3.50 (m, 3H), 3.50-3.34 (m, 3H), 3.15-2.85 (m, 12H), 2.83 (s, 6H), 2.44-2.24 (m, 1H), 2.18-2.09 (m, 3H), 2.03-1.90 (m, 2H), 1.89-1.64 (m, 2H), 1.64-1.47 (m, 5H), 1.45-1.25 (m, 13H), 1.12-0.97 (m, 10H).

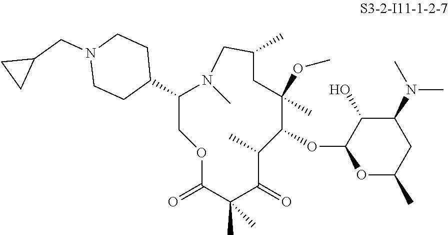

### (3S,6R,8R,9R,10R)-3-(1-(cyclopropylmethyl)piperidin-4-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-2-I11-1-2-7) (Compound 108)

Prepared according to the methods of S3-2-I5-1-2-1 from I11 and cyclopropanecarboxaldehyde to provide 13.5 mg of the title compound as a formate salt. MS (ESI+) m/z: 638.43 [M+H]⁺; ¹H NMR (400 MHz, Methanol-d₄) δ 8.51 (s, 3H), 4.50-4.42 (m, 1H), 4.32-4.24 (m, 1H), 4.21-4.03 (m, 1H), 3.77-3.67 (m, 1H), 3.68-3.56 (m, 2H), 3.50-3.33 (m, 4H), 3.05-2.85 (m, 9H), 2.85-2.62 (m, 9H), 2.08-1.93 (m, 4H), 1.93-1.60 (m, 3H), 1.58-1.45 (m, 5H), 1.42-1.23 (m, 13H), 1.16-1.06 (m, 1H), 1.06-0.91 (m, 3H), 0.78-0.69 (m, 2H), 0.44-0.35 (m, 2H).

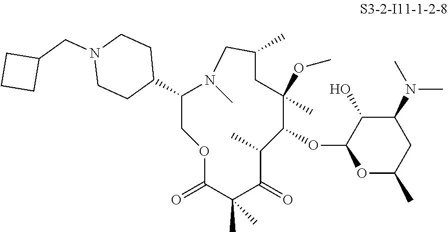

### (3S,6R,8R,9R,10R)-3-(1-(cyclobutylmethyl)piperidin-4-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-2-I11-1-2-8) (Compound 69)

Prepared according to the methods of S3-2-I5-1-2-1 from I11 and cyclobutanecarboxaldehyde to provide 9.81 mg of the title compound as a formate salt. MS (ESI+) m/z: 652.43 [M+H]⁺; ¹H NMR (400 MHz, Methanol-d₄) δ 8.51 (s, 3H), 4.46 (d, 1H), 4.26 (dd, 1H), 4.12 (s, 1H), 3.71 (dtd, 1H), 3.53-3.33 (m, 5H), 3.17-2.86 (m, 7H), 2.81 (s, 10H), 2.63 (s, 2H), 2.26-2.11 (m, 2H), 2.09-1.73 (m, 10H), 1.51 (s, 4H), 1.42-1.21 (m, 12H), 0.99 (s, 3H).

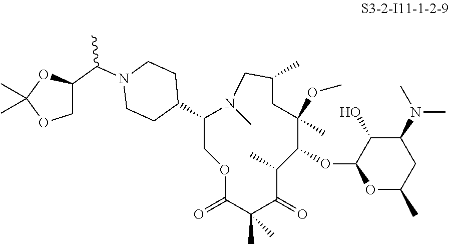

### (3S,6R,8R,9R,10R)-3-(1-(1-((S)-2,2-dimethyl-1,3-dioxolan-4-yl)ethyl)piperidin-4-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-2-I11-1-2-9) (Compound 172)

Prepared according to the methods of S3-2-I5-1-2-1 from I11 and (R)-1-(2,2-dimethyl-1,3-dioxolan-4-yl)ethan-1-one to provide 7.86 mg of the title compound as a formate salt. MS (ESI+) m/z: 712.41 [M+H]⁺; ¹H NMR (400 MHz, Methanol-d₄) δ 8.49 (s, 3H), 4.46 (d, 1H), 4.43-4.33 (m, 1H), 4.33-4.22 (m, 1H), 4.22-4.02 (m, 2H), 3.79-3.62 (m, 2H), 3.51-3.33 (m, 4H), 3.27-2.85 (m, 9H), 2.85-2.58 (m, 9H), 2.21-1.96 (m, 3H), 1.96-1.81 (m, 2H), 1.81-1.62 (m, 2H), 1.63-1.46 (m, 6H), 1.46-1.24 (m, 19H), 1.24-1.09 (m, 3H), 1.09-0.87 (in, 3H).

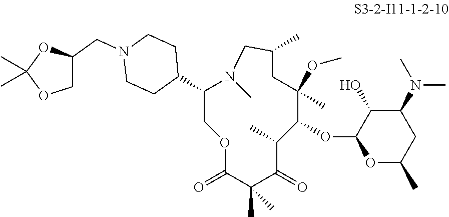

### (3S,6R,8R,9R,10R)-3-(1-(((S)-2,2-dimethyl-1,3-dioxolan-4-yl)methyl)piperidin-4-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-2-I11-1-2-10) (Compound 86)

Prepared according to the methods of S3-2-I5-1-2-1 from I11 and (R)-2,2-dimethyl-1,3-dioxolane-4-carboxaldehyde to provide 9.26 mg of the title compound as a formate salt. MS (ESI+) m/z: 698.47 [M+H]⁺; ¹H NMR (400 MHz, Methanol-d₄) δ 8.46 (s, 3H), 4.46 (d, 1H), 4.44-4.34 (m, 1H), 4.28 (d, 1H), 4.24-4.15 (m, 1H), 4.15-4.09 (m, 1H), 4.09-3.89 (m, 2H), 3.78-3.66 (m, 1H), 3.65-3.55 (m, 2H), 3.51-3.33 (m, 5H), 3.15-2.88 (m, 6H), 2.88-2.65 (m, 7H), 2.08-1.95 (m, 2H), 1.95-1.81 (m, 2H), 1.81-1.68 (m, 1H), 1.68-1.45 (m, 6H), 1.45-1.23 (m, 24H), 1.13-0.94 (m, 3H).

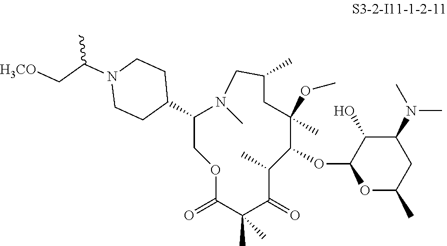

### (3S,6R,8R,9R,10R)-9-(((2S,3R,4S,6R)-4-(dimethylamino)-3-hydroxy-6-methyltetrahydro-2H-pyran-2-yl)oxy)-8-methoxy-3-(1-(1-methoxypropan-2-yl)piperidin-4-yl)-4,6,8,10,12,12-hexamethyl-1-oxa-4-azacyclotridecane-11,13-dione (S3-2-I11-1-2-11) (Compound 124)

Prepared according to the methods of S3-2-I5-1-2-1 from I11 and 1-methoxypropan-2-one to provide 4.95 mg of the title compound as a formate salt. MS (ESI+) m/z: 656.43 [M+H]⁺; ¹H NMR (400 MHz, Methanol-d₄) δ 8.42 (s, 3H), 4.46 (d), 4.28 (d, 1H), 4.22-4.07 (m, 1H), 3.79-3.61 (m, 2H), 3.61-3.34 (m, 11H), 3.19-2.88 (m, 7H), 2.88-2.66 (m, 8H), 2.09-1.94 (m, 4H), 1.94-1.59 (m, 4H), 1.59-1.46 (m, 5H), 1.45-1.24 (m, 16H), 1.10-0.81 (m, 3H).

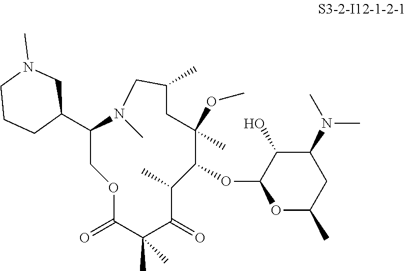

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((S)-1-methylpiperidin-3-yl)-1-oxa-4-azacyclotridecane-11,13-dione (S3-2-I12-1-2-1) (Compound 171)

Prepared according to the methods of S3-2-I5-1-2-1 from I12 and formaldehyde to provide 14.5 mg of the title compound as a formate salt. MS (ESI+) m/z: 200.29 [M+3H]³⁺, 299.82 [M+2H]²⁺, 598.40 [M+H]⁺; ¹H NMR (400 MHz, Methanol-d₄) δ 8.48 (s, 2.6H), 4.49 (dd, 2H), 4.26 (t, 1H), 4.14 (d, 1H), 3.73 (tdd, 1H), 3.59-3.32 (m, 5H), 3.02-2.86 (m, 4H), 2.83 (d, 6H), 2.70 (d, 8H), 2.39-2.23 (m, 1H), 2.07-1.90 (m, 4H), 1.87-1.69 (m, 2H), 1.51 (s, 4H), 1.49-1.40 (m, 2H), 1.38 (d, 3H), 1.35-1.22 (m, 9H), 0.95 (d, 3H).

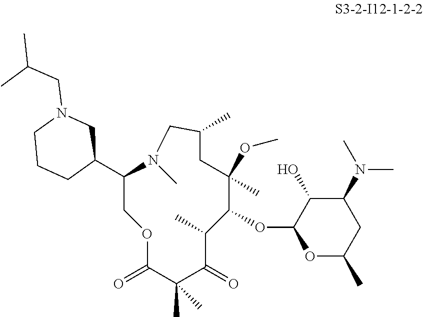

### (3R,6R,8R,9R,10R)-9-(((2S,3R,4S,6R)-4-(Dimethylamino)-3-hydroxy-6-methyltetrahydro-2H-pyran-2-yl)oxy)-3-((S)-1-isobutylpiperidin-3-yl)-8-methoxy-4,6,8,10,12,12-hexamethyl-1-oxa-4-azacyclotridecane-11,13-dione (S3-2-I12-1-2-2) (Compound 39)

Prepared according to the methods of S3-2-I5-1-2-1 from I12 and isobutyraldehyde to provide 10.86 mg of the title compound as a formate salt. MS (ESI+) m/z: 214.34 [M+3H]³⁺, 320.93 [M+2H]²⁺, 640.54 [M+H]⁺; 1H NMR (400 MHz, Methanol-d₄) δ 8.48 (s, 2.5H), 4.56-4.39 (m, 1.6H), 4.33-4.19 (m, 1H), 4.20-4.05 (m, 1H), 3.73 (ddd, 1H), 3.59-3.33 (m, 4H), 3.29-3.16 (m, 1H), 3.00-2.86 (m, 4H), 2.82 (d, 7H), 2.74-2.44 (m, 6H), 2.42-2.19 (m, 2H), 2.14-1.98 (m, 3H), 1.93 (d, 2H), 1.87-1.69 (m, 2H), 1.60-1.49 (m, 4H), 1.52-1.39 (m, 1H), 1.37 (s, 3H), 1.32 (t, 9H), 1.01 (dd, 6H), 0.94 (d, 3H).

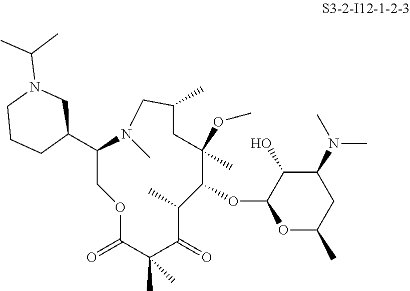

### (3R,6R,8R,9R,10R)-9-(((2S,3R,4S,6R)-4-(dimethylamino)-3-hydroxy-6-methyltetrahydro-2H-pyran-2-yl)oxy)-3-((S)-1-isopropylpiperidin-3-yl)-8-methoxy-4,6,8,10,12,12-hexamethyl-1-oxa-4-azacyclotridecane-11,13-dione (S3-2-I12-1-2-3) (Compound 104)

Prepared according to the methods of S3-2-I5-1-2-1 from I12 and acetone to provide 5.09 mg of the title compound as a formate salt. MS (ESI+) m/z: 209.64 [M+3H]³⁺, 313.83 [M+2H]²⁺, 626.49 [M+H]⁺; ¹H NMR (400 MHz, Methanol-d₄) δ 8.50 (s, 2.6H), 4.49 (d, 1H), 4.41-4.15 (m, 2H), 4.10 (d, 1H), 3.72 (ddd, 11H), 3.62-3.54 (m, 1H), 3.46 (ddd, 2H), 3.42-3.33 (m, 2H), 3.26 (d, 1H), 2.93 (s, 3H), 2.80 (s, 9H), 2.53-2.35 (m, 3H), 2.25-2.14 (m, 1H), 2.04-1.93 (m, 3H), 1.90-1.71 (m, 3H), 1.60-1.49 (m, 4H), 1.40-1.22 (m, 20H), 0.91 (t, 3H).

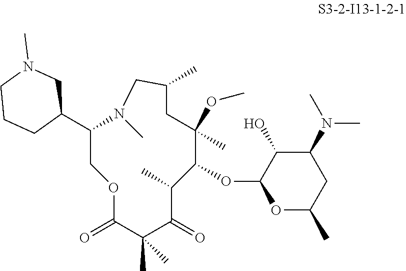

### (3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((S)-1-methylpiperidin-3-yl)-1-oxa-4-azacyclotridecane-11,13-dione (S3-2-I13-1-2-1) (Compound 89)

Prepared according to the methods of S3-2-I5-1-2-1 from I13 and formaldehyde to provide 12.06 mg of the title compound as a formate salt. MS (ESI+) m/z: 200.32 [M+3H]³⁺, 299.86 [M+2H]²⁺, 598.42 [M+H]⁺; ¹H NMR (400 MHz, Methanol-d₄) δ 8.47 (s, 3H), 4.50-4.41 (m, 1H), 4.27 (dd, 1H), 4.17-3.98 (m, 1H), 3.72 (dtd, 1H), 3.53-3.32 (m, 5H), 2.95 (s, 3H), 2.83 (d, 8H), 2.78-2.51 (m, 6H), 2.03 (ddd, 1H), 2.00-1.89 (m, 3H), 1.86-1.76 (m, 2H), 1.68 (s, 1H), 1.59-1.47 (m, 4H), 1.41-1.24 (m, 13H), 1.00 (d, 3H).

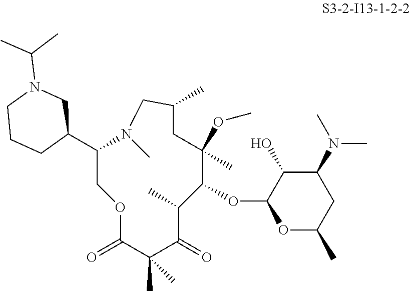

### (3S,6R,8R,9R,10R)-9-(((2S,3R,4S,6R)-4-(dimethylamino)-3-hydroxy-6-methyltetrahydro-2H-pyran-2-yl)oxy)-3-((S)-1-isopropylpiperidin-3-yl)-8-methoxy-4,6,8,10,12,12-hexamethyl-1-oxa-4-azacyclotridecane-11,13-dione (S3-2-I13-1-2-2) (Compound 56)

Prepared according to the methods of S3-2-I5-1-2-1 from I13 and acetone to provide 9.13 mg of the title compound as a formate salt. MS (ESI+) m/z: 209.66 [M+3H]³⁺, 313.83 [M+2H]²⁺, 626.48 [M+H]⁺; ¹H NMR (400 MHz, Methanol-d₄) δ 8.48 (s, 2.5H), 4.47 (d, 1H), 4.35-4.24 (m, 1H), 4.17-3.99 (m, 1H), 3.71 (dtd, 1H), 3.42 (ddt, 5H), 3.28 (s, 1H), 3.04-2.83 (m, 5H), 2.82 (d, 7H), 2.72-2.24 (m, 4H), 2.06-1.91 (m, 3H), 1.91-1.59 (m, 3H), 1.59-1.46 (m, 4H), 1.33 (ddd, 19H), 1.08-0.82 (m, 3H).

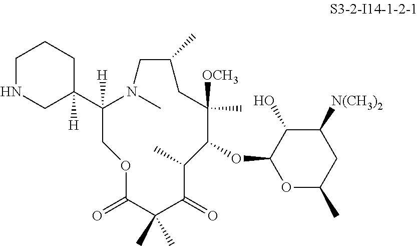

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((R)-piperidin-3-yl)-1-oxa-4-azacyclotridecane-11,13-dione (S3-2-I14-1-2-1)

Prepared according to the methods of S3-2-I5-1-2-1 from I14 and no reductive alkylation to provide the title compound as a formate salt. MS (ESI+) m/z: 584.16 [M+H]⁺; ¹H NMR (400 MHz, Chloroform-d) δ 4.56-4.43 (m, 1H), 4.19 (dt, 2H), 4.08 (d, 1H), 3.83-3.64 (m, 2H), 3.60 (p, 1H), 3.44 (ddd, 2H), 3.37-3.24 (m, 2H), 3.01-2.81 (m, 5H), 2.74 (d, 9H), 2.48-2.16 (m, 4H), 2.08-1.78 (m, 6H), 1.69 (tdd, 1H), 1.57 (s, 2H), 1.52-1.42 (m, 2H), 1.38-1.22 (m, 14H), 0.97-0.79 (m, 3H).

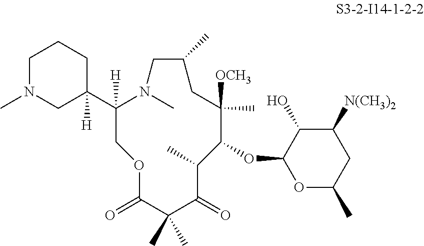

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((R)-1-methylpiperidin-3-yl)-1-oxa-4-azacyclotridecane-11,13-dione (S3-2-I14-1-2-2) (Compound 113)

Prepared according to the methods of S3-2-I5-1-2-1 from I14 and formaldehyde to provide the title compound as a formate salt. MS (ESI+) m/z: 598.37 [M+H]⁺; ¹H NMR (400 MHz, Chloroform-d) δ 4.49 (d, 1H), 4.27 (s, 2H), 4.15 (d, 1H), 3.72 (ddt, 1H), 3.60-3.33 (m, 5H), 2.95 (s, 3H), 2.82 (d, 13H), 2.59 (d, 3H), 2.40-2.11 (m, 2H), 2.11-1.64 (m, 7H), 1.63-1.43 (m, 5H), 1.42-1.18 (m, 13H), 0.94 (d, 3H).

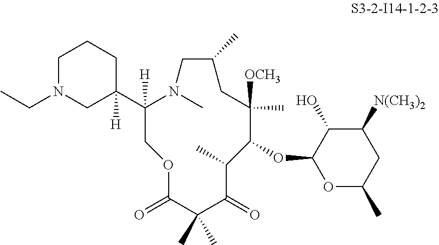

### (3R,6R,8R,9R,10R)-9-(((2S,3R,4S,6R)-4-(dimethylamino)-3-hydroxy-6-methyltetrahydro-2H-pyran-2-yl)oxy)-3-((R)-1-ethylpiperidin-3-yl)-8-methoxy-4,6,8,10,12,12-hexamethyl-1-oxa-4-azacyclotridecane-11,13-dione (S3-2-I14-1-2-3) (Compound 117)

Prepared according to the methods of S3-2-I5-1-2-1 from I14 and acetaldehyde to provide the title compound as a formate salt. MS (ESI+) m/z: 612.41 [M+H]⁺; ¹H NMR (400 MHz, Chloroform-d) δ 4.50 (d, 1H), 4.4-4.28 (m, 2H), 4.15 (d, 1H), 3.72 (dtd, 1H), 3.46 (tdd, 4H), 3.35 (s, 2H), 3.26-3.03 (m, 3H), 2.95 (s, 3H), 2.82 (s, 7H), 2.73 (t, 3H), 2.54 (d, 3H), 2.31-2.14 (m, 1H), 2.11-1.67 (m, 7H), 1.61-1.48 (m, 4H), 1.42-1.23 (m, 16H), 0.94 (d, 3H).

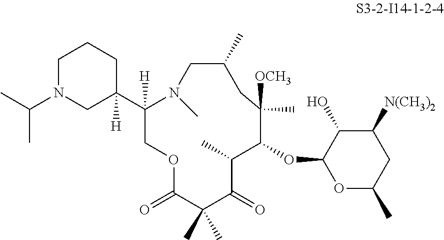

### (3R,6R,8R,9R,10R)-9-(((2S,3R,4S,6R)-4-(dimethylamino)-3-hydroxy-6-methyltetrahydro-2H-pyran-2-yl)oxy)-3-((R)-1-isopropylpiperidin-3-yl)-8-methoxy-4,6,8,10,12,12-hexamethyl-1-oxa-4-azacyclotridecane-11,13-dione (S3-2-I14-1-2-4) (Compound 90)

Prepared according to the methods of S3-2-I5-1-2-1 from I14 and acetone to provide the title compound as a formate salt. MS (ESI+) m/z: 626.43 [M+H]⁺; ¹H NMR (400 MHz, Chloroform-d) δ 4.53 (d, 1H), 4.29-4.08 (m, 2H), 4.01 (d, 1H), 3.68-3.22 (m, 6H), 3.23-3.02 (m, 1H), 2.91 (s, 3H), 2.74 (s, 8H), 2.51-2.16 (m, 7H), 2.18-1.70 (m, 6H), 1.64-1.49 (m, 4H), 1.40-1.14 (m, 21H), 0.87 (d, 3H).

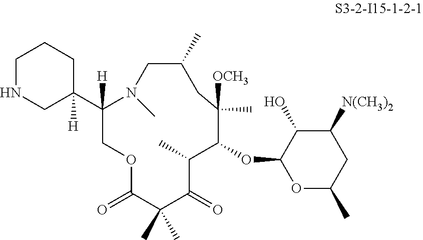

### (3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((R)-piperidin-3-yl)-1-oxa-4-azacyclotridecane-11,13-dione (S3-2-I15-1-2-1)

Prepared according to the methods of S3-2-I5-1-2-1 from I15 and no reductive alkylation to provide the title compound as a formate salt. MS (ESI+) m/z: 584.10 [M+H]⁺; ¹H NMR (400 MHz, Chloroform-d) δ 4.52-4.37 (m, 1H), 4.25 (dd, 1H), 4.15-3.82 (m, 2H), 3.78-3.62 (m, 1H), 3.57-3.22 (m, 6H), 3.12-2.66 (m, 12H), 2.66-2.18 (m, 5H), 2.15-1.60 (m, 7H), 1.61-1.40 (m, 5H), 1.40-1.09 (m, 14H), 1.07-0.73 (m, 3H).

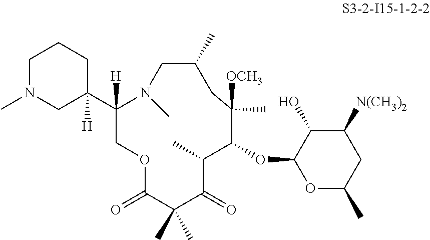

### (3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((R)-1-methylpiperidin-3-yl)-1-oxa-4-azacyclotridecane-11,13-dione (S3-2-I15-1-2-2) (Compound 23)

Prepared according to the methods of S3-2-I5-1-2-1 from I15 and formaldehyde to provide the title compound as a formate salt. MS (ESI+) m/z: 598.16 [M+H]⁺; ¹H NMR (400 MHz, Chloroform-d) δ 4.46 (dd, 1H), 4.30 (dd, 1H), 4.07 (dd, 1H), 3.79-3.62 (m, 1H), 3.56-3.25 (m, 5H), 3.20 (s, 1H), 3.11 (t, 1H), 2.87 (m 16H), 2.56-2.32 (m, 3H), 2.25 (s, 1H), 2.12-1.66 (m, 7H), 1.66-1.41 (m, 5H), 1.42-1.19 (m, 13H), 0.98 (dd, 3H).

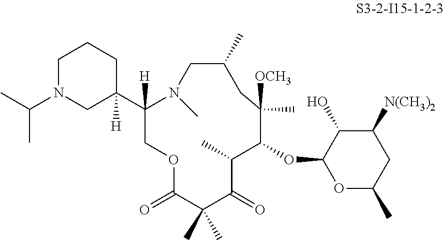

### (3S,6R,8R,9R,10R)-9-(((2S,3R,4S,6R)-4-(dimethylamino)-3-hydroxy-6-methyltetrahydro-2H-pyran-2-yl)oxy)-3-((R)-1-isopropylpiperidin-3-yl)-8-methoxy-4,6,8,10,12,12-hexamethyl-1-oxa-4-azacyclotridecane-11,13-dione (S3-2-I15-1-2-3) (Compound 193)

Prepared according to the methods of S3-2-I5-1-2-1 from I15 and acetone to provide the title compound as a formate salt. MS (ESI+) m/z: 626.15 [M+H]⁺; ¹H NMR (400 MHz, Chloroform-d) δ 4.46 (dd, 1H), 4.36-4.24 (m, 1H), 4.20-3.95 (m, 1H), 3.77-3.65 (m, 1H), 3.55-3.31 (m, 7H), 3.12-2.65 (m, 14H), 2.65-2.34 (m, 4H), 2.25 (s, 1H), 2.10-1.93 (m, 3H), 1.94-1.64 (m, 4H), 1.62-1.42 (m, 6H), 1.40-1.23 (m, 17H), 1.02-0.82 (m, 3H).

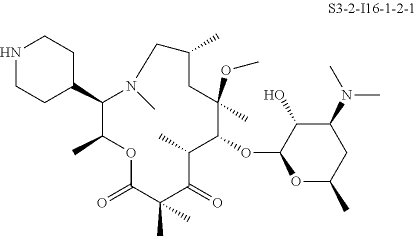

### (2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-(piperidin-4-yl)-1-oxa-4-azacyclotridecane-11,13-dione (S3-2-I16-1-2-1) (Compound 47)

Prepared according to the methods of S3-2-I5-1-2-1 from I16 and no reductive alkylation to provide 4.13 mg of the title compound as a formate salt. MS (ESI+) m/z: 204.49 [M+3H]³⁺, 306.91 [M+2H]²⁺, 612.37 [M+H]⁺; ¹H NMR (400 MHz, Methanol-d₄) δ 8.49 (s, 2H), 5.14 (d, 1H), 4.56 (d, 1H), 4.28 (d, 1H), 3.77 (ddd, 1H), 3.55-3.34 (m, 5H), 3.19 (s, 3H), 2.98 (t, 2H), 2.82 (s, 6H), 2.52 (s, 5H), 2.18-1.82 (m, 6H), 1.63-1.44 (m, 6H), 1.37 (s, 3H), 1.33 (d, 6H), 1.28 (dd, 6H), 0.95 (d, 3H).

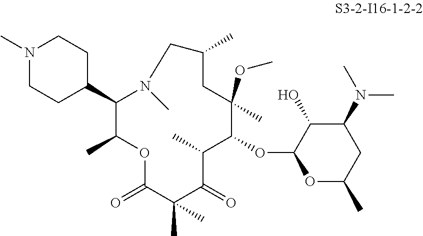

### (2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-(1-methylpiperidin-4-yl)-1-oxa-4-azacyclotridecane-11,13-dione (S3-2-I16-1-2-2) (Compound 130)

Prepared according to the methods of S3-2-I5-1-2-1 from I16 and formaldehyde to provide 6.47 mg of the title compound as a formate salt. MS (ESI+) m/z: 215.00 [M+3H]³⁺, 299.94 [M+2H]²⁺, 598.41 [M+H]⁺; ¹H NMR (400 MHz, Methanol-d₄) δ 8.38 (s, 3H), 5.25 (s, 1H), 4.54 (d, 1H), 4.26 (d, 1H), 3.76 (dd, 1H), 3.58-3.36 (m, 5H), 3.15 (s, 3H), 2.94 (s, 3H), 2.84 (s, 7H), 2.81 (s, 3H), 2.74 (s, 3H), 2.24 (s, 1H), 2.17-2.00 (m, 3H), 1.95-1.66 (m, 4H), 1.61-1.50 (m, 4H), 1.40-1.28 (m, 15H), 1.00 (d, 3H).

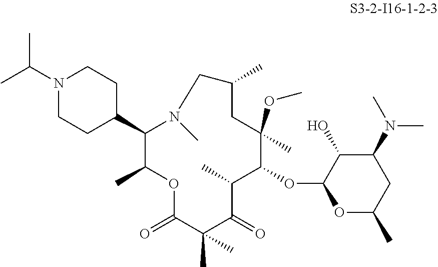

### (2S,3R,6R,8R,9R,10R)-9-(((2S,3R,4S,6R)-4-(dimethylamino)-3-hydroxy-6-methyltetrahydro-2H-pyran-2-yl)oxy)-3-(1-isopropylpiperidin-4-yl)-8-methoxy-2,4,6,8,10,12,12-heptamethyl-1-oxa-4-azacyclotridecane-11,13-dione (S3-2-I16-1-2-3) (Compound 147)

Prepared according to the methods of S3-2-I5-1-2-1 from I16 and acetone to provide 7.32 mg of the title compound as a formate salt. MS (ESI+) m/z: 214.33 [M+3H]³⁺, 320.94 [M+2H]²⁺, 640.61 [M+H]⁺; ¹H NMR (400 MHz, Methanol-d₄) δ 8.52 (s, 2.4H), 5.10 (s, 1H), 4.56 (d, 1H), 4.29 (d, 1H), 3.83-3.70 (m, 1H), 3.55-3.33 (m, 6H), 3.19 (s, 3H), 3.03-2.90 (m, 2H), 2.81 (s, 6H), 2.68-2.39 (m, 5H), 2.14-1.87 (m, 6H), 1.69-1.46 (m, 6H), 1.41-1.30 (m, 14H), 1.27 (dd, 6H), 1.12 (s, 1H), 0.94 (d, 3H).

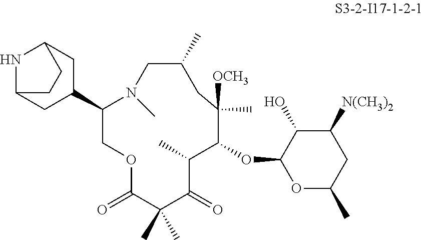

### (3R,6R,8R,9R,10R)-3-(8-azabicyclo[3.2.1]octan-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-2-I17-1-2-1)

Prepared according to the methods of S3-2-I5-1-2-1 from I17 and no reductive alkylation to provide the title compound as a formate salt. MS (ESI+) m/z: 610.42 [M+H]⁺; ¹H NMR (400 MHz, Chloroform-d) δ 4.50 (d, 1H), 4.37-3.88 (m, 5H), 3.88-3.65 (m, 2H), 3.62-3.34 (m, 4H), 3.00-2.86 (m, 3H), 2.82 (s, 7H), 2.46 (t, 3H), 2.34-2.16 (m, 1H), 2.16-1.62 (m, 12H), 1.61-1.41 (m, 5H), 1.41-1.13 (m, 13H), 0.93 (h, 3H).

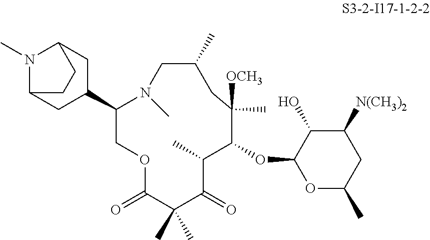

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(8-methyl-8-azabicyclo[3.2.1]octan-3-yl)-1-oxa-4-azacyclotridecane-11,13-dione (S3-2-I17-1-2-2) (Compound 129)

Prepared according to the methods of S3-2-I5-1-2-1 from I17 and formaldehyde to provide the title compound as a formate salt. MS (ESI+) m/z: 624.47 [M+H]⁺; ¹H NMR (400 MHz, Chloroform-d) δ 4.50 (d, 1H), 4.18 (d, 3H), 3.97-3.82 (m, 2H), 3.80-3.65 (m, 1H), 3.62-3.34 (m, 3H), 2.95 (s, 4H), 2.83 (s, 7H), 2.76 (s, 4H), 2.65-2.41 (m, 3H), 2.30 (t, 4H), 2.13-1.72 (m, 10H), 1.54 (d, 4H), 1.42-1.15 (m, 12H), 1.04-0.81 (m, 3H).

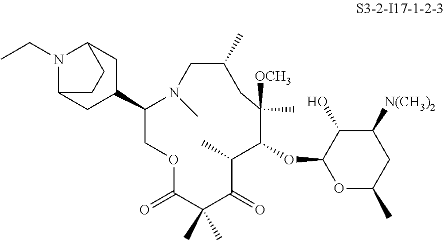

### (3R,6R,8R,9R,10R)-9-(((2S,3R,4S,6R)-4-(dimethylamino)-3-hydroxy-6-methyltetrahydro-2H-pyran-2-yl)oxy)-3-(8-ethyl-8-azabicyclo[3.2.1]octan-3-yl)-8-methoxy-4,6,8,10,12,12-hexamethyl-1-oxa-4-azacyclotridecane-11,13-dione (S3-2-I17-1-2-3) (Compound 15)

Prepared according to the methods of S3-2-I5-1-2-1 from I17 and acetaldehyde to provide the title compound as a formate salt. MS (ESI+) m/z: 638.40 [M+H]⁺; ¹H NMR (400 MHz, Chloroform-d) δ 4.50 (d, 1H), 4.36-4.06 (m, 3H), 4.05-3.93 (m, 2H), 3.72 (dddd, 1H), 3.63-3.51 (m, 1H), 3.50-3.34 (m, 3H), 3.03 (d, 2H), 2.94 (s, 3H), 2.82 (s, 8H), 2.40 (d, 3H), 2.24 (d, 3H), 2.12-1.68 (m, 9H), 1.64-1.43 (m, 5H), 1.42-1.17 (m, 16H), 0.91 (d, 3H).

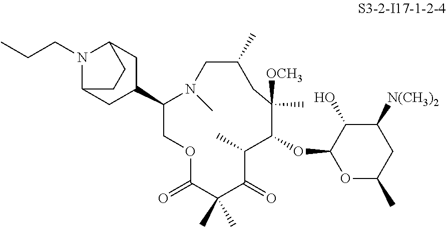

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(8-propyl-8-azabicyclo[3.2.1]octan-3-yl)-1-oxa-4-azacyclotridecane-11,13-dione (S3-2-I17-1-2-4) (Compound 79)

Prepared according to the methods of S3-2-I5-1-2-1 from I17 and propionaldehyde to provide the title compound as a formate salt. MS (ESI+) m/z: 652.53 [M+H]⁺; ¹H NMR (400 MHz, Chloroform-d) δ 4.49 (d, 1H), 4.41-4.09 (m, 3H), 4.00 (dd, 2H), 3.74 (ttd, 1H), 3.62-3.35 (m, 3H), 2.94 (d, 6H), 2.83 (s, 7H), 2.54 (dd, 3H), 2.27 (q, 4H), 2.13-1.69 (m, 12H), 1.64-1.41 (m, 5H), 1.43-1.19 (m, 12H), 1.02 (t, 3H), 0.99-0.79 (m, 3H).

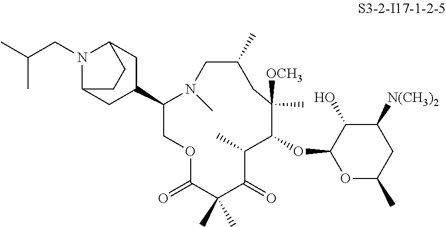

### (3R,6R,8R,9R,10R)-9-(((2S,3R,4S,6R)-4-(dimethylamino)-3-hydroxy-6-methyltetrahydro-2H-pyran-2-yl)oxy)-3-(8-isobutyl-8-azabicyclo[3.2.1]octan-3-yl)-8-methoxy-4,6,8,10,12,12-hexamethyl-1-oxa-4-azacyclotridecane-11,13-dione (S3-2-I17-1-2-5) (Compound 87)

Prepared according to the methods of S3-2-I5-1-2-1 from I17 and isobutyraldehyde to provide the title compound as a formate salt. MS (ESI+) m/z: 666.34 [M+H]⁺; ¹H NMR (400 MHz, Chloroform-d) δ 4.49 (d, 1H), 4.37-4.10 (m, 2H), 4.06-3.95 (m, 2H), 3.80-3.65 (m, 1H), 3.61-3.34 (m, 3H), 3.01 (d, 3H), 2.91-2.71 (m, 10H), 2.55 (t, 3H), 2.27 (q, 4H), 2.18-1.88 (m, 9H), 1.82 (d, 2H), 1.64-1.42 (m, 5H), 1.41-1.18 (m, 13H), 1.07 (d, 6H), 1.01-0.85 (m, 3H).

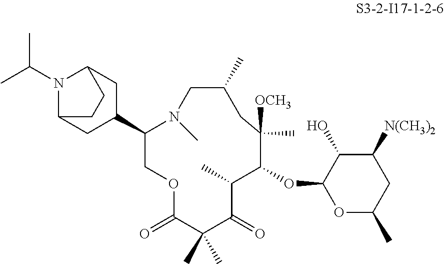

### (3R,6R,8R,9R,10R)-9-(((2S,3R,4S,6R)-4-(dimethylamino)-3-hydroxy-6-methyltetrahydro-2H-pyran-2-yl)oxy)-3-(8-isopropyl-8-azabicyclo[3.2.1]octan-3-yl)-8-methoxy-4,6,8,10,12,12-hexamethyl-1-oxa-4-azacyclotridecane-11,13-dione (S3-2-I17-1-2-6) (Compound 170)

Prepared according to the methods of S3-2-I5-1-2-1 from I17 and acetone to provide the title compound as a formate salt. MS (ESI+) m/z: 652.45 [M+H]⁺; ¹H NMR (400 MHz, Chloroform-d) δ 4.50 (d, 1H), 4.17 (d, 5H), 3.84-3.64 (m, 1H), 3.62-3.33 (m, 4H), 3.11-2.88 (m, 4H), 2.82 (s, 7H), 2.56 (d, 3H), 2.39-2.15 (m, 4H), 2.14-1.68 (m, 10H), 1.53 (q, 4H), 1.47-1.17 (m, 19H), 1.08-0.78 (m, 3H).

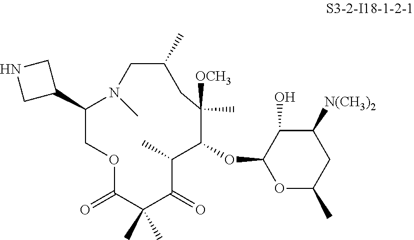

### (3R,6R,8R,9R,10R)-3-(azetidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-2-I18-1-2-1) (Compound 134)

Prepared according to the methods of S3-2-I5-1-2-1 from I18 and no reductive alkylation to provide the title compound as a formate salt. MS (ESI+) m/z: 556.25 [M+H]⁺; ¹H NMR (400 MHz, Methanol-d) δ 8.54 (s, 3H), 4.47 (d, 1H), 4.13-3.89 (m, 7H), 3.67 (t, 1H), 3.60 (s, 2H), 3.44-3.34 (m, 2H), 3.26-3.10 (m, 3H), 2.90 (s, 3H), 2.67 (s, 6H), 2.50 (s, 1H), 2.31 (s, 4H), 1.93 (d, 2H), 1.76 (s, 1H), 1.53 (s, 3H), 1.44 (q, 1H), 1.35 (s, 3H), 1.33-1.22 (m, 10H), 0.88 (d, 3H).

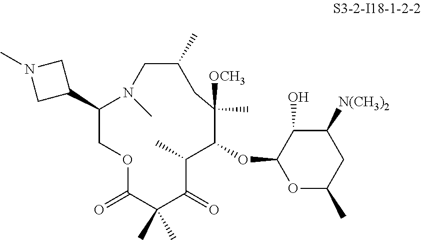

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-methylazetidin-3-yl)-1-oxa-4-azacyclotridecane-11,13-dione (S3-2-I18-1-2-2) (Compound 190)

Prepared according to the methods of S3-2-I5-1-2-1 from I18 and formaldehyde to provide the title compound as a formate salt. MS (ESI+) m/z: 570.33 [M+H]⁺; ¹H NMR (400 MHz, Methanol-d) δ 8.52 (s, 3H), 4.61 (d, 1H), 4.35-4.23 (m, 1H), 4.19-3.82 (m, 3H), 3.72 (d, 1H), 3.60 (s, 1H), 3.51-3.41 (m, 1H), 3.07 (s, 3H), 3.04-2.90 (m, 1H), 2.85-2.67 (m, 12H), 2.41 (s, 3H), 2.07-1.90 (m, 3H), 1.67 (d, 1H), 1.56-1.41 (m, 6H), 1.39-1.19 (m, 15H), 0.93 (d, 3H).

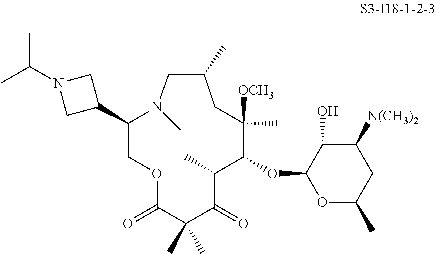

### (3R,6R,8R,9R,10R)-9-(((2S,3R,4S,6R)-4-(dimethylamino)-3-hydroxy-6-methyltetrahydro-2H-pyran-2-yl)oxy)-3-(1-isopropylazetidin-3-yl)-8-methoxy-4,6,8,10,12,12-hexamethyl-1-oxa-4-azacyclotridecane-11,13-dione (S3-2-I18-1-2-3) (Compound 35)

Prepared according to the methods of S3-2-I5-1-2-1 from I18 and acetone to provide the title compound as a formate salt. MS (ESI+) m/z: 598.37 [M+H]⁺; ¹H NMR (400 MHz, Methanol-d) δ 8.33 (s, 4H), 4.67 (d, 1H), 4.44 (dd, 1H), 4.30-4.16 (m, 2H), 4.07 (ddd, 3H), 3.85 (q, 2H), 3.71 (ddd, 1H), 3.49-3.30 (m, 3H), 3.29-3.16 (m, 3H), 3.03 (d, 3H), 3.00-2.86 (m, 2H), 2.80 (d, 6H), 2.73 (s, 3H), 2.14 (s, 1H), 2.05-1.97 (m, 1H), 1.59 (dd, 2H), 1.49 (d, 4H), 1.36 (d, 6H), 1.31 (dd, 6H), 1.17 (dd, 6H), 1.00 (d, 3H).

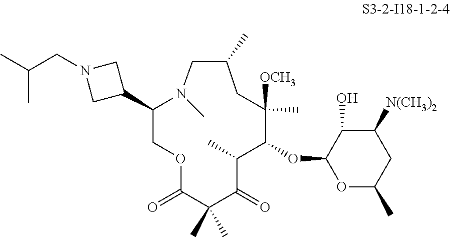

### (3R,6R,8R,9R,10R)-9-(((2S,3R,4S,6R)-4-(dimethylamino)-3-hydroxy-6-methyltetrahydro-2H-pyran-2-yl)oxy)-3-(1-isobutylazetidin-3-yl)-8-methoxy-4,6,8,10,12,12-hexamethyl-1-oxa-4-azacyclotridecane-11,13-dione (S3-2-I18-1-2-4)

Prepared according to the methods of S3-2-I5-1-2-1 from I18 and isobutyraldehyde to provide the title compound as a formate salt. MS (ESI+) m/z: 612.44 [M+H]⁺; ¹H NMR (400 MHz, Methanol-d) δ 8.33 (s, 6H), 4.69 (d, 1H), 4.42 (d, 1H), 4.27 (d, 1H), 4.18 (d, 1H), 4.15-4.04 (m, 3H), 3.99-3.83 (m, 2H), 3.70 (ddd, 1H), 3.47-3.28 (m, 4H), 3.01 (s, 5H), 2.92 (d, 3H), 2.79 (s, 6H), 2.72 (s, 3H), 2.21-2.06 (m, 1H), 2.00 (ddd, 1H), 1.88 (p, 1H), 1.67 (d, 1H), 1.59-1.42 (m, 5H), 1.35 (d, 6H), 1.29 (dd, 6H), 0.99 (d, 3H), 0.95 (d, 6H).

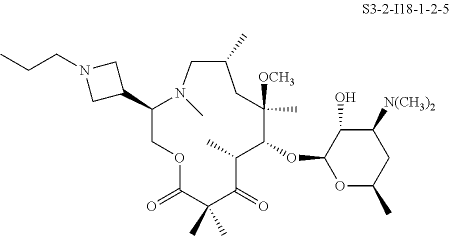

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-propylazetidin-3-yl)-1-oxa-4-azacyclotridecane-11,13-dione (S3-2-I18-1-2-5) (Compound 76)

Prepared according to the methods of S3-2-I5-1-2-1 from I18 and propionaldehyde to provide the title compound as a formate salt. MS (ESI+) m/z: 598.33 [M+H]⁺; ¹H NMR (400 MHz, Methanol-d) ¹H NMR (400 MHz, Chloroform-d) δ 8.54 (s, 2H), 4.46 (d, 1H), 4.24-3.97 (m, 3H), 3.97-3.76 (m, 2H), 3.69 (ddd, 2H), 3.63-3.45 (m, 2H), 3.44-3.34 (m, 3H), 3.27-3.16 (m, 2H), 3.07-2.75 (m, 6H), 2.71 (s, 6H), 2.58-2.20 (m, 4H), 2.04-1.88 (m, 2H), 1.90-1.71 (m, 1H), 1.60-1.41 (m, 7H), 1.41-1.24 (m, 12H), 1.04-0.85 (m, 6H).

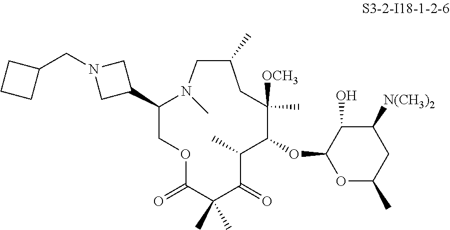

### (3R,6R,8R,9R,10R)-3-(1-(cyclobutylmethyl)azetidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-2-I18-1-2-6) (Compound 17)

Prepared according to the methods of S3-2-I5-1-2-1 from I18 and cyclobutanecarboxaldehyde to provide the title compound as a formate salt. MS (ESI+) m/z: 624.37 [M+H]⁺; ¹H NMR (400 MHz, Methanol-d) δ 8.49 (s, 2H), 4.48 (d, 1H), 4.15 (d, 1H), 4.04 (dd, 11H), 4.01-3.95 (m, 1H), 3.92-3.81 (m, 1H), 3.72 (ddd, 2H), 3.45 (dd, 4H), 3.38 (td, 1H), 3.06 (q, 2H), 3.01-2.88 (m, 5H), 2.80 (s, 6H), 2.77-2.62 (m, 2H), 2.62-2.43 (m, 4H), 2.10 (q, 2H), 2.05-1.92 (m, 3H), 1.92-1.85 (m, 1H), 1.85-1.71 (m, 3H), 1.57-1.46 (m, 4H), 1.37 (s, 3H), 1.35-1.25 (m, 9H), 1.00-0.88 (m, 3H).

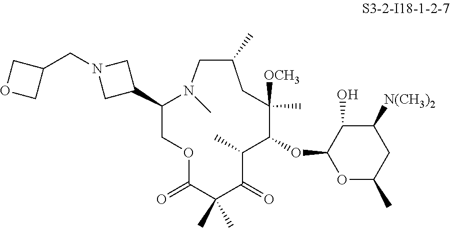

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-(oxetan-3-ylmethyl)azetidin-3-yl)-1-oxa-4-azacyclotridecane-11,13-dione (S3-2-I18-1-2-7) (Compound 91)

Prepared according to the methods of S3-2-I5-1-2-1 from I18 and oxetane-3-carboxaldehyde to provide the title compound as a formate salt. MS (ESI+) m/z: 626.36 [M+H]⁺; ¹H NMR (400 MHz, Methanol-d) ¹H NMR (400 MHz, Chloroform-d) δ 8.54 (s, 2H), 4.77 (dd, 2H), 4.45 (d, 1H), 4.39 (td, 2H), 4.26-4.17 (m, 1H), 4.14 (d, 1H), 4.11-4.01 (m, 1H), 3.75-3.64 (m, 3H), 3.61 (t, 2H), 3.57-3.46 (m, 1H), 3.46-3.37 (m, 2H), 3.25-3.12 (m, 2H), 3.11-2.96 (m, 5H), 2.85 (s, 4H), 2.72 (s, 7H), 2.06-1.94 (m, 3H), 1.58-1.43 (m, 5H), 1.43-1.34 (m, 6H), 1.34-1.26 (m, 7H), 1.06-0.89 (m, 4H).

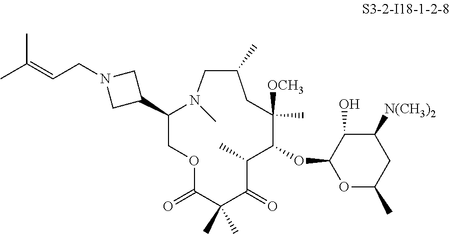

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-(3-methylbut-2-en-1-yl)azetidin-3-yl)-1-oxa-4-azacyclotridecane-11,13-dione (S3-2-I18-1-2-8) (Compound 155)

Prepared according to the methods of S3-2-I5-1-2-1 from I18 and 4-methylpent-3-enal to provide the title compound as a formate salt. MS (ESI+) m/z: 624.40 [M+H]⁺; ¹H NMR (400 MHz, Methanol-d) ¹H NMR (400 MHz, Chloroform-d) δ 8.46 (s, 3H), 5.19 (t, 1H), 4.62-4.26 (m, 2H), 4.16 (d, 2H), 4.13-3.98 (m, 3H), 3.97-3.81 (m, 2H), 3.81-3.55 (m, 5H), 3.55-3.36 (m, 3H), 3.20-3.08 (m, 1H), 3.02-2.95 (m, 3H), 2.86-2.79 (m, 6H), 2.74 (s, 1H), 2.67-2.45 (m, 3H), 2.11-1.91 (m, 2H), 1.89-1.68 (m, 7H), 1.61-1.42 (m, 5H), 1.42-1.16 (m, 12H), 0.96 (d, 3H).

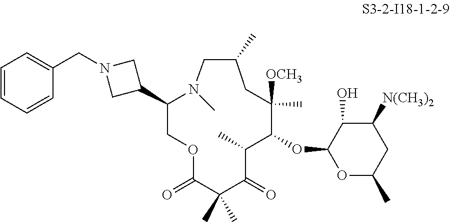

### (3R,6R,8R,9R,10R)-3-(1-benzylazetidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-2-I18-1-2-9) (Compound 101)

Prepared according to the methods of S3-2-I5-1-2-1 from I18 and benzaldehyde to provide the title compound as a formate salt. MS (ESI+) m/z: 646.35 [M+H]⁺; ¹H NMR (400 MHz, Methanol-d) δ 8.42 (s, 3H), 7.45-7.30 (m, 6H), 4.64 (s, 1H), 4.46 (d, 1H), 4.21 (d, 1H), 4.11 (dd, 1H), 3.94-3.60 (m, 6H), 3.59-3.34 (m, 5H), 3.03 (s, 4H), 3.00-2.85 (m, 1H), 2.82 (s, 6H), 2.78-2.58 (m, 3H), 2.20-2.08 (m, 1H), 2.03 (ddd, 1H), 1.73-1.58 (m, 2H), 1.58-1.42 (m, 4H), 1.37 (s, 6H), 1.33 (dd, 7H), 1.01 (d, 3H).

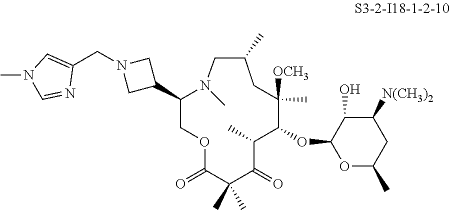

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-((1-methyl-1H-imidazol-4-yl)methyl)azetidin-3-yl)-1-oxa-4-azacyclotridecane-11,13-dione (S3-2-I18-1-2-10) (Compound 173)

Prepared according to the methods of S3-2-I5-1-2-1 from I18 and 1-methyl-1H-imidazole-4-carboxaldehyde to provide the title compound as a formate salt. MS (ESI+) m/z: 650.36 [M+H]⁺; ¹H NMR (400 MHz, Methanol-d) δ 8.40 (s, 3H), 7.63 (s, 1H), 7.14 (s, 1H), 4.47 (d, 1H), 4.35-4.15 (m, 1H), 4.15-3.96 (m, 2H), 3.93-3.79 (m, 2H), 3.79-3.56 (m, 6H), 3.53-3.35 (m, 4H), 3.15-2.92 (m, 5H), 2.90-2.77 (m, 7H), 2.73-2.53 (m, 3H), 2.38 (s, 1H), 2.16-1.89 (m, 2H), 1.73-1.58 (m, 2H), 1.52 (s, 4H), 1.45-1.29 (m, 11H), 1.29-1.21 (m, 2H), 0.99 (d, 3H), 0.91 (d, 1H).

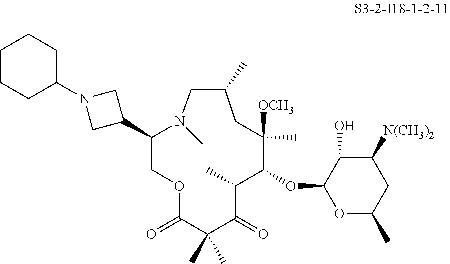

### (3R,6R,8R,9R,10R)-3-(1-cyclohexylazetidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-2-I18-1-2-11) (Compound 73)

Prepared according to the methods of S3-2-I5-1-2-1 from I18 and cyclohexanone to provide the title compound as a formate salt. MS (ESI+) m/z: 638.41 [M+H]⁺; ¹H NMR (400 MHz, Methanol-d) ¹H NMR δ 8.44 (s, 3H), 4.64-4.41 (m, 2H), 4.23-4.05 (m, 3H), 3.99 (d, 1H), 3.91 (d, 1H), 3.83-3.60 (m, 3H), 3.55-3.35 (m, 3H), 3.23-2.92 (m, 5H), 2.88-2.76 (m, 8H), 2.71-2.54 (m, 2H), 2.49-2.22 (m, 1H), 2.13-1.91 (m, 4H), 1.91-1.78 (m, 2H), 1.76-1.63 (m, 2H), 1.62-1.44 (m, 5H), 1.44-1.08 (m, 18H), 0.96 (dd, 3H).

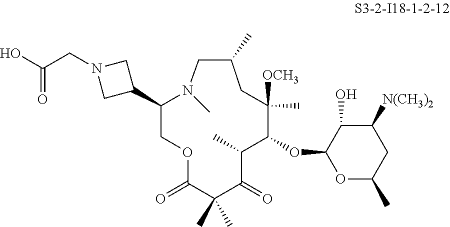

### 2-(3-((3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-11,13-dioxo-1-oxa-4-azacyclotridecan-3-yl)azetidin-1-yl)acetic acid (S3-2-I18-1-2-12)

Prepared according to the methods of S3-2-I5-1-2-1 from I18 and glyoxalic acid to provide the title compound as a formate salt. MS (ESI+) m/z: 614.29 [M+H]⁺; ¹H NMR (400 MHz, Methanol-d) δ 8.40 (s, 2H), 4.58-4.48 (m, 1H), 4.25 (ddt, 2H), 4.06 (dt, 4H), 3.91 (s, 1H), 3.83-3.65 (m, 3H), 3.61-3.34 (m, 4H), 3.22-3.10 (m, 1H), 3.09-2.90 (m, 4H), 2.82 (d, 6H), 2.39 (d, 3H), 2.09-1.97 (m, 2H), 1.94-1.78 (m, 2H), 1.48 (d, 5H), 1.41-1.18 (m, 13H), 1.03-0.79 (m, 3H).

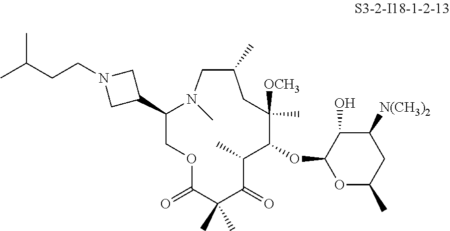

### (3R,6R,8R,9R,10R)-9-(((2S,3R,4S,6R)-4-(dimethylamino)-3-hydroxy-6-methyltetrahydro-2H-pyran-2-yl)oxy)-3-(1-isopentylazetidin-3-yl)-8-methoxy-4,6,8,10,12,12-hexamethyl-1-oxa-4-azacyclotridecane-11,13-dione (S3-2-I18-1-2-13) (Compound 51)

Prepared according to the methods of S3-2-I5-1-2-1 from I18 and 3-methylbutanal to provide the title compound as a formate salt. MS (ESI+) m/z: 626.52 [M+H]⁺; ¹H NMR (400 MHz, Methanol-d) δ 8.47 (s, 3H), 4.48 (d, 1H), 4.19-3.89 (m, 5H), 3.89-3.59 (m, 4H), 3.54-3.34 (m, 3H), 3.24-3.05 (m, 2H), 3.05-2.90 (m, 4H), 2.87-2.78 (m, 7H), 2.77-2.47 (m, 4H), 2.10-1.93 (m, 2H), 1.83-1.70 (m, 0H), 1.64 (p, 1H), 1.48 (d, 5H), 1.43-1.20 (m, 16H), 0.95 (t, 9H).

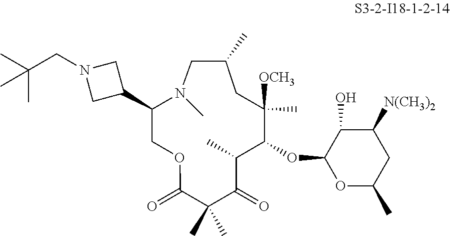

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-neopentylazetidin-3-yl)-1-oxa-4-azacyclotridecane-11,13-dione (S3-2-I18-1-2-14) (Compound 114)

Prepared according to the methods of S3-2-I5-1-2-1 from I18 and pivaldehyde to provide the title compound as a formate salt. MS (ESI+) m/z: 626.57 [M+H]⁺; ¹H NMR (400 MHz, Methanol-d) δ 8.46 (s, 3H), 4.49 (d, 1H), 4.42-4.24 (m, 1H), 4.21-3.94 (m, 4H), 3.93-3.79 (m, 2H), 3.79-3.68 (m, 2H), 3.65-3.36 (m, 4H), 3.20-3.07 (m, 1H), 2.98 (s, 3H), 2.87 (s, 2H), 2.82 (s, 6H), 2.76-2.35 (m, 5H), 2.13-1.89 (m, 2H), 1.84-1.72 (m, 1H), 1.66-1.45 (m, 5H), 1.38 (s, 3H), 1.36-1.23 (m, 10H), 0.95 (d, 4H), 0.63 (d, 3H), 0.32 (d, 3H).

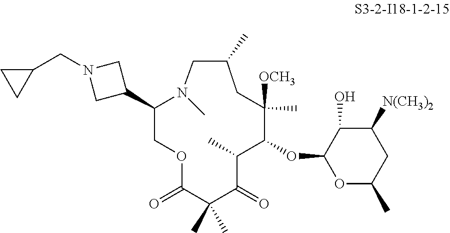

### (3R,6R,8R,9R,10R)-3-(1-(cyclopropylmethyl)azetidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-2-I18-1-2-I5) (Compound 37)

Prepared according to the methods of S3-2-I5-1-2-1 from I18 and cyclopropanecarboxaldehyde to provide the title compound as a formate salt. MS (ESI+) m/z: 610.40 [M+H]⁺, formate salt ¹H NMR (400 MHz, Methanol-d) δ 8.45 (s, 3H), 4.65-4.53 (m, 1H), 4.46 (d, 1H), 4.19 (d, 1H), 4.14-4.03 (m, 2H), 3.91 (s, 1H), 3.80-3.65 (m, 3H), 3.55 (s, 1H), 3.50-3.33 (m, 3H), 3.09 (q, 1H), 3.02 (s, 3H), 2.90-2.78 (m, 7H), 2.73-2.59 (m, 4H), 2.16-1.98 (m, 2H), 1.69-1.59 (m, 2H), 1.57-1.47 (m, 4H), 1.40-1.26 (m, 12H), 1.05-0.97 (m, 3H), 0.96 (d, 8H).

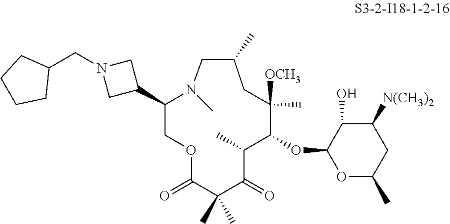

### (3R,6R,8R,9R,10R)-3-(1-(cyclopentylmethyl)azetidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-2-I18-1-2-16) (Compound 103)

Prepared according to the methods of S3-2-I5-1-2-1 from I18 and cyclopentanecarboxaldehyde to provide the title compound as a formate salt. MS (ESI+) m/z: 638.49 [M+H]⁺; ¹H NMR (400 MHz, Methanol-d) δ 8.46 (s, 3H), 4.48 (d, 1H), 4.44-4.29 (m, 1H), 4.22-4.02 (m, 3H), 4.02-3.93 (m, 1H), 3.86 (s, 1H), 3.79-3.57 (m, 3H), 3.55-3.36 (m, 3H), 3.13 (q, 1H), 3.05-2.91 (m, 5H), 2.82 (s, 6H), 2.65 (d, 5H), 2.12-1.94 (m, 3H), 1.91-1.77 (m, 2H), 1.78-1.56 (m, 5H), 1.56-1.44 (m, 5H), 1.37 (s, 3H), 1.36-1.28 (m, 9H), 1.28-1.15 (m, 2H), 0.96 (d, 3H).

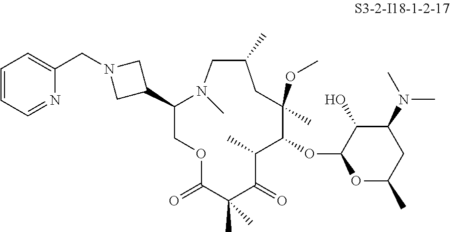

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-(pyridin-2-ylmethyl)azetidin-3-yl)-1-oxa-4-azacyclotridecane-11,13-dione (S3-2-I18-1-2-17) (Compound 115)

Prepared according to the methods of S3-2-I5-1-2-1 from I18 and picolinaldehyde to provide 8.21 mg of the title compound as a formate salt. MS (ESI+) m/z: 324.33 [M+2H]²⁺, 647.39 [M+H]⁺; ¹H NMR (400 MHz, Methanol-d₄) δ 8.69-8.34 (m, 3H), 7.83 (td, 1H), 7.45 (s, 1H), 7.34 (dd, 1H), 4.46 (d, 1H), 4.24 (d, 1H), 4.14 (dd, 11H), 3.92 (s, 2H), 3.83 (s, 2H), 3.77-3.61 (m, 2H), 3.54-3.34 (m, 4H), 3.05 (s, 4H), 3.01-2.85 (m, 2H), 2.81 (d, 6H), 2.75 (s, 3H), 2.17 (s, 1H), 2.08-1.98 (m, 1H), 1.77-1.64 (m, 1H), 1.64-1.55 (m, 2H), 1.54-1.50 (m, 3H), 1.39 (d, 6H), 1.34 (dd, 6H), 1.03 (d, 3H).

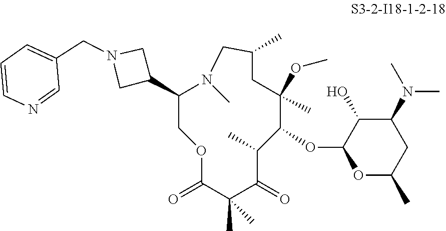

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-(pyridin-3-ylmethyl)azetidin-3-yl)-1-oxa-4-azacyclotridecane-11,13-dione (S3-2-I18-1-2-18) (Compound 119)

Prepared according to the methods of S3-2-I5-1-2-1 from I18 and nicotinaldehyde to provide 15.5 mg of the title compound as a formate salt. MS (ESI+) m/z: 324.32 [M+2H]²⁺, 647.43 [M+H]⁺; ¹H NMR (400 MHz, Methanol-d₄) δ 8.49 (d, 4H), 7.82 (d, 1H), 7.43 (dd, 1H), 4.77 (d, 1H), 4.46 (d, 1H), 4.25 (d, 1H), 4.15 (dd, 1H), 3.86 (s, 1H), 3.81-3.64 (m, 4H), 3.56 (t, 1H), 3.52-3.33 (m, 4H), 3.17 (t, 11H), 3.06 (d, 3H), 2.97 (dd, 2H), 2.82 (s, 6H), 2.79 (s, 3H), 2.21 (s, 1H), 2.04 (tq, 1H), 1.77 (d, 1H), 1.62-1.53 (m, 2H), 1.52 (s, 3H), 1.39 (d, 6H), 1.34 (t, 6H), 1.04 (d, 3H).

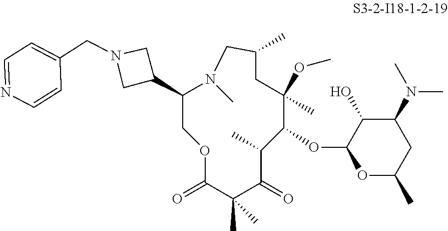

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-(pyridin-4-ylmethyl)azetidin-3-yl)-1-oxa-4-azacyclotridecane-11,13-dione (S3-2-I18-1-2-19) (Compound 176)

Prepared according to the methods of S3-2-I5-1-2-1 from I18 and isonicotinaldehyde to provide 14.2 mg of the title compound as a formate salt. MS (ESI+) m/z: 324.31 [M+2H]²⁺, 647.36 [M+H]⁺; ¹H NMR (400 MHz, Methanol-d₄) δ 8.48 (d, 4H), 7.47-7.33 (m, 2H), 4.80 (d, 1H), 4.46 (dd, 1H), 4.26 (d, 1H), 4.16 (dd, 1H), 3.87 (d, 1H), 3.81-3.63 (m, 4H), 3.58 (td, 11H), 3.51-3.32 (m, 3H), 3.26 (d, 1H), 3.13 (t, 2H), 3.07 (d, 3H), 3.01 (t, 2H), 2.81 (d, 9H), 2.23 (s, 1H), 2.08-2.00 (m, 1H), 1.84-1.72 (m, 1H), 1.63-1.53 (m, 2H), 1.51 (s, 3H), 1.40 (s, 6H), 1.34 (t, 6H), 1.05 (d, 3H).

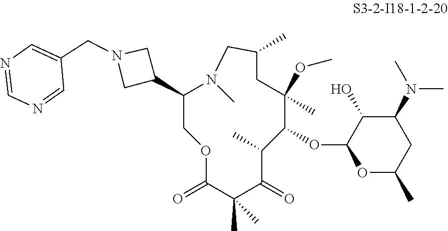

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-(pyrimidin-5-ylmethyl)azetidin-3-yl)-1-oxa-4-azacyclotridecane-11,13-dione (S3-2-I18-1-2-20) (Compound 177)

Prepared according to the methods of S3-2-I5-1-2-1 from I18 and pyrimidine-5-carboxaldehyde to provide 11.1 mg of the title compound as a formate salt. MS (ESI+) m/z: 324.86 [M+2H]²⁺, 648.36 [M+H]⁺; ¹H NMR (400 MHz, Methanol-d₄) δ 9.08 (s, 1H), 8.75 (s, 2H), 8.50 (s, 2H), 4.77 (s, 11H), 4.46 (d, 1H), 4.25 (d, 1H), 4.16 (dd, 1H), 3.84 (s, 11H), 3.79-3.60 (m, 4H), 3.54 (td, 11H), 3.51-3.42 (m, 11H), 3.38 (ddt, 2H), 3.26 (t, 11H), 3.12 (t, 11H), 3.06 (s, 3H), 2.97 (dd, 2H), 2.81 (s, 9H), 2.21 (s, 11H), 2.08-1.97 (m, 11H), 1.76 (d, 11H), 1.65-1.54 (m, 1H), 1.54-1.45 (m, 4H), 1.40 (d, 6H), 1.34 (t, 6H), 1.04 (d, 3H).

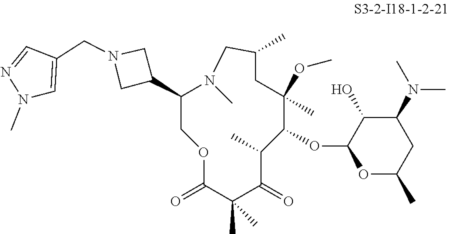

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-((1-methyl-1H-pyrazol-4-yl)methyl)azetidin-3-yl)-1-oxa-4-azacyclotridecane-11,13-dione (S3-2-I18-1-2-21) (Compound 49)

Prepared according to the methods of S3-2-I5-1-2-1 from I18 and 1-methyl-1H-pyrazole-4-carbaldehyde to provide 9.13 mg of the title compound as a formate salt. MS (ESI+) m/z: 278.83 [M+3H]³⁺, 325.81 [M+2H]²⁺, 650.43 [M+H]⁺; ¹H NMR (400 MHz, Methanol-d₄) δ 8.48 (s, 2H), 7.67 (s, 1H), 7.51 (s, 1H), 4.60-4.50 (m, 1H), 4.47 (d, 1H), 4.19 (d, 1H), 4.09 (dd, 1H), 3.88 (s, 6H), 3.80-3.56 (m, 4H), 3.55-3.35 (m, 4H), 3.01 (s, 4H), 2.82 (d, 7H), 2.64 (s, 3H), 2.15-1.95 (m, 2H), 1.65 (s, 2H), 1.58-1.46 (m, 4H), 1.37 (d, 6H), 1.33 (d, 6H), 0.99 (d, 3H).

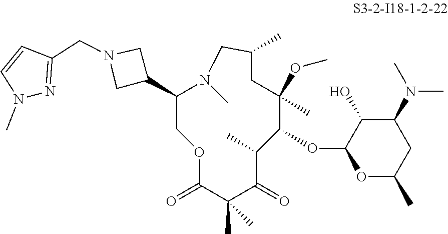

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-((1-methyl-1H-pyrazol-3-yl)methyl)azetidin-3-yl)-1-oxa-4-azacyclotridecane-11,13-dione (S3-2-I18-1-2-22) (Compound 16)

Prepared according to the methods of S3-2-I5-1-2-1 from I18 and 1-methyl-1H-pyrazole-3-carbaldehyde to provide 10.5 mg of the title compound as a formate salt. MS (ESI+) m/z: 325.85 [M+2H]²⁺, 650.39 [M+H], ¹H NMR (400 MHz, Methanol-d₄) δ 8.48 (s, 2H), 7.56 (d, 1H), 6.27 (d, 1H), 4.59 (s, 1H), 4.47 (d, 1H), 4.21 (d, 1H), 4.14-4.01 (m, 1H), 3.94-3.78 (m, 6H), 3.78-3.62 (m, 3H), 3.62-3.49 (m, 1H), 3.49-3.34 (m, 4H), 3.03 (s, 4H), 2.82 (s, 7H), 2.69 (s, 2H), 2.18-1.97 (m, 2H), 1.58 (s, 2H), 1.55-1.48 (m, 4H), 1.38 (d, 6H), 1.33 (dd, 6H), 1.01 (d, 3H).

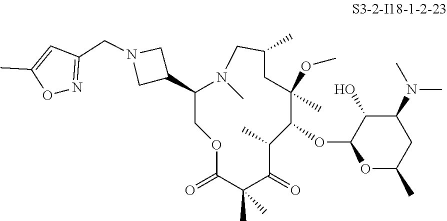

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-((5-methylisoxazol-3-yl)methyl)azetidin-3-yl)-1-oxa-4-azacyclotridecane-11,13-dione (S3-2-I18-1-2-23) (Compound 132)

Prepared according to the methods of S3-2-I5-1-2-1 from I18 and 5-methylisoxazole-3-carbaldehyde to provide 14.52 mg of the title compound as a formate salt. MS (ESI+) m/z: 326.34 [M+2H]²⁺, 651.35 [M+H]⁺; ¹H NMR (400 MHz, Methanol-d₄) δ 8.50 (s, 2H), 6.11 (s, 1H), 4.75 (s, 1H), 4.46 (d, 1H), 4.25 (d, 1H), 4.14 (dd, 1H), 3.93-3.77 (m), 3.77-3.62 (m, 4H), 3.58 (td, 1H), 3.51-3.33 (m, 3H), 3.33-3.26 (m, 1H), 3.14 (t, 1H), 3.06 (s, 3H), 3.02-2.92 (m, 2H), 2.80 (s, 9H), 2.40 (s, 3H), 2.22 (s, 1H), 2.09-1.95 (m, 1H), 1.76 (d, 1H), 1.62-1.47 (m, 5H), 1.40 (s, 6H), 1.34 (t, 6H), 1.04 (d, 3H).

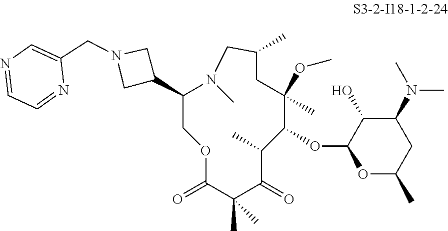

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-(pyrazin-2-ylmethyl)azetidin-3-yl)-1-oxa-4-azacyclotridecane-11,13-dione (S3-2-I18-1-2-24) (Compound 55)

Prepared according to the methods of S3-2-I5-1-2-1 from I18 and pyrazine-2-carboxaldehyde to provide 5.1 mg of the title compound as a formate salt. MS (ESI+) m/z: 324.85 [M+2H]²⁺, 648.32 [M+H]⁺; ¹H NMR (400 MHz, Methanol-d₄) δ 8.70-8.41 (m, 5H), 4.71 (s, 1H), 4.46 (d, 1H), 4.24 (d, 1H), 4.19-4.09 (m, 1H), 3.89 (s, 2H), 3.85-3.68 (m, 3H), 3.63 (t, 1H), 3.49-3.34 (m, 4H), 3.23 (t, 1H), 3.05 (s, 3H), 3.03-2.90 (m, 2H), 2.79 (s, 9H), 2.18 (s, 1H), 2.02 (dt, 1H), 1.55 (s, 1H), 1.47 (s, 4H), 1.39 (s, 6H), 1.33 (t, 6H), 1.03 (d, 3H).

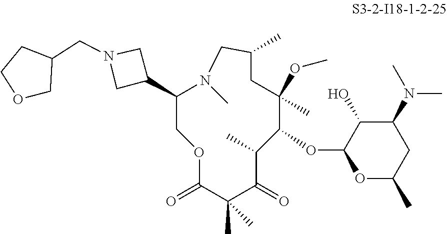

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-((tetrahydrofuran-3-yl)methyl)azetidin-3-yl)-1-oxa-4-azacyclotridecane-11,13-dione (S3-2-I18-1-2-25) (Compound 127)

Prepared according to the methods of S3-2-I5-1-2-1 from I18 and tetrahydrofuran-3-carboxaldehyde to provide the title compound as a formate salt. MS (ESI+) m/z: 320.84 [M+2H]²⁺, 640.45 [M+H]⁺; ¹H NMR (400 MHz, Methanol-d₄) δ 8.51 (s, 2H), 4.62 (s, 1H), 4.47 (d, 1H), 4.22 (d, 1H), 4.11 (dd, 1H), 4.00-3.79 (m, 3H), 3.74 (dq, 4H), 3.44 (ddd, 5H), 3.03 (s, 4H), 2.96-2.86 (m, 2H), 2.82 (s, 7H), 2.77-2.59 (m, 4H), 2.34 (p, 1H), 2.20-1.95 (m, 3H), 1.72-1.54 (m, 3H), 1.52 (s, 4H), 1.38 (d, 6H), 1.33 (dd, 6H), 1.01 (d, 3H).

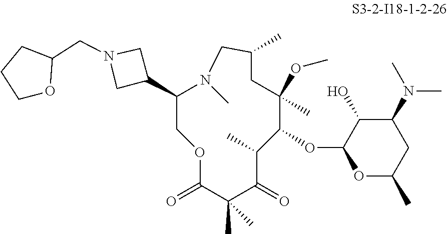

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-((tetrahydrofuran-2-yl)methyl)azetidin-3-yl)-1-oxa-4-azacyclotridecane-11,13-dione (S3-2-I18-1-2-26) (Compound 164)

Prepared according to the methods of S3-2-I5-1-2-1 from I18 and tetrahydrofuran-2-carboxaldehyde to provide the title compound as a formate salt. MS (ESI+) m/z: 320.85 [M+2H]²⁺, 640.46 [M+H]⁺; ¹H NMR (400 MHz, Methanol-d₄) δ 8.48 (s, 2H), 4.48 (d, 2H), 4.19 (d, 1H), 4.08 (dd, 1H), 3.99 (s, 2H), 3.86 (q, 2H), 3.73 (dq, 4H), 3.59-3.35 (m, 4H), 3.12-2.91 (m, 5H), 2.82 (s, 8H), 2.63 (s, 3H), 2.04 (dq, 3H), 1.91 (dq, 2H), 1.61-1.43 (m, 1H), 1.61-1.43 (m, 6H), 1.37 (d, 6H), 1.33 (d, 6H), 0.99 (d, 3H).

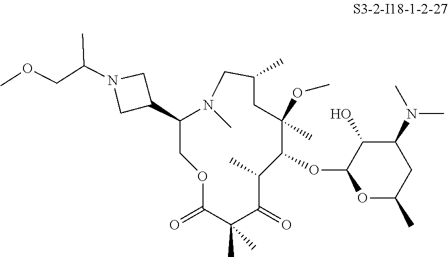

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-3-(1-(1-methoxypropan-2-yl)azetidin-3-yl)-4,6,8,10,12,12-hexamethyl-1-oxa-4-azacyclotridecane-11,13-dione (S3-2-I18-1-2-27) (Compound 32)

Prepared according to the methods of S3-2-I5-1-2-1 from I18 and methoxyacetone to provide 13.14 mg of the title compound as a formate salt. MS (ESI+) m/z: 210.28 [M+3H]³⁺, 314.85 [M+2H]²⁺, 628.44 [M+H]⁺; ¹H NMR (400 MHz, Methanol-d₄) δ 8.49 (s, 2.5H), 4.52 (s, 0.5H), 4.47 (d, 1H), 4.19 (d, 1H), 4.13-4.04 (m, 1H), 4.03-3.96 (m, 1H), 3.85 (s, 1H), 3.79-3.60 (m, 3H), 3.54 (s, 1H), 3.50-3.33 (m, 8H), 3.11-2.94 (s, 5H), 2.82 (s, 7H), 2.65 (s, 3H), 2.17-1.99 (m, 2H), 1.74-1.51 (m, 2H), 1.55-1.46 (m, 4H), 1.37 (d, 6H), 1.33 (d, 6H), 1.09 (d, 3H), 0.99 (d, 3H).

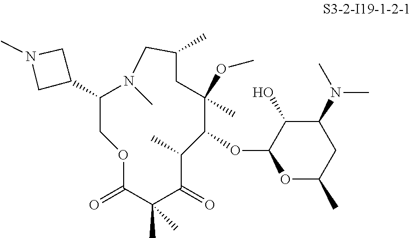

### (3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-methylazetidin-3-yl)-1-oxa-4-azacyclotridecane-11,13-dione (S3-2-I19-1-2-1) (Compound 99)

Prepared according to the methods of S3-2-I5-1-2-1 from I19 and formaldehyde to provide the title compound as a formate salt. MS (ESI+) m/z: 570.36 [M+H]⁺, formate salt, ¹H NMR (400 MHz, Methanol-d₄) δ 8.44 (s, 3H), 4.46 (d, 1H), 4.15-4.04 (m, 3H), 4.04-3.93 (m, 1H), 3.93-3.77 (m, 2H), 3.77-3.66 (m, 1H), 3.66-3.49 (m, 2H), 3.49-3.34 (m, 2H), 3.16 (h, 1H), 2.93 (s, 3H), 2.86-2.69 (m, 9H), 2.51 (s, 5H), 2.08-2.00 (m, 1H), 1.96 (d, 2H), 1.60-1.47 (m, 4H), 1.42-1.24 (m, 13H), 0.97 (d, 3H).

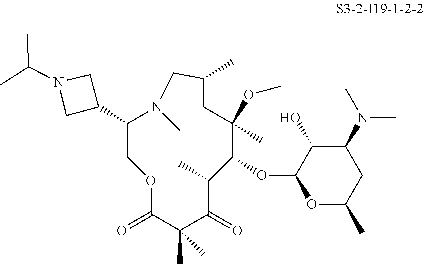

### (3S,6R,8R,9R,10R)-9-(((2S,3R,4S,6R)-4-(dimethylamino)-3-hydroxy-6-methyltetrahydro-2H-pyran-2-yl)oxy)-3-(1-isopropylazetidin-3-yl)-8-methoxy-4,6,8,10,12,12-hexamethyl-1-oxa-4-azacyclotridecane-11,13-dione (S3-2-I19-1-2-2) (Compound 186)

Prepared according to the methods of S3-2-I5-1-2-1 from I19 and acetone to provide the title compound as a formate salt. MS (ESI+) m/z: 598.37 [M+H]⁺; ¹H NMR (400 MHz, Methanol-d₄) δ 8.45 (s, 2H), 4.46 (d, 1H), 4.10 (t, 6H), 3.72 (td, 2H), 3.44 (ddd, 4H), 3.06 (q, 2H), 2.92 (d, 3H), 2.82 (s, 6H), 2.63-2.33 (m, 5H), 2.09-1.82 (m, 3H), 1.53 (d, 4H), 1.41-1.23 (m, 14H), 1.16 (d, 3H), 0.97 (dd, 3H).

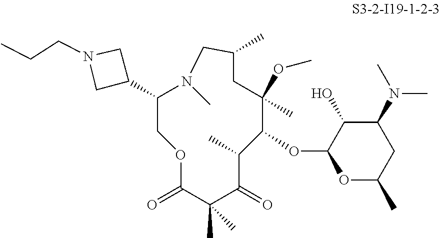

### (3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-propylazetidin-3-yl)-1-oxa-4-azacyclotridecane-11,13-dione (S3-2-I19-1-2-3) (Compound 29)

Prepared according to the methods of S3-2-I5-1-2-1 from I19 and propionaldehyde to provide the title compound as a formate salt. MS (ESI+) m/z: 598.45 [M+H]⁺; ¹H NMR (400 MHz, Methanol-d₄) δ 8.48 (s, 3H), 4.46 (d, 1H), 4.40-4.20 (m, 1H), 4.20-3.99 (m, 4H), 3.97-3.78 (m, 2H), 3.78-3.56 (m, 2H), 3.56-3.34 (m, 3H), 3.21 (h, 1H), 3.01 (t, 2H), 2.94 (s, 3H), 2.83 (s, 6H), 2.55 (s, 4H), 2.07-1.90 (m, 2H), 1.90-1.74 (m, 1H), 1.63-1.45 (m, 6H), 1.44-1.25 (m, 13H), 1.04-0.92 (m, 6H).

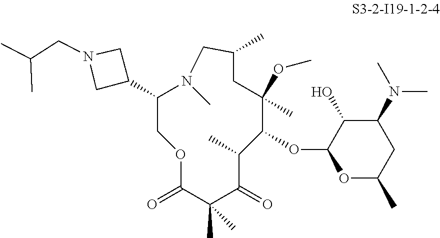

### (3S,6R,8R,9R,10R)-9-(((2S,3R,4S,6R)-4-(dimethylamino)-3-hydroxy-6-methyltetrahydro-2H-pyran-2-yl)oxy)-3-(1-isobutylazetidin-3-yl)-8-methoxy-4,6,8,10,12,12-hexamethyl-1-oxa-4-azacyclotridecane-11,13-dione (S3-2-I19-1-2-4) (Compound 81)

Prepared according to the methods of S3-2-I5-1-2-1 from I19 and isobutyraldehyde to provide the title compound as a formate salt. MS (ESI+) m/z: 612.41 [M+H]⁺; ¹H NMR (400 MHz, Methanol-d₄) δ 8.47 (s, 3H), 4.46 (d, 1H), 4.43-4.24 (m, 1H), 4.22-3.97 (m, 4H), 3.95-3.77 (m, 2H), 3.77-3.62 (m, 2H), 3.55-3.33 (m, 3H), 3.22 (h, 1H), 2.95 (s, 3H), 2.88 (d, 2H), 2.83 (s, 6H), 2.72-2.45 (m, 4H), 2.08-1.94 (m, 2H), 1.89 (d, 1H), 1.84-1.70 (m, 1H), 1.60-1.40 (m, 5H), 1.40-1.26 (m, 12H), 1.04-0.92 (m, 9H).

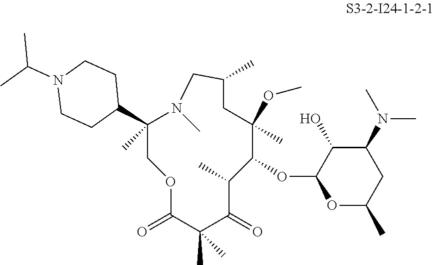

### (3R,6R,8R,9R,10R)-9-(((2S,3R,4S,6R)-4-(dimethylamino)-3-hydroxy-6-methyltetrahydro-2H-pyran-2-yl)oxy)-3-(1-isopropylpiperidin-4-yl)-8-methoxy-3,4,6,8,10,12,12-heptamethyl-1-oxa-4-azacyclotridecane-11,13-dione (S3-2-I24-1-2-1) (Compound 158)

Prepared according to the methods of S3-2-I5-1-2-1 from I24 and acetone to provide the title compound as a formate salt. MS (ESI+) m/z: 640.30 [M+H]⁺; ¹H NMR (400 MHz, Methanol-d) δ 8.55 (s, 1H), 4.44 (d, 1H), 4.26-3.97 (m, 3H), 3.68-3.55 (m, 1H), 3.55-3.42 (m, 1H), 3.26-3.15 (m, 1H), 3.13-2.81 (m, 5H), 2.81-2.60 (m, 2H), 2.60-2.39 (m, 7H), 2.39-2.13 (m, 4H), 2.04-1.70 (m, 6H), 1.70-1.46 (m, 6H), 1.46-1.17 (m, 20H), 1.17-0.98 (m, 5H), 0.89 (d, 3H).

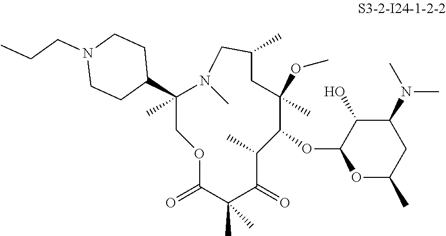

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-3,4,6,8,10,12,12-heptamethyl-3-(1-propylpiperidin-4-yl)-1-oxa-4-azacyclotridecane-11,13-dione (S3-2-I24-1-2-2) (Compound 143)

Prepared according to the methods of S3-2-I5-1-2-1 from I24 and propionaldehyde to provide the title compound as a formate salt. MS (ESI+) m/z: 640.32 [M+H]⁺; ¹H NMR (400 MHz, Methanol-d) δ 8.55 (s, 2H), 4.50 (d, 1H), 4.22-4.06 (m, 2H), 3.72 (ddt, Hz, 1H), 3.58-3.33 (m, 5H), 3.04 (s, 3H), 2.95-2.85 (m, 3H), 2.85-2.54 (m, 11H), 2.02 (ddd, 4H), 1.83 (s, 3H), 1.72 (tq, 3H), 1.58-1.44 (m, 5H), 1.43-1.27 (m, 13H), 1.22 (s, 3H), 0.99 (dd, 6H).

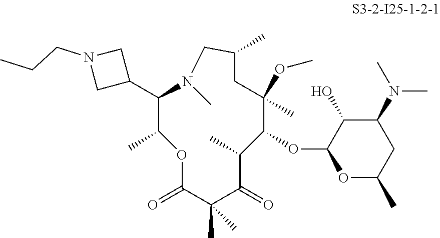

### (2R,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-(1-propylazetidin-3-yl)-1-oxa-4-azacyclotridecane-11,13-dione (S3-2-I25-1-2-1) (Compound 136)

Prepared according to the methods of S3-2-I5-1-2-1 from I25 and propionaldehyde to provide 12.7 mg of the title compound as a formate salt. MS (ESI+) m/z: 204.8 [M+3H]³⁺, 306.6 [M+2H]²⁺, 612.3 [M+H]⁺; ¹H NMR (400 MHz, Methanol-d₄) δ 8.48 (s, 2H), 4.52 (d, 1H), 4.30-3.76 (m, 5H), 3.76-3.61 (m, 1H), 3.60-3.51 (m, 1H), 3.47 (dd, 1H), 3.39 (ddd, 1H), 3.20-2.96 (m, 4H), 2.93 (s, 3H), 2.82 (d, 6H), 2.61-2.05 (m, 4H), 2.01 (ddd, 1H), 1.91-1.66 (m, 2H), 1.66-1.44 (m, 6H), 1.34 (s, 4H), 1.30 (d, 3H), 1.28-1.17 (m, 7H), 1.14 (d, 3H), 0.97 (t, 4H), 0.94-0.84 (m, 2H).

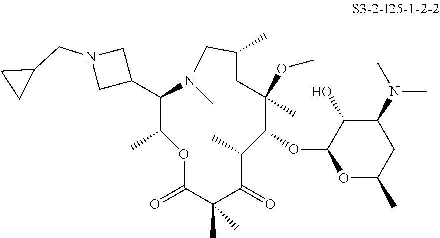

### (2R,3R,6R,8R,9R,10R)-3-(1-(cyclopropylmethyl)azetidin-3-yl)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-1-oxa-4-azacyclotridecane-11,13-dione (S3-2-I25-1-2-2) (Compound 146)

Prepared according to the methods of S3-2-I5-1-2-1 from I25 and cyclopropane carboxaldehyde to provide 11.7 mg of the title compound as a formate salt. MS (ESI+) m/z: 208.8 [M+3H]³⁺, 312.6 [M+2H]²⁺, 624.3 [M+H]⁺; ¹H NMR (400 MHz, Methanol-d₄) δ 8.47 (s, 3H), 4.52 (d, 1H), 4.25-3.84 (m, 5H), 3.77-3.62 (m, 1H), 3.59-3.51 (m, 1H), 3.48 (dd, 1H), 3.39 (ddd, 1H), 3.23-3.09 (m, 1H), 3.09-2.86 (m, 3H), 2.94 (s, 3H) 2.82 (s, 6H), 2.45 (s, 2H), 2.21 (s, 2H), 2.01 (ddd, 1H), 1.90-1.65 (m, 2H), 1.59 (s, 3H), 1.56-1.45 (m, 1H), 1.35 (s, 3H), 1.30 (dd, 4H), 1.28-1.19 (m, 6H), 1.15 (d, 3H), 1.06-0.81 (m, 4H), 0.64 (d, 2H), 0.35 (d, 2H).

The following examples were prepared according to the methods of S3-2-I5-1-2-1, substituting the appropriate intermediate (Table 2) in Scheme 1, the appropriate aldehyde for formaldehyde in Scheme 1 to give S1-3-I—R₅, and the appropriate aldehyde or ketone for formaldehyde in Scheme 3:

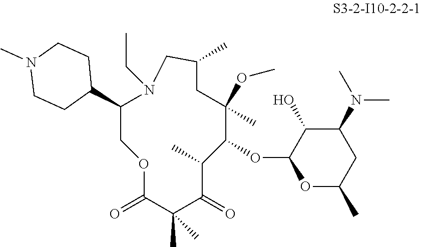

### (3R,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-penta methyl-3-(1-methylpiperidin-4-yl)-1-oxa-4-azacyclotridecane-11,13-dione (S3-2-I10-2-2-1)

Prepared according to the methods of S3-2-I5-1-2-1 from S1-3-I10-2 and formaldehyde to provide the title compound as a formate salt. MS (ESI+) m/z: 612.26 [M+H]⁺. ¹H NMR (400 MHz, Methanol-d₄) δ 4.46 (d, 1H), 4.25 (d, 1H), 4.02 (d, 2H), 3.77-3.60 (m, 2H), 3.49-3.23 (m, 4H), 2.91-2.57 (m, 18H), 2.44-2.14 (m, 2H), 2.12-1.93 (m, 2H), 1.86 (d, 3H), 1.63-1.43 (m, 6H), 1.40-1.22 (m, 12H), 1.08 (t, 4H), 0.87 (d, 3H).

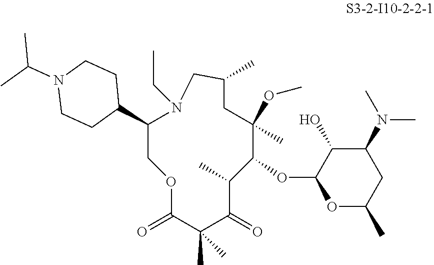

### (3R,6R,8R,9R,10R)-9-(((2S,3R,4S,6R)-4-(dimethylamino)-3-hydroxy-6-methyltetrahydro-2H-pyran-2-yl)oxy)-4-ethyl-3-(1-isopropylpiperidin-4-yl)-8-methoxy-6,8,10,12,12-pentamethyl-1-oxa-4-azacyclotridecane-11,13-dione (S3-2-I10-2-2-2) (Compound 195)

Prepared according to the methods of S3-2-I5-1-2-1 from S1-3-I10-2 and acetone to provide the title compound as a formate salt. MS (ESI+) m/z: 640.12 [M+H]⁺. ¹H NMR (400 MHz, Methanol-d₄) δ 4.45 (d, 1H), 4.30 (s, 1H), 4.02 (d, 2H), 3.81-3.55 (m, 2H), 3.43 (dtt, 5H), 3.07-2.62 (m, 15H), 2.34 (s, 1H), 2.22-2.05 (m, 2H), 2.07-1.88 (m, 3H), 1.73 (d, 3H), 1.60-1.45 (m, 4H), 1.31 (dt, 18H), 1.11 (q, 4H), 0.87 (d, 3H).

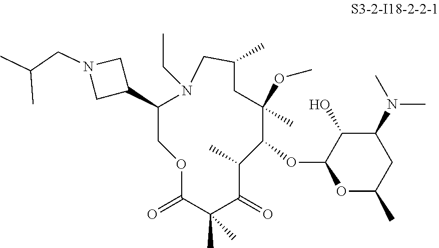

### (3R,6R,8R,9R,10R)-9-(((2S,3R,4S,6R)-4-(dimethylamino)-3-hydroxy-6-methyltetrahydro-2H-pyran-2-yl)oxy)-4-ethyl-3-(1-isobutylazetidin-3-yl)-8-methoxy-6,8,10,12,12-pentamethyl-1-oxa-4-azacyclotridecane-11,13-dione (S3-2-I18-2-2-1) (Compound 70)

Prepared according to the methods of S3-2-I5-1-2-1 from S1-3-I18-2 and isobutyraldehyde to provide the title compound as a formate salt. MS (ESI+) m/z: 626.46 [M+H]⁺, formate salt, ¹H NMR (400 MHz, Methanol-d) δ 8.46 (s, 1H), 4.46 (d, 1H), 4.19 (t, 1H), 4.14-3.98 (m, 4H), 3.92 (t, 2H), 3.76-3.62 (m, 2H), 3.55-3.35 (m, 3H), 3.23-3.08 (m, 1H), 2.99 (d, 2H), 2.90-2.76 (m, 11H), 2.73-2.62 (m, 1H), 2.37-2.15 (m, 2H), 2.02 (ddd, 1H), 1.92 (hept, 1H), 1.81-1.71 (m, 1H), 1.58 (s, 3H), 1.57-1.45 (m, 1H), 1.36-1.26 (m, 12H), 1.11 (t, 4H), 0.99 (d, 6H), 0.88 (d, 3H).

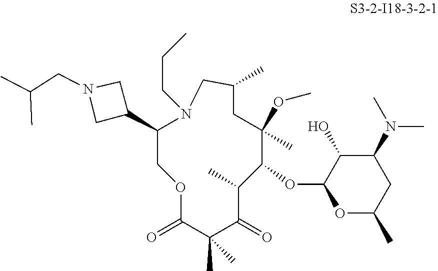

### (3R,6R,8R,9R,10R)-9-(((2S,3R,4S,6R)-4-(dimethylamino)-3-hydroxy-6-methyltetrahydro-2H-pyran-2-yl)oxy)-3-(1-isobutylazetidin-3-yl)-8-methoxy-6,8,10,12,12-pentamethyl-4-propyl-1-oxa-4-azacyclotridecane-11,13-dione (S3-2-I18-3-2-1) (Compound 105)

Prepared according to the methods of S3-2-I5-1-2-1 from S1-3-I18-3 and isobutyraldehyde to provide the title compound as a formate salt. MS (ESI+) m/z: 640.44 [M+H]f; ¹H NMR (400 MHz, Methanol-d) δ 8.45 (s, 3H), 4.46 (d, 1H), 4.21 (t, 1H), 4.17-3.84 (m, 6H), 3.77-3.63 (m, 2H), 3.54-3.36 (m, 3H), 3.14 (q, 1H), 3.02 (d, 2H), 2.82 (s, 6H), 2.79 (s, 3H), 2.76-2.69 (m, 11H), 2.68-2.60 (m, 2H), 2.36-2.19 (m, 2H), 2.02 (ddd, 1H), 1.93 (p, 11H), 1.73 (s, 1H), 1.59 (s, 3H), 1.56-1.46 (m, 3H), 1.36-1.25 (m, 13H), 1.14-1.03 (m, 1H), 1.02-0.97 (m, 6H), 0.92 (t, 3H), 0.86 (d, 3H).

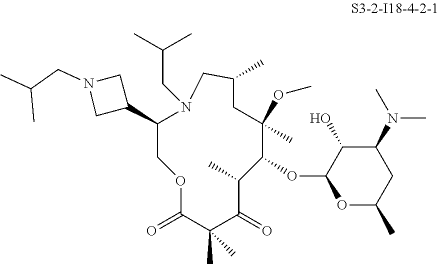

### (3R,6R,8R,9R,10R)-9-(((2S,3R,4S,6R)-4-(dimethylamino)-3-hydroxy-6-methyltetrahydro-2H-pyran-2-yl)oxy)-4-isobutyl-3-(1-isobutylazetidin-3-yl)-8-methoxy-6,8,10,12,12-pentamethyl-1-oxa-4-azacyclotridecane-11,13-dione (S3-2-I18-4-2-1) (Compound 21)

Prepared according to the methods of S3-2-I5-1-2-1 from S1-3-I18-4 and isobutyraldehyde to provide the title compound as a formate salt. MS (ESI+) m/z: 654.42 [M+H]⁺, formate salt, ¹H NMR (400 MHz, Methanol-d) δ 8.56 (s, 2H), 4.44 (d, 11H), 4.09-3.98 (m, 3H), 3.94 (t, 1H), 3.90-3.78 (m, 2H), 3.75-3.63 (m, 3H), 3.48-3.33 (m, 2H), 3.28-3.22 (m, 1H), 3.08-2.99 (m, 1H), 2.83 (d, 2H), 2.76 (s, 3H), 2.74 (s, 6H), 2.59 (dd, 1H), 2.53-2.43 (m, 11H), 2.39-2.31 (m, 1H), 2.31-2.20 (m, 2H), 1.97 (ddd, 1H), 1.85 (p, 11H), 1.78-1.63 (m, 2H), 1.56 (s, 3H), 1.47 (q, 1H), 1.37-1.19 (m, 13H), 1.05 (dd, 1H), 1.01-0.89 (m, 12H), 0.84 (d, 3H).

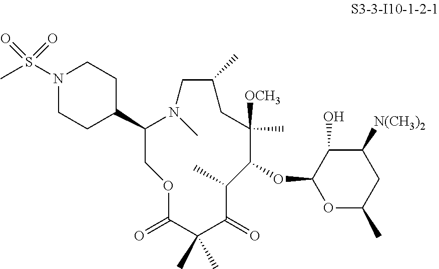

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-(methylsulfonyl)piperidin-4-yl)-1-oxa-4-azacyclotridecane-11,13-dione (S3-3-I10-1-2-1) (Compound 142)

S3-1-I10-1-2 (20 mg, 0.029 mmol) was dissolved in dichloromethane (0.29 mL). Et₃N (14.7 mg, 0.145 mmol) and methane sulfonyl chloride (3.4 μL, 0.044 mmol) were added at rt. The reaction mixture was allowed to stir at rt for 1 h. The reaction was quenched by adding saturated NaHCO₃ (2 mL) and the aqueous layer was extracted with dichloromethane three times (2 mL). The combined organic layers were dried over MgSO₄, were filtered, and were concentrated. The material was dissolved in MeOH (1 mL) and the reaction mixture was heated at 60° C. for 16 h. The reaction mixture was concentrated and was purified by HPLC (MeCN-water-0.1% HCO₂H) to yield 7.70 mg of the title compound as a formate salt. MS (ESI+) m/z: 662.42 [M+H]⁺. ¹H NMR (400 MHz, Methanol-d4) 1H NMR (400 MHz, Chloroform-d) δ 4.80-4.61 (m, 1H), 4.47 (d, 1H), 4.41-4.29 (m, 1H), 4.23 (d, 1H), 3.87-3.67 (m, 3H), 3.54-3.34 (m, 4H), 3.22-2.92 (m, 5H), 2.92-2.62 (m, 14H), 2.31-1.97 (m, 3H), 1.97-1.78 (m, 2H), 1.75-1.61 (m, 2H), 1.60-1.42 (m, 6H), 1.42-1.16 (m, 13H), 1.10-0.96 (m, 3H).

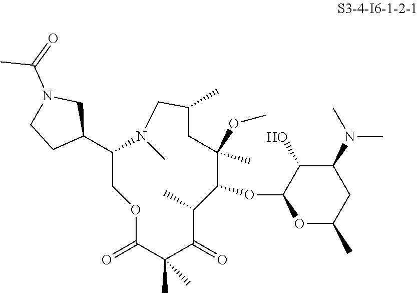

### (3S,6R,8R,9R,10R)-3-((S)-1-acetylpyrrolidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-4-I6-1-2-1) (Compound 140)

S3-1-I6-1-2 (45 mg, 0.0667 mmol) was dissolved in dichloromethane (1 mL) and acetic anhydride (0.00754 mL, 0.08 mmol) was added. After 45 min, the reaction mixture was quenched with NaHCO₃ (sat., aq. solution) and was extracted with dichloromethane (2 times). The combined extracts were concentrated. The crude material was dissolved in methanol (1 mL), and the reaction mixture was heated to 45° C. external temperature. After 16 h, the reaction was allowed to cool to rt and was concentrated. The residue was purified by HPLC (Atlantis T3 column, 5-30% MeCN-water-0.1% HCO₂H) to give 17.7 mg of the title compound as a formate salt. MS (ESI+) m/z: 306.79 [M+2H]²⁺, 612.40 [M+H]⁺; ¹H NMR (400 MHz, Methanol-d₄) δ 8.53 (s, 1.4H), 4.46 (dd, 1H), 4.27 (dd, 1H), 4.21-3.97 (m, 1.6H), 3.97-3.75 (m, 1H), 3.75-3.57 (m, 2.5H), 3.56-3.32 (m, 4H), 3.28-3.21 (m, 1H), 3.15 (t, 1H), 2.99-2.84 (m, 3H), 2.79 (s, 7H), 2.63-2.26 (m, 4H), 2.17-2.06 (m, 2H), 2.06-2.01 (m, 3H), 2.01-1.95 (m, 1H), 1.94-1.63 (m, 3H), 1.59-1.44 (m, 4H), 1.37 (s, 3H), 1.35-1.19 (m, 10H), 1.08-0.84 (m, 3H).

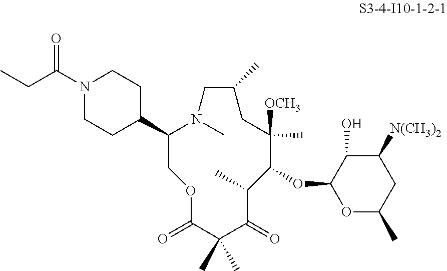

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-propionylpiperidin-4-yl)-1-oxa-4-azacyclotridecane-11,13-dione (S3-4-I10-1-2-1) (Compound 13)

Prepared according to the methods of S3-4-I6-1-2-1, substituting propionyl chloride to provide the title compound as a formate salt. MS (ESI+) m/z: 640.42 [M+H]⁺; ¹H NMR (400 MHz, Methanol-d₄) δ 8.63 (s, 1H), 4.74-4.53 (m, 2H), 4.47 (d, 1H), 4.39-4.27 (m, 1H), 4.23 (d, 1H), 4.03 (t, 1H), 3.80-3.67 (m, 1H), 3.51-3.42 (m, 2H), 3.42-3.33 (m, 2H), 3.21-3.04 (m, 2H), 3.01 (s, 3H), 2.90-2.72 (m, 9H), 2.72-2.54 (m, 2H), 2.41 (qd, 2H), 2.33-2.08 (m, 2H), 2.08-1.97 (m, 1H), 1.94-1.61 (m, 4H), 1.61-1.43 (m, 5H), 1.43-1.25 (m, 13H), 1.11 (t), 1.07-0.95 (m, 3H).

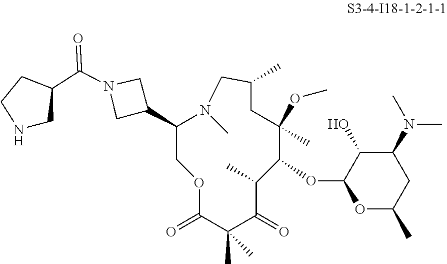

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-((R)-pyrrolidine-3-carbonyl)azetidin-3-yl)-1-oxa-4-azacyclotridecane-11,13-dione (S3-4-I18-1-2-1) (Compound 22). To a solution of S3-1-I18-1-2 (prepared according to the methods of S3-1-I5-1-2, substituting I18 in Scheme 1) (125 mg, 0.189 mmol) and (R)-1-(tert-butoxycarbonyl)pyrrolidine-3-carboxylic acid (40.6 mg, 0.189 mmol) in dichloromethane (1.88 mL) was added N,N-diisopropylethylamine (0.066 mL, 0.378 mmol) followed by HATU (71.8 mg, 0.189 mmol). The reaction mixture was stirred at room temperature for 1 h and the solvent and excess reagent were removed in vacuo. The residue was purified on 12 g of silica gel (elution with 0-10% MeOH-dichloromethane gradient) to give a white solid (128 mg, 80%). This was dissolved in dichloromethane (1 mL) and trifluoroacetic acid (0.25 mL) was added. The reaction mixture was stirred at room temperature for 2 h and was concentrated. The residue was suspended in ethyl acetate and washed with sat. aq. NaHCO₃ (2 times). The washed solution was dried over sodium sulfate, was filtered, and was concentrated to give the amine intermediate (110 mg, 98%). MS (ESI+) m/z: 253.42 [M+3H]³⁺, 379.55 [M+2H]²⁺, 757.30 [M+H]⁺. The crude amine (30 mg, 0.04 mmol) was dissolved in methanol (1.5 mL), and the reaction mixture was heated to 60° C. external temperature for 5 hr. Solvent was removed in vacuo and the residue was purified by HPLC (Atlantis T3 column, 2-40% MeCN-water-0.1% HCO₂H) to give 7.35 mg of the title compound as a formate salt. MS (ESI+) m/z: 218.46 [M+3H]³⁺, 327.12 [M+2H]²⁺, 653.25 [M+H]⁺; ¹H NMR (400 MHz, Methanol-d₄) δ 8.50 (s, 2.5H), 4.49 (d, 1H), 4.46-4.27 (m, 2H), 4.24-4.05 (m, 4H), 4.01 (t, 0.5H), 3.88 (t, 0.5H), 3.72 (dtt, 1H), 3.68-3.53 (m, 1H), 3.53-3.33 (m, 7H), 3.25 (t, 1H), 3.15 (s, 1H), 3.00 (s, 3H), 2.80 (s, 7H), 2.61 (s, 3H), 2.30 (dq, 1H), 2.17-1.96 (m, 3H), 1.76 (s, 1H), 1.58-1.45 (m, 5H), 1.38 (d, 3H), 1.37-1.24 (m, 9H), 0.97 (d, 3H).

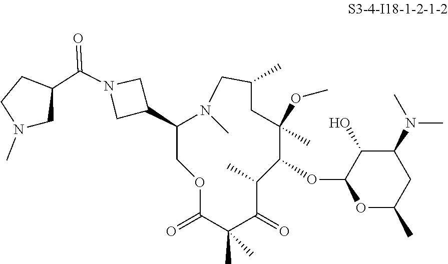

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-((R)-1-methylpyrrolidine-3-carbonyl)azetidin-3-yl)-1-oxa-4-azacyclotridecane-11,13-dione (S3-4-I18-1-2-1-2) (Compound 111). The amine intermediate from S3-4-I18-1-2-1-1 above (30 mg, 0.0396 mmol) was dissolved in dichloromethane (1 mL), and Na(OAc)₃BH (16.7 mg, 0.079 mmol) followed by formaldehyde (37 wt % aqueous solution, 0.0265 mL, 0.396 mmol) were added. After 15 min, the reaction mixture was quenched with sat., aq. NaHCO₃ and extracted with dichloromethane (3 times). The combined extracts were concentrated in vacuo. The residue was dissolved in methanol (1.5 mL), and the reaction mixture was heated to 45° C. external temperature for 16 h. The solvent was removed in vacuo and the residue was purified by HPLC (Atlantis T3 column, 2-40% MeCN-water-0.1% HCO₂H) to give 15.8 mg of the title compound as a formate salt. MS (ESI+) m/z: 223.12 [M+3H]³⁺, 334.11 [M+2H]²⁺, 667.26 [M+H]⁺; ¹H NMR (400 MHz, Methanol-d₄) δ 8.51 (s, 2.5H), 4.47 (d, 1H), 4.45-4.24 (m, 2H), 4.11 (dd, 4H), 3.99 (t, 0.5H), 3.86 (t, 0.5H), 3.76-3.66 (m, 2H), 3.52-3.32 (m, 6H), 3.26 (d, 1H), 3.13 (s, 1H), 2.99 (s, 3H), 2.85 (s, 4H), 2.79 (s, 7H), 2.60 (s, 3H), 2.34 (q, 1H), 2.17-2.05 (m, 1H), 2.01 (ddd, 2H), 1.74 (s, 1H), 1.57-1.45 (m, 5H), 1.37 (d, 3H), 1.35-1.23 (m, 9H), 0.96 (d, 3H).

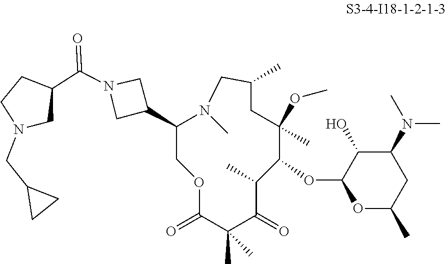

### (3R,6R,8R,9R,10R)-3-(1-((R)-1-(cyclopropylmethyl)pyrrolidine-3-carbonyl)azetidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-4-I18-1-2-1-3) (Compound 145)

Prepared according to the methods of S3-4-I18-1-2-1-2, substituting cyclopropanecarboxaldehyde to provide 14.25 mg of the title compound as a formate salt. MS (ESI+) m/z: 236.48 [M+3H]³⁺, 354.15 [M+2H]²⁺, 707.28 [M+H]⁺; ¹H NMR (400 MHz, Methanol-d₄) δ 8.52 (s, 2.5H), 4.48 (d, 2H), 4.41-4.26 (m, 1H), 4.25-4.05 (m, 3.5H), 4.02 (t, 1H), 3.88 (t, 1H), 3.72 (ddt, 1H), 3.58 (t, 2H), 3.51-3.33 (m, 6H), 3.15 (s, 1H), 3.05 (dd, 2H), 3.00 (s, 3H), 2.81 (s, 7H), 2.62 (s, 3H), 2.36 (dq, 1H), 2.19-1.98 (m, 3H), 1.74 (s, 1H), 1.58-1.42 (m, 5H), 1.39 (d, 3H), 1.35 (s, 3H), 1.34-1.29 (m, 6H), 1.12 (dd, 1H), 0.98 (d, 3H), 0.78-0.66 (m, 2H), 0.42 (d, 2H).

The following examples were prepared according to the methods of S3-4-I18-1-2-1-2, substituting the appropriate intermediate (Table 2, I) for I18 in Scheme 1, the appropriate carboxylic acid for (R)-1-(tert-butoxycarbonyl)pyrrolidine-3-carboxylic acid, and the appropriate aldehyde or ketone for formaldehyde:

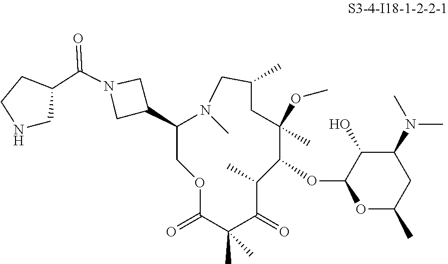

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-((S)-pyrrolidine-3-carbonyl)azetidin-3-yl)-1-oxa-4-azacyclotridecane-11,13-dione (S3-4-I18-1-2-2-1) (Compound 181)

Prepared according to the methods of S3-4-I18-1-2-1-1, substituting (S)-1-(tert-butoxycarbonyl)pyrrolidine-3-carboxylic acid to provide 7.26 mg of the title compound as a formate salt. MS (ESI+) m/z: 218.47 [M+3H]³⁺, 327.12 [M+2H]²⁺, 653.28 [M+H]⁺; ¹H NMR (400 MHz, Methanol-d₄) δ 8.50 (s, 3H), 4.58-4.41 (m, 2H), 4.40-4.26 (m, 1H), 4.18 (d, 3H), 4.10 (t, 1H), 4.02 (t, 0.5H), 3.88 (t, 0.5H), 3.78-3.69 (m, 1H), 3.69-3.55 (m, 1H), 3.52-3.32 (m, 7H), 3.25 (q, 1H), 3.15 (s, 1H), 3.01 (s, 3H), 2.81 (s, 7H), 2.62 (s, 3H), 2.30 (dq, 1H), 2.15-1.96 (m, 3H), 1.75 (s, 1H), 1.58-1.46 (m, 5H), 1.39 (d, 3H), 1.37-1.28 (m, 9H), 0.99 (d, 3H).

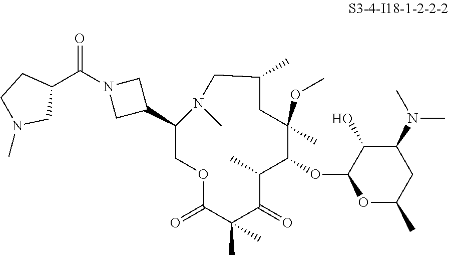

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-((S)-1-methylpyrrolidine-3-carbonyl)azetidin-3-yl)-1-oxa-4-azacyclotridecane-11,13-dione (S3-4-I18-1-2-2-2) (Compound 168)

Prepared according to the methods of S3-4-I18-1-2-1-2 from I18, (S)-1-(tert-butoxycarbonyl)pyrrolidine-3-carboxylic acid, and formaldehyde to provide 15.47 mg of the title compound as a formate salt. MS (ESI+) m/z: 223.12 [M+3H]³⁺, 334.10 [M+2H]²⁺, 667.29 [M+H]⁺; ¹H NMR (400 MHz, Methanol-d₄) δ 8.53 (s, 2.5H), 4.47 (d, 2H), 4.39-4.22 (m, 1.5H), 4.21-4.04 (m, 4H), 4.00 (t, 1H), 3.87 (t, 1H), 3.72 (ddt, 1H), 3.62 (s, 1H), 3.54-3.33 (m, 5H), 3.29-3.19 (m, 2H), 3.12 (s, 1H), 2.99 (s, 3H), 2.84 (s, 3H), 2.80 (s, 7H), 2.58 (s, 3H), 2.35 (t, 1H), 2.21-2.07 (m, 1H), 2.02 (ddd, 2H), 1.78 (s, 1H), 1.60-1.44 (m, 5H), 1.38 (d, 3H), 1.36-1.19 (m, 9H), 0.96 (d, 3H).

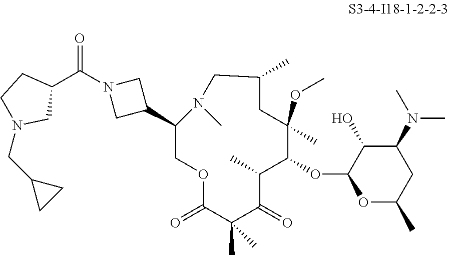

### (3R,6R,8R,9R,10R)-3-(1-((S)-1-(cyclopropylmethyl)pyrrolidine-3-carbonyl)azetidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-4-I18-1-2-2-3) (Compound 126)

Prepared according to the methods of S3-4-I18-1-2-1-2 from I18, (S)-1-(tert-butoxycarbonyl)pyrrolidine-3-carboxylic acid, and cyclopropanecarboxaldehyde to provide 20.2 mg of the title compound as a formate salt. MS (ESI+) m/z: 236.48 [M+3H]³⁺, 354.15 [M+2H]²⁺, 707.28 [M+H]⁺; ¹H NMR (400 MHz, Methanol-d₄) δ 8.51 (s, 2.5H), 4.46 (dd, 2H), 4.31 (q, 1H), 4.16 (d, 3H), 4.08 (t, 0.5H), 4.00 (t, 0.5H), 3.86 (t, 0.5H), 3.77-3.51 (m, 3H), 3.45 (ddd, 4H), 3.41-3.31 (m, 2H), 3.13 (s, 1H), 3.03 (d, 2H), 2.98 (s, 3H), 2.79 (s, 7H), 2.60 (s, 3H), 2.34 (dq, 1H), 2.20-2.05 (m, 1H), 2.00 (ddd, 2H), 1.73 (s, 1H), 1.56-1.44 (m, 5H), 1.36 (d, 3H), 1.35-1.25 (m, 9H), 1.11 (ddt, 1H), 1.00-0.90 (m, 3H), 0.73-0.66 (m, 2H), 0.40 (t, 2H).

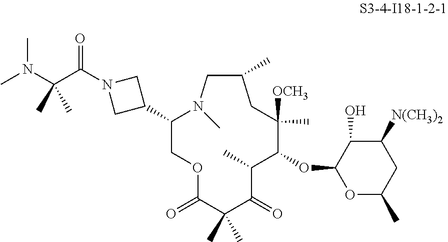

### (3S,6R,8R,9R,10R)-3-(1-(2-(dimethylamino)-2-methylpropanoyl)azetidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-4-I19-1-2-1)

Prepared according to the methods of S3-4-I18-1-2-1-2 from I19, α-(Boc-amino)isobutyric acid, and formaldehyde to provide 14.3 mg of the title compound as a formate salt. MS (ESI+) m/z: 669.40 [M+H]⁺. ¹H NMR (400 MHz, Methanol-d4) δ 8.45 (s, 3H), 4.76-4.64 (m, 1H), 4.58 (t, 1H), 4.50-4.38 (m, 2H), 4.36-4.04 (m, 3H), 4.04-3.91 (m, 1H), 3.78-3.67 (m, 1H), 3.58-3.33 (m, 3H), 3.26-3.09 (m, 1H), 2.97 (s, 3H), 2.86-2.56 (m, 11H), 2.43 (s, 6H), 2.16-1.94 (m, 2H), 1.85-1.63 (m, 1H), 1.63-1.46 (m, 5H), 1.44-1.25 (m, 18H), 1.02 (d, 3H).

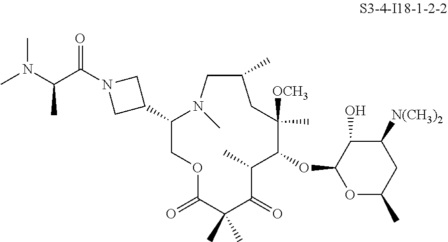

### (3S,6R,8R,9R,10R)-3-(1-(dimethyl-D-alanyl)azetidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-4-I19-1-2-2) (Compound 52)

Prepared according to the methods of S3-4-I18-1-2-1-2 from I19, D-alanine, and formaldehyde to provide 14.2 mg of the title compound as a formate salt. MS (ESI+) m/z: 655.37 [M+H]⁺. ¹H NMR (400 MHz, Methanol-d4) δ 8.46 (s, 3H), 4.51-4.39 (m, 2H), 4.39-4.28 (m, 1H), 4.28-4.18 (m, 2H), 4.18-4.04 (m, 2H), 4.04-3.94 (m, 1H), 3.80-3.66 (m, 2H), 3.66-3.57 (m, 1H), 3.57-3.33 (m, 3H), 3.26-3.07 (m, 1H), 2.93 (s, 3H), 2.87-2.77 (m, 7H), 2.75-2.36 (m, 10H), 2.09-1.91 (m, 2H), 1.91-1.71 (m, 1H), 1.60-1.41 (m, 5H), 1.41-1.23 (m, 15H), 0.99 (d, 3H).

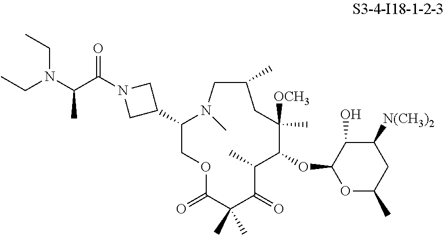

### (3S,6R,8R,9R,10R)-3-(1-(diethyl-D-alanyl)azetidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-4-I19-1-2-3) (Compound 74)

Prepared according to the methods of S3-4-I18-1-2-1-2 from I19, D-alanine, and acetaldehyde to provide 16.5 mg of the title compound as a formate salt. MS (ESI+) m/z: 683.40 [M+H]⁺. ¹H NMR (400 MHz, Methanol-d4) 1H NMR (400 MHz, Chloroform-d) δ 8.47 (s, 3H), 4.54-4.36 (m, 3H), 4.32 (d, 1H), 4.28-4.16 (m, 2H), 4.16-4.05 (m, 2H), 4.05-3.84 (m, 2H), 3.79-3.66 (m, 1H), 3.62-3.50 (m, 1H), 3.50-3.33 (m, 2H), 3.21-2.85 (m, 8H), 2.82 (s, 6H), 2.71-2.34 (m, 4H), 2.08-2.00 (m, 1H), 1.98-1.76 (m, 2H), 1.60-1.42 (m, 5H), 1.42-1.27 (m, 16H), 1.23 (q, 6H), 0.98 (d, 3H).

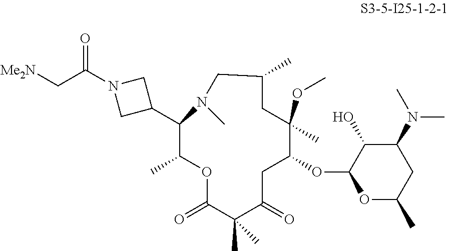

### (2R,3R,6R,8R,9R,10R)-9-(((2S,3R,4S,6R)-4-(dimethylamino)-3-hydroxy-6-methyltetrahydro-2H-pyran-2-yl)oxy)-3-(1-(dimethylglycyl)azetidin-3-yl)-8-methoxy-2,4,6,8,10,12,12-heptamethyl-1-oxa-4-azacyclotridecane-11,13-dione (S3-5-I25-1-2-1) (Compound 120)

Prepared according to the methods of S3-4-I18-1-2-1-2 from I25 and N,N-dimethylglycine to provide 14.9 mg of the title compound as a formate salt. MS (ESI+) m/z: 219.1 [M+3H]³, 328.1 [M+2H]²⁺, 655.3 [M+H]⁺; ¹H NMR (400 MHz, Methanol-d₄) δ 8.48 (s, 2H), 4.98 (s, 1H), 4.51 (d, 1H), 4.39-4.22 (m, 1H), 4.22-4.00 (m, 3H), 4.00-3.82 (m, 1H), 3.70 (dtt, 1H), 3.65-3.51 (m, 3H), 3.47 (dd, 1H), 3.39 (ddd, 1H), 2.92 (s, 3H), 2.82 (s, 6H), 2.67 (s, 7H), 2.54-2.11 (m, 3H), 2.01 (ddd, 1H), 1.74 (s, 2H), 1.61 (s, 3H), 1.57-1.45 (m, 2H), 1.36 (s, 4H), 1.30 (dd, Hz, 4H), 1.29-1.09 (m, 8H), 1.09-0.86 (m, 3H).

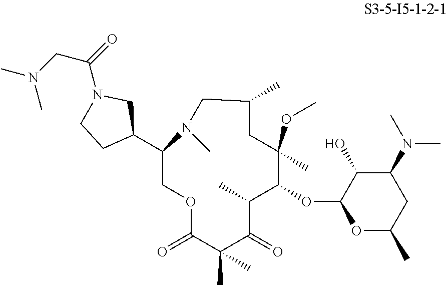

### (3R,6R,8R,9R,10R)-9-(((2S,3R,4S,6R)-4-(Dimethylamino)-3-hydroxy-6-methyltetrahydro-2H-pyran-2-yl)oxy)-3-((S)-1-(dimethylglycyl)pyrrolidin-3-yl)-8-methoxy-4,6,8,10,12,12-hexamethyl-1-oxa-4-azacyclotridecane-11,13-dione (S3-5-I5-1-2-1) (Compound 100)

S3-1-I5-1-2 (47 mg, 0.0697 mmol) was dissolved in dichloromethane (1 mL) and chloroacetyl chloride (0.0061 mL, 0.0766 mmol) was added. After 15 min, dimethylamine (2 M solution in tetrahydrofuran, 0.348 mL, 697 mmol) was added. After 15 min, the reaction mixture was heated to 70° C. After 4 h, the reaction mixture was allowed to cool to rt. The reaction mixture was diluted with dichloromethane, was washed with water (1 time), and was concentrated. The crude material was dissolved in methanol (1 mL), and the reaction mixture was heated to 60° C. external temperature. After 5 h, the reaction was allowed to cool to rt and was concentrated. The residue was purified by HPLC (Atlantis T3 column, 5-40% MeCN-water-0.1% HCO₂H) to give 12.8 mg of the title compound as a formate salt. MS (ESI+) m/z: 219.27 [M+3H]³⁺, 328.28 [M+2H]²⁺, 655.50 [M+H]⁺; ¹H NMR (400 MHz, Methanol-d₄) δ 8.42 (s, 1H), 4.48 (dd, 1H), 4.38 (s, 1H), 4.24 (s, 1H), 4.18-3.95 (m, 3H), 3.88-3.61 (m, 4H), 3.58-3.32 (m, 5H), 3.28-3.10 (m, 2H), 3.01 (s, 3H), 2.90 (d, 7H), 2.84 (s, 7H), 2.77-2.56 (m, 2H), 2.50-2.25 (m, 1H), 2.10-1.99 (m, 2H), 1.99-1.83 (m, 1H), 1.76 (s, 1H), 1.57 (d, 3H), 1.54 (d, 2H), 1.39 (d, 6H), 1.33 (dd, 7H), 1.03 (d, 3H).

The following examples were prepared according to the methods of S3-5-I5-1-2-1, substituting the appropriate intermediate (Table 2, I) for I5 in Scheme 1 and the appropriate amine for dimethylamine:

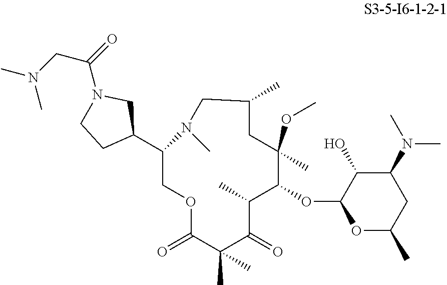

### (3S,6R,8R,9R,10R)-9-(((2S,3R,4S,6R)-4-(dimethylamino)-3-hydroxy-6-methyltetrahydro-2H-pyran-2-yl)oxy)-3-((S)-1-(dimethylglycyl)pyrrolidin-3-yl)-8-methoxy-4,6,8,10,12,12-hexamethyl-1-oxa-4-azacyclotridecane-11,13-dione (S3-5-I6-1-2-1) (Compound 67)

Prepared according to the methods of S3-5-I5-1-2-1 from I6 and dimethylamine to provide 8.88 mg of the title compound as a formate salt. MS (ESI+) m/z: 219.29 [M+3H]³⁺, 328.30 [M+2H]²⁺, 655.41 [M+H]⁺; ¹H NMR (400 MHz, Methanol-d₄) δ 8.44 (s, 2H), 4.46 (dd, 1H), 4.34-4.23 (m, 1H), 4.23-4.04 (m, 2H), 3.95 (q, 2.5H), 3.78-3.59 (m, 2.5H), 3.52-3.33 (m, 5H), 3.23 (t, 1H), 3.10-2.91 (m, 4H), 2.85 (s, 4H), 2.83 (d, 9H), 2.23-2.07 (m, 1.5H), 2.07-1.99 (m, 1.5H), 1.85 (td, 1H), 1.79-1.65 (m, 1H), 1.58-1.47 (m, 4H), 1.43-1.28 (m, 12H), 1.03 (s, 3H).

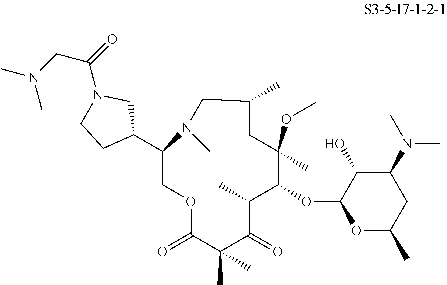

### (3R,6R,8R,9R,10R)-9-(((2S,3R,4S,6R)-4-(Dimethylamino)-3-hydroxy-6-methyltetrahydro-2H-pyran-2-yl)oxy)-3-((R)-1-(dimethylglycyl)pyrrolidin-3-yl)-8-methoxy-4,6,8,10,12,12-hexamethyl-1-oxa-4-azacyclotridecane-11,13-dione (S3-5-I7-1-2-1) (Compound 148)

Prepared according to the methods of S3-5-I5-1-2-1 from I7 and dimethylamine to provide 17.27 mg of the title compound as a formate salt. MS (ESI+) m/z: 219.30 [M+3H]³⁺, 328.31 [M+2H]²⁺, 655.43 [M+H]⁺; ¹H NMR (400 MHz, Methanol-d₄) δ 8.48 (s, 2H), 4.48 (d, 1H), 4.34 (s, 1H), 4.24-4.10 (m, 1H), 4.07-3.92 (m, 2H), 3.81-3.69 (m, 2H), 3.65 (t, 1H), 3.60-3.33 (m, 5H), 3.26 (t, 1H), 2.99 (s, 4H), 2.88 (s, 3H), 2.86 (s, 3H), 2.83 (s, 7H), 2.68 (d, 4H), 2.22-1.99 (m, 3H), 1.95-1.67 (m, 2H), 1.60-1.48 (m, 4H), 1.39 (s, 3H), 1.36 (s, 3H), 1.33 (dd, 7H), 1.00 (s, 3H).

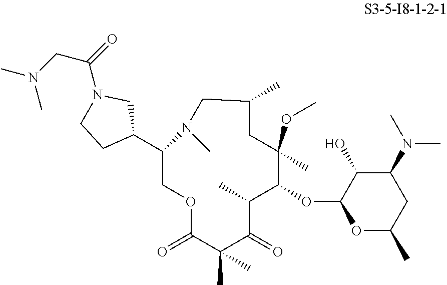

### (3S,6R,8R,9R,10R)-9-(((2S,3R,4S,6R)-4-(Dimethylamino)-3-hydroxy-6-methyltetrahydro-2H-pyran-2-yl)oxy)-3-((R)-1-(dimethylglycyl)pyrrolidin-3-yl)-8-methoxy-4,6,8,10,12,12-hexamethyl-1-oxa-4-azacyclotridecane-11,13-dione (S3-5-I8-1-2-1) (Compound 169)

Prepared according to the methods of S3-5-I5-1-2-1 from I8 and dimethylamine to provide 12.17 mg of the title compound as a formate salt. MS (ESI+) m/z: 219.30 [M+3H]³⁺, 328.39 [M+2H]²⁺, 655.44 [M+H]⁺; ¹H NMR (400 MHz, Methanol-d₄) δ 8.47 (s, 1.7H), 4.46 (d, 1H), 4.21 (d, 2H), 4.12-3.92 (m, 3H), 3.86-3.58 (m, 4H), 3.52-3.32 (m, 5H), 3.25-3.09 (m, 2H), 3.09-2.94 (m, 4H), 2.87 (s, 4H), 2.85 (s, 4H), 2.83 (s, 7H), 2.04 (ddd, 2H), 1.51 (d, 5H), 1.38 (d, 5H), 1.33 (t, 8H), 1.11-0.95 (m, 3H).

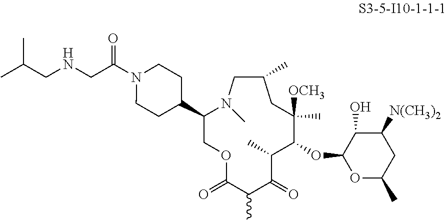 [SMILES: CO[C@]1(C)C[C@@H](C)CN(C)[C@H](C2CCN(C(=O)CNCC(C)C)CC2)COC(=O)C(C)C(=O)[C@H](C)[C@H]1O[C@@H]1O[C@H](C)C[C@H](N(C)C)[C@H]1O]

### (3R,6R,8R,9R,10R)-9-(((2S,3R,4S,6R)-4-(dimethylamino)-3-hydroxy-6-methyltetrahydro-2H-pyran-2-yl)oxy)-3-(1-(isobutylglycyl)piperidin-4-yl)-8-methoxy-4,6,8,10,12-pentamethyl-1-oxa-4-azacyclotridecane-11,13-dione (S3-5-I10-1-1-1) (Compound 107)

Prepared according to the methods of S3-5-I5-1-2-1 from S3-1-I10-1-1 and isobutylamine to provide the title compound as a formate salt. MS (ESI+) m/z: 683.46 [M+H]⁺; ¹H NMR (400 MHz, Methanol-d₄) δ 8.54 (s, 2H), 4.59-4.35 (m, 4H), 4.06-3.86 (m, 3H), 3.86-3.64 (m, 3H), 3.49-3.38 (m, 2H), 3.38-3.33 (m, 1H), 3.22-2.85 (m, 6H), 2.85-2.65 (m, 12H), 2.09-1.96 (m, 3H), 1.95-1.71 (m, 3H), 1.50 (q, 2H), 1.43-1.12 (m, 15H), 1.08-0.97 (m, 10H), 0.98-0.82 (m, 4H).

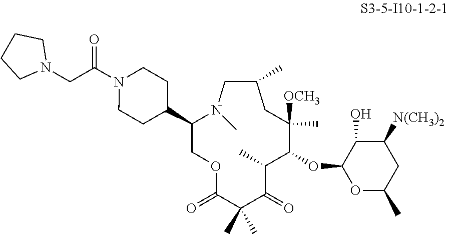

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-(2-(pyrrolidin-1-yl)acetyl)piperidin-4-yl)-1-oxa-4-azacyclotridecane-11,13-dione (S3-5-I10-1-2-1) (Compound 9)

Prepared according to the methods of S3-5-I5-1-2-1 from I10 and pyrrolidine to provide 15.9 mg of the title compound as a formate salt. MS (ESI+) m/z: 695.46 [M+H]⁺; ¹H NMR (400 MHz, Methanol-d₄) δ 4.64-4.50 (m, 2H), 4.47 (d, 1H), 4.38-4.12 (m, 4H), 3.83-3.66 (m, 2H), 3.56-3.42 (m, 2H), 3.43-3.33 (m, 5H), 3.22-3.04 (m, 2H), 3.04-2.91 (m, 4H), 2.88-2.58 (m, 11H), 2.14-1.97 (m, 6H), 1.94-1.84 (m, 1H), 1.84-1.65 (m, 2H), 1.62-1.45 (m, 6H), 1.44-1.21 (m, 14H), 1.09-0.89 (m, 3H).

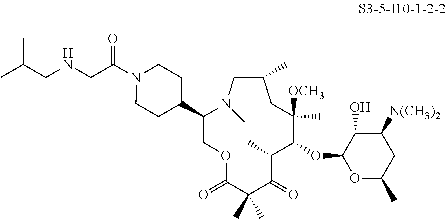

### (3R,6R,8R,9R,10R)-9-(((2S,3R,4S,6R)-4-(dimethylamino)-3-hydroxy-6-methyltetrahydro-2H-pyran-2-yl)oxy)-3-(1-(isobutylglycyl)piperidin-4-yl)-8-methoxy-4,6,8,10,12,12-hexamethyl-1-oxa-4-azacyclotridecane-11,13-dione (S3-5-I10-1-2-2) (Compound 66)

Prepared according to the methods of S3-5-I5-1-2-1 from I10 and isobutylamine pyrrolidine to provide 13.0 mg of the title compound as a formate salt. MS (ESI+) m/z: 697.46 [M+H]⁺; ¹H NMR (400 MHz, Methanol-d₄) δ 4.64-4.50 (m, 11H), 4.48 (d, 1H), 4.32-4.06 (m, 2H), 4.07-3.86 (m, 2H), 3.86-3.63 (m, 2H), 3.62-3.48 (m, 1H), 3.45 (dd, 11H), 3.19-3.03 (m, 1H), 2.95 (s, 3H), 2.87-2.32 (m, 13H), 2.11-1.96 (m, 3H), 1.95-1.69 (m, 4H), 1.60-1.41 (m, 5H), 1.41-1.11 (m, 16H), 1.04 (d, 6H), 0.98-0.69 (m, 5H).

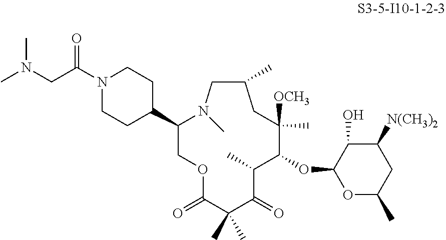

### (3R,6R,8R,9R,10R)-9-(((2S,3R,4S,6R)-4-(dimethylamino)-3-hydroxy-6-methyltetrahydro-2H-pyran-2-yl)oxy)-3-(1-(dimethylglycyl)piperidin-4-yl)-8-methoxy-4,6,8,10,12,12-hexamethyl-1-oxa-4-azacyclotridecane-11,13-dione (S3-5-I10-1-2-3) (Compound 85)

Prepared according to the methods of S3-5-I5-1-2-1 from I10 and dimethylamine to provide 15.5 mg of the title compound as a formate salt. MS (ESI+) m/z: 669.47 [M+H]⁺; ¹H NMR (400 MHz, Methanol-d₄) δ 8.57 (br s, 2H), 4.58-4.42 (m, 3H), 4.39-4.13 (m, 2H), 4.08-3.87 (m, 2H), 3.88-3.68 (m, 2H), 3.55-3.42 (m, 2H), 3.41-3.34 (m, 1H), 3.25-3.06 (m, 2H), 3.05-2.92 (m, 4H), 2.90-2.58 (m, 17H), 2.37-2.09 (br m, 1H), 2.08-1.99 (m, 1H), 1.89 (br d, 1H), 1.85-1.60 (m, 3H), 1.60-1.44 (m, 6H), 1.43-1.22 (m, 14H), 1.01 (br s, 3H).

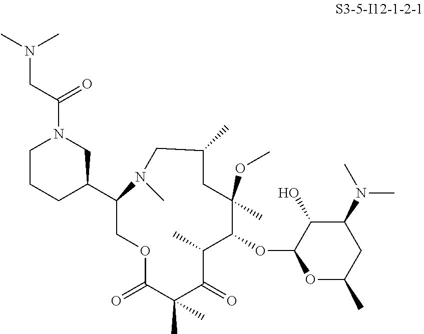

### (3R,6R,8R,9R,10R)-9-(((2S,3R,4S,6R)-4-(dimethylamino)-3-hydroxy-6-methyltetrahydro-2H-pyran-2-yl)oxy)-3-((S)-1-(dimethylglycyl)piperidin-3-yl)-8-methoxy-4,6,8,10,12,12-hexamethyl-1-oxa-4-azacyclotridecane-11,13-dione (S3-5-I12-1-2-1) (Compound 96)

Prepared according to the methods of S3-5-I5-1-2-1 from I12 and dimethylamine to provide 17.36 mg of the title compound as a formate salt. MS (ESI+) m/z: 223.93 [M+3H]³, 335.35 [M+2H]²⁺, 669.42 [M+H]⁺; ¹H NMR (400 MHz, Methanol-d₄) δ 8.49 (s, 1.7H), 4.47 (d, 2H), 4.40-4.25 (m, 1H), 4.21 (d, 1H), 4.15-4.02 (m, 2H), 3.80-3.67 (m, 2H), 3.56-3.33 (m, 4H), 3.20-2.87 (m, 8H), 2.83 (d, 12H), 2.77-2.57 (m, 3H), 2.24-2.12 (m, 1H), 2.04 (ddd, 2H), 1.98-1.85 (m, 2H), 1.78-1.60 (m, 3H), 1.59-1.51 (m, 4H), 1.43-1.26 (m, 12H), 1.11-0.87 (m, 3H).

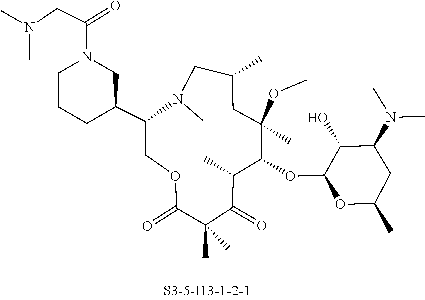

### (3S,6R,8R,9R,10R)-9-(((2S,3R,4S,6R)-4-(dimethylamino)-3-hydroxy-6-methyltetrahydro-2H-pyran-2-yl)oxy)-3-((S)-1-(dimethylglycyl)piperidin-3-yl)-8-methoxy-4,6,8,10,12,12-hexamethyl-1-oxa-4-azacyclotridecane-11,13-dione (S3-5-I13-1-2-1) (Compound 34)

Prepared according to the methods of S3-5-I5-1-2-1 from I13 and dimethylamine to provide 11.74 mg of the title compound as a formate salt. MS (ESI+) m/z: 224.01 [M+3H]³⁺, 335.36 [M+2H]²⁺, 669.45 [M+H]⁺; ¹H NMR (400 MHz, Methanol-d₄) δ 8.47 (s, 1H), 4.54 (d, 1H), 4.46 (dd, 1H), 4.33 (dd, 1H), 4.13 (q, 3H), 3.98-3.78 (m, 1H), 3.73 (q, 2H), 3.51-3.34 (m, 3H), 3.23-2.91 (m, 7H), 2.86 (s, 5H), 2.82 (s, 7H), 2.80 (s, 3H), 2.75-2.57 (m, 2H), 2.08-1.92 (m, 2H), 1.88-1.74 (m, 2H), 1.57-1.48 (m, 5H), 1.43-1.36 (m, 6H), 1.33 (d, 8H), 1.12-0.96 (m, 3H).

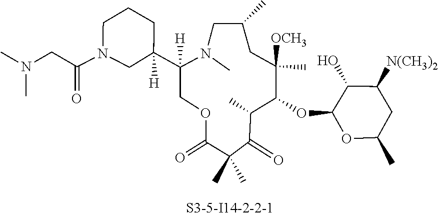

### (3R,6R,8R,9R,10R)-9-(((2S,3R,4S,6R)-4-(dimethylamino)-3-hydroxy-6-methyltetrahydro-2H-pyran-2-yl)oxy)-3-((R)-1-(dimethylglycyl)piperidin-3-yl)-8-methoxy-4,6,8,10,12,12-hexamethyl-1-oxa-4-azacyclotridecane-11,13-dione (S3-5-I14-2-2-1) (Compound 191)

Prepared according to the methods of S3-5-I5-1-2-1 from I14 and dimethylamine to provide the title compound as a formate salt. MS (ESI+) m/z: 697.45 [M+H]⁺; ¹H NMR (400 MHz, Chloroform-d) δ 4.69-4.55 (m, 1H), 4.49 (dd, 2H), 4.37-3.80 (m, 5H), 3.73 (tdd, 2H), 3.58-3.34 (m, 3H), 3.26-2.88 (m, 6H), 2.88-2.72 (m, 12H), 2.72-2.35 (m, 4H), 2.27 (d, 1H), 2.10-1.71 (m, 6H), 1.67-1.42 (m, 6H), 1.42-1.13 (m, 13H), 1.12-0.79 (m, 3H).

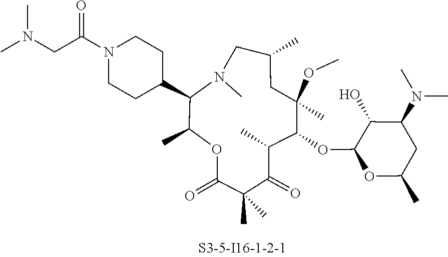

### (2S,3R,6R,8R,9R,10R)-9-(((2S,3R,4S,6R)-4-(dimethylamino)-3-hydroxy-6-methyltetrahydro-2H-pyran-2-yl)oxy)-3-(1-(dimethylglycyl)piperidin-4-yl)-8-methoxy-2,4,6,8,10,12,12-heptamethyl-1-oxa-4-azacyclotridecane-11,13-dione (S3-5-I16-1-2-1) (Compound 159)

Prepared according to the methods of S3-5-I5-1-2-1 from I16 and dimethylamine to provide 4.55 mg of the title compound as a formate salt. MS (ESI+) m/z: 228.69 [M+3H]³⁺, 342.38 [M+2H]²⁺, 683.36 [M+H]⁺; ¹H NMR (400 MHz, Methanol-d₄) δ 8.51 (s, 2.6H), 5.16 (s, 1H), 4.62-4.47 (m, 2H), 4.29 (d, 1H), 4.02-3.70 (m, 4H), 3.55-3.33 (m, 4H), 3.19 (s, 3H), 3.15-3.06 (m, 1H), 2.81 (s, 6H), 2.78-2.65 (m, 8H), 2.60 (s, 3H), 2.20-2.07 (m, 1H), 2.07-1.88 (m, 4H), 1.78 (d, 1H), 1.60-1.48 (m, 4H), 1.37 (s, 4H), 1.33 (d, 6H), 1.29 (d, 7H), 0.96 (d, 3H).

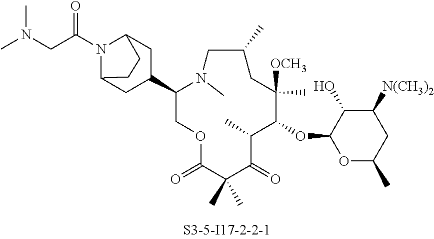

### (3R,6R,8R,9R,10R)-9-(((2S,3R,4S,6R)-4-(dimethylamino)-3-hydroxy-6-methyltetrahydro-2H-pyran-2-yl)oxy)-3-(8-(dimethylglycyl)-8-azabicyclo[3.2.1]octan-3-yl)-8-methoxy-4,6,8,10,12,12-hexamethyl-1-oxa-4-azacyclotridecane-11,13-dione (S3-5-I17-2-2-1) (Compound 6)

Prepared according to the methods of S3-5-I5-1-2-1 from I17 and dimethylamine to provide the title compound as a formate salt. MS (ESI+) m/z: 695.32 [M+H]⁺; ¹H NMR (400 MHz, Chloroform-d) δ 4.61 (d, 1H), 4.48 (dd, 1H), 4.35 (d, 1H), 4.29-3.90 (m, 3H), 3.83-3.50 (m, 3H), 3.51-3.34 (m, 3H), 3.21 (d, 1H), 2.91 (d, 3H), 2.72 (d, 7H), 2.54-2.14 (m, 10H), 2.14-1.68 (m, 9H), 1.67-1.40 (m, 7H), 1.40-1.08 (m, 13H), 1.08-0.76 (m, 4H).

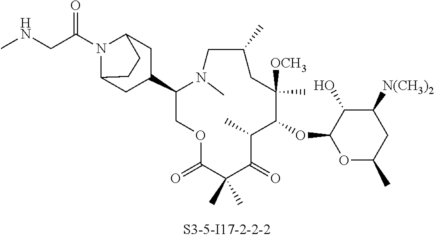

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(8-(methylglycyl)-8-azabicyclo[3.2.1]octan-3-yl)-1-oxa-4-azacyclotridecane-11,13-dione (S3-5-I17-2-2-2) (Compound 44)

Prepared according to the methods of S3-5-I5-1-2-1 from I17 and methylamine to provide the title compound as a formate salt. MS (ESI+) m/z: 681.45 [M+H]⁺; ¹H NMR (400 MHz, Chloroform-d) δ 4.62 (s, 1H), 4.49 (dd, 1H), 4.38-3.80 (m, 5H), 3.79-3.62 (m, 2H), 3.56 (s, 1H), 3.44 (ddd, 1H), 3.38-3.11 (m, 1H), 3.06 (d, 1H), 2.92 (d, 3H), 2.85-2.63 (m, 10H), 2.54 (s, 1H), 2.52-2.19 (m, 4H), 2.18-1.66 (m, 10H), 1.64-1.41 (m, 7H), 1.40-1.11 (m, 13H), 0.90 (q, 3H).

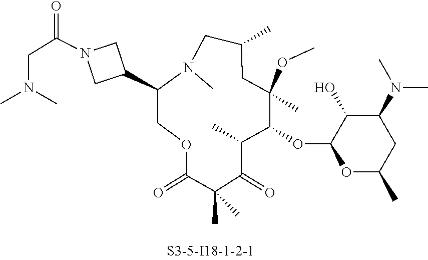

### (3R,6R,8R,9R,10R)-9-(((2S,3R,4S,6R)-4-(dimethylamino)-3-hydroxy-6-methyltetrahydro-2H-pyran-2-yl)oxy)-3-(1-(dimethylglycyl)azetidin-3-yl)-8-methoxy-4,6,8,10,12,12-hexamethyl-1-oxa-4-azacyclotridecane-11,13-dione (S3-5-I18-1-2-1) (Compound 28)

Prepared according to the methods of S3-5-I5-1-2-1 from I18 and dimethylamine to provide the title compound as a formate salt. MS (ESI+) m/z: 641.41 [M+H]⁺; ¹H NMR (400 MHz, Methanol-d) δ 8.08 (s, 1H), 4.57-4.49 (m, 1H), 4.46 (d, 1H), 4.42-4.14 (m, 4H), 4.07 (d, 2H), 3.76 (ddd, 1H), 3.53-3.33 (m, 3H), 3.30-3.25 (m, 1H), 3.22-3.13 (m, 1H), 3.10 (s, 3H), 2.96 (d, 6H), 2.93-2.76 (m, 10H), 2.36-2.24 (m, 1H), 2.11-1.98 (m, 5H), 1.85 (d, 1H), 1.54 (q, 4H), 1.45-1.40 (m, 7H), 1.35 (dd, 6H), 1.09 (d, 3H).

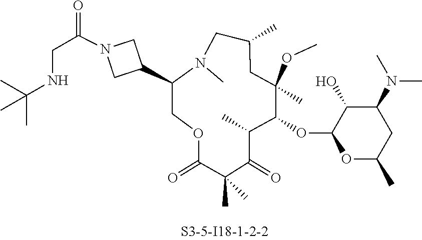

### (3R,6R,8R,9R,10R)-3-(1-(tert-butylglycyl)azetidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-5-I18-1-2-2) (Compound 41)

Prepared according to the methods of S3-5-I5-1-2-1 from I18 and tert-butylamine to provide the title compound as a formate salt. MS (ESI+) m/z: 669.49 [M+H]⁺; ¹H NMR (400 MHz, Methanol-d) δ 8.49 (s, 3H), 4.48 (d, 1H), 4.46-4.39 (m, 1H), 4.37-4.02 (m, 5H), 3.93 (s, 1H), 3.72 (d, 3H), 3.55-3.43 (m, 2H), 3.43-3.34 (m, 1H), 3.25-3.11 (m, 1H), 2.99 (s, 3H), 2.91-2.82 (m, 1H), 2.80 (s, 6H), 2.67-2.52 (m, 3H), 2.13-1.94 (m, 2H), 1.88-1.69 (m, 1H), 1.54 (s, 5H), 1.43-1.21 (m, 22H), 0.97 (d, 3H).

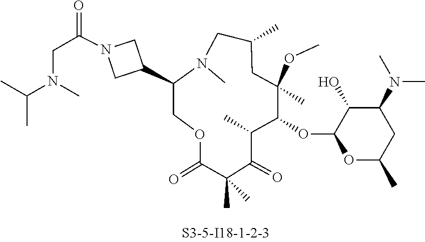

### (3R,6R,8R,9R,10R)-9-(((2S,3R,4S,6R)-4-(dimethylamino)-3-hydroxy-6-methyltetrahydro-2H-pyran-2-yl)oxy)-3-(1-(N-isopropyl-N-methylglycyl)azetidin-3-yl)-8-methoxy-4,6,8,10,12,12-hexamethyl-1-oxa-4-azacyclotridecane-11,13-dione (S3-5-I18-1-2-3) (Compound 194)

Prepared according to the methods of S3-5-I5-1-2-1 from I18 and N-methylpropan-2-amine to provide the title compound as a formate salt. MS (ESI+) m/z: 669.52 [M+H]⁺; ¹H NMR (400 MHz, Methanol-d) δ 8.49 (s, 2H), 4.68-4.41 (m, 2H), 4.41-3.89 (m, 5H), 3.81-3.56 (m, 4H), 3.55-3.34 (m, 4H), 3.27-3.12 (m, 1H), 3.10-2.94 (m, 3H), 2.92-2.75 (m, 7H), 2.73-2.42 (m, 5H), 2.19-1.93 (m, 2H), 1.83-1.65 (m, 1H), 1.63-1.43 (m, 5H), 1.43-1.10 (m, 20H), 1.05-0.84 (m, 3H).

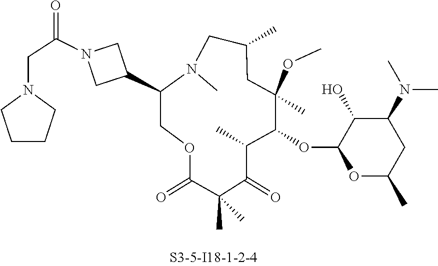

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-(2-(pyrrolidin-1-yl)acetyl)azetidin-3-yl)-1-oxa-4-azacyclotridecane-11,13-dione (S3-5-I18-1-2-4) (Compound 138)

Prepared according to the methods of S3-5-I5-1-2-1 from I18 and pyrrolidine to provide the title compound as a formate salt. MS (ESI+) m/z 667.53 [M+H]⁺; ¹H NMR (400 MHz, Methanol-d) δ 8.47 (s, 3H), 4.65-4.37 (m, 2H), 4.36-4.03 (m, 5H), 3.99-3.81 (m, 3H), 3.79-3.63 (m, 2H), 3.53-3.34 (m, 3H), 3.28-3.11 (m, 5H), 3.01 (s, 4H), 2.93-2.75 (m, 8H), 2.75-2.39 (m, 3H), 2.22-1.93 (m, 6H), 1.84-1.64 (m, 11H), 1.63-1.42 (m, 5H), 1.42-1.20 (m, 12H), 1.09-0.84 (m, 3H).

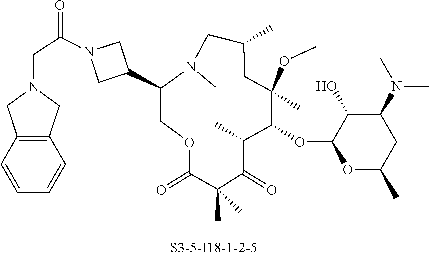

### (3R,6R,8R,9R,10R)-9-(((2S,3R,4S,6R)-4-(dimethylamino)-3-hydroxy-6-methyltetrahydro-2H-pyran-2-yl)oxy)-3-(1-(2-(isoindolin-2-yl)acetyl)azetidin-3-yl)-8-methoxy-4,6,8,10,12,12-hexamethyl-1-oxa-4-azacyclotridecane-11,13-dione (S3-5-I18-1-2-5) (Compound 10)

Prepared according to the methods of S3-5-I5-1-2-1 from I18 and isoindoline to provide the title compound as a formate salt. MS (ESI+) m/z: 715.42 [M+H]⁺; ¹H NMR (400 MHz, Methanol-d) δ 8.43 (s, 2H), 7.33-7.15 (m, 4H), 4.79-4.63 (m, 1H), 4.60-4.33 (m, 3H), 4.32-4.04 (m, 7H), 4.02-3.79 (m, 1H), 3.79-3.68 (m, 1H), 3.57 (s, 2H), 3.51-3.34 (m, 3H), 3.28-3.19 (m, 1H), 3.04 (s, 3H), 3.02-2.91 (m, 1H), 2.81 (s, 6H), 2.78-2.66 (m, 2H), 2.36-2.10 (m, 1H), 2.03 (ddd, 1H), 1.80-1.57 (m, 2H), 1.53 (d, 4H), 1.47-1.20 (m, 14H), 1.03 (t, 3H).

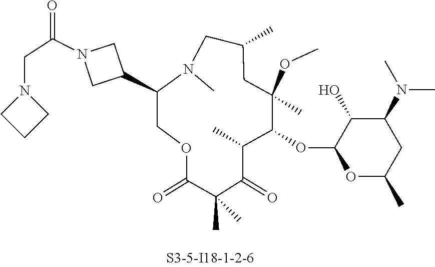

### (3R,6R,8R,9R,10R)-3-(1-(2-(azetidin-1-yl)acetyl)azetidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-5-I18-1-2-6) (Compound 84)

Prepared according to the methods of S3-5-I5-1-2-1 from I18 and azetidine to provide the title compound as a formate salt. MS (ESI+) m/z: 653.44 [M+H]⁺; ¹H NMR (400 MHz, Methanol-d₄) δ 8.36 (s, 3H), 4.80-4.68 (m, 1H), 4.52-4.38 (m, 1H), 4.39-4.07 (m, 8H), 4.07-3.99 (m, 2H), 3.81-3.67 (m, 1H), 3.52-3.34 (m, 3H), 3.06 (d, 3H), 3.00 (m, 4H), 2.90-2.80 (m, 10H), 2.80-2.70 (m, 2H), 2.51 (p, 1H), 2.28-2.12 (m, 1H), 2.04 (ddd, 1H), 1.81-1.64 (m, 1H), 1.64-1.47 (m, 5H), 1.47-1.21 (m, 12H), 1.10-0.92 (m, 3H).

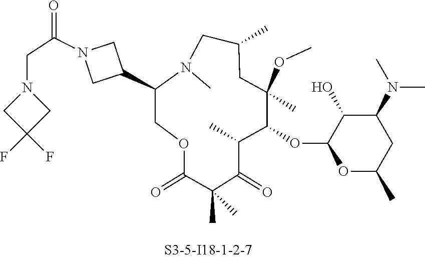

### (3R,6R,8R,9R,10R)-3-(1-(2-(3,3-difluoroazetidin-1-yl)acetyl)azetidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S3-5-I18-1-2-7) (Compound 154)

Prepared according to the methods of S3-5-I5-1-2-1 from I18 and 2,2-difluoroazetidine to provide the title compound as a formate salt. MS (ESI+) m/z: 689.42 [M+H]⁺; ¹H NMR (400 MHz, Methanol-d₄) δ 8.44 (s, 2H), 4.79-4.59 (m, 1H), 4.46 (d, 1H), 4.39-3.98 (m, 5H), 3.98-3.81 (m, 1H), 3.52-3.36 (m, 3H), 3.36-3.27 (m, 8H), 3.27-3.15 (m, 1H), 3.11-2.91 (m, 4H), 2.82 (s, 6H), 2.75 (s, 3H), 2.28-2.08 (m, 1H), 2.08-1.98 (m, 1H), 1.81-1.59 (m, 2H), 1.59-1.45 (m, 4H), 1.45-1.19 (m, 13H), 1.03 (d, 3H).

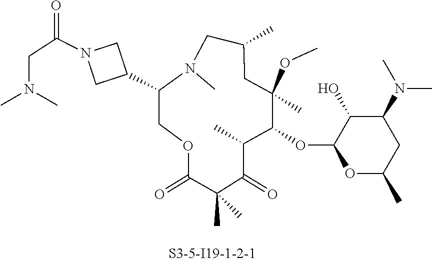

### (3S,6R,8R,9R,10R)-9-(((2S,3R,4S,6R)-4-(dimethylamino)-3-hydroxy-6-methyltetrahydro-2H-pyran-2-yl)oxy)-3-(1-(dimethylglycyl)azetidin-3-yl)-8-methoxy-4,6,8,10,12,12-hexamethyl-1-oxa-4-azacyclotridecane-11,13-dione (S3-5-I19-1-2-1) (Compound 184)

Prepared according to the methods of S3-5-I5-1-2-1 from I19 and dimethylamine to provide the title compound as a formate salt. MS (ESI+) m/z: 641.38 [M+H]⁺; ¹H NMR (400 MHz, Methanol-d) δ 8.22 (s, 2H), 4.47 (t, 0.5H), 4.45-4.40 (m, 1H), 4.38-4.27 (m, 2.5H), 4.24-4.04 (m, 3H), 3.97 (d, 2H), 3.72 (ddd, 1H), 3.50-3.31 (m, 4H), 3.03 (d, 3H), 3.00 (d, 4H), 2.90 (s, 6H), 2.89-2.83 (m, 1H), 2.82 (s, 6H), 2.26-2.12 (m, 1H), 2.04 (dt, 1H), 2.02 (s, 3H), 1.74 (d, 1H), 1.60-1.44 (m, 5H), 1.39 (d, 6H), 1.32 (t, 6H), 1.10-1.01 (m, 3H).

The following examples were prepared according to the methods of S3-5-I5-1-2-1, substituting the appropriate intermediate (Table 2) for I5 in Scheme 1, the appropriate aldehyde for formaldehyde in Scheme 1 to give S1-3-I—R₅, and the appropriate amine for dimethylamine in Scheme 3:

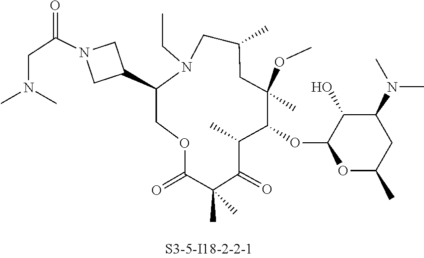

### (3R,6R,8R,9R,10R)-9-(((2S,3R,4S,6R)-4-(dimethylamino)-3-hydroxy-6-methyltetrahydro-2H-pyran-2-yl)oxy)-3-(1-(dimethylglycyl)azetidin-3-yl)-4-ethyl-8-methoxy-6,8,10,12,12-pentamethyl-1-oxa-4-azacyclotridecane-11,13-dione (S3-5-I18-2-2-1) (Compound 38)

Prepared according to the methods of S3-5-I5-1-2-1 from S1-3-I18-2 and dimethylamine to provide the title compound as a formate salt. MS (ESI+) m/z: 655.38 [M+H]⁺; ¹H NMR (400 MHz, Methanol-d) δ 8.44 (s, 2H), 4.47 (d, 1H), 4.31 (dt, 1H), 4.22-3.92 (m, 5H), 3.87-3.59 (m, 5H), 3.52-3.34 (m, 3H), 3.16-2.98 (m, 1H), 2.96-2.77 (m, 11H), 2.77-2.66 (m, 7H), 2.25 (s, 2H), 2.02 (ddd, 1H), 1.77 (s, 1H), 1.60 (s, 3H), 1.52 (q, 1H), 1.39-1.24 (m, 13H), 1.13 (t, 4H), 0.89 (dd, 3H).

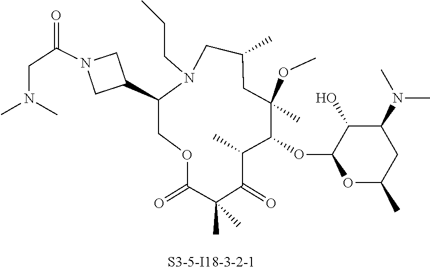

### (3R,6R,8R,9R,10R)-9-(((2S,3R,4S,6R)-4-(dimethylamino)-3-hydroxy-6-methyltetrahydro-2H-pyran-2-yl)oxy)-3-(1-(dimethylglycyl)azetidin-3-yl)-8-methoxy-6,8,10,12,12-pentamethyl-4-propyl-1-oxa-4-azacyclotridecane-11,13-dione (S3-5-I18-3-2-1) (Compound 68)

Prepared according to the methods of S3-5-I5-1-2-1 from S1-3-I18-3 and dimethylamine to provide the title compound as a formate salt. MS (ESI+) m/z: 669.38 [M+H]⁺, formate salt, ¹H NMR (400 MHz, Methanol-d) δ 8.52 (s, 2H), 4.46 (d, 1H), 4.30 (dt, 1H), 4.06 (qd, 6H), 3.85-3.66 (m, 3H), 3.60-3.34 (m, 5H), 2.98 (d, 1H), 2.80 (d, 10H), 2.71-2.58 (m, 8H), 2.28 (d, 2H), 2.07-1.82 (m, 1H), 1.72 (s, 1H), 1.60 (d, 3H), 1.57-1.47 (m, 3H), 1.38-1.22 (m, 12H), 1.08 (d, 1H), 0.96-0.88 (m, 3H), 0.86 (dd, 3H).

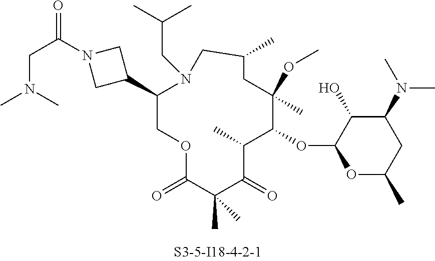

### (3R,6R,8R,9R,10R)-9-(((2S,3R,4S,6R)-4-(dimethylamino)-3-hydroxy-6-methyltetrahydro-2H-pyran-2-yl)oxy)-3-(1-(dimethylglycyl)azetidin-3-yl)-4-isobutyl-8-methoxy-6,8,10,12,12-pentamethyl-1-oxa-4-azacyclotridecane-11,13-dione (S3-5-I18-4-2-1) (Compound 27)

Prepared according to the methods of S3-5-I5-1-2-1 from S1-3-I18-4 and dimethylamine to provide the title compound as a formate salt. MS (ESI+) m/z: 683.36 [M+H]⁺, ¹H NMR (400 MHz, Methanol-d) δ 8.48 (s, 3H), 4.56-4.43 (m, 1H), 4.38-4.17 (m, 1H), 4.16-3.89 (m, 5H), 3.84-3.61 (m, 3H), 3.52-3.34 (m, 4H), 3.06-2.91 (m, 1H), 2.82-2.76 (m, 9H), 2.65-2.49 (m, 6H), 2.40-2.23 (m, 2H), 2.17-1.97 (m, 2H), 1.89-1.83 (m, 1H), 1.80-1.64 (m, 3H), 1.63-1.48 (m, 3H), 1.38-1.21 (m, 15H), 1.13-1.01 (m, 1H), 0.94 (dd, 6H), 0.84 (dd, 3H).

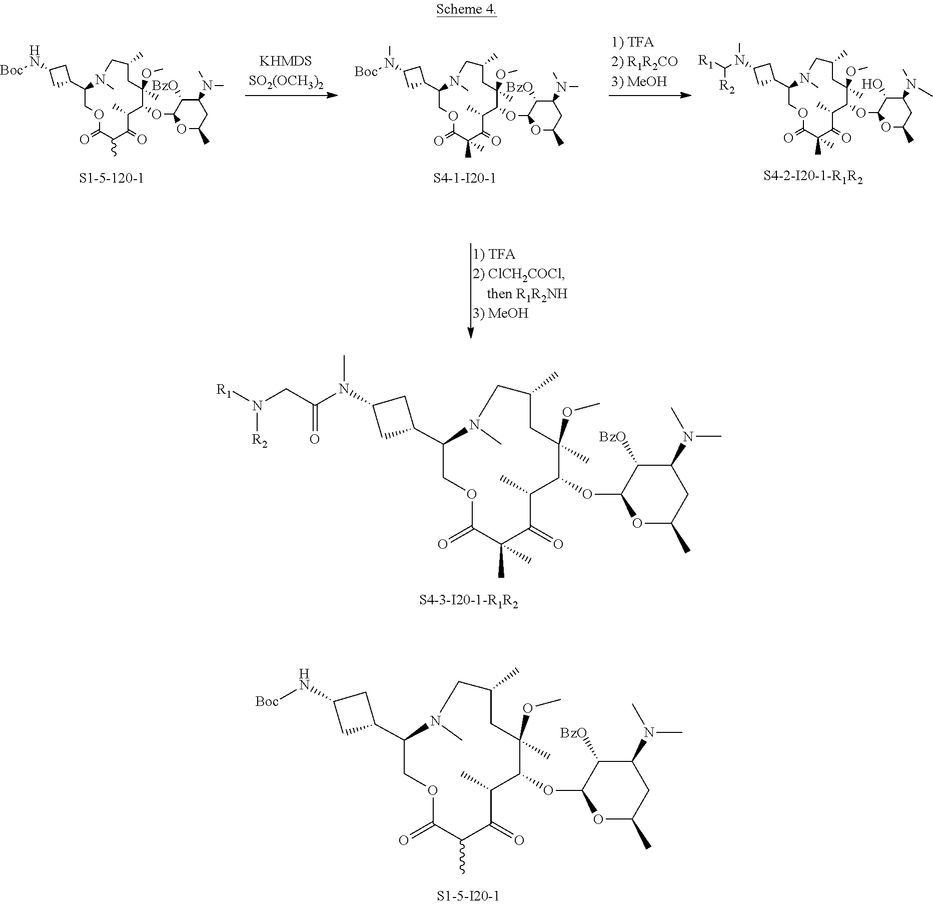

### tert-Butyl ((1S,3s)-3-((3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12-pentamethyl-11,13-dioxo-1-oxa-4-azacyclotridecan-3-yl)cyclobutyl)(methyl)carbamate (S1-5-I20-1)

Prepared according to the methods of S1-5-I1-1, substituting I20 gave 243 mg of the title compound. MS (ESI+) m/z: 380.71 [M+2H]²⁺, 760.18 [M+H]⁺; ¹H NMR (400 MHz, Chloroform-d) δ 8.10-7.96 (m, 2H), 7.57 (t, 1H), 7.45 (t, 2H), 5.05 (dd, 1H), 4.56 (t, 1H), 4.38-4.24 (m, 1H), 4.06 (d, 1H), 4.01-3.86 (m, 1H), 3.62-3.50 (m, 2H), 3.47-3.33 (m, 1H), 3.35-3.17 (m, 1H), 2.89-2.78 (m, 1H), 2.76 (s, 2H), 2.68 (t, 1H), 2.55-2.40 (m, 2H), 2.41-2.29 (m, 2H), 2.26 (d, 4H), 2.18 (d, 2H), 2.01 (q, 2H), 1.92-1.73 (m, 2H), 1.67 (q, 1H), 1.57 (s, 11H), 1.42 (s, 9H), 1.27 (d, 3H), 1.22-1.13 (m, 4H), 1.07-0.92 (m, 3H), 0.82 (d, 3H).

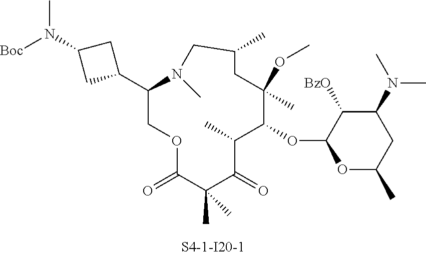

### tert-Butyl ((1S,3s)-3-((3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-11,13-dioxo-1-oxa-4-azacyclotridecan-3-yl)cyclobutyl)(methyl)carbamate (S4-1-I20-1)

S1-5-I20-1 (243 mg, 0.319 mmol) was dissolved in 1,2-dimethoxyethane (3.18 mL), and the reaction mixture was cooled to −78° C. in a dry ice/acetone bath. Potassium bis(trimethylsilyl)amide (1.0 M solution in THF; 0.829 mL, 0.829 mmol) was added. After 5 min, dimethyl sulfate (0.12 mL, 1.27 mmol) was added. The dry ice was removed from the acetone bath, and the reaction mixture was allowed to slowly warm to −10° C. over 50 min. Triethylamine (0.443 mL, 3.19 mmol) was added and the reaction was warmed to room temperature over 30 min. The reaction was quenched by the addition of NH₄Cl (sat., aq. solution) and was diluted with EtOAc. The EtOAc layer was washed with water (2 times) and brine (1 time), was dried over Na₂SO₄, filtered and concentrated. The residue was purified on 12 g of silica gel (elution with 0-12% MeOH-dichloromethane 0.5% NH₄OH gradient) to give the title compound (170 mg, 68%) as a white solid. MS (ESI+) m/z: 394.73 [M+2H]²⁺, 788.23 [M+H]⁺; ¹H NMR (400 MHz, Chloroform-d) δ 8.10-7.98 (m, 2H), 7.56 (t, 1H), 7.44 (t, 2H), 5.04 (dd, 1H), 4.61 (d, 1H), 4.01-3.92 (m, 2H), 3.90-3.76 (m, 1H), 3.64-3.52 (m, 1H), 3.51-3.40 (m, 1H), 2.93-2.85 (m, 1H), 2.84 (s, 4H), 2.76 (s, 3H), 2.43 (d, 1H), 2.25 (s, 6H), 2.23 (s, 3H), 2.15-2.03 (m, 2H), 2.03-1.89 (m, 2H), 1.85 (d, 1H), 1.80-1.68 (m, 2H), 1.61 (s, 1H), 1.44 (s, 9H), 1.38 (d, 4H), 1.31 (s, 3H), 1.27 (d, 4H), 1.22 (s, 3H), 1.06 (d, 3H), 0.96 (dd, 1H), 0.82 (d, 3H).

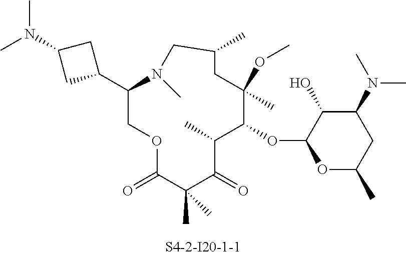

(3R,6R,8R,9R,10R)-9-(((2S,3R,4S,6R)-4-(dimethylamino)-3-hydroxy-6-methyltetrahydro-2H-pyran-2-yl)oxy)-3-((1s,3S)-3-(dimethylamino)cyclobutyl)-8-methoxy-4,6,8,10,12,12-hexamethyl-1-oxa-4-azacyclotridecane-11,13-dione (S4-2-I20-1-1) (Compound 128).

A solution of S4-1-I20-1 (170 mg, 0.215 mmol) in dichloromethane (1 mL) and trifluoroacetic acid (0.25 mL) was stirred at room temperature for 2 h and concentrated. The residue was suspended in ethyl acetate and washed with sat. aq. NaHCO₃ (2 times), the washed solution was dried over sodium sulfate, filtered and concentrated in vacuo. The resulting secondary amine (28 mg, 0.0407 mmol) was dissolved in dichloromethane (1 mL), Na(OAc)₃BH (17.2 mg, 0.0814 mmol) followed by formaldehyde (37 wt % aqueous solution, 0.0274 mL, 0.407 mmol) was added. After 15 min, the reaction mixture was quenched with sat. aq. NaHCO₃ and extracted with dichloromethane (3 times). The combined extracts were concentrated in vacuo. The residue was dissolved in methanol (1.5 mL), and the reaction mixture was heated to 45° C. external temperature for 16 hr. Solvent was removed in vacuo and the residue was purified by HPLC (Atlantis T3 column, 2-40% MeCN-water-0.1% HCO₂H) to give 2.23 mg of the title compound as a formate salt. MS (ESI+) m/z: 200.1 [M+3H]³⁺, 299.62 [M+2H]²⁺, 598.25 [M+H]⁺; ¹H NMR (400 MHz, Methanol-d₄) δ 8.51 (s, 2.6H), 4.45 (d, 1H), 4.24 (s, 2H), 3.73 (q, 8.2, 6:1 Hz, 2H), 3.48-3.33 (m, 3H), 3.05 (s, 5H), 2.78 (s, 8H), 2.56 (s, 2H), 2.41 (s, 8H), 2.13 (t, 2H), 2.05-1.86 (m, 2H), 1.50 (s, 4H), 1.40 (s, 6H), 1.34 (t, 6H), 1.04 (s, 3H).

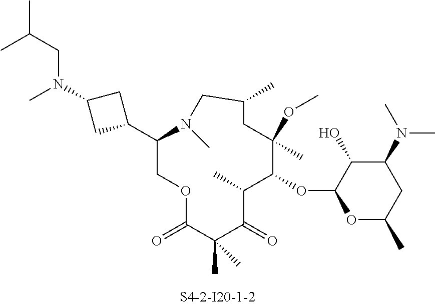

### (3R,6R,8R,9R,10R)-9-(((2S,3R,4S,6R)-4-(dimethylamino)-3-hydroxy-6-methyltetrahydro-2H-pyran-2-yl)oxy)-3-((1S,3S)-3-(isobutyl(methyl)amino)cyclobutyl)-8-methoxy-4,6,8,10,12,12-hexamethyl-1-oxa-4-azacyclotridecane-11,13-dione (S4-2-I20-1-2) (Compound 187)

Prepared according to the methods of S4-2-I20-1-1, substituting isobutyraldehyde to provide 15.5 mg of the title compound as a formate salt. MS (ESI+) m/z: 214.14 [M+3H]³⁺, 320.63 [M+2H]²⁺, 640.31 [M+H]⁺; ¹H NMR (400 MHz, Methanol-d₄) δ 8.53 (s, 2H), 4.67 (s, 1H), 4.45 (d, 1H), 4.31-4.10 (m, 2H), 3.72 (ddt, 1H), 3.63 (s, 1H), 3.49-3.33 (m, 3H), 3.04 (s, 6H), 2.89-2.80 (m, 2H), 2.77 (s, 6H), 2.63-2.47 (m, 1H), 2.35 (s, 7H), 2.15 (q, 2H), 2.06-1.85 (m, 3H), 1.84-1.65 (m, 1H), 1.53 (s, 3H), 1.39 (s, 6H), 1.33 (t, 6H), 1.04 (d, 3H), 0.98 (d, 6H).

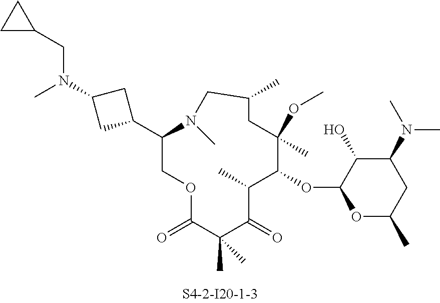

### (3R,6R,8R,9R,10R)-3-((1s,3S)-3-((cyclopropylmethyl)(methyl)amino)cyclobutyl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S4-2-I20-1-3) (Compound 18)

Prepared according to the methods of S4-2-I20-1-1, substituting cyclopropanecarboxaldehyde to provide 14.79 mg of the title compound as a formate salt. MS (ESI+) m/z: 213.47 [M+3H]³⁺, 319.63 [M+2H]²⁺, 638.30 [M+H]⁺; ¹H NMR (400 MHz, Methanol-d₄) δ 8.53 (s, 2.5H), 4.66 (s, 1H), 4.46 (d, 1H), 4.33-4.05 (m, 2H), 3.79-3.54 (m, 2H), 3.48-3.34 (m, 4H), 3.04 (s, 5H), 2.79 (s, 7H), 2.75-2.66 (m, 3H), 2.64 (s, 4H), 2.45 (q, 2H), 2.34 (t, 1H), 2.14 (d, 2H), 2.06-1.96 (m, 1H), 1.81-1.57 (m, 2H), 1.53 (s, 3H), 1.51-1.43 (m, 1H), 1.39 (s, 6H), 1.34 (t, 6H), 1.04 (d, 4H), 0.76-0.62 (m, 2H), 0.35 (d, 2H).

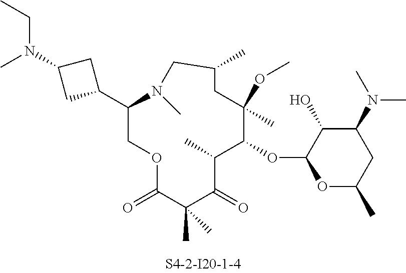

### (3R,6R,8R,9R,10R)-9-(((2S,3R,4S,6R)-4-(dimethylamino)-3-hydroxy-6-methyltetrahydro-2H-pyran-2-yl)oxy)-3-((1s,3S)-3-(ethyl(methyl)amino)cyclobutyl)-8-methoxy-4,6,8,10,12,12-hexamethyl-1-oxa-4-azacyclotridecane-11,13-dione (S4-2-I20-1-4) (Compound 165)

Prepared according to the methods of S4-2-I20-1-1, substituting acetaldehyde to provide 13.63 mg of the title compound as a formate salt. MS (ESI+) m/z: 204.79 [M+3H]³⁺, 306.63 [M+2H]²⁺, 612.23 [M+H]⁺; ¹H NMR (400 MHz, Methanol-d₄) δ 8.54 (s, 2H), 4.45 (d, 1H), 4.28-4.05 (m, 2H), 3.72 (ddd, 1H), 3.44 (dd, 2H), 3.34 (d, 0.6H), 3.29-3.16 (m, 1.4H), 3.03 (s, 5H), 2.76 (s, 10H), 2.58 (s, 2H), 2.48-2.29 (m, 5H), 2.19 (q, 2H), 2.06-1.94 (m, 2H), 1.55-1.47 (m, 4H), 1.39 (s, 6H), 1.33 (t, 6H), 1.27 (d, 1H), 1.22 (t, 3H), 1.03 (s, 3H).

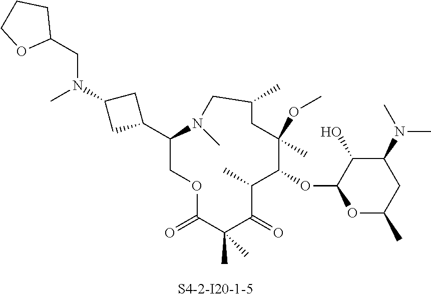

### (3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((1s,3S)-3-(methyl((tetrahydrofuran-2-yl)methyl)amino)cyclobutyl)-1-oxa-4-azacyclotridecane-11,13-dione (S4-2-I20-1-5) (Compound 31)

Prepared according to the methods of S4-2-I20-1-1, substituting tetrahydrofuran-2-carboxaldehyde to provide 20.49 mg of the title compound as a formate salt. MS (ESI+) m/z: 223.48 [M+3H]³⁺, 334.64 [M+2H]²⁺, 668.28 [M+H]⁺; ¹H NMR (400 MHz, Methanol-d₄) δ 8.52 (s, 2H), 4.68 (s, 1H), 4.45 (d, 1H), 4.25 (d, 1H), 4.20 (dd, 1H), 4.12 (qd, 1H), 3.87 (p, 1H), 3.82-3.69 (m, 2H), 3.64 (s, 1H), 3.50-3.32 (m, 3H), 3.24 (s, 1H), 3.05 (s, 4H), 2.90-2.82 (m, 2H), 2.80 (s, 6H), 2.68 (s, 2H), 2.56 (dt, 1H), 2.48 (s, 3H), 2.39 (q, 2H), 2.17 (dt, 2H), 2.10-1.97 (m, 2.5H), 1.97-1.83 (m, 2.5H), 1.82-1.63 (m, 1H), 1.64-1.43 (m, 7H), 1.40 (d, 6H), 1.34 (t, 6H), 1.05 (d, 3H).

The following examples were prepared according to the methods of S4-2-I20-1-1, substituting the appropriate intermediate (Table 2, I) for I20 in Scheme 1 and the appropriate aldehyde or ketone for formaldehyde.

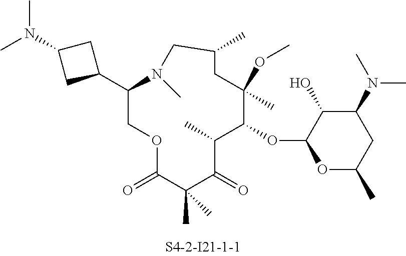

### (3R,6R,8R,9R,10R)-9-(((2S,3R,4S,6R)-4-(dimethylamino)-3-hydroxy-6-methyltetrahydro-2H-pyran-2-yl)oxy)-3-((1r,3R)-3-(dimethylamino)cyclobutyl)-8-methoxy-4,6,8,10,12,12-hexamethyl-1-oxa-4-azacyclotridecane-11,13-dione (S4-2-I21-1-1) (Compound 30)

Prepared according to the methods of S4-2-I20-1-1 from I21 and formaldehyde to provide 7.41 mg of the title compound as a formate salt. MS (ESI+) m/z: 200.12 [M+3H]³⁺, 299.65 [M+2H]²⁺, 598.25 [M+H]⁺; ¹H NMR (400 MHz, Methanol-d₄) δ 8.53 (s, 2.6H), 4.69 (s, 1H), 4.46 (d, 1H), 4.24 (d, 2H), 3.81-3.55 (m, 2H), 3.51-3.34 (m, 3H), 3.21 (s, 1H), 3.05 (s, 4H), 2.90 (s, 1H), 2.79 (s, 8H), 2.49 (s, 8H), 2.31 (td, 2H), 2.18 (s, 1H), 2.07-1.96 (m, 1H), 1.83-1.66 (m, 1H), 1.66-1.56 (m, 1H), 1.57-1.46 (m, 4H), 1.40 (s, 6H), 1.33 (t, 6H), 1.04 (d, 3H).

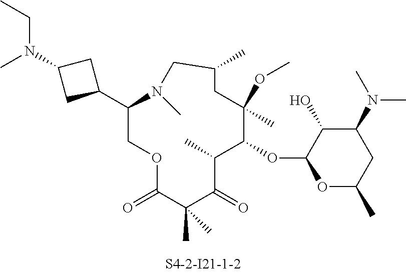

### (3R,6R,8R,9R,10R)-9-(((2S,3R,4S,6R)-4-(dimethylamino)-3-hydroxy-6-methyltetrahydro-2H-pyran-2-yl)oxy)-3-((1r,3R)-3-(ethyl(methyl)amino)cyclobutyl)-8-methoxy-4,6,8,10,12,12-hexamethyl-1-oxa-4-azacyclotridecane-11,13-dione (S4-2-I21-1-2) (Compound 71)

Prepared according to the methods of S4-2-I20-1-1 from I21 and acetaldehyde to provide 5.35 mg of the title compound as a formate salt. MS (ESI+) m/z: 204.80 [M+3H]³⁺, 306.65 [M+2H]²⁺, 612.30 [M+H]⁺; ¹H NMR (400 MHz, Methanol-d₄) δ 8.52 (s, 2.6H), 4.65 (s, 1H), 4.47 (d, 1H), 4.23 (d, 2H), 3.72 (dtd, 2H), 3.50-3.32 (m, 4H), 3.03 (s, 5H), 2.79 (s, 12H), 2.51 (s, 4H), 2.48-2.26 (m, 4H), 2.25-2.07 (m, 1H), 2.02 (d, 1H), 1.71-1.62 (m, 1H), 1.55 (s, 3H), 1.53-1.45 (m, 1H), 1.39 (s, 6H), 1.35-1.29 (m, 6H), 1.23 (t, 3H), 1.11-0.95 (m, 3H).

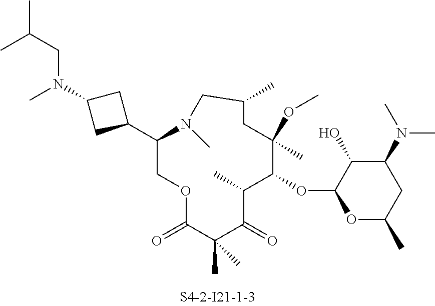

### (3R,6R,8R,9R,10R)-9-(((2S,3R,4S,6R)-4-(dimethylamino)-3-hydroxy-6-methyltetrahydro-2H-pyran-2-yl)oxy)-3-((1r,3R)-3-(isobutyl(methyl)amino)cyclobutyl)-8-methoxy-4,6,8,10,12,12-hexamethyl-1-oxa-4-azacyclotridecane-11,13-dione (S4-2-I21-1-3) (Compound 131)

Prepared according to the methods of S4-2-I20-1-1 from I21 and isobutyraldehyde to provide 14.45 mg of the title compound as a formate salt. MS (ESI+) m/z: 214.16 [M+3H]³⁺, 320.66 [M+2H]²⁺, 640.34 [M+H]⁺; ¹H NMR (400 MHz, Methanol-d₄) δ 8.52 (s, 2.6H), 4.68 (s, 1H), 4.46 (d, 1H), 4.33-4.18 (m, 2H), 3.80-3.61 (m, 2H), 3.51-3.34 (m, 3H), 3.04 (s, 5H), 2.80 (s, 10H), 2.58-2.33 (s, 8H), 2.33-2.08 (m, 2H), 2.08-1.88 (m, 2H), 1.84-1.68 (s, 1H), 1.57-1.44 (m, 4H), 1.39 (s, 6H), 1.34 (t, 6H), 1.04 (d, 3H), 1.00 (d, 6H).

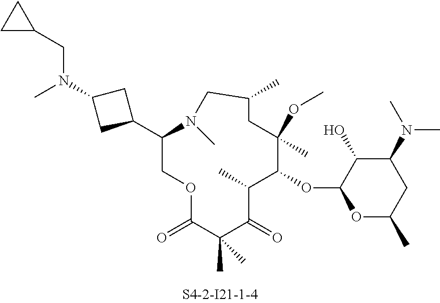

### (3R,6R,8R,9R,10R)-3-((1r,3R)-3-((cyclopropylmethyl)(methyl)amino)cyclobutyl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S4-2-I21-1-4) (Compound 24)

Prepared according to the methods of S4-2-I20-1-1 from I21 and cyclopropanecarboxaldehyde to provide 15.6 mg of the title compound as a formate salt. MS (ESI+) m/z: 213.49 [M+3H]³⁺, 319.64 [M+2H]²⁺, 638.35 [M+H]⁺; ¹H NMR (400 MHz, Methanol-d₄) δ 8.52 (s, 2.6H), 4.66 (s, 1H), 4.51-4.41 (m, 1H), 4.24 (d, 2H), 3.82-3.62 (m, 2H), 3.57 (s, 1H), 3.49-3.33 (m, 3H), 3.04 (s, 4H), 2.89 (s, 2H), 2.81 (d, 6H), 2.77 (d, 4H), 2.70 (s, 3H), 2.63 (s, 1H), 2.56-2.42 (m, 2H), 2.42-2.27 (m, 1H), 2.19 (s, 1H), 2.03 (ddd, 1H), 1.83-1.58 (m, 2H), 1.58-1.46 (m, 4H), 1.40 (s, 5H), 1.33 (dd, 6H), 1.04 (d, 4H), 0.78-0.63 (m, 2H), 0.36 (t, 2H).

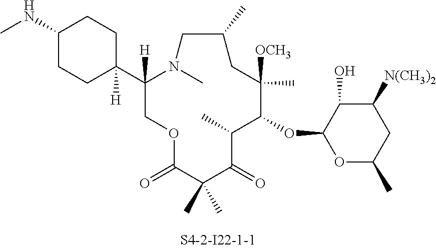

### (3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((1r,4S)-4-(methylamino)cyclohexyl)-1-oxa-4-azacyclotridecane-11,13-dione (S4-2-I22-1-1) (Compound 26)

Prepared according to the methods of S4-2-I20-1-1 from I22 and eliminating the reductive alkylation step, to provide the title compound as a formate salt. MS (ESI+) m/z: 612.40 [M+H]⁺; ¹H NMR (400 MHz, Chloroform-d) δ 4.45 (d, 1H), 4.35-4.04 (m, 2H), 3.83-3.57 (m, 2H), 3.49-3.32 (m, 3H), 3.11-2.92 (m, 5H), 2.78 (s, 8H), 2.67 (s, 4H), 2.16 (d, 4H), 2.05-1.88 (m, 4H), 1.60-1.19 (m, 24H), 1.11-0.86 (m, 4H).

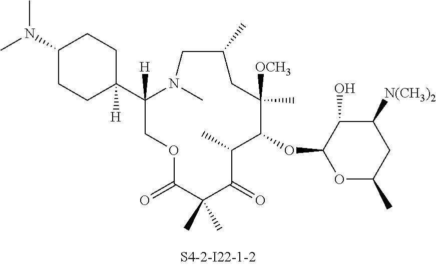

### (3S,6R,8R,9R,10R)-9-(((2S,3R,4S,6R)-4-(dimethylamino)-3-hydroxy-6-methyltetrahydro-2H-pyran-2-yl)oxy)-3-((1r,4S)-4-(dimethylamino)cyclohexyl)-8-methoxy-4,6,8,10,12,12-hexamethyl-1-oxa-4-azacyclotridecane-11,13-dione (S4-2-I22-1-2) (Compound 137)

Prepared according to the methods of S4-2-I20-1-1 from I22 and formaldehyde to provide the title compound as a formate salt. MS (ESI+) m/z: 626.43 [M+H]⁺; ¹H NMR (400 MHz, Chloroform-d) δ 4.45 (d, 1H), 4.36-4.10 (m, 2H), 3.80-3.59 (m, 2H), 3.50-3.33 (m, 3H), 3.22-2.88 (m, 7H), 2.79 (s, 14H), 2.25-1.88 (m, 7H), 1.74-1.43 (m, 9H), 1.43-1.18 (m, 14H), 1.15-0.81 (m, 4H).

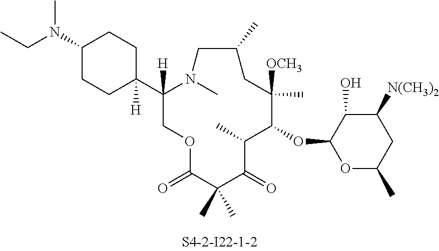

### (3S,6R,8R,9R,10R)-9-(((2S,3R,4S,6R)-4-(dimethylamino)-3-hydroxy-6-methyltetrahydro-2H-pyran-2-yl)oxy)-3-((1r,4S)-4-(ethyl(methyl)amino)cyclohexyl)-8-methoxy-4,6,8,10,12,12-hexamethyl-1-oxa-4-azacyclotridecane-11,13-dione (S4-2-I22-1-3) (Compound 157)

Prepared according to the methods of S4-2-I20-1-1 from I22 and acetaldehyde to provide the title compound as a formate salt. MS (ESI+) m/z: 640.46 [M+H]⁺; ¹H NMR (400 MHz, Chloroform-d) δ 4.44 (d, 1H), 4.28 (d, 1H), 4.22-4.07 (m, 1H), 3.70 (tdd, 2H), 3.51 (s, 1H), 3.47-3.33 (m, 3H), 3.26-3.12 (m, 3H), 2.98 (s, 5H), 2.87-2.71 (m, 11H), 2.66 (s, 1H), 2.23-1.84 (m, 7H), 1.81-1.42 (m, 9H), 1.42-1.19 (m, 17H), 1.03 (s, 3H).

The following examples were prepared according to the methods of S4-2-I20-1-1, substituting the appropriate intermediate (Table 2) for I20 in Scheme 1, the appropriate aldehyde for formaldehyde in Scheme 1 to give S1-3-I—R₅, and the appropriate aldehyde or ketone for formaldehyde in Scheme 4.

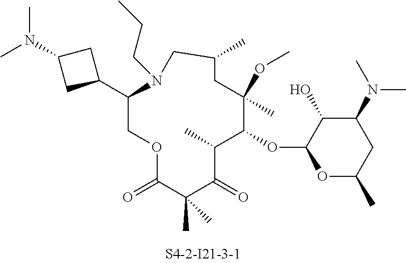

### (3R,6R,8R,9R,10R)-9-(((2S,3R,4S,6R)-4-(dimethylamino)-3-hydroxy-6-methyltetrahydro-2H-pyran-2-yl)oxy)-3-((1r,3R)-3-(dimethylamino)cyclobutyl)-8-methoxy-6,8,10,12,12-pentamethyl-4-propyl-1-oxa-4-azacyclotridecane-11,13-dione (S4-2-I21-3-1)

Prepared according to the methods of S4-2-I20-1-1 from S1-3-I21-3 and formaldehyde to provide 9.74 mg of the title compound as a formate salt. MS (ESI+) m/z: 209.50 [M+3H]³⁺, 313.67 [M+2H]²⁺, 626.35 [M+H]⁺; ¹H NMR (400 MHz, Methanol-d₄) δ 8.50 (s, 2H), 4.46 (d, 11H), 4.07 (s, 2H), 3.91 (s, 1H), 3.76-3.50 (m, 3H), 3.50-3.34 (m, 2H), 2.81 (s, 12H), 2.68 (s, 8H), 2.49-2.35 (m, 4H), 2.23 (s, 2H), 2.02 (ddd, 1H), 1.81 (s, 1H), 1.63-1.44 (m, 6H), 1.40-1.19 (m, 12H), 0.94 (t, 3H), 0.91-0.78 (m, 3H).

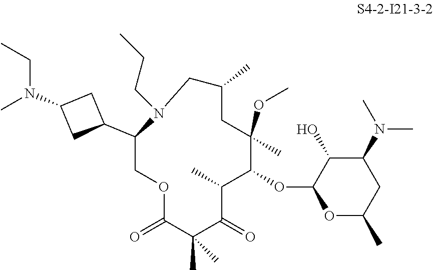

### (3R,6R,8R,9R,10R)-9-(((2S,3R,4S,6R)-4-(dimethylamino)-3-hydroxy-6-methyltetrahydro-2H-pyran-2-yl)oxy)-3-((1r,3R)-3-(ethyl(methyl)amino)cyclobutyl)-8-methoxy-6,8,10,12,12-pentamethyl-4-propyl-1-oxa-4-azacyclotridecane-11,13-dione (S4-2-I21-3-2) (Compound 94)

Prepared according to the methods of S4-2-I20-1-1 from S1-3-I21-3 and acetaldehyde to provide 4.40 mg of the title compound as a formate salt. MS (ESI+) m/z: 214.19 [M+3H]³⁺, 320.67 [M+2H]²⁺, 640.34 [M+H]⁺; ¹H NMR (400 MHz, Methanol-d₄) δ 8.51 (s, 2H), 4.46 (d, 1H), 4.04 (d, 2H), 3.84 (s, 1H), 3.78-3.61 (m, 3H), 3.52-3.33 (m, 2H), 3.20 (s, 1H), 3.01 (s, 2H), 2.79 (s, 11H), 2.66 (s, 3H), 2.48-2.36 (m, 3H), 2.26-2.12 (s, 2H), 2.04-1.97 (m, 1H), 1.78-1.66 (m, 1H), 1.61 (s, 3H), 1.58-1.45 (m, 3H), 1.39-1.21 (m, 15H), 1.14-0.98 (m, 1H), 0.93 (t, 3H), 0.89-0.81 (m, 3H).

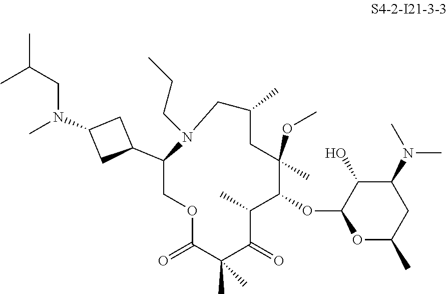

### (3R,6R,8R,9R,10R)-9-(((2S,3R,4S,6R)-4-(dimethylamino)-3-hydroxy-6-methyltetrahydro-2H-pyran-2-yl)oxy)-3-((1r,3R)-3-(isobutyl(methyl)amino)cyclobutyl)-8-methoxy-6,8,10,12,12-pentamethyl-4-propyl-1-oxa-4-azacyclotridecane-11,13-dione (S4-2-I21-3-3) (Compound 60)

Prepared according to the methods of S4-2-I20-1-1 from S1-3-I21-3 and isobutyraldehyde to provide 4.98 mg of the title compound as a formate salt. MS (ESI+) m/z: 223.53 [M+3H]³⁺, 334.67 [M+2H]²⁺, 668.38 [M+H]⁺; ¹H NMR (400 MHz, Methanol-d₄) δ 8.52 (s, 2H), 4.46 (d, 1H), 4.05 (s, 2H), 3.83 (s, 1H), 3.77-3.49 (m, 3H), 3.45 (dd, 1H), 3.41-3.33 (m, 1H), 3.18 (s, 1H), 2.79 (s, 11H), 2.69-2.46 (m, 6H), 2.39 (t, 4H), 2.16 (s, 2H), 2.10-1.93 (m, 2H), 1.84-1.67 (m, 1H), 1.61 (s, 3H), 1.58-1.40 (m, 3H), 1.39-1.22 (m, 11H), 1.03 (d, 6H), 0.98-0.89 (m, 3H), 0.86 (s, 2H).

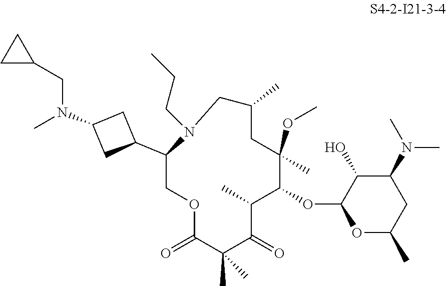

### (3R,6R,8R,9R,10R)-3-((1r,3R)-3-((cyclopropylmethyl)(methyl)amino)cyclobutyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S4-2-I21-3-4) (Compound 95)

Prepared according to the methods of S4-2-I20-1-1 from S1-3-I21-3 and cyclopropanecarboxaldehyde to provide 7.9 mg of the title compound as a formate salt. MS (ESI+) m/z: 222.83 [M+3H]³⁺, 333.67 [M+2H]²⁺, 666.36 [M+H]⁺; ¹H NMR (400 MHz, Methanol-d₄) δ 8.50 (s, 2H), 4.46 (d, 1H), 4.05 (d, 2H), 3.81 (s, 2H), 3.76-3.62 (m, 2H), 3.53-3.33 (m, 2H), 3.28-3.07 (m, 1H), 2.88 (d, 3H), 2.81 (s, 7H), 2.79 (s, 6H), 2.59 (s, 1H), 2.45 (t, 3H), 2.37-2.13 (m, 3H), 2.02 (dt, 1H), 1.72 (s, 1H), 1.66 (s, 1H), 1.58 (s, 3H), 1.57-1.44 (m, 3H), 1.36-1.24 (m, 12H), 1.15-1.10 (m, 2H), 0.93 (t, 3H), 0.86 (d, 3H), 0.80-0.67 (m, 2H), 0.41 (t, 2H).

The following examples were prepared according to the methods of S4-2-I20-1, substituting I22 for I20 in Scheme 1, to give S4-2-I22-1. S4-2-I22-1 was further elaborated according to the methods of S3-5-I5-1-2-1, substituting the appropriate amine for dimethylamine.

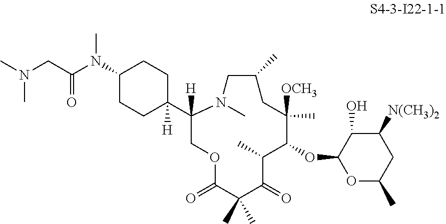

### 2-(dimethylamino)-N-((1S,4r)-4-((3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-11,13-dioxo-1-oxa-4-azacyclotridecan-3-yl)cyclohexyl)-N-methylacetamide (S4-3-I22-1-1) (Compound 82)

Prepared according to the methods of S3-5-I5-1-2-1 from S4-1-I22-1 and dimethylamine to provide the title compound as a formate salt. MS (ESI+) m/z: 697.45 [M+H]⁺; ¹H NMR (400 MHz, Chloroform-d) δ 4.45 (d, 1H), 4.25 (d, 2H), 3.95 (d, 2H), 3.73 (dt, 1H), 3.40 (ddt, 3H), 3.13-2.94 (m, 5H), 2.88 (d, 4H), 2.77 (q, 13H), 2.44-2.12 (m, 3H), 2.07-1.70 (m, 8H), 1.58-1.45 (m, 6H), 1.44-1.21 (m, 16H), 1.12-0.89 (m, 4H).

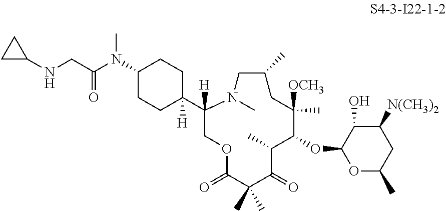

### 2-(cyclopropylamino)-N-((1S,4r)-4-((3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-11,13-dioxo-1-oxa-4-azacyclotridecan-3-yl)cyclohexyl)-N-methylacetamide (S4-3-I22-1-2) (Compound 180)

Prepared according to the methods of S3-5-I5-1-2-1 from S4-1-I22-1 and cyclopropylamine to provide the title compound as a formate salt. MS (ESI+) m/z: 709.46 [M+H]⁺; ¹H NMR (400 MHz, Chloroform-d) δ 4.46 (d, 1H), 4.26 (dd, 3H), 3.95 (s, 1H), 3.88-3.53 (m, 3H), 3.52-3.32 (m, 4H), 2.98 (d, 7H), 2.88 (d, 3H), 2.80 (s, 7H), 2.68 (d, 1H), 2.47 (dq, 1H), 2.26 (d, 2H), 2.08-1.62 (m, 9H), 1.60-1.45 (m, 6H), 1.45-1.20 (m, 14H), 1.15-0.96 (m, 3H), 0.74-0.58 (m, 3H).

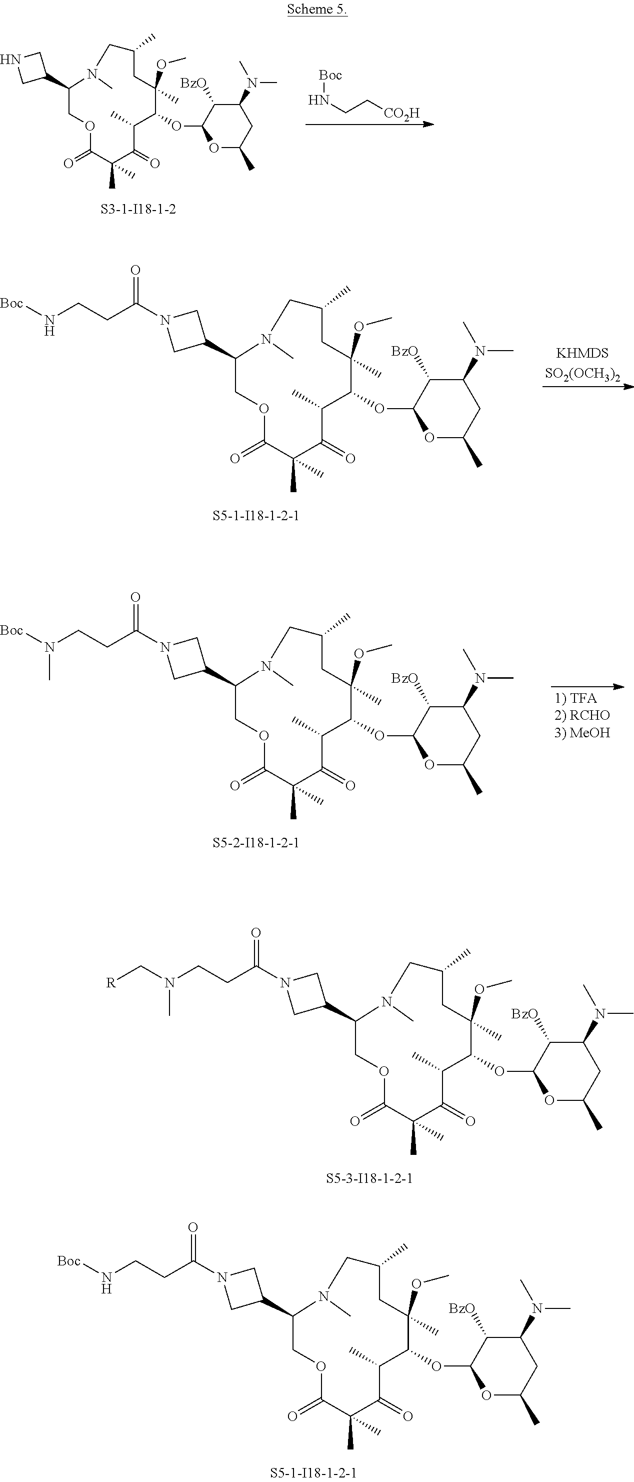

(2S,3R,4S,6R)-2-(((3R,6R,8R,9R,10R)-3-(1-(3-((tert-butoxycarbonyl)amino)propanoyl)azetidin-3-yl)-8-methoxy-4,6,8,10,12,12-hexamethyl-11,13-dioxo-1-oxa-4-azacyclotridecan-9-yl)oxy)-4-(dimethylamino)-6-methyltetrahydro-2H-pyran-3-yl benzoate (S5-1-I18-1-2-1). To a solution of S3-1-I18-1-2 (230 mg, 0.348 mmol) and 3-((tert-butoxycarbonyl)amino)propanoic acid (65.8 mg, 0.348 mmol) in dichloromethane (3.48 mL) was added N,N-diisopropylethylamine (0.121 mL, 0.696 mmol) followed by HATU (132 mg, 0.348 mmol). The reaction mixture was stirred at room temperature for 1 hr, solvent and excess reagent were removed in vacuo. The residue was purified on 12 g of silica gel (elution with 0-10% MeOH-dichloromethane gradient) to give the title compound as a white solid (252 mg, 87%). MS (ESI+) m/z: 416.47 [M+2H]²⁺, 831.40 [M+H]⁺; ¹H NMR (400 MHz, Chloroform-d) δ 8.09-7.99 (m, 2H), 7.60-7.51 (m, 1H), 7.45 (q, 2H), 5.27-5.08 (m, 1H), 5.02 (t, 1H), 4.59 (d, 1H), 4.34-4.16 (m, 1H), 4.16-3.75 (m, 5H), 3.71 (q, 1H), 3.63-3.51 (m, 1H), 3.48-3.28 (m, 2H), 3.28-3.09 (m, 2H), 3.03 (s, 1H), 2.88-2.72 (m, 4H), 2.50-2.39 (m, 1H), 2.33-2.20 (m, 8H), 2.21-2.03 (m, 1H), 1.81 (dd, 2H), 1.56 (s, 3H), 1.52-1.35 (m, 15H), 1.35-1.15 (m, 8H), 1.15-0.94 (m, 4H), 0.84 (dd, 3H).

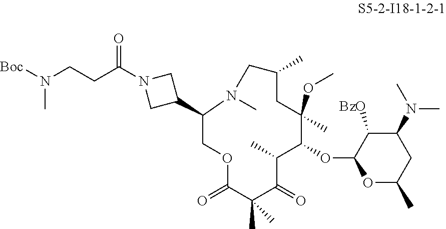

tert-Butyl (3-(3-((3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-11,13-dioxo-1-oxa-4-azacyclotridecan-3-yl)azetidin-1-yl)-3-oxopropyl)(methyl)carbamate (S5-2-I18-1-2-1). S5-1-I18-1-2-1 (252 mg, 0.303 mmol) was dissolved in 1,2-dimethoxyethane (3.03 mL), and the reaction mixture was cooled to −78° C. in a dry ice/acetone bath. Potassium bis(trimethylsilyl)amide (1.0 M solution in THF; 0.393 mL, 0.393 mmol) was added. After 5 min, dimethyl sulfate (0.057 mL, 0.606 mmol) was added. The dry ice was removed from the acetone bath, and the reaction mixture was allowed to slowly warm to −10° C. over 50 min. The reaction was quenched by the addition of NH₄Cl (sat., aq. solution) and was diluted with EtOAc. The EtOAc layer was washed with water (2 times) and brine (1 time), was dried over Na₂SO₄, filtered and concentrated. The residue was purified on 12 g of silica gel (elution with 0-12% MeOH-dichloromethane-0.5% NH₄OH gradient) to give the title compound (133 mg, 52%) as a white solid. MS (ESI+) m/z: 423.46 [M+2H]²⁺, 845.48 [M+H]⁺.

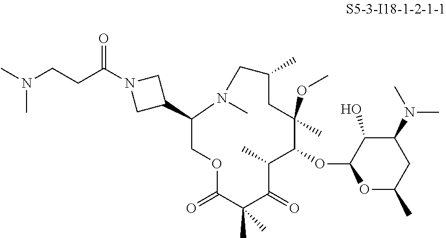

### (3R,6R,8R,9R,10R)-9-(((2S,3R,4S,6R)-4-(dimethylamino)-3-hydroxy-6-methyltetrahydro-2H-pyran-2-yl)oxy)-3-(1-(3-(dimethylamino)propanoyl)azetidin-3-yl)-8-methoxy-4,6,8,10,12,12-hexamethyl-1-oxa-4-azacyclotridecane-11,13-dione (S5-3-I18-1-2-1-1) (Compound 50)

A solution of S5-2-I18-1-2-1 (133 mg, 0.29 mmol) in dichloromethane (0.6 mL) and trifluoroacetic acid (0.3 mL) was stirred at room temperature for 2 hr and concentrated. The residue was suspended in ethyl acetate and washed with sat. aq. NaHCO₃ (2 times), the washed solution was dried over sodium sulfate, filtered and concentrated in vacuo. The resulting secondary amine (30 mg, 0.0395 mmol) was dissolved in dichloromethane (1 mL), Na(OAc)₃BH (16.7 mg, 0.079 mmol) followed by formaldehyde (37 wt % aqueous solution, 0.0265 mL, 0.394 mmol) was added. After 15 min, the reaction mixture was quenched with sat., aq. NaHCO₃ and extracted with dichloromethane (3 times). The combined extracts were concentrated in vacuo. The residue was dissolved in methanol (1.5 mL), and the reaction mixture was heated to 45° C. external temperature for 16 hr. Solvent was removed in vacuo and the residue was purified by HPLC (Atlantis T3 column, 2-40% MeCN-water-0.1% HCO₂H) to give the title compound as a formate salt (1.67 mg, 0.0026 mmol, 6.47%). MS (ESI+) m/z: 219.30 [M+3H]³⁺, 328.40 [M+2H]²⁺, 655.36 [M+H]⁺; ¹H NMR (400 MHz, Methanol-d₄) δ 8.50 (s, 2H), 4.49 (d, 1H), 4.44-4.26 (m, 1.5H), 4.26-4.02 (m, 4.5H), 3.97 (s, 0.5H), 3.83 (s, 0.5H), 3.72 (t, 1H), 3.53 (s, 1H), 3.45 (dd, 2H), 3.39-3.33 (m, 2H), 3.18-3.05 (m, 3H), 2.97 (s, 3H), 2.78 (s, 6H), 2.68 (s, 7H), 2.54 (q, 3H), 2.50-2.38 (m, 2H), 2.05-1.97 (m, 1H), 1.88 (s, 2H), 1.57-1.45 (m, 5H), 1.37 (d, 3H), 1.31 (t, 9H), 0.94 (s, 3H).

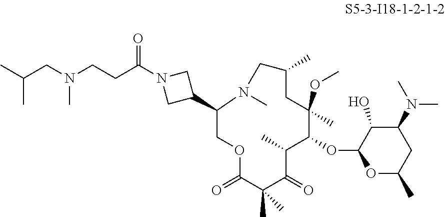

### (3R,6R,8R,9R,10R)-9-(((2S,3R,4S,6R)-4-(dimethylamino)-3-hydroxy-6-methyltetrahydro-2H-pyran-2-yl)oxy)-3-(1-(3-(isobutyl(methyl)amino)propanoyl)azetidin-3-yl)-8-methoxy-4,6,8,10,12,12-hexamethyl-1-oxa-4-azacyclotridecane-11,13-dione (S5-3-I18-1-2-1-2) (Compound 125)

Prepared according to the methods of S5-3-I18-1-2-1-1, substituting isobutyraldehyde to provide 12.28 mg of the title compound as a formate salt. MS (ESI+) m/z: 233.32 [M+3H]³⁺, 349.44 [M+2H]²⁺, 697.45 [M+H]⁺; ¹H NMR (400 MHz, Methanol-d₄) δ 8.53 (s, 2H), 4.49 (d, 1H), 4.40 (t, 1H), 4.35-4.23 (m, 1H), 4.22-4.05 (m, 4H), 4.01 (t, 0.5H), 3.86 (t, 0.5H), 3.72 (ddd, 1H), 3.65-3.42 (m, 3H), 3.38 (dd, 1H), 3.21 (dt, 2H), 3.11 (s, 1H), 2.99 (s, 3H), 2.80 (d, 9H), 2.70 (d, 3H), 2.60 (q, 5H), 2.13-1.94 (m, 3H), 1.75 (s, 1H), 1.57-1.46 (m, 5H), 1.38 (d, 3H), 1.36-1.25 (m, 10H), 1.02 (d, 6H), 0.96 (d, 3H).

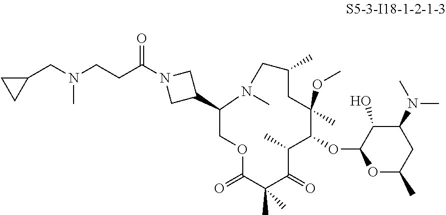

### (3R,6R,8R,9R,10R)-3-(1-(3-((cyclopropylmethyl)(methyl)amino)propanoyl)azetidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S5-3-I18-1-2-1-2) (Compound 144)

Prepared according to the methods of S5-3-I18-1-2-1-1, substituting cyclopropanecarboxaldehyde to provide 12.29 mg of the title compound as a formate salt. MS (ESI+) m/z: 232.65 [M+3H]³⁺, 348.41 [M+2H]²⁺, 695.35 [M+H]⁺; ¹H NMR (400 MHz, Methanol-d₄) δ 8.52 (s, 2.5H), 4.48 (d), 4.41 (t, 0.7H), 4.30 (q, 1.3H), 4.24-4.06 (m, 3.5H), 4.01 (t, 0.5H), 3.89 (t, 0.5H), 3.72 (ddt, 2H), 3.54-3.43 (m, 2H), 3.38 (dt, 3H), 3.14 (s, 1H), 2.99 (d, 5H), 2.84 (s, 3H), 2.81 (s, 7H), 2.69-2.47 (m, 5H), 2.10-1.94 (m, 2H), 1.73 (s, 1H), 1.57-1.46 (m, 5H), 1.38 (d, Hz, 3H), 1.37-1.27 (m, 9H), 1.14 (ddt, 1H), 0.98 (d, 3H), 0.83-0.65 (m, 2H), 0.49-0.34 (m, 2H).

The following examples were prepared according to the methods of S5-3-I18-1-2-1-1, substituting the appropriate carboxylic acid for 3-((tert-butoxycarbonyl)amino)propanoic acid, and the appropriate aldehyde or ketone for formaldehyde.

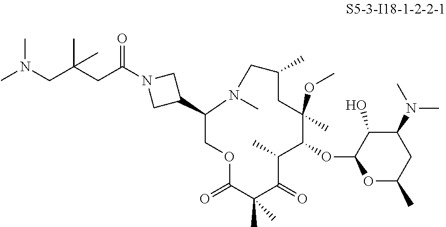

### (3R,6R,8R,9R,10R)-3-(1-(4-(dimethylamino)-3,3-dimethylbutanoyl)azetidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S5-3-I18-1-2-2-1) (Compound 183)

Prepared according to the methods of S5-3-I18-1-2-1-1, substituting 4-((tert-butoxycarbonyl)amino)-3,3-dimethylbutanoic acid and formaldehyde to provide 11.05 mg of the title compound as a formate salt. MS (ESI+) m/z: 233.35 [M+3H]³⁺, 349.44 [M+2H]²⁺, 697.42 [M+H]⁺; ¹H NMR (400 MHz, Methanol-d₄) δ 8.55 (s, 2H), 4.49 (d, 1H), 4.36 (dt, 1H), 4.21 (s, 1H), 4.11 (p, 4H), 3.98 (t, 0.5H), 3.85 (t, 1H), 3.76-3.65 (m, 1H), 3.55 (s, 1H), 3.50-3.37 (m, 1.5H), 3.35 (d, 0.5H), 3.05 (s, 3H), 2.95 (s, 3H), 2.85 (s, 6H), 2.77 (s, 6H), 2.47 (s, 3H), 2.39 (d, 2H), 2.04-1.96 (m, 1H), 1.89 (s, 2H), 1.58-1.43 (m, 4H), 1.37 (d, 3H), 1.34-1.22 (m, 10H), 1.14 (d, 6H), 1.00-0.81 (m, 3H).

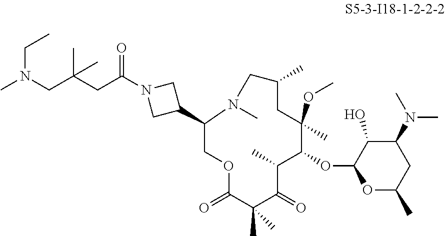

### (3R,6R,8R,9R,10R)-9-(((2S,3R,4S,6R)-4-(dimethylamino)-3-hydroxy-6-methyltetrahydro-2H-pyran-2-yl)oxy)-3-(1-(4-(ethyl(methyl)amino)-3,3-dimethylbutanoyl)azetidin-3-yl)-8-methoxy-4,6,8,10,12,12-hexamethyl-1-oxa-4-azacyclotridecane-11,13-dione (S5-3-I18-1-2-2-2) (Compound 122)

Prepared according to the methods of S5-3-I18-1-2-1-1, substituting 4-((tert-butoxycarbonyl)amino)-3,3-dimethylbutanoic acid and acetaldehyde to provide 11.05 mg of the title compound as a formate salt. MS (ESI+) m/z: 238.03 [M+3H]³⁺, 356.44 [M+2H]²⁺, 711.46 [M+H]⁺; ¹H NMR (400 MHz, Methanol-d₄) δ 8.52 (s, 2H), 4.50 (d, 1H), 4.44-4.30 (m, 1H), 4.29-4.03 (m, 5H), 3.99 (s, 0.5H), 3.85 (s, 0.5H), 3.78-3.66 (m, 1H), 3.55 (s, 1H), 3.48-3.36 (m, 2H), 3.18 (q, 2H), 3.06 (s, 3H), 2.96 (s, 3H), 2.86 (s, 3H), 2.78 (s, 6H), 2.44 (d, 5H), 2.05-1.97 (m, 1H), 1.89 (s, 2H), 1.54 (s, 3H), 1.53-1.45 (m, 1H), 1.37 (d, 3H), 1.35-1.24 (m, 12H), 1.16 (d, 6H), 0.92 (d, 3H).

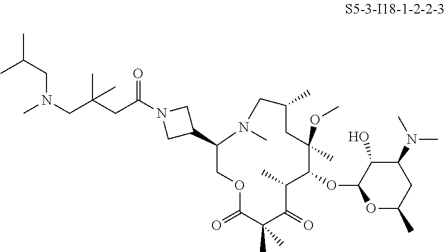

### (3R,6R,8R,9R,10R)-9-(((2S,3R,4S,6R)-4-(dimethylamino)-3-hydroxy-6-methyltetrahydro-2H-pyran-2-yl)oxy)-3-(1-(4-(isobutyl(methyl)amino)-3,3-dimethylbutanoyl)azetidin-3-yl)-8-methoxy-4,6,8,10,12,12-hexamethyl-1-oxa-4-azacyclotridecane-11,13-dione (S5-3-I18-1-2-2-3) (Compound 167)

Prepared according to the methods of S5-3-I18-1-2-1-1, substituting 4-((tert-butoxycarbonyl)amino)-3,3-dimethylbutanoic acid and isobutyraldehyde to provide 12.64 mg of the title compound as a formate salt. MS (ESI+) m/z: 247.39 [M+3H]³⁺, 370.50 [M+2H]²⁺, 739.48 [M+H]⁺; ¹H NMR (400 MHz, Methanol-d₄) δ 8.53 (s, 2H), 4.49 (d, 1H), 4.46-4.24 (m, 2H), 4.25-4.07 (m, 3.5H), 4.03 (s, 0.5H), 3.89 (s, 0.5H), 3.72 (dtt, 1H), 3.66-3.42 (m, 2.5H), 3.37 (ddd, 1H), 3.04 (s, 3H), 2.98 (s, 3H), 2.90 (d, 2H), 2.81 (s, 10H), 2.56 (s, 3H), 2.45 (d, 2H), 2.17-1.87 (m, 3H), 1.75 (s, 1H), 1.54 (s, 3H), 1.51-1.42 (m, 1H), 1.38 (d, 3H), 1.35-1.24 (m, 9H), 1.15 (s, 6H), 1.05 (d, 6H), 0.96 (d, 3H).

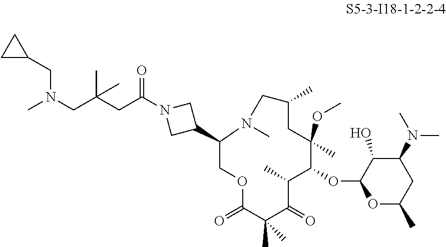

### (3R,6R,8R,9R,10R)-3-(1-(4-((cyclopropylmethyl)(methyl)amino)-3,3-dimethylbutanoyl)azetidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S5-3-I18-1-2-2-4) (Compound 25)

Prepared according to the methods of S5-3-I18-1-2-1-1, substituting 4-((tert-butoxycarbonyl)amino)-3,3-dimethylbutanoic acid and cyclopropanecarboxaldehyde to provide 13.38 mg of the title compound as a formate salt. MS (ESI+) m/z: 246.71 [M+3H]³⁺, 369.47 [M+2H]²⁺, 737.41 [M+H]⁺; ¹H NMR (400 MHz, Methanol-d₄) δ 8.52 (s, 2H), 4.49 (d, 1H), 4.45-4.26 (m, 2H), 4.25-4.10 (m, 3.5H), 4.05 (t, 0.5H), 3.92 (t, 1H), 3.72 (ddt, 1H), 3.63 (s, 0.5H), 3.54-3.43 (m, 2H), 3.38 (ddd, 1H), 3.14 (s, 3H), 3.06 (d, 2H), 2.99 (s, 3H), 2.96 (s, 3H), 2.81 (s, 7H), 2.59 (s, 3H), 2.47 (d, 2H), 2.03 (ddd, 2H), 1.77 (s, 1H), 1.60-1.44 (m, 5H), 1.38 (d, 3H), 1.37-1.27 (m, 9H), 1.17 (d, 7H), 0.97 (d, 3H), 0.81-0.67 (m, 2H), 0.51-0.40 (m, 2H).

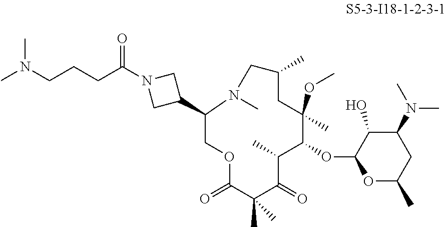

### (3R,6R,8R,9R,10R)-9-(((2S,3R,4S,6R)-4-(dimethylamino)-3-hydroxy-6-methyltetrahydro-2H-pyran-2-yl)oxy)-3-(1-(4-(dimethylamino)butanoyl)azetidin-3-yl)-8-methoxy-4,6,8,10,12,12-hexamethyl-1-oxa-4-azacyclotridecane-11,13-dione (S5-3-I18-1-2-3-1) (Compound 139)

Prepared according to the methods of S5-3-I18-1-2-1-1, substituting 4-((tert-butoxycarbonyl)amino)butanoic acid and formaldehyde to provide 3.61 mg of the title compound as a formate salt. MS (ESI+) m/z: 223.94 [M+3H]³⁺, 335.35 [M+2H]²⁺, 669.42 [M+H]⁺; ¹H NMR (400 MHz, Methanol-d₄) δ 8.52 (s, 2.4H), 4.49 (d, 1H), 4.41-4.23 (m, 1.5H), 4.23-4.01 (m, 4.5H), 3.96 (s, 0.5H), 3.82 (s, 0.5H), 3.77-3.66 (m, 1H), 3.53 (s, 1H), 3.45 (dd, 2H), 3.40-3.33 (m, 1H), 3.15-2.91 (m, 7H), 2.78 (s, 12H), 2.55 (s, 3H), 2.29 (q, 2H), 2.03-1.89 (m, 5H), 1.53 (s, 3H), 1.49 (q, 1H), 1.37 (s, 3H), 1.31 (dd, 9H), 0.94 (d, 3H).

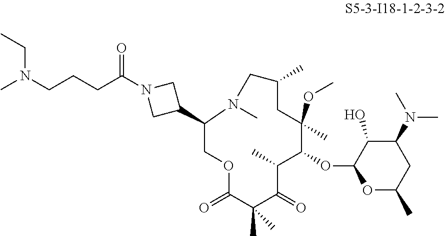

### (3R,6R,8R,9R,10R)-9-(((2S,3R,4S,6R)-4-(dimethylamino)-3-hydroxy-6-methyltetrahydro-2H-pyran-2-yl)oxy)-3-(1-(4-(ethyl(methyl)amino)butanoyl)azetidin-3-yl)-8-methoxy-4,6,8,10,12,12-hexamethyl-1-oxa-4-azacyclotridecane-11,13-dione (S5-3-I18-1-2-3-2) (Compound 135)

Prepared according to the methods of S5-3-I18-1-2-1-1, substituting 4-((tert-butoxycarbonyl)amino)butanoic acid and acetaldehyde to provide 9.565 mg of the title compound as a formate salt. MS (ESI+) m/z: 228.64 [M+3H]³⁺, 342.38 [M+2H]²⁺, 683.37 [M+H]⁺; ¹H NMR (400 MHz, Methanol-d₄) δ 8.55 (s, 2H), 4.49 (d, 1H), 4.31 (dt, 2H), 4.22-4.01 (m, 4H), 4.00-3.78 (m, 1H), 3.71 (ddt, 1H), 3.53 (s, 1H), 3.45 (dd, 1H), 3.39-3.32 (m, 1H), 3.20-3.00 (m, 5H), 2.96 (s, 3H), 2.78 (d, 10H), 2.50 (s, 3H), 2.31 (dt, 2H), 2.07-1.74 (m, 5H), 1.54 (s, 3H), 1.48 (q, 8.6 Hz, 1H), 1.39-1.35 (m, 3H), 1.31 (dd, 12H), 0.93 (d, 3H).

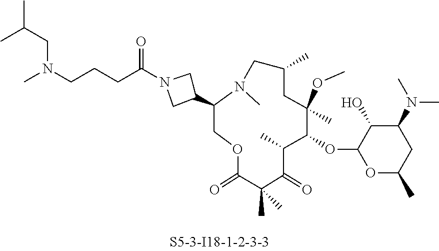

### (3R,6R,8R,9R,10R)-9-(((2S,3R,4S,6R)-4-(dimethylamino)-3-hydroxy-6-methyltetrahydro-2H-pyran-2-yl)oxy)-3-(1-(4-(isobutyl(methyl)amino)butanoyl)azetidin-3-yl)-8-methoxy-4,6,8,10,12,12-hexamethyl-1-oxa-4-azacyclotridecane-11,13-dione (S5-3-I18-1-2-3-3) (Compound 178)

Prepared according to the methods of S5-3-I18-1-2-1-1, substituting 4-((tert-butoxycarbonyl)amino)butanoic acid and isobutyraldehyde to provide 12.22 mg of the title compound as a formate salt. MS (ESI+) m/z: 237.98 [M+3H]³⁺, 356.41 [M+2H]²⁺, 711.35 [M+H]⁺; ¹H NMR (400 MHz, Methanol-d₄) δ 8.53 (s, 2.5H), 4.48 (d, 1H), 4.44-4.05 (m, 5H), 4.04-3.79 (m, 1H), 3.72 (ddt, 1H), 3.63 (s, 0.5H), 3.53-3.43 (m, 2H), 3.37 (ddd, 1H), 3.10 (t, 3H), 2.99 (s, 3H), 2.91 (d, 2H), 2.80 (d, 10H), 2.59 (s, 3H), 2.35 (q, 2H), 2.11 (dq, 1H), 2.07-1.91 (m, 4H), 1.73 (d, 1H), 1.54 (s, 5H), 1.38 (d, 3H), 1.36-1.25 (m, 9H), 1.04 (d, 6H), 0.97 (d, 3H).

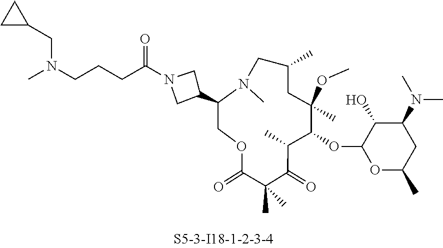

### (3R,6R,8R,9R,10R)-3-(1-(4-((cyclopropylmethyl)(methyl)amino)butanoyl)azetidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S5-3-I18-1-2-3-4) (Compound 61)

Prepared according to the methods of S5-3-I18-1-2-1-1, substituting 4-((tert-butoxycarbonyl)amino)butanoic acid and cyclopropanecarboxaldehyde to provide 13.35 mg of the title compound as a formate salt. MS (ESI+) m/z: 237.32 [M+3H]³⁺, 355.40 [M+2H]²⁺, 709.41 [M+H]⁺; ¹H NMR (400 MHz, Methanol-d₄) δ 8.53 (s, 2.5H), 4.49 (dd, 1H), 4.38 (t, 0.5H), 4.31-4.04 (m, 5H), 4.04-3.78 (m, 1H), 3.72 (ddt, 1H), 3.57 (s, 1H), 3.47 (dt, 2H), 3.42-3.33 (m, 1H), 3.23-3.04 (m, 3H), 3.02 (d, 2H), 2.99 (s, 3H), 2.89 (d, 3H), 2.80 (d, 7H), 2.71-2.46 (m, 3H), 2.37-2.28 (m, 2H), 2.10-1.91 (m, 4H), 1.74 (s, 1H), 1.57-1.46 (m, 4H), 1.38 (s, 3H), 1.36-1.27 (m, 9H), 1.19-1.08 (m, 1H), 0.97 (d, 3H), 0.82-0.69 (m, 2H), 0.48-0.38 (m, 2H).

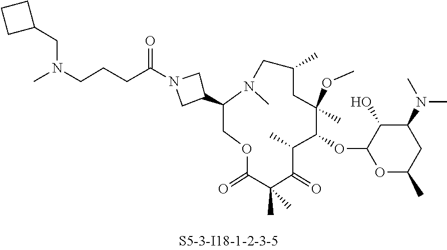

### (3R,6R,8R,9R,10R)-3-(1-(4-((cyclobutylmethyl)(methyl)amino)butanoyl)azetidin-3-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S5-3-I18-1-2-3-5) (Compound 156)

Prepared according to the methods of S5-3-I18-1-2-1-1, substituting 4-((tert-butoxycarbonyl)amino)butanoic acid and cyclobutanecarboxaldehyde to provide 10.12 mg of the title compound as a formate salt. MS (ESI+) m/z: 241.97 [M+3H]³⁺, 362.45 [M+2H]²⁺, 723.40 [M+H]⁺; ¹H NMR (400 MHz, Methanol-d₄) δ 8.54 (s, 2H), 4.49 (d, 1H), 4.38-4.24 (m, 1H), 4.10 (dd, 4H), 3.97 (s, 0.5H), 3.86-3.77 (m, 0.5H), 3.71 (ddt, 1H), 3.52 (s, 1H), 3.45 (dd, 2H), 3.41-3.33 (m, 1H), 3.11 (d, 2.5H), 3.04 (t, 3.5H), 2.97 (s, 3H), 2.78 (d, 7H), 2.75 (d, 4H), 2.55 (d, 3H), 2.31 (td, 2H), 2.22-2.14 (m, 2H), 2.08-1.80 (m, 9H), 1.55-1.45 (m, 4H), 1.39-1.35 (m, 3H), 1.31 (dd, 9H), 0.94 (d, 3H).

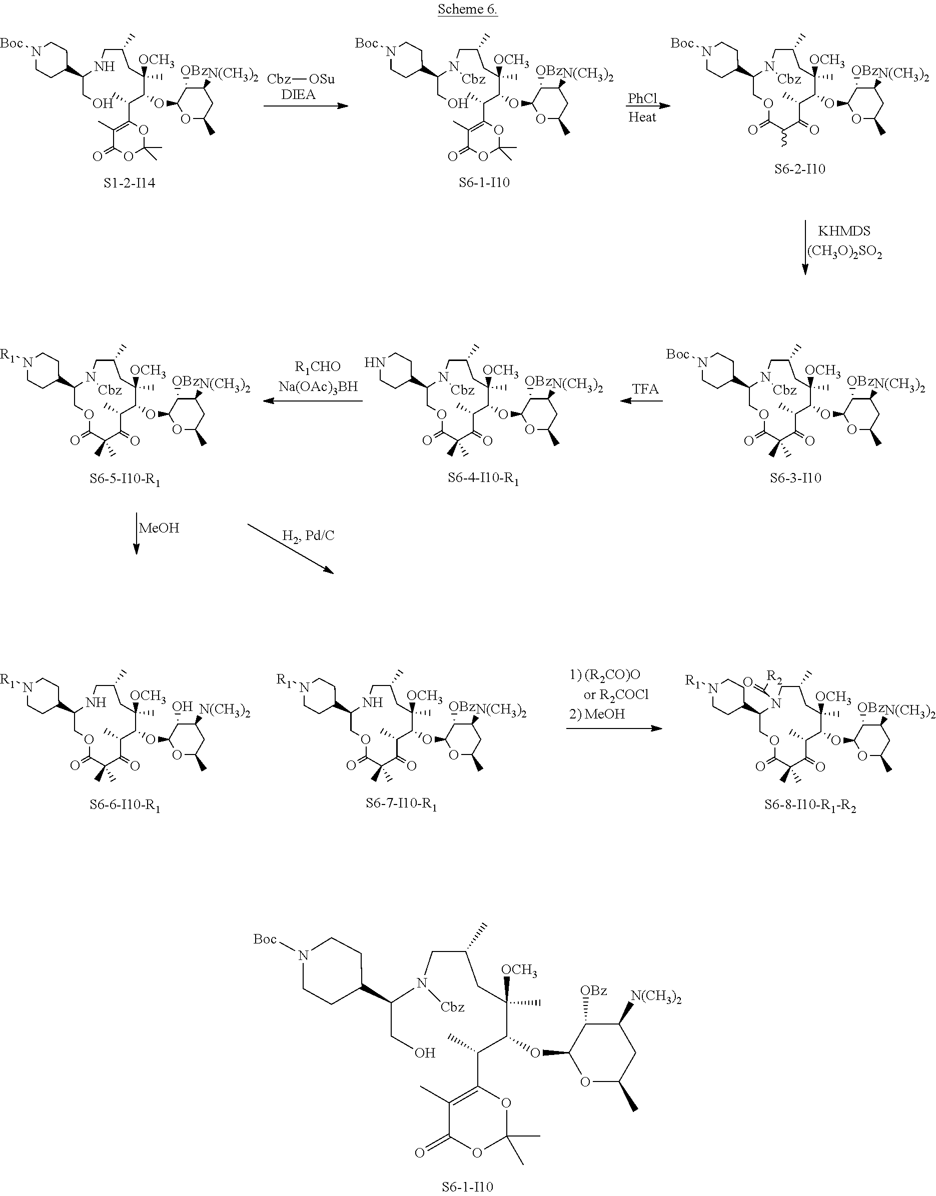

tert-Butyl 4-((R)-1-(((2R,4R,5R,6R)-5-(((2S,3R,4S,6R)-3-(benzoyloxy)-4-(dimethylamino)-6-methyltetrahydro-2H-pyran-2-yl)oxy)-4-methoxy-2,4-dimethyl-6-(2,2,5-trimethyl-4-oxo-4H-1,3-dioxin-6-yl)heptyl)((benzyloxy)carbonyl)amino)-2-hydroxyethyl)piperidine-1-carboxylate (S6-1-I10). In a 100 mL flask was a solution of S1-2-I10 (1.741 g, 2.12 mmol, prepared according to Scheme 1 using I10) in dichloromethane (10 mL) at 0° C. Diisopropylethylamine (0.41 mL, 2.33 mL) was added followed by N-(benzyloxycarbonyloxy)succinimide (553 mg, 2.22 mmol), and the mixture was stirred while the ice bath was allowed to warm to rt over 1 h. The mixture was stirred at rt for 3.5 h, then additional N-(benzyloxycarbonyloxy)succinimide (100 mg, 0.40 mmol) was added. The mixture was stirred for 1 h, then was diluted with dichloromethane and poured into satd aq NaHCO₃. The aqueous phase was extracted three times dichloromethane and the combined organic phases were dried over MgSO₄, filtered and concentrated. The residue was purified on 80 g of silica gel, elution with 0-15% MeOH-dichloromethane-0.5% NH₄OH to give the title compound (1.801 g white solid, 90%). MS (ESI+) m/z: 426.9 [M+2H]²⁺, 952.4 [M+H]⁺.

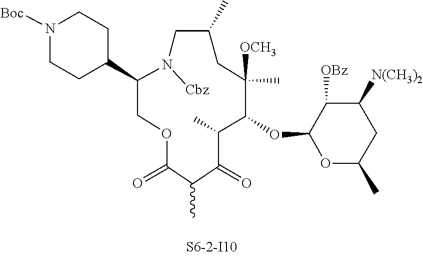

Benzyl (3R,6R,8R,9R,10R,12R)-9-(((2S,3R,4S,6R)-3-(benzoyloxy)-4-(dimethylamino)-6-methyltetrahydro-2H-pyran-2-yl)oxy)-3-(1-(tert-butoxycarbonyl)piperidin-4-yl)-8-methoxy-6,8,10,12-tetramethyl-11,13-dioxo-1-oxa-4-azacyclotridecane-4-carboxylate (S6-2-I10). In a 1 L flask was alcohol S6-1-I10 (1.80 g, 1.89 mmol) which was azeotroped twice from toluene and dried under vacuum. The residue was dissolved in chlorobenzene (600 mL), the atmosphere was purged with nitrogen and the solution was degassed by sonication under vacuum. The atmosphere was purged and backfilled with nitrogen twice, then the mixture was heated at gentle reflux (bath temperature 148° C.) for 23 h. The reaction mixture was concentrated and the residue was purified on 120 g of silica gel, elution with 0-15% MeOH-dichloromethane-0.5% NH₄OH to give the title compound (486 mg white solid, 29%). MS (ESI+) m/z: 894.0 [M+H]⁺.

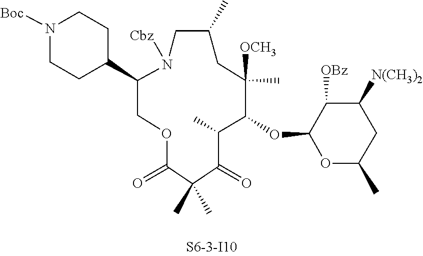

Benzyl (3R,6R,8R,9R,10R)-9-(((2S,3R,4S,6R)-3-(benzoyloxy)-4-(dimethylamino)-6-methyltetrahydro-2H-pyran-2-yl)oxy)-3-(1-(tert-butoxycarbonyl)piperidin-4-yl)-8-methoxy-6,8,10,12,12-pentamethyl-11,13-dioxo-1-oxa-4-azacyclotridecane-4-carboxylate (S6-3-I10) In a 25 mL flask was a solution of S6-2-I10 (486 mg, 0.54 mmol) in DME (5.5 mL) which was cooled at −58° C. in a dry ice-acetone bath. KHMDS solution (0.71 mL, 1.0 M in THF, 0.71 mmol) was added dropwise and the mixture was stirred at −58° C. for 30 minutes. Dimethyl sulfate (102 μmol, 1.08 mmol) was added dropwise and the resulting mixture was stirred and allowed to warm to −10° C. and held between −10 and −15° C. for 1 h. Triethylamine (224 μL, 1.62 mmol) was added and the flask was removed from the bath and stirred at rt for 1 h. The reaction mixture was diluted with dichloromethane and poured into satd aq NaHCO₃. The aqueous phase was extracted three times with dichloromethane and the combined organic phases were dried over Na₂SO₄, filtered and concentrated. The residue was purified on 20 g silica gel, elution with 0-8% MeOH-dichloromethane-0.5% NH₄OH to give the title compound (402 mg white solid, 82%). MS (ESI+) m/z: 908.1 [M+H]t.

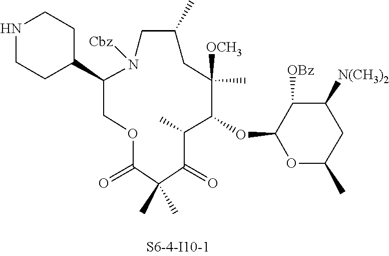

Benzyl (3R,6R,8R,9R,10R)-9-(((2S,3R,4S,6R)-3-(benzoyloxy)-4-(dimethylamino)-6-methyltetrahydro-2H-pyran-2-yl)oxy)-8-methoxy-6,8,10,12,12-pentamethyl-11,13-dioxo-3-(piperidin-4-yl)-1-oxa-4-azacyclotridecane-4-carboxylate (S6-2-I10-1). In a 20 mL vial was a solution of S6-3-I10 in dichloromethane (1.33 mL) and TFA (0.33 mL) was added, then the mixture was stirred at rt for 4 h. The reaction mixture was diluted with EtOAc and poured into satd aq NaHCO₃ and the aqueous phase was extracted twice with EtOAc. The combined organic phases were washed with brine, then dried over MgSO₄, filtered and concentrated. The residue was dissolved in dichloromethane (1.33 mL) and TFA (0.33 mL) was added, then the mixture was stirred at rt for 2 h. The reaction mixture was diluted with EtOAc and poured into satd aq NaHCO₃ and the aqueous phase was extracted twice with EtOAc. The combined organic phases were washed with brine, then dried over MgSO₄, filtered and concentrated to give the title compound (357 mg white solid). MS (ESI+) m/z: 404.7 [M+2H]²⁺, 808.0 [M+H]⁺.

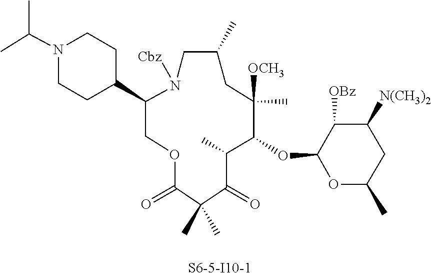

Benzyl (3R,6R,8R,9R,10R,12R)-9-(((2S,3R,4S,6R)-3-(benzoyloxy)-4-(dimethylamino)-6-methyltetrahydro-2H-pyran-2-yl)oxy)-3-(1-isopropylpiperidin-4-yl)-8-methoxy-6,8,10,12,12-pentamethyl-11,13-dioxo-1-oxa-4-azacyclotridecane-4-carboxylate (S6-5-I10-1). In a 20 mL vial was a solution of S6-4-I10 (357 mg, 0.44 μmmol) in dichloromethane (1.7 mL) which was stirred at rt. Acetone (161 μL, 2.2 mmol), acetic acid (25 μL, 0.44 mmol) and sodium triacetoxyborohydride (186 mg, 0.882 mmol) were added sequentially and the mixture was stirred at rt for 23 h. The reaction mixture was diluted with dichloromethane and poured into satd aq NaHCO₃ and the aqueous phase was extracted three times with dichloromethane. The combined organic phases were dried over MgSO₄, filtered and concentrated to give the title compound (296 mg white solid). MS (ESI+) m/z: 425.8 [M+2H]²⁺, 850.0 [M+H]⁺.

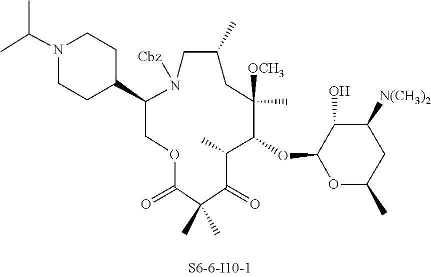

Benzyl (3R,6R,8R,9R,10R)-9-(((2S,3R,4S,6R)-4-(dimethylamino)-3-hydroxy-6-methyltetrahydro-2H-pyran-2-yl)oxy)-3-(1-isopropylpiperidin-4-yl)-8-methoxy-6,8,10,12,12-pentamethyl-11,13-dioxo-1-oxa-4-azacyclotridecane-4-carboxylate (S6-6-I10-1) (Compound 118). In a 5 mL flask was a solution of S6-5-I10-1 (24 mg, 28 μmol) in 0.5 mL of MeOH which was heated at 65° C. for 6 h, then the reaction mixture was concentrated. The residue was purified by HPLC (Atlantis T3 column, 5-50% MeCN-water-0.1% HCO₂H) to give 13.4 mg of the title compound as a formate salt. MS (ESI+) m/z: 373.8 [M+2H]²⁺, 746.3 [M+H]⁺. ¹H NMR (400 MHz, Methanol-d₄) rotomers, reported collectively δ 7.48-7.22 (m, 5H), 5.19 (q, 2H), 4.61-4.39 (m, 1H), 4.37 (d, 1H), 4.20 (dd, 1H), 3.93 (t, 2H), 3.66-3.58 (m, 1H), 3.58-3.44 (m, 2H), 3.38-3.29 (m, 1H), 3.24 (t, 1H), 3.16-3.04 (m, 1H), 3.04-3.01 (m, 1H), 2.94-2.80 (m, 2H), 2.69 (d, 3H), 2.47 (s, 3H), 2.44-2.33 (m, 5H), 2.04-1.93 (m, 2H), 1.93-1.74 (m, 4H), 1.69 (d, 1H), 1.52 (s, 1H), 1.46 (d, 1H), 1.42 (s, 2H), 1.33-1.28 (m, 3H), 1.28-1.19 (m, 11H), 1.16 (d, 3H), 1.13 (d, 5H), 0.98 (d, 1.5H), 0.92 (br s, 1.5H).

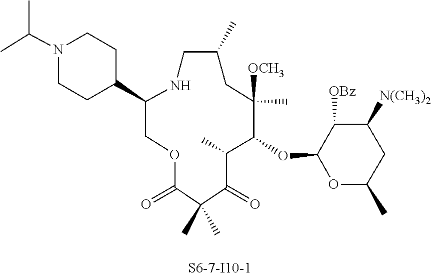

(2S,3R,4S,6R)-4-(Dimethylamino)-2-(((3R,6R,8R,9R,10R)-3-(1-isopropylpiperidin-4-yl)-8-methoxy-6,8,10,12,12-pentamethyl-11,13-dioxo-1-oxa-4-azacyclotridecan-9-yl)oxy)-6-methyltetrahydro-2H-pyran-3-yl benzoate (S6-7-I10-1). In a 20 mL flask was a solution of S6-5-I10-1 (296 mg, 0.348 mmol) in MeOH and aq HCl (3.0 M, 0.23 mL, 0.696 mmol) was added. The mixture was concentrated and dried under vacuum, then diluted in MeOH (1.5 mL). The resulting solution was degassed under vacuum and backfilled with nitrogen. Pd/C (5% Pd, 74 mg, 0.35 mmol) was added and the flask was purged with hydrogen five times then stirred vigorously under static hydrogen at rt for 4 h. The mixture was filtered through a plug of Celite® and concentrated, and the residue was purified on 12 g of silica gel, elution with 0-20% MeOH-dichloromethane-0.5% of 30% aq NH₄OH to give the 164 mg of the title compound. MS (ESI+) m/z: 239.5 [M+3H]³⁺, 358.7 [M+2H]²⁺, 716.1 [M+H]⁺.

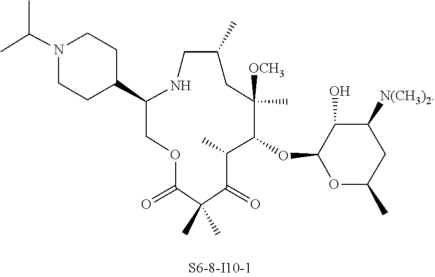

### (3R,6R,8R,9R,10R)-9-(((2S,3R,4S,6R)-4-(Dimethylamino)-3-hydroxy-6-methyltetrahydro-2H-pyran-2-yl)oxy)-3-(1-isopropylpiperidin-4-yl)-8-methoxy-6,8,10,12,12-pentamethyl-1-oxa-4-azacyclotridecane-11,13-dione (S6-3-I10-1-2) (Compound 7)

Prepared from S6-7-I10-1 according to the method of S6-6-I10-1 to give the title compound as a formate salt. MS (ESI+) m/z: 204.5 [M+3H]³⁺, 306.9 [M+2H]²⁺, 612.4 [M+H]⁺. ¹H NMR (400 MHz, Methanol-d₄) δ 8.50 (s, 2H), 4.45 (d, 1H), 4.12 (d, 1H), 3.99 (dd, 1H), 3.78-3.67 (m, 1H), 3.56-3.40 (m, 5H), 3.40-3.33 (m, 1H), 3.04-2.95 (m, 2H), 2.94 (s, 3H), 2.82 (d, 1H), 2.80 (s, 6H), 2.75-2.67 (m, 1H), 2.48-2.33 (m, 1H), 2.27 (d, 1H), 2.11-1.91 (m, 3H), 1.89-1.79 (m, 2H), 1.78-1.61 (m, 3H), 1.61-1.50 (m, 2H), 1.49 (s, 3H), 1.48-1.40 (m, 1H), 1.38 (s, 3H), 1.35 (s, 5H), 1.33 (d, 6H), 1.32 (s, 4H), 1.30 (s, 1H), 0.99 (d, 3H).

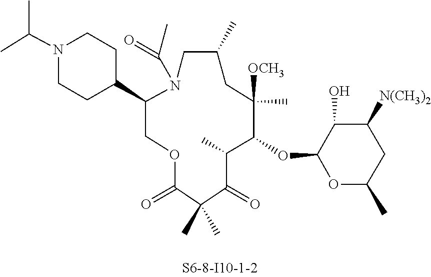

(3R,6R,8R,9R,10R)-4-Acetyl-9-(((2S,3R,4S,6R)-4-(dimethylamino)-3-hydroxy-6-methyltetrahydro-2H-pyran-2-yl)oxy)-3-(1-isopropylpiperidin-4-yl)-8-methoxy-6,8,10,12,12-pentamethyl-1-oxa-4-azacyclotridecane-11,13-dione (S6-8-I10-1-2) (Compound 174). In a 5 mL flask was a solution of S6-7-I10-1 (40 mg, 0.056 mmol) in dichloromethane (0.2 mL) and diisopropylethylamine (0.019 mL, 0.111 mmol) was added followed by acetic anhydride (0.010 mL, 0.111 mmol). The resulting mixture was stirred at rt for 1.5 h, then the reaction mixture was diluted with dichloromethane and poured into saturated aqueous sodium bicarbonate. The aqueous phase was extracted three times with dichloromethane and the combined organic phases were dried over magnesium sulfate. The residue was purified on 4 g of silica gel, elution with 0-20% MeOH-dichloromethane-0.5% of 30% aq NH₄OH, to give the intermediate acetamide as a white solid. (34 mg, 81%) MS (ESI+) m/z: 379.8 [M+2H]²⁺, 758.1 [M+H]⁺ Benzoate removal as described for S6-6-I10-1 gave the title compound as a formate salt. MS (ESI+) m/z: 328.0 [M+2H]²⁺, 654.4 [M+H]⁺. ¹H NMR (400 MHz, Methanol-d₄) rotomers, reported collectively a 8.55 (s, 2H), 4.47-4.39 (m, 1H), 4.32-4.23 (m, 1H), 4.13-3.91 (m, 3H), 3.87 (t, 1H), 3.69 (dd, 1H), 3.65-3.52 (m, 1H), 3.47-3.32 (m, 4H), 3.22 (s, 1H), 3.00-2.81 (m, 3H), 2.77 (s, 3H), 2.75 (d, 1H), 2.70 (d, 3H), 2.69 (s, 2H), 2.36 (s, 2H), 2.22 (s, 1H), 2.12-1.91 (m, 5H), 1.81 (d, 2H), 1.64 (s, 2H), 1.60 (s, 1H), 1.50 (q, 2H), 1.38-1.25 (m, 16H), 1.24 (d, 3H), 1.17-1.06 (m, 1H), 1.04 (d, 1H), 0.99 (d, 2H).

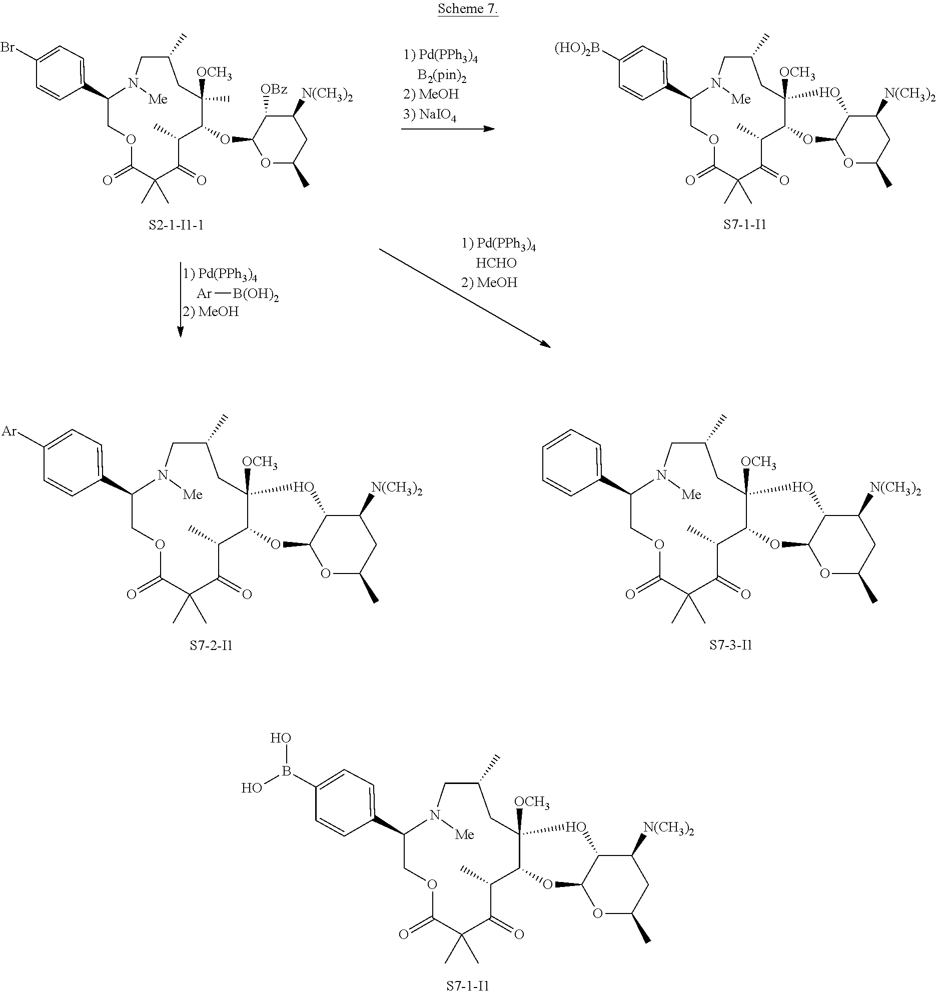

(4-((3R,6R,8R,9R,10R)-9-(((2S,3R,4S,6R)-4-(Dimethylamino)-3-hydroxy-6-methyltetrahydro-2H-pyran-2-yl)oxy)-8-methoxy-4,6,8,10,12,12-hexamethyl-11,13-dioxo-1-oxa-4-azacyclotridecan-3-yl)phenyl)boronic acid (S7-1-I1) (Compound 133). S2-1-I1-1 (27 mg, 0.036 mmol) and Bis(pinacolato)diboron (B₂pin₂) (13.6 mg, 0.054 mmol) were dissolved in DMSO (0.5 mL). Pd(PPh₃)₄ (7.6 mg, 0.006 mmol) was added. The reaction mixture was degassed and allowed to stir at rt for 10 min. Then the reaction mixture was heated at 80° C. for 3 h. Then it was cooled and diluted with dichloromethane and aqueous NaHCO₃ (10 mL) was added. The aqueous layer was extracted with dichloromethane and the combined organic layers were dried over MgSO₄, filtered and concentrated. MS (ESI+) m/z: 404.3 [M+2H]²⁺, 807.5 [M+H]⁺. The crude material (25 mg, 0.031 mmol) was dissolved in MeOH (0.5 mL) and heated at 60° C. until LC/MS indicated complete consumption of starting material (16 hours). The reaction mixture was filtered through a syringe filter with the aid of methanol and concentrated. The residue was dissolved in THF/H₂O (0.8 mL/0.2 mL), and NaIO₄ (22.6 mg, 0.11 mmol) was added at rt. The reaction mixture was stirred at rt for 20 min. HCl (1M, 0.11 mL, 0.11 mmol) was added. The reaction mixture was stirred at rt for 16 h. The residue was purified by HPLC (MeCN-water-0.1% HCO₂H) to yield 8.42 mg of the title compound as a formate salt. MS (ESI+) m/z: 207.5 [M+3H]³⁺, 310.7 [M+2H]²⁺, 620.4 [M+H]⁺; ¹H NMR (400 MHz, Methanol-d₄) δ 8.54 (s, 2H), 7.70 (d, 2H), 7.36 (s, 2H). 4.75-4.60 (m, 1H), 4.47 (d, 1H), 4.21 (s, 1H), 3.76-3.64 (m, 1H), 3.46-3.27 (m, 2H), 3.17 (s, 1H), 3.06 (s, 3H), 2.67 (s, 6H), 1.96 (dt, 1H), 1.48 (d, 1H), 1.43 (s, 3H), 1.41-1.26 (m, 10H), 1.10-0.79 (m, 4H).

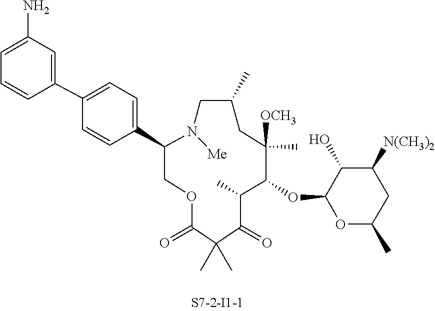 [SMILES: CO[C@]1(C)C[C@@H](C)CN(C)[C@H](c2ccc(-c3cccc(N)c3)cc2)COC(=O)C(C)(C)C(=O)[C@H](C)[C@H]1O[C@@H]1O[C@H](C)C[C@H](N(C)C)[C@H]1O]

(3R,6R,8R,9R,10R)-3-(3′-Amino-[1,1′-biphenyl]-4-yl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S7-2-I1-1) (Compound 93). S2-1-I1-1 (25 mg, 0.033 mmol) and (3-aminophenyl)boronic acid (7.5 mg, 0.049 mmol) were suspended in toluene (0.66 mL) and NaCO₃ solution (2M, 0.33 mL). Pd(PPh₃)₄ (7.6 mg, 0.006 mmol) was added. The reaction mixture was degassed and allowed to stir at rt for 10 min and was then heated at 80° C. for 16 h. The reaction mixture was cooled and diluted with dichloromethane, and aqueous NaHCO₃ (10 mL) was added. The aqueous layer was extracted with dichloromethane and the combined organic layers were dried over MgSO₄, filtered and concentrated. The residue was purified on 4 g of silica gel (elution with 0-10% MeOH-dichloromethane+0.5% of 30% aq NH₄OH) to give a white solid (20 mg, 79%). MS (ESI+) m/z: 258.1 [M+3H]³⁺, 386.7 [M+2H]²⁺, 772.4 [M+H]⁺. The material (20 mg, 0.026 mmol) was dissolved in MeOH (0.5 mL) and heated at 60° C. until LC/MS indicated complete consumption of starting material (16 hours). The reaction mixture was filtered through a syringe filter with the aid of methanol and concentrated. The residue was purified by HPLC (MeCN-water-0.1% HCO₂H) to yield 6.77 mg of the title compound as a formate salt. MS (ESI+) m/z: 233.5 [M+3H]³⁺, 334.7 [M+2H]²⁺, 668.4 [M+H]⁺; ¹H NMR (400 MHz, Methanol-d₄) δ 8.56 (d, 1H), 7.59 (s, 2H), 7.17 (t, 1H), 7.03-6.91 (m, 2H), 6.72 (dd, 1H), 4.54 (s, 1H), 4.45 (d, 1H), 4.34 (s, 1H), 4.12 (d, 1H), 3.81 (s, 1H), 3.64 (dt, 1H), 3.39-3.28 (m, 2H), 2.92 (d, 2H), 2.49 (s, 5H), 2.34 (s, 2H), 2.15 (d, 3H), 1.84 (d, 1H), 1.41 (s, 3H), 1.31 (dd, 10H), 0.87-0.80 (m, 2H).

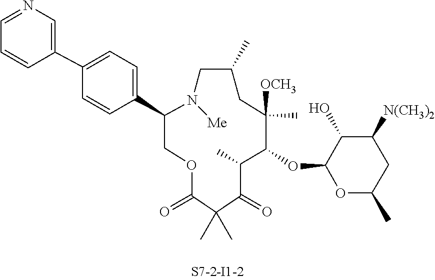 [SMILES: CO[C@]1(C)C[C@@H](C)CN(C)[C@H](c2ccc(-c3cccnc3)cc2)COC(=O)C(C)(C)C(=O)[C@H](C)[C@H]1O[C@@H]1O[C@H](C)C[C@H](N(C)C)[C@H]1O]

### (3R,6R,8R,9R,10R)-9-(((2S,3R,4S,6R)-4-(Dimethylamino)-3-hydroxy-6-methyltetrahydro-2H-pyran-2-yl)oxy)-8-methoxy-4,6,8,10,12,12-hexamethyl-3-(4-(pyridin-3-yl)phenyl)-1-oxa-4-azacyclotridecane-11,13-dione (S7-2-I1-2) (Compound 189)

Prepared according to the methods of S7-2-I1-1, substituting pyridin-3-ylboronic acid to provide 2.47 mg of the title compound as a formate salt. MS (ESI+) m/z: 218.8 [M+3H]³⁺, 327.7 [M+2H]²⁺, 654.4 [M+H]⁺; ¹H NMR (400 MHz, Methanol-d₄) δ 8.83 (d, 1H), 8.54 (d, 2H), 8.13 (dt, 1H), 7.72 (s, 2H), 7.54 (dd, 1H), 4.59 (s, 1H), 4.50 (d, 1H), 4.16 (d, 1H), 3.81 (s, 1H), 3.69 (dt, 1H), 3.41 (t, 1H), 3.32 (h, 2H), 3.17 (s, 1H), 2.96 (s, 1H), 2.67 (s, 5H), 2.37 (s, 1H), 2.28-2.05 (m, 2H), 1.94 (d, 1H), 1.42 (s, 3H), 1.32 (dt, 8H), 0.87 (s, 3H).

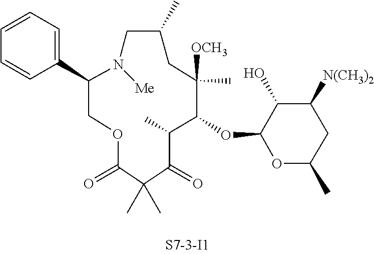 [SMILES: CO[C@]1(C)C[C@@H](C)CN(C)[C@H](c2ccccc2)COC(=O)C(C)(C)C(=O)[C@H](C)[C@H]1O[C@@H]1O[C@H](C)C[C@H](N(C)C)[C@H]1O]

### (3R,6R,8R,9R,10R)-9-(((2S,3R,4S,6R)-4-(Dimethylamino)-3-hydroxy-6-methyltetrahydro-2H-pyran-2-yl)oxy)-8-methoxy-4,6,8,10,12,12-hexamethyl-3-phenyl-1-oxa-4-azacyclotridecane-11,13-dione (S7-3-I1) (Compound 42)

S2-1-I1-1 (25 mg, 0.033 mmol), Cs₂CO₃ (32 mg, 0.099 mmol) and HCHO (48% in H₂O, 3 μL, 0.049 mmol) were dissolved in DMSO (0.5 mL). Pd(PPh₃)₄ (7.6 mg, 0.006 mmol) was added. The reaction mixture was degassed and allowed to stir at rt for 10 min. The reaction mixture was heated at 80° C. for 5 h, at which point it was cooled, was diluted with dichloromethane, and aqueous NaHCO₃ (10 mL) was added. The aqueous layer was extracted with dichloromethane and the combined organic layers were dried over MgSO₄, filtered and concentrated. The residue was purified on 4 g of silica gel (elution with 0-10% MeOH-dichloromethane+0.5% of 30% aq NH₄OH) to give a white solid (20 mg, 79%). MS (ESI+) m/z: 341.2 [M+2H]²⁺, 681.4 [M+H]⁺. The material (20 mg, 0.026 mmol) was dissolved in MeOH (0.5 mL) and the reaction mixture was heated at 60° C. until LC/MS indicated complete consumption of starting material (16 hours). The reaction mixture was filtered through a syringe filter with the aid of methanol and concentrated. The residue was purified by HPLC (MeCN-water-0.1% HCO₂H) to yield 6.07 mg of the title compound as a formate salt. MS (ESI+) m/z: 233.5 [M+3H]³⁺, 334.7 [M+2H]²⁺, 668.4 [M+H]⁺; ¹H NMR (400 MHz, Methanol-d₄) δ 8.56 (s, 1H), 7.72-7.12 (m, 5H), 4.49 (t, 2H), 4.21 (d, 2H), 3.86-3.58 (m, 2H), 3.45-3.24 (m, 2H), 3.14-2.82 (m, 4H), 2.59 (s, 6H), 2.33 (s, 3H), 2.25-1.98 (m, 2H), 1.89 (t, 2H), 1.50-1.16 (m, 14H), 0.84 (d, 3H).

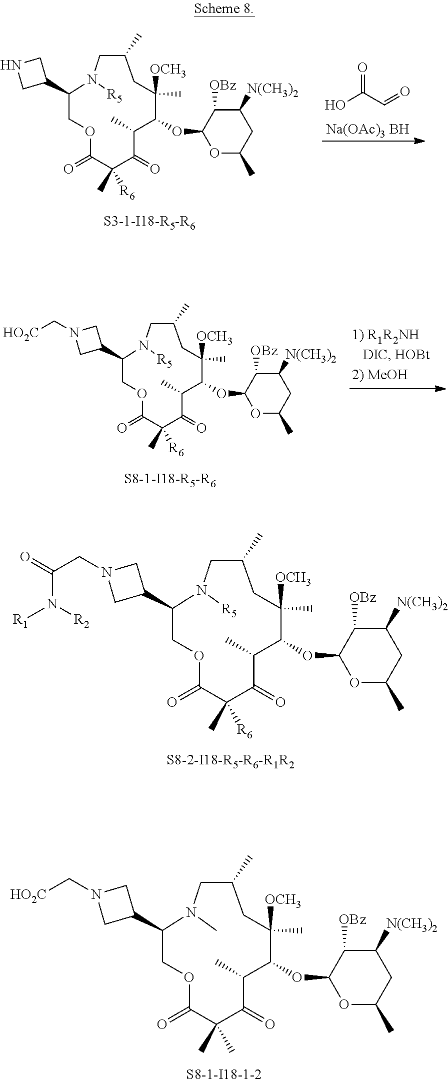

### 2-(3-((3R,6R,8R,9R,10R)-9-(((2S,3R,4S,6R)-3-(benzoyloxy)-4-(dimethylamino)-6-methyltetrahydro-2H-pyran-2-yl)oxy)-8-methoxy-4,6,8,10,12,12-hexamethyl-11,13-dioxo-1-oxa-4-azacyclotridecan-3-yl)azetidin-1-yl)acetic acid (S8-18-I1-1-2)

S3-1-I18-1-2 (152 mg, 0.23 mmol) and glyoxylic acid monohydrate (63.5 mg, 0.690 mmol) were dissolved in dichloromethane (3 mL), and Na(OAc)₃BH (146 mg, 0.690 mmol) and acetic acid (0.0394 mL, 0.690 mmol) were added. After stirring overnight, the reaction mixture was quenched with the slow addition of NaHCO₃ (sat, aq) until pH 7-8 was achieved. The organic layer was separated, and the aqueous layer was extracted with EtOAc (4 times). The combined extracts were dried over Na₂SO₄, were filtered, and were concentrated under reduced pressure to give the crude title compound (155.8 mg, 94%) as a white solid. The material was used without further purification.

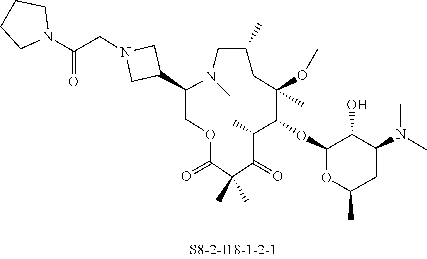

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(1-(2-oxo-2-(pyrrolidin-1-yl)ethyl)azetidin-3-yl)-1-oxa-4-azacyclotridecane-11,13-dione (S8-2-I18-1-2-1) (Compound 45). Crude S8-1-I18-1-2 (37 mg, 0.0515 mmol) was dissolved in dry EtOAc (0.6 mL) and N,N-diisopropylethylamine (17.9 μL, 0.103 mmol). Pyrrolidine (5.06 μL, 0.0618 mmol) was added followed by propylphosphonic anhydride (60 μL; 50% w/w soln in EtOAc), and the reaction mixture was stirred at room temperature overnight and then at 40° C. for ˜4 h. The reaction mixture was quenched with NaHCO₃ (sat., aq.), was extracted with EtOAc (1 mL×3), was dried over Na₂SO₄, was filtered, and was concentrated. The crude material was dissolved in MeOH (1 mL), and the reaction mixture was heated to 40° C. for ˜21 h. The reaction mixture was concentrated. The residue was dissolved in 0.1% aqueous formic acid (0.2 mL) and MeCN (0.3 mL) and was purified by HPLC (MeCN-water-0.1% HCO₂H) to give the title compound as a formate salt. MS (ESI+) m/z: 667.35 [M+H]⁺, ¹H NMR (400 MHz, Methanol-d₄) δ 8.51 (s, 2H), 4.75-4.62 (m, 1H), 4.51-4.43 (m, 2H), 4.33-3.97 (m, 3H), 3.94-3.56 (m, 6H), 3.56-3.35 (m, 8H), 3.26-3.17 (m, 1H), 3.11-2.86 (m, 6H), 2.82-2.68 (m, 9H), 2.07-1.93 (m, 3H), 1.88 (p, 2H), 1.49 (d, 5H), 1.42-1.28 (m, 12H), 1.28-1.19 (m, 1H), 1.07-0.85 (m, 3H).

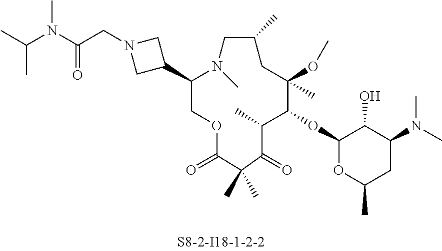

### 2-(3-((3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-11,13-dioxo-1-oxa-4-azacyclotridecan-3-yl)azetidin-1-yl)-N-isopropyl-N-methylacetamide (S8-2-I18-1-2-2) (Compound 192)

Prepared according to the methods of S8-2-I18-1-2-1 and N-methylisopropylamine to give the title compound as a formate salt. MS (ESI+) m/z: 669.40 [M+H]⁺; ¹H NMR (400 MHz, Methanol-d4) δ 8.49 (s, 2H), 4.76-4.60 (m, 1H), 4.46 (d, 1H), 4.36-4.18 (m, 1H), 4.15-3.85 (m, 5H), 3.82-3.68 (m, 3H), 3.65-3.57 (m, 1H), 3.52-3.33 (m, 4H), 3.10-2.95 (m, 4H), 2.87-2.74 (m, 10H), 2.73-2.56 (m, 2H), 2.45 (s, 1H), 2.01 (d, 2H), 1.69 (t, 1H), 1.49 (d, 4H), 1.43-1.17 (m, 16H), 1.16-1.06 (m, 4H), 1.04-0.89 (m, 3H).

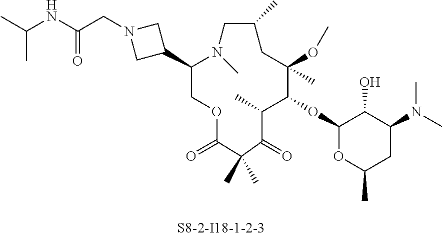

### 2-(3-((3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-11,13-dioxo-1-oxa-4-azacyclotridecan-3-yl)azetidin-1-yl)-N-isopropylacetamide (S8-2-I18-1-2-3) (Compound 36)

Prepared according to the methods of S8-2-I18-1-2-1 and isopropylamine to give the title compound as a formate salt. MS (ESI+) m/z: 655.40 [M+H]⁺; ¹H NMR (400 MHz, Methanol-d4) δ 8.47 (s, 3H), 4.46 (d, 1H), 4.34-4.18 (m, 1H), 4.11 (dd, 1H), 4.05-3.91 (m, 2H), 3.84-3.68 (m, 3H), 3.68-3.59 (m, 1H), 3.52-3.34 (m, 5H), 3.21-3.12 (m, 2H), 3.06 (d, 3H), 3.03-2.91 (m, 2H), 2.86-2.73 (m, 9H), 2.10-1.96 (m, 2H), 1.73 (d, 1H), 1.49 (d, 5H), 1.40 (d, 4H), 1.33 (q, 8H), 1.28-1.19 (m, 3H), 1.14 (dd, 6H), 1.03 (d, 2H), 0.93 (d, 1H).

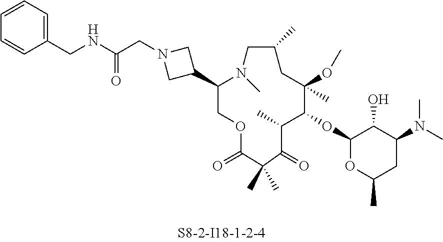

### N-benzyl-2-(3-((3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-11,13-dioxo-1-oxa-4-azacyclotridecan-3-yl)azetidin-1-yl)acetamide (S8-2-I18-1-2-4) (Compound 20)

Prepared according to the methods of S8-2-I18-1-2-1 and benzylamine to give the title compound as a formate salt. MS (ESI+) m/z: 703.41 [M+H]⁺; ¹H NMR (400 MHz, Methanol-d4) δ 8.53 (s, 1H), 7.40-7.14 (m, 5H), 4.62-4.42 (m, 1H), 4.38 (s, 2H), 4.28-4.02 (m, 2H), 4.00-3.84 (m, 1H), 3.78-3.56 (m, 4H), 3.49-3.34 (m, 3H), 3.27-3.16 (m, 4H), 3.15-3.09 (m, 1H), 3.07-2.86 (m, 5H), 2.81-2.66 (m, 8H), 1.98 (d, 2H), 1.48 (d, 5H), 1.42-1.18 (m, 15H), 1.09-0.86 (m, 4H).

The following compounds were prepared using synthetic procedures analogous to those described above for the preparation of S1-5-I1-1 in Scheme 1 employing the indicated amino alcohol. The syntheses were completed by deprotection of the benzoyl group as described above.

| Compound | Amino alcohol | Characterization |
|---|---|---|
| 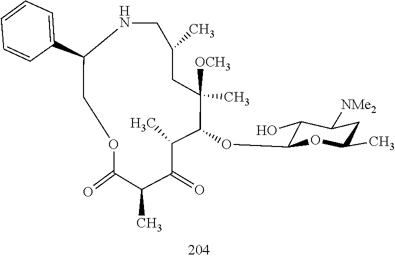 | 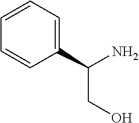 | ¹H NMR (600 MHz, Methanol-d₄) δ 8.48 (s, 1H), 7.51 − 7.38 (m, 5H), 4.75 (ddd, 1H), 4.43 (d, 1H), 4.37 (dd, 1H), 4.31 (d, 1H), 4.25 (ddd, 1H), 3.77 − 3.69 (m, 1H), 3.48 (ddt, 2H), 3.40 (ddd, 1H), 2.96 (s, 2H), 2.85 (dd, 1H), 2.81 (s, 5H), 2.37 (t, 1H), 2.03 (ddd, 1H), 1.86 (ddp, 1H), 1.70 (d, 2H), 1.63 − 1.46 (m, 2H), 1.37 (s, 3H), 1.33 (dd, 5H), 1.28 (s, 2H), 0.97 (d, 3H). |
| 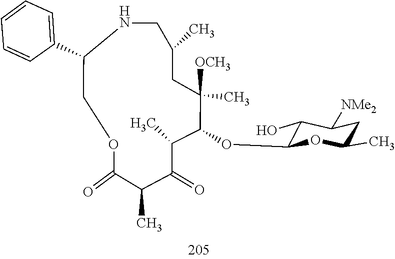 | 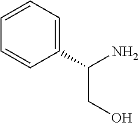 | ¹H NMR (600 MHz, Methanol-d₄) δ 8.40 (s, 2H), 7.51 − 7.43 (m, 5H), 4.61 (td, 3H), 4.45 (d, 1H), 4.18 (d, 1H), 3.78 − 3.70 (m, 1H), 3.61 (dq, 1H), 3.47 (dd, 1H), 3.42 (ddd, 1H), 3.34 (s, 2H), 2.98 − 2.95 (m, 2H), 2.83 (s, 5H), 2.69 (dd, 1H), 2.60 (dd, 1H), 2.07 − 1.99 (m, 2H), 1.83 (dd, 1H), 1.57 − 1.49 (m, 2H), 1.37 (s, 3H), 1.33 (d, 3H), 1.31 − 1.27 (m, 5H), 0.98 − 0.91 (m, 3H). |

### Biological Activity

Minimum inhibitory concentrations (MICs) for macrolides described herein have been determined for the following strains using similar test procedures as published in US Pat. Pub. No. 2017/0305953

| | |
|---|---|
| S. aureus | MP-12 |
| E. coli | MP-4 |
| K. pneumoniae | MP-546 |
| K. pneumoniae | MP-648 |
| P. aeruginosa | MP-3 |
| A. baumannii | MP-15 |

Several exemplary macrolides demonstrated potent activity against these Gram negative strains, including multidrug-resistant strains. CLSI standard procedures for broth dilution MIC determination were used. MIC data is represented as “+++” for values less than or equal to 4 mg/L, “++” for values of greater than 4 mg/L and less than or equal to 32 mg/L, and “+” for values greater than 32 mg/L. “−−” indicates the compound was not tested for a particular strain. Data is provided in Table B.

TABLE B

| | S. | E. | K. | K. | P | A. |
|---|---|---|---|---|---|---|
| Cmpd | aureus | coli | pneumoniae | pneumoniae | aeruginosa | baumanii |
| # | MP-12 | MP-4 | MP-546 | MP-648 | MP-3 | MP-15 |
| 1 | + | + | + | −− | + | + |
| 2 | + | ++ | + | −− | + | + |
| 4 | + | + | + | −− | + | + |
| 5 | + | +++ | ++ | −− | + | + |
| 6 | + | ++ | ++ | −− | + | + |
| 7 | ++ | +++ | +++ | −− | + | + |
| 8 | + | +++ | ++ | −− | + | + |
| 9 | + | ++ | ++ | −− | + | + |
| 10 | ++ | +++ | ++ | −− | + | ++ |
| 11 | ++ | +++ | +++ | −− | + | + |
| 12 | ++ | +++ | +++ | −− | + | + |
| 13 | ++ | ++ | ++ | −− | + | + |
| 14 | + | +++ | + | −− | + | + |
| 15 | ++ | +++ | ++ | −− | + | + |
| 16 | ++ | +++ | ++ | ++ | + | + |
| 17 | ++ | +++ | +++ | −− | + | ++ |
| 18 | + | +++ | ++ | ++ | + | + |
| 19 | + | + | + | −− | + | + |
| 20 | ++ | +++ | ++ | −− | + | + |
| 21 | + | + | + | −− | + | + |
| 22 | ++ | ++ | + | + | + | + |
| 23 | + | ++ | ++ | −− | + | + |
| 24 | ++ | +++ | +++ | +++ | + | + |
| 25 | ++ | +++ | +++ | ++ | + | + |
| 26 | + | ++ | + | −− | + | + |
| 27 | + | + | + | −− | + | + |
| 28 | ++ | +++ | +++ | −− | + | + |
| 29 | + | +++ | ++ | −− | + | + |
| 30 | ++ | +++ | +++ | +++ | + | + |
| 31 | + | +++ | +++ | ++ | + | + |
| 32 | + | +++ | ++ | ++ | + | + |
| 33 | + | ++ | + | −− | + | + |
| 34 | + | ++ | + | −− | + | + |
| 35 | + | +++ | ++ | −− | + | + |
| 36 | + | ++ | ++ | −− | + | + |
| 37 | ++ | +++ | +++ | ++ | + | + |
| 38 | ++ | +++ | ++ | −− | + | + |
| 39 | ++ | ++ | ++ | −− | + | + |
| 40 | ++ | +++ | +++ | −− | + | + |
| 41 | ++ | +++ | +++ | ++ | + | + |
| 42 | + | ++ | + | −− | + | + |
| 43 | ++ | +++ | +++ | −− | + | + |
| 44 | + | ++ | + | −− | + | + |
| 45 | ++ | +++ | ++ | −− | + | + |
| 46 | + | +++ | ++ | −− | + | + |
| 47 | + | ++ | + | −− | + | + |
| 48 | ++ | +++ | +++ | −− | + | + |
| 49 | ++ | +++ | +++ | ++ | + | + |
| 50 | ++ | ++ | ++ | ++ | + | + |
| 51 | ++ | +++ | +++ | ++ | + | ++ |
| 52 | + | +++ | ++ | −− | + | + |
| 53 | ++ | +++ | +++ | −− | + | + |
| 55 | ++ | ++ | ++ | + | + | + |
| 56 | + | + | + | −− | + | + |
| 57 | ++ | +++ | ++ | −− | + | + |
| 60 | ++ | +++ | ++ | ++ | + | + |
| 61 | + | ++ | ++ | + | + | + |
| 62 | ++ | +++ | ++ | −− | + | + |
| 63 | ++ | +++ | +++ | −− | + | + |
| 64 | ++ | +++ | +++ | −− | + | + |
| 65 | + | ++ | + | −− | + | + |
| 66 | + | +++ | ++ | −− | + | + |
| 67 | + | +++ | ++ | −− | + | + |
| 68 | ++ | +++ | ++ | −− | + | + |
| 69 | ++ | +++ | +++ | ++ | + | + |
| 70 | ++ | +++ | ++ | −− | + | + |
| 71 | ++ | ++ | +++ | +++ | + | + |
| 73 | ++ | ++ | +++ | −− | + | ++ |
| 74 | + | +++ | ++ | −− | + | + |
| 76 | ++ | +++ | +++ | −− | + | + |
| 77 | + | ++ | ++ | −− | + | + |
| 79 | +++ | +++ | ++ | −− | + | + |
| 80 | ++ | +++ | ++ | −− | + | + |
| 81 | + | +++ | ++ | −− | + | + |
| 82 | + | + | + | −− | + | + |
| 85 | + | +++ | ++ | −− | + | + |
| 86 | ++ | +++ | +++ | ++ | + | + |
| 87 | +++ | +++ | ++ | −− | + | + |
| 88 | + | + | + | −− | + | + |
| 89 | + | ++ | + | −− | + | + |
| 90 | + | ++ | ++ | −− | + | + |
| 91 | + | +++ | ++ | −− | + | + |
| 92 | ++ | ++ | + | −− | + | + |
| 93 | ++ | ++ | + | −− | + | + |
| 94 | ++ | +++ | +++ | ++ | + | + |
| 95 | ++ | +++ | +++ | ++ | + | + |
| 96 | + | ++ | + | −− | + | + |
| 98 | ++ | +++ | ++ | −− | + | + |
| 99 | + | +++ | +++ | −− | + | + |
| 100 | + | ++ | ++ | −− | + | + |
| 101 | ++ | +++ | +++ | −− | + | ++ |
| 102 | + | + | + | −− | + | + |
| 103 | ++ | +++ | +++ | ++ | + | ++ |
| 104 | ++ | +++ | ++ | −− | + | + |
| 105 | + | ++ | + | −− | + | + |
| 106 | + | + | + | −− | + | + |
| 107 | + | ++ | ++ | −− | + | + |
| 108 | ++ | +++ | +++ | +++ | + | + |
| 109 | + | +++ | +++ | −− | + | + |
| 110 | ++ | +++ | +++ | −− | + | + |
| 111 | ++ | ++ | ++ | ++ | + | + |
| 112 | ++ | +++ | +++ | −− | + | ++ |
| 113 | + | ++ | + | −− | + | + |
| 114 | ++ | +++ | +++ | ++ | + | + |
| 115 | ++ | +++ | ++ | ++ | + | + |
| 116 | ++ | +++ | +++ | −− | + | + |
| 117 | + | ++ | ++ | −− | + | + |
| 118 | + | + | + | −− | + | + |
| 119 | ++ | +++ | ++ | ++ | + | + |
| 120 | + | ++ | ++ | + | + | + |
| 121 | ++ | +++ | +++ | −− | + | + |
| 122 | ++ | +++ | ++ | ++ | + | + |
| 123 | ++ | +++ | ++ | −− | + | + |
| 124 | ++ | +++ | +++ | ++ | + | + |
| 125 | + | +++ | ++ | ++ | + | + |
| 126 | ++ | +++ | ++ | ++ | + | + |
| 127 | ++ | +++ | ++ | ++ | + | + |
| 128 | + | +++ | ++ | ++ | + | + |
| 129 | + | +++ | ++ | −− | + | + |
| 130 | + | ++ | + | −− | + | + |
| 131 | ++ | +++ | +++ | +++ | + | ++ |
| 132 | ++ | ++ | ++ | + | + | + |
| 133 | + | ++ | + | −− | + | + |
| 134 | + | +++ | ++ | −− | + | + |
| 135 | + | ++ | + | ++ | + | + |
| 136 | + | ++ | + | + | + | + |
| 137 | + | ++ | ++ | −− | + | + |
| 138 | ++ | +++ | +++ | ++ | + | + |
| 139 | + | ++ | + | + | + | + |
| 140 | + | ++ | + | −− | + | + |
| 142 | ++ | ++ | ++ | −− | + | + |
| 143 | + | +++ | +++ | +++ | + | + |
| 144 | + | +++ | ++ | + | + | + |
| 145 | + | +++ | ++ | ++ | + | + |
| 146 | + | ++ | + | + | + | + |
| 147 | + | ++ | ++ | −− | + | + |
| 148 | + | +++ | ++ | −− | + | + |
| 149 | + | +++ | ++ | −− | + | + |
| 151 | + | +++ | +++ | +++ | + | + |
| 154 | ++ | +++ | ++ | −− | + | + |
| 155 | ++ | +++ | +++ | −− | + | ++ |
| 156 | + | +++ | ++ | ++ | + | + |
| 157 | + | ++ | ++ | −− | + | + |
| 158 | ++ | +++ | +++ | +++ | + | + |
| 159 | + | ++ | + | −− | + | + |
| 162 | + | ++ | ++ | −− | + | ++ |
| 164 | ++ | +++ | +++ | ++ | + | + |
| 165 | + | +++ | ++ | ++ | + | + |
| 166 | + | +++ | +++ | −− | + | + |
| 167 | ++ | +++ | ++ | −− | + | + |
| 168 | ++ | +++ | ++ | + | + | + |
| 169 | ++ | +++ | ++ | −− | + | + |
| 170 | ++ | ++ | ++ | −− | + | + |
| 171 | + | ++ | ++ | −− | + | + |
| 172 | ++ | +++ | +++ | +++ | + | + |
| 173 | + | ++ | + | −− | + | + |
| 174 | + | + | + | −− | + | + |
| 175 | ++ | +++ | +++ | −− | + | + |
| 176 | ++ | ++ | ++ | + | + | + |
| 177 | ++ | ++ | ++ | + | + | + |
| 178 | + | +++ | ++ | ++ | + | + |
| 179 | ++ | +++ | ++ | −− | + | + |
| 180 | + | ++ | + | −− | + | + |
| 181 | ++ | ++ | + | + | + | + |
| 182 | + | +++ | +++ | −− | + | + |
| 183 | ++ | +++ | ++ | ++ | + | + |
| 184 | ++ | +++ | +++ | −− | + | + |
| 186 | + | +++ | ++ | −− | + | + |
| 187 | + | +++ | +++ | ++ | + | + |
| 188 | + | + | + | −− | + | + |
| 189 | + | ++ | + | −− | + | + |
| 190 | + | ++ | ++ | −− | + | + |
| 191 | + | ++ | + | −− | + | + |
| 192 | ++ | ++ | ++ | −− | + | + |
| 193 | + | +++ | ++ | −− | + | + |
| 194 | ++ | +++ | +++ | ++ | + | + |
| 195 | ++ | +++ | −− | ++ | + | + |
| 196 | + | ++ | −− | + | + | + |
| 197 | + | ++ | −− | + | + | + |
| 198 | + | + | −− | + | + | + |
| 199 | ++ | +++ | +++ | −− | + | + |
| 200 | ++ | ++ | −− | + | + | + |
| 201 | + | ++ | −− | + | + | + |
| 202 | + | ++ | −− | + | + | + |
| 203 | + | ++ | −− | + | + | + |
| 204 | + | + | + | | + | + |
| 205 | + | + | + | | + | + |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1-120. (canceled)

121. A compound of Formula I: 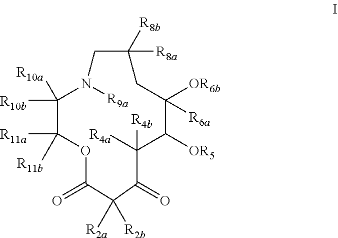
or a pharmaceutically acceptable salt thereof, wherein:
one of R₂ₐ and R_{2b} is selected from the group consisting of H, halo, optionally substituted C₁₋₁₀ alkyl, optionally substituted C₁₋₁₀ alkoxy, and optionally substituted C₁₋₁₀ alkenyl, wherein C₁₋₁₀ alkyl, C₁₋₁₀ alkoxy, and C₁₋₁₀ alkenyl are optionally substituted with one or more groups selected from the group consisting of halo, aryl, amino, alkyl, heteroalkyl, heteroalkenyl, heterocycloalkyl, and heteroaryl;
and the other of R₂ₐ and R_{2b} is selected from the group consisting of halo, optionally substituted C₁₋₁₀ alkyl, optionally substituted C₁₋₁₀ alkoxy, and optionally substituted C₁₋₁₀ alkenyl, wherein C₁₋₁₀ alkyl, C₁₋₁₀ alkoxy, and C₁₋₁₀ alkenyl are optionally substituted with one or more groups selected from the group consisting of halo, aryl, amino, alkyl, heteroalkyl, heteroalkenyl, heterocycloalkyl, and heteroaryl;
each of R₄ₐ and R_{4b} is independently selected from the group consisting of —H, and optionally substituted C₁₋₁₀ alkyl;
R₅ is selected from the group consisting of —H, an oxygen protecting group, and 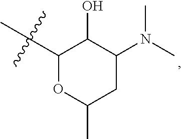 [SMILES: *C1OC(C)CC(N(C)C)C1O]
wherein “ [symbol] ” indicates a point of attachment
R₆ₐ is optionally substituted C₁₋₁₀ alkyl;
R_{6b} is —H, optionally substituted C₁₋₁₀ alkyl, optionally substituted C₁₋₁₀ hydroxyalkyl, and optionally substituted allyl;
R₈ₐ and R_{8b} are each independently selected from the group consisting of —H and optionally substituted C₁₋₁₀ alkyl;
R₉ₐ is selected from the group consisting of —H, —CO₂-alkylene-aryl, —C(═O)-alkyl, and optionally substituted C₁₋₁₀ alkyl;
one of R₁₀ₐ and R_{10b} is selected from the group consisting of —H, optionally substituted C₁₋₁₀ alkyl, —CO₂H, and —CO₂-alkyl; and
the other of R₁₀ₐ and R_{10b} is selected from the group consisting of optionally substituted saturated or partially unsaturated cycloalkyl containing at least one double bond, optionally substituted saturated or partially unsaturated heterocycloalkyl, optionally substituted aryl, and optionally substituted heteroaryl; and
R₁₁ₐ and R_{11b} are each independently selected from the group consisting of —H and optionally substituted C₁₋₁₀ alkyl.

122. The compound of claim 121, or a pharmaceutically acceptable salt thereof, which is a compound of formula IG:

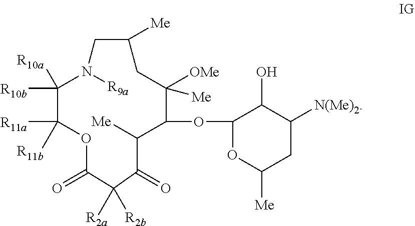

123. The compound of claim 121, or a pharmaceutically acceptable salt thereof, wherein R₉ₐ is —H or C₁₋₄ alkyl.

124. The compound of claim 123, or a pharmaceutically acceptable salt thereof, wherein R₁₁ₐ and R_{11b} are —H, or one of R₁₁ₐ and R_{11b} is —H and the other is optionally substituted C₁₋₁₀ alkyl such as methyl, or R₁₁ₐ and R_{11b} are each independently optionally substituted C₁₋₁₀ alkyl such as methyl.

125. The compound of claim 124, or a pharmaceutically acceptable salt thereof, wherein one of R₂ₐ and R_{2b} is methyl and the other of R₂ₐ and R_{2b} is H, or both of R₂ₐ and R_{2b} are methyl or one of R₂ₐ and R_{2b} is methyl and the other is fluoro or chloro or one of R₂ₐ and R_{2b} is methyl and the other is optionally substituted C₁₋₁₀ alkyl.

126. The compound of claim 125, or a pharmaceutically acceptable salt thereof, which is a compound of formula IH: 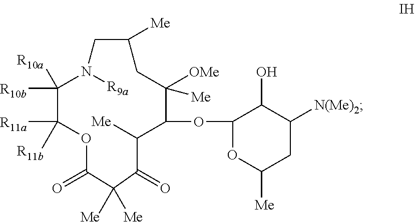
R_{10b} is selected from the group consisting of —H, optionally substituted C₁₋₁₀ alkyl, —CO₂H, and —CO₂-alkyl;
and R₁₀ₐ is selected from the group consisting of optionally substituted saturated or partially unsaturated cycloalkyl, optionally substituted saturated or partially unsaturated heterocycloalkyl, optionally substituted aryl, and an optionally substituted heteroaryl.

127. The compound of claim 126, or a pharmaceutically acceptable salt thereof, wherein:
R₁₀ₐ is optionally substituted -arylene-R₁₀₁;
R₁₀₁ is selected from the group consisting of —H, halo, optionally substituted aryl, optionally substituted heteroaryl; —B(OH)₂, —B(O-alkyl)₂, optionally substituted phenyl, and optionally substituted pyridyl.

128. The compound of claim 126, or a pharmaceutically acceptable salt thereof, wherein:
R₁₀ₐ is optionally substituted -heteroarylene-R_{101b};
R_{101b} is selected from the group consisting of —H, halo, optionally substituted aryl, and optionally substituted heteroaryl such as optionally substituted pyridyl.

129. The compound of claim 126, or a pharmaceutically acceptable salt thereof, wherein:
R₁₀ₐ is optionally substituted saturated or partially unsaturated cycloalkyl selected from the group consisting of optionally substituted cyclopropyl, optionally substituted cyclobutyl, optionally substituted saturated or partially unsaturated cyclopentyl, optionally substituted saturated or partially unsaturated cyclohexyl, and optionally substituted saturated or partially unsaturated cycloheptyl.

130. The compound of claim 129, or a pharmaceutically acceptable salt thereof, wherein:
R₁₀ₐ is 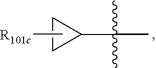
wherein “ [symbol] ” indicates a point of attachment;
R_{101c} is selected from the group consisting of —H, halo, —OH, alkoxy, —NRₓR_{x′}, and alkylene-R_{101c}′, wherein R₁₀₁ₑ, is selected from the group consisting of —H, halo, —OH, alkoxy, and NRₓR_{x′}, wherein:
at each occurrence Rₓ and R_{x′} are each independently selected from the group consisting of —H, optionally substituted alkyl, —C(═O)-alkyl, and —C(═O)-alkylene-N(R_{y′})(R_{y″}); or
Rₓ and R_{x′} together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl; and wherein
R_{y′} and R_{y″} are each independently selected from the group consisting of —H and optionally substituted C₁₋₁₀ alkyl; or
R_{y′} and R_{y″}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

131. The compound of claim 129, or a pharmaceutically acceptable salt thereof, wherein:
R₁₀ₐ is 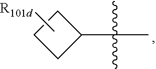
wherein “ [symbol] ” indicates a point of attachment;
R₁₀₁ₐ is selected from the group consisting of —H, halo, —OH, alkoxy, —NRₓR_{x′}, and -alkylene-R_{101a′}, wherein R_{101a′} is selected the group consisting of —H, halo, —OH, alkoxy, and —NRₓR_{x′}, wherein:
at each occurrence Rₓ and R_{x′} are each independently selected from the group consisting of —H, optionally substituted alkyl, —C(═O)-alkyl, and —C(═O)-alkylene-N(R_{y′})(R_{y″}); or
Rₓ and R_{x′} together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl; and wherein
R_{y′} and R_{y″} are each independently selected from the group consisting of —H and optionally substituted C₁₋₁₀ alkyl; or
R_{y′} and R_{y″}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

132. The compound of claim 129, or a pharmaceutically acceptable salt thereof, wherein:
R₁₀ₐ is optionally substituted saturated or partially unsaturated cyclopentyl or optionally substituted saturated or partially unsaturated cyclohexyl or optionally substituted saturated or partially unsaturated cycloheptyl.

133. The compound of claim 132, or a pharmaceutically acceptable salt thereof, wherein:
R₁₀ₐ is 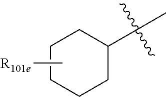
wherein “ [symbol] ” indicates a point of attachment;
R₁₀₁ₑ is selected from the group consisting of —H, halo, —OH, alkoxy, —NRₓR_{x′}, halo, —OH, alkoxy, —NR_{x′}R_{x′}, and -alkylene-R_{101e′}, wherein R_{101e′} is selected from the group consisting of —H, halo, —OH, alkoxy, and —NR_{x′}R_{x′}, wherein:
at each occurrence Rₓ and R_{x′} are each independently selected from the group consisting of —H, optionally substituted alkyl, —C(═O)-alkyl, and —C(═O)-alkylene-N(R_{y′})(R_{y″}); or
Rₓ and R_{x′} together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl; and wherein
R_{y′} and R_{y″} are each independently selected from the group consisting of —H and optionally substituted C₁₋₁₀ alkyl; or
R_{y′} and R_{y″}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

134. The compound of claim 126, or a pharmaceutically acceptable salt thereof, wherein:
R₁₀ₐ is optionally substituted heterocycloalkyl selected from the group consisting of optionally substituted aziridinyl, optionally substituted saturated or partially unsaturated pyrrolidinyl, and optionally substituted saturated or partially unsaturated piperidinyl.

135. The compound of claim 134, or a pharmaceutically acceptable salt thereof, wherein:
R₁₀ₐ is azetidinyl optionally substituted with R_{101f}, wherein the point of attachment is on the azetidinyl;
R_{101f} is selected from the group consisting of —H, halo, optionally substituted alkyl, —OH, —CO₂H, —CO₂-alkyl, alkoxy, optionally substituted cycloalkyl, optionally substituted heterocycloalkyl, NRₓR_{x′}, —C(═O)-alkyl, —C(═O)-optionally substituted heterocycloalkyl, alkenyl, —C(═O)-optionally substituted alkylene-R_{101f′}, -alkylene-C(═O)—R_{101f′} and -alkylene-R_{101f′}, wherein R_{101f′} is selected from the group consisting of —H, halo, —OH, alkoxy, —CO₂H, CO₂-optionally substituted alkyl, optionally substituted cycloalkyl, optionally substituted hetercycloalkyl, optionally substituted aryl, optionally substituted heteroaryl, and —NRₓR_{x′}, wherein:
Rₓ and R_{x′} are each independently selected from the group consisting of —H, optionally substituted alkyl, —C(═O)-alkyl, and —C(═O)-alkylene-N(R_{y})₂; or
Rₓ and R_{x′} together with the atom to which they are attached form a 3-, 4-, 5-, 6-, or 7-membered ring, optionally containing an additional heteroatom selected from the group consisting of O, S, SO, SO₂, NR_{y}, and N—C₁-C₁₀ alkyl; and wherein
each R_{y} is independently selected from the group consisting of —H and optionally substituted C₁₋₁₀ alkyl; or
each R_{y}, together with the atom to which they are attached form a 3-, 4-, 5-, 6-, 7, 8-, 9-, or 10-membered monocyclic or bicyclic ring optionally containing an additional heteroatom selected from the group consisting of O, S, SO, SO₂, NH, and N—C₁-C₁₀ alkyl.

136. The compound of claim 134, or a pharmaceutically acceptable salt thereof, wherein:
R₁₀ₐ is saturated or partially unsaturated pyrrolidinyl optionally substituted with R_{101g}, wherein the point of attachment is the pyrrolidinyl;
R_{101g} is selected from the group consisting of —H, alkyl and —C(═O)-alkylene-NRₓR_{x′}; wherein:
R_{101g} is selected from the group consisting of —H, optionally substituted alkyl, —C(═O)-alkyl, and —C(═O)-alkylene-NRₓR_{x′}, wherein:
Rₓ and R_{x′} are each independently selected from the group consisting of —H, optionally substituted alkyl, —C(═O)-alkyl, —C(═O)-alkyl, and —C(═O)-alkylene-N(R_{y})₂; or
Rₓ and R_{x′} together with the atom to which they are attached form a 3-, 4-, 5-, 6-, or 7-membered ring optionally containing an additional heteroatom selected from the group consisting of O, S, SO, SO₂, NR_{y}, and N—C₁-C₁₀ alkyl; and wherein
each R_{y} is independently selected from the group consisting of —H and optionally substituted C₁₋₁₀ alkyl; or
each R_{y}, together with the atom to which they are attached, form a 3-, 4-, 5-, 6-, or 7-membered ring optionally containing an additional heteroatom selected from the group consisting of O, S, SO, SO₂, NH, and N—C₁-C₁₀ alkyl.

137. The compound of claim 136, or a pharmaceutically acceptable salt thereof, wherein:
R₁₀ₐ is 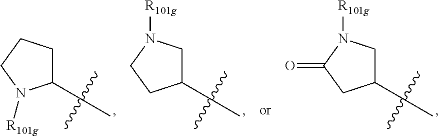
wherein “ [symbol] ” indicates a point of attachment and R_{101g} is selected from the group consisting of —H, methyl, ethyl, isopropyl, butyl, isobutyl, —C(═O)-methyl, —C(═O)—CH₂—N(Me)₂, and —C(═O)—CH₂—NHCH₂CH(Me)₂.

138. The compound of claim 134, or a pharmaceutically acceptable salt thereof, wherein:
R₁₀ₐ is saturated or partially unsaturated piperidinyl, 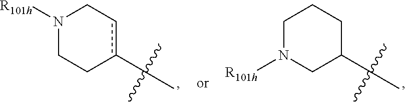
wherein “ [symbol] ” indicates a point of attachment
R₁₀₁ₕ is selected from the group consisting of —H, optionally substituted alkyl, optionally substituted haloalkyl, optionally substituted alkoxy, optionally substituted hydroxyalkyl, optionally substituted alkenyl, optionally substituted -alkylene-cycloalkyl, optionally substituted -alkylene-heterocycloalkyl, optionally substituted alkylene-aryl, optionally substituted alkylene-heteroaryl, —SO₂-optionally substituted alkyl, —C(═O)-optionally substituted alkyl, —C(═O)-optionally substituted alkylene-cycloalkyl, —C(═O)-optionally substituted alkylene-heterocycloalkyl, and —C(═O)-optionally substituted alkylene-NRₓR_{x′}; wherein
Rₓ and R_{x′} are each independently selected from the group consisting of —H, optionally substituted alkyl, —C(═O)-alkyl, —C(═O)-alkyl, and —C(═O)-alkylene-N(R_{y})₂; or
Rₓ and R_{x′} together with the atom to which they are attached form a 3-, 4-, 5-, 6-, or 7-membered ring optionally containing an additional heteroatom selected from the group consisting of O, S, SO, SO₂, NR_{y}, and N—C₁-C₁₀ alkyl; and wherein
each R_{y} is independently selected from the group consisting of —H and optionally substituted C₁₋₁₀ alkyl; or
each R_{y}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

139. The compound of claim 134, or a pharmaceutically acceptable salt thereof, wherein:
R₁₀ₐ is 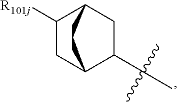
wherein “ [symbol] ” indicates a point of attachment;
R₁₀₁ⱼ is selected from the group consisting of —H, optionally substituted alkyl, haloalkyl, alkoxy, hydroxyalkyl, optionally substituted alkenyl, -alkylene-optionally substituted cycloalkyl, -alkylene-optionally substituted heterocycloalkyl, alkylene-optionally substituted aryl, alkylene-optionally substituted heteroaryl, —SO₂-alkyl, —C(═O)-alkyl, —C(═O)-alkylene-optionally substituted cycloalkyl, —C(═O)-alkylene-heterocycloalkyl, and —C(═O)-alkylene-NRₓR_{x′}; wherein
Rₓ and R_{x′} are each independently selected from the group consisting of —H, optionally substituted alkyl, —C(═O)-alkyl, —C(═O)-alkyl, and —C(═O)-alkylene-N(R_{y})₂; or
Rₓ and R_{x′} together with the atom to which they are attached form a 3-, 4-, 5-, 6-, or 7-membered ring optionally containing an additional heteroatom selected from the group consisting of O, S, SO, SO₂, NR_{y}, and N—C₁-C₁₀ alkyl; and wherein
each R_{y} is independently selected from the group consisting of —H and optionally substituted C₁₋₁₀ alkyl; or
each R_{y}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

140. The compound of claim 126, or a pharmaceutically acceptable salt thereof, wherein R₁₀ₐ is:
phenyl, bromophenyl, aminophenyl, OH 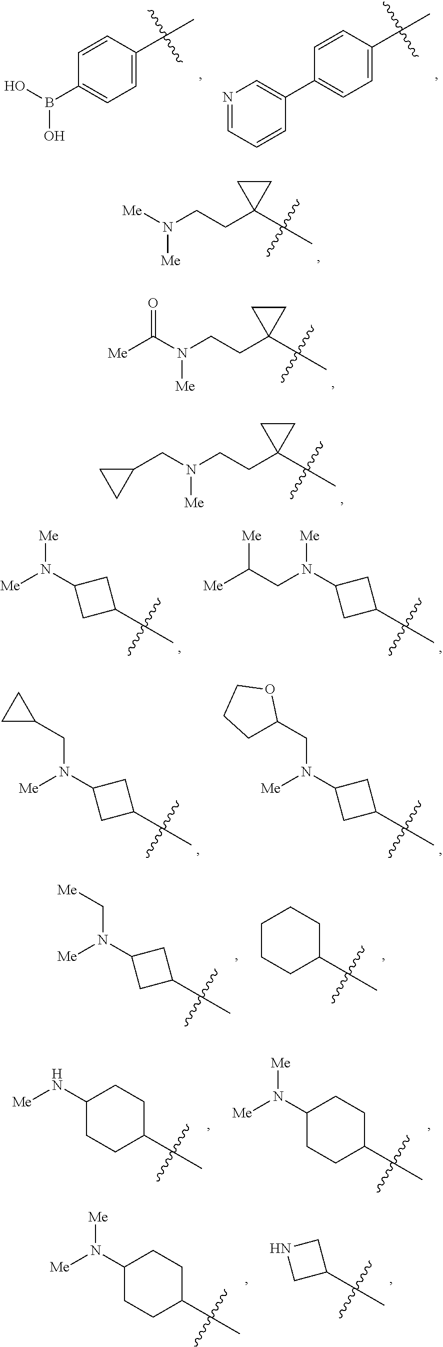 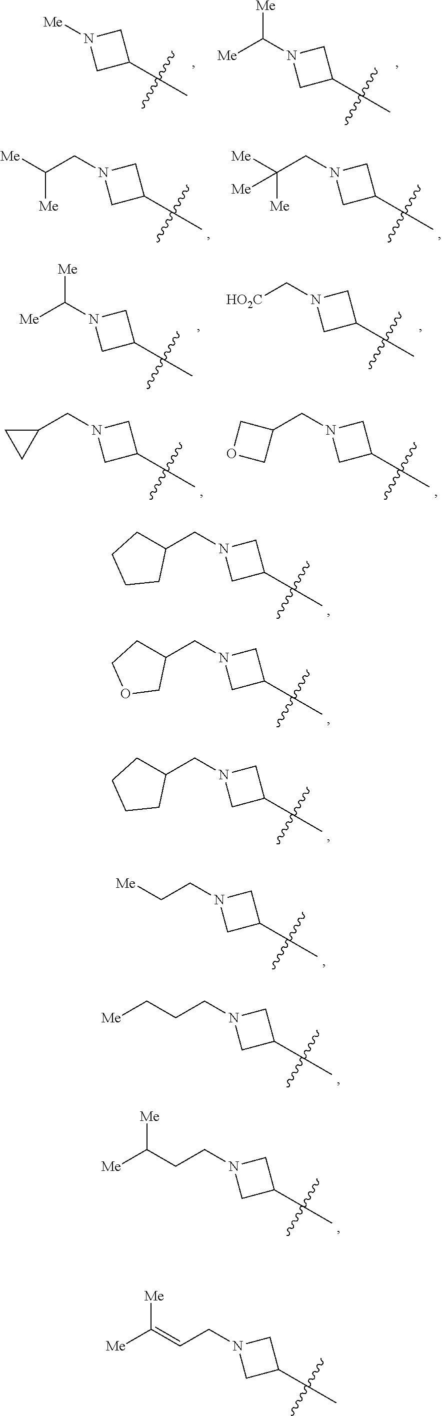 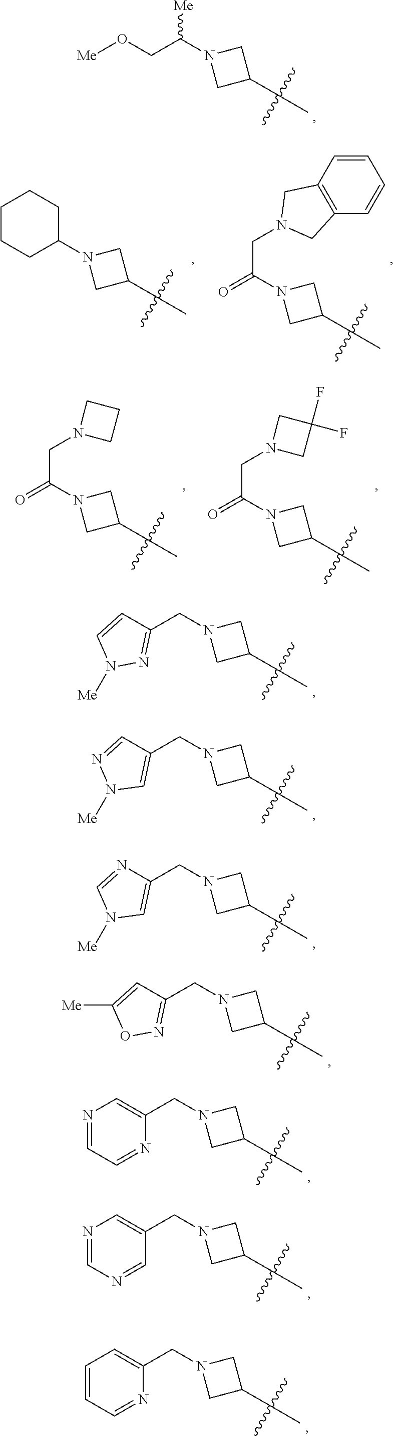 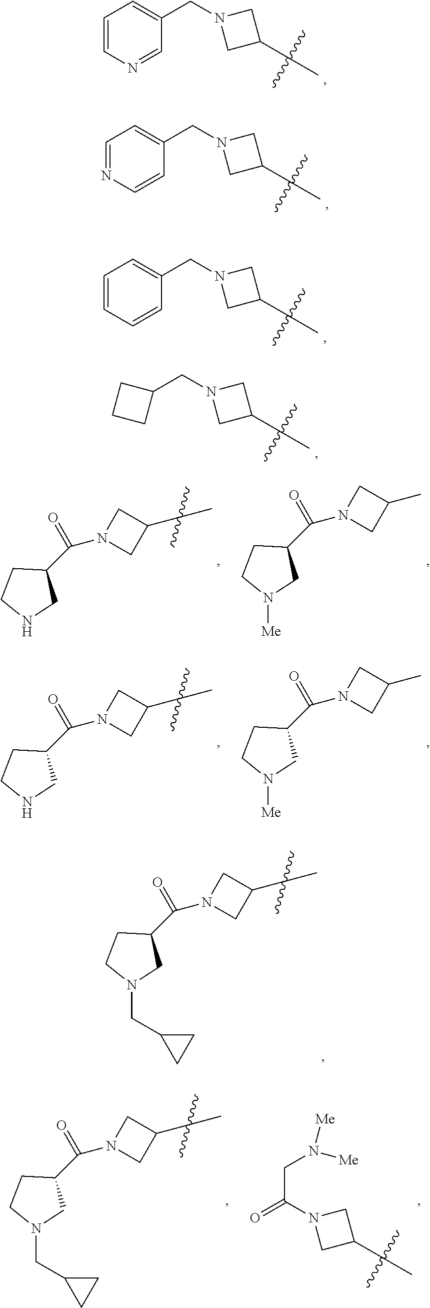 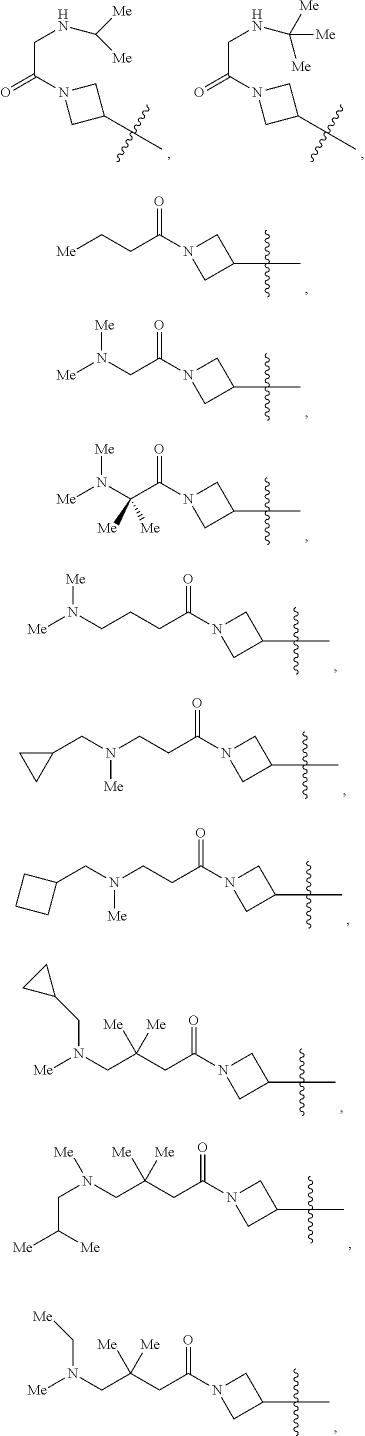 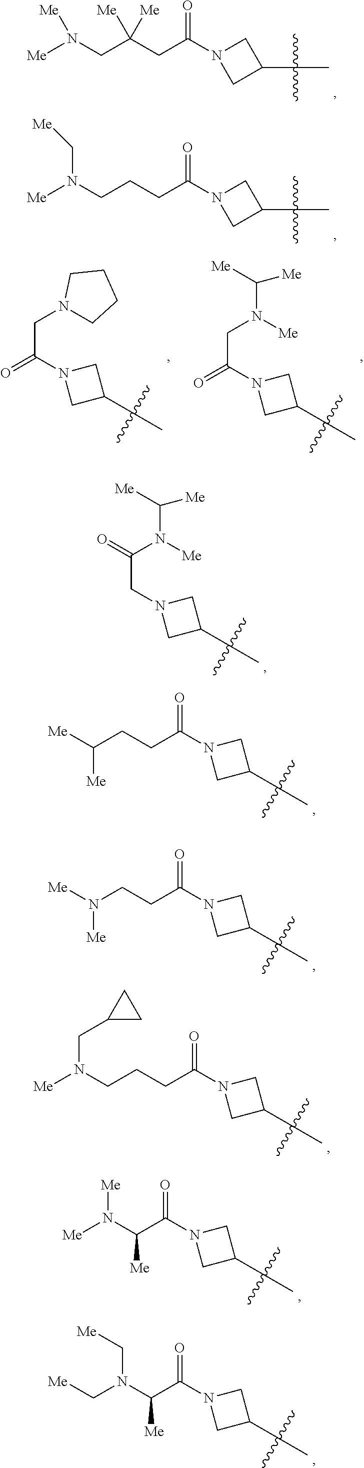 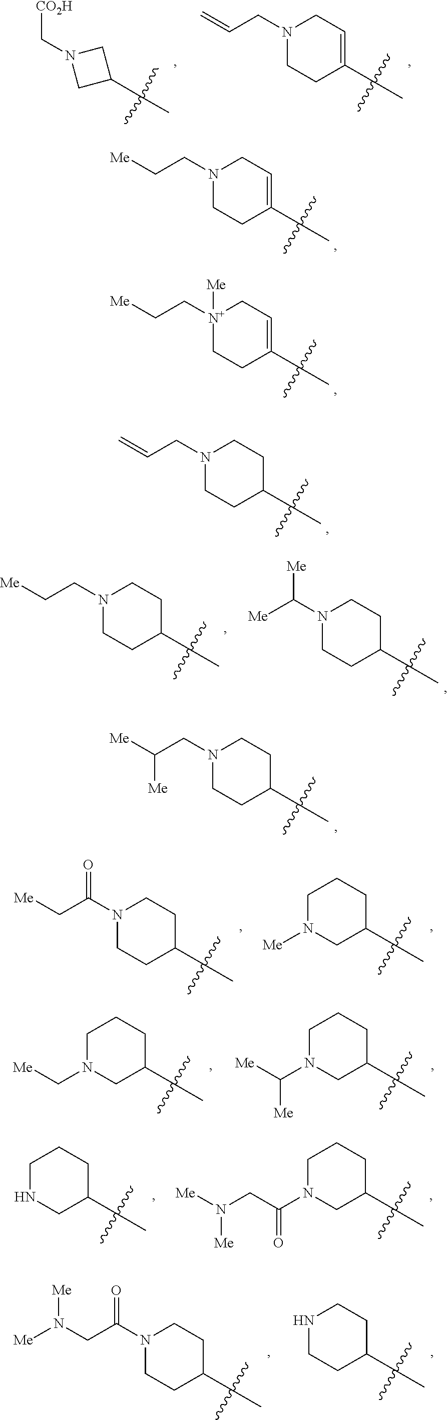 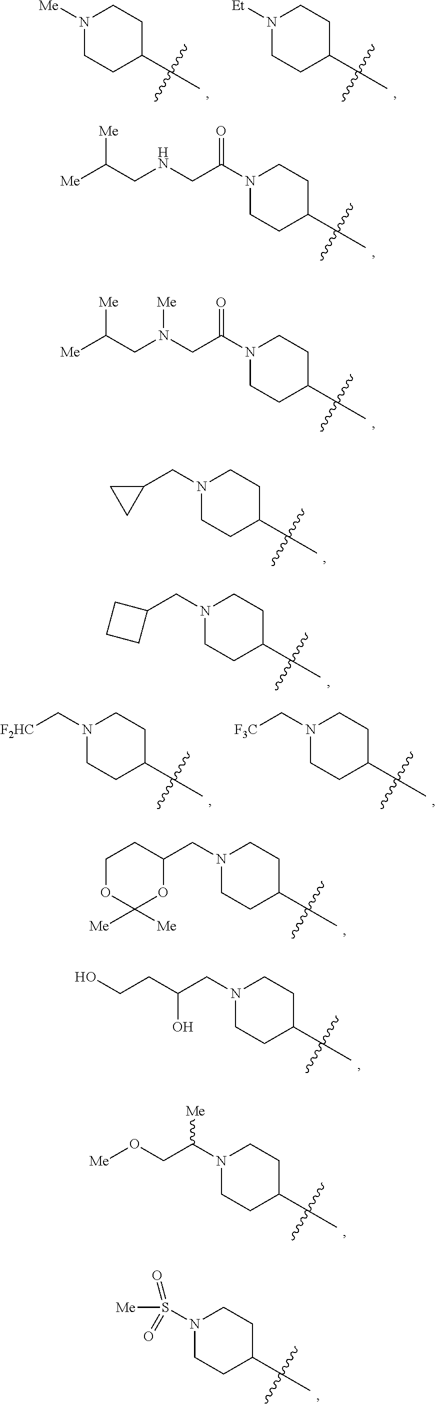 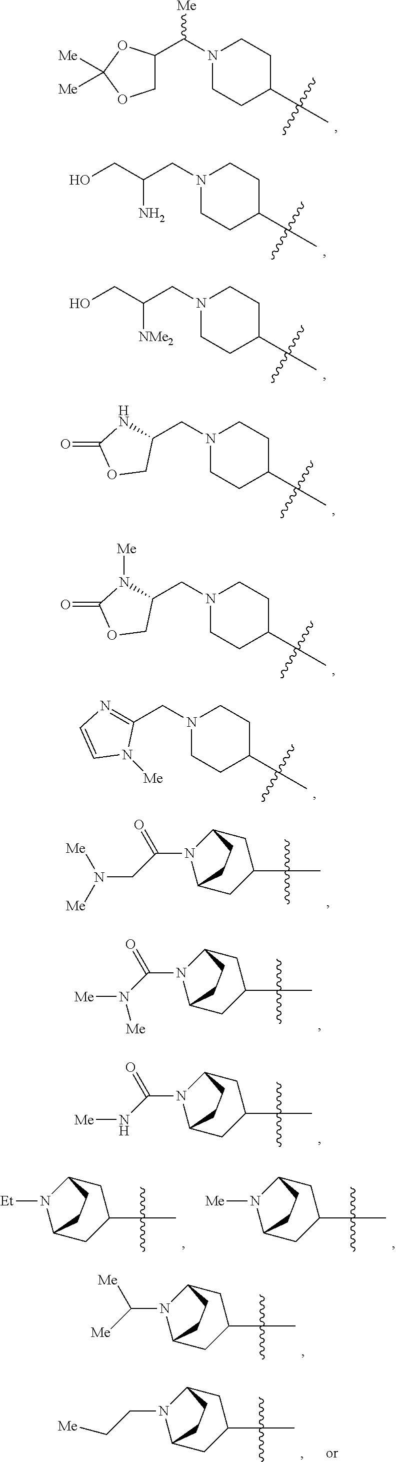 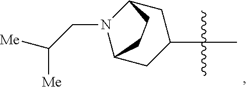 [SMILES: *C1C[C@H]2CC[C@@H](C1)N2CC(C)C]
wherein “ [symbol] ” indicates a point of attachment.

141. The compound of claim 121, which is a compound of formula III: 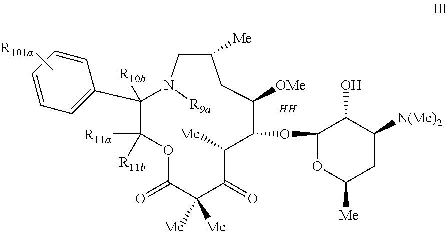
or a pharmaceutically acceptable salt thereof, wherein R₁₀₁ₐ is selected from the group consisting of —H, halo, optionally substituted aryl, and optionally substituted heteroaryl wherein R₁₀₁ₐ is selected from the group consisting of —H, halo, —B(OH)₂, —B(O-alkyl)₂, optionally substituted phenyl, and optionally substituted heteroaryl; or a compound of formula IV: 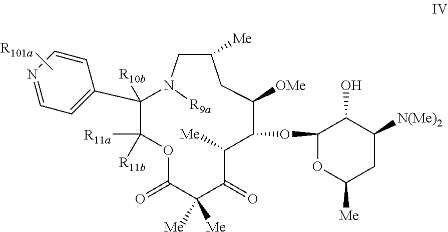
or a pharmaceutically acceptable salt thereof, wherein R_{101b} is selected from the group consisting of H, halo, optionally substituted aryl, and optionally substituted heteroaryl; or a compound of formula V: 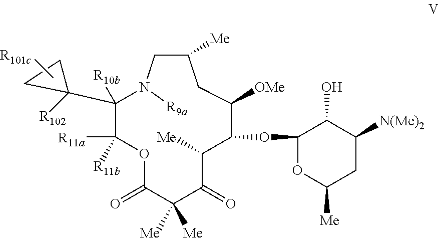
or a pharmaceutically acceptable salt thereof, wherein
R_{101c} is selected from the group consisting of —H, halo, —OH, alkoxy, —NRₓR_{x′}, and alkylene-R₁₀₁ₐ, wherein R_{101d} is selected from the group consisting of —H, halo, —OH, alkoxy, and —NRₓR_{x′}, wherein:
Rₓ and R_{x′} are each independently selected from the group consisting of —H, optionally substituted alkyl, —C(═O)-alkyl, and —C(═O)-alkylene-N(R_{y})₂; or
Rₓ and R_{x′} together with the atom to which they are attached form a 3-, 4-, 5-, 6-, or 7-membered ring optionally containing an additional heteroatom selected from the group consisting of O, S, SO, SO₂, NR_{y}, and N—C₁-C₁₀ alkyl; and wherein
each R_{y} is independently selected from the group consisting of —H and optionally substituted C₁₋₁₀ alkyl; or
each R_{y}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl; and
R₁₀₂ is H or alkyl; or a compound of formula VI: 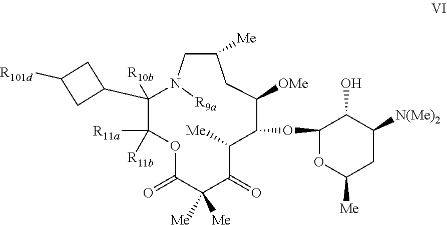
or a pharmaceutically acceptable salt thereof, wherein:
R_{101d} is selected from the group consisting of —H, halo, —OH, alkoxy, and —NRₓR_{x′}, wherein:
Rₓ and R_{x′} are each independently selected from the group consisting of —H, optionally substituted alkyl, —C(═O)-alkyl, and —C(═O)-alkylene-N(R_{y})₂; or
Rₓ and R_{x′} together with the atom to which they are attached form a 3-, 4-, 5-, 6-, or 7-membered ring optionally containing an additional heteroatom selected from the group consisting of O, S, SO, SO₂, NR_{y}, and N—C₁-C₁₀ alkyl; and wherein
each R_{y} is independently selected from the group consisting of —H and optionally substituted C₁₋₁₀ alkyl; or
each R_{y}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl; or a compound of formula VII: 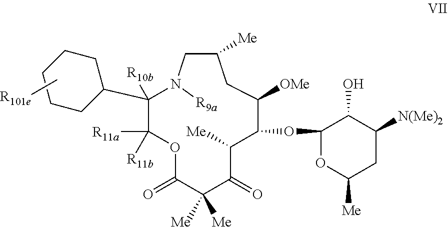
or a pharmaceutically acceptable salt thereof, wherein:
R₁₀₁ₑ is selected from the group consisting of —H, halo, —OH, alkoxy, —NRₓR_{x′}, and alkylene-R_{101e′}, wherein R_{101e′}, is selected from the group consisting of H, halo, —OH, alkoxy, and NRₓR_{x′}, wherein:
Rₓ and R_{x′} are each independently selected from the group consisting of H, optionally substituted alkyl, —C(═O)-alkyl, and —C(═O)-alkylene-N(R_{y})₂; or
Rₓ and R_{x′} together with the atom to which they are attached form a 3-, 4-, 5-, 6-, or 7-membered ring optionally containing an additional heteroatom selected from the group consisting of O, S, SO, SO₂, NR_{y}, and N—C₁-C₁₀ alkyl; and wherein
each R_{y} is independently selected from the group consisting of —H and optionally substituted C₁₋₁₀ alkyl; or
each R_{y}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl; or a compound of formula VIII: 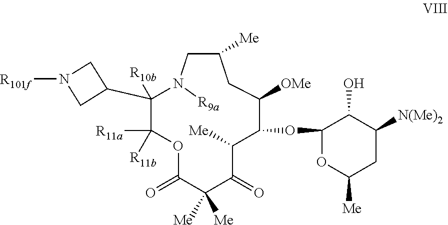
or a pharmaceutically acceptable salt thereof, wherein:
R_{101f} is selected from the group consisting of —H, halo, optionally substituted alkyl, —OH, —CO₂H, —CO₂-alkyl, alkoxy, optionally substituted cycloalkyl, optionally substituted heterocycloalkyl, NRₓR_{x′}, —C(═O)-alkyl, —C(═O)-optionally substituted heterocycloalkyl, alkenyl, —C(═O)-optionally substituted alkylene-R_{101f′}, -alkylene-C(═O)—R_{101f′} and -alkylene-R_{101f′}, wherein R_{101f′} is selected from the group consisting of —H, halo, —OH, alkoxy, —CO₂H, CO₂-optionally substituted alkyl, optionally substituted cycloalkyl, optionally substituted hetercycloalkyl, optionally substituted aryl, optionally substituted heteroaryl, and —NRₓR_{x′}, wherein:
Rₓ and R_{x′} are each independently selected from the group consisting of —H, optionally substituted alkyl, —C(═O)-alkyl, and —C(═O)-alkylene-N(R_{y})₂; or
Rₓ and R_{x′} together with the atom to which they are attached form a 3-, 4-, 5-, 6-, or 7-membered ring, optionally containing an additional heteroatom selected from the group consisting of O, S, SO, SO₂, NR_{y}, and N—C₁-C₁₀ alkyl; and wherein
each R_{y} is independently selected from the group consisting of —H and optionally substituted C₁₋₁₀ alkyl; or
each R_{y}, together with the atom to which they are attached form a 3-, 4-, 5-, 6-, 7-, 8-, 9-, or 10-membered monocyclic or bicyclic ring optionally containing an additional heteroatom selected from the group consisting of O, S, SO, SO₂, NH, and N—C₁-C₁₀ alkyl; or a compound of formula IXa, IXb, or IXc: 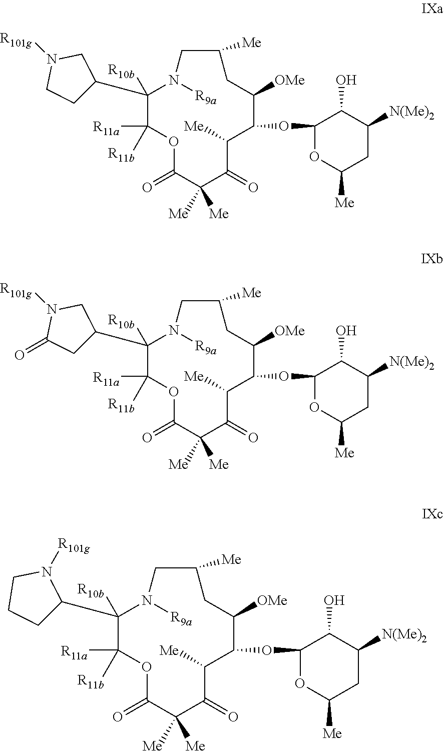
or a pharmaceutically acceptable salt thereof, wherein:
R_{101g} is selected from the group consisting of —H, optionally substituted alkyl, —C(═O)-alkyl, and —C(═O)-alkylene-NRₓR_{x′}, wherein:
Rₓ and R_{x′} are each independently selected from the group consisting of —H, optionally substituted alkyl, —C(═O)-alkyl, —C(═O)-alkyl, and —C(═O)-alkylene-N(R_{y})₂; or
Rₓ and R_{x′} together with the atom to which they are attached form a 3-, 4-, 5-, 6-, or 7-membered ring optionally containing an additional heteroatom selected from the group consisting of O, S, SO, SO₂, NR_{y}, and N—C₁-C₁₀ alkyl; and wherein
each R_{y} is independently selected from the group consisting of —H and optionally substituted C₁₋₁₀ alkyl; or
each R_{y}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl; or a compound of formula Xa or Xb: 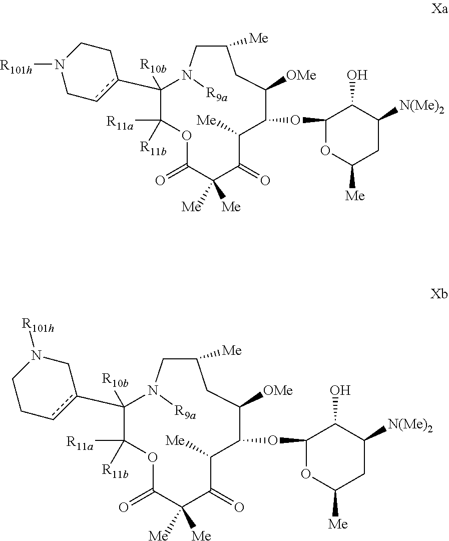
or a pharmaceutically acceptable salt thereof, wherein:
R₁₀₁ₕ is selected from the group consisting of —H, optionally substituted alkyl, optionally substituted haloalkyl, optionally substituted alkoxy, optionally substituted hydroxyalkyl, optionally substituted alkenyl, optionally substituted -alkylene-cycloalkyl, optionally substituted -alkylene-heterocycloalkyl, optionally substituted alkylene-aryl, optionally substituted alkylene-heteroaryl, —SO₂-optionally substituted alkyl, —C(═O)-optionally substituted alkyl, —C(═O)-optionally substituted alkylene-cycloalkyl, —C(═O)-optionally substituted alkylene-heterocycloalkyl, and —C(═O)-optionally substituted alkylene-NRₓR_{x′}; wherein
Rₓ and R_{x′} are each independently selected from the group consisting of —H, optionally substituted alkyl, —C(═O)-alkyl, —C(═O)-alkyl, and —C(═O)-alkylene-N(R_{y})₂; or
Rₓ and R_{x′} together with the atom to which they are attached form a 3-, 4-, 5-, 6-, or 7-membered ring optionally containing an additional heteroatom selected from the group consisting of O, S, SO, SO₂, NR_{y}, and N—C₁-C₁₀ alkyl; and wherein
each R_{y} is independently selected from the group consisting of —H and optionally substituted C₁₋₁₀ alkyl; or
each R_{y}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl; or a compound of formula XI: 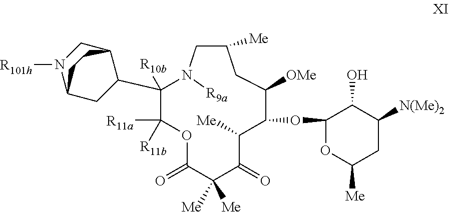
or a pharmaceutically acceptable salt thereof, wherein:
R₁₀₁ⱼ is selected from the group consisting of —H, optionally substituted alkyl, haloalkyl, alkoxy, hydroxyalkyl, optionally substituted alkenyl, -alkylene-optionally substituted cycloalkyl, -alkylene-optionally substituted heterocycloalkyl, alkylene-optionally substituted aryl, alkylene-optionally substituted heteroaryl, —SO₂-alkyl, —C(═O)-alkyl, —C(═O)-alkylene-optionally substituted cycloalkyl, —C(═O)-alkylene-heterocycloalkyl, and —C(═O)-alkylene-NRₓR_{x′}; wherein
Rₓ and R_{x′} are each independently selected from the group consisting of —H, optionally substituted alkyl, —C(═O)-alkyl, —C(═O)-alkyl, and —C(═O)-alkylene-N(R_{y})₂; or
Rₓ and R_{x′} together with the atom to which they are attached form a 3-, 4-, 5-, 6-, or 7-membered ring optionally containing an additional heteroatom selected from the group consisting of O, S, SO, SO₂, NR_{y}, and N—C₁-C₁₀ alkyl; and wherein
each R_{y} is independently selected from the group consisting of —H and optionally substituted C₁₋₁₀ alkyl; or
each R_{y}, together with the atom to which they are attached form a 3-, 4-, 5-, 6-, or 7-membered ring optionally containing an additional heteroatom selected from the group consisting of O, S, SO, SO₂, NH, and N—C₁-C₁₀ alkyl.

142. A compound selected from the following table or a pharmaceutically acceptable salt thereof.

| Compound # | Structure |
|---|---|
| 1 | 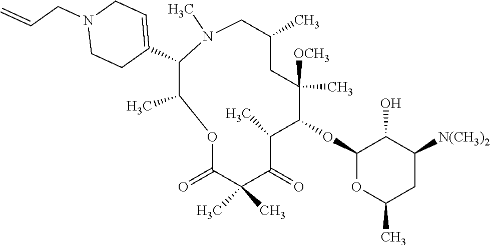 |
| 2 | 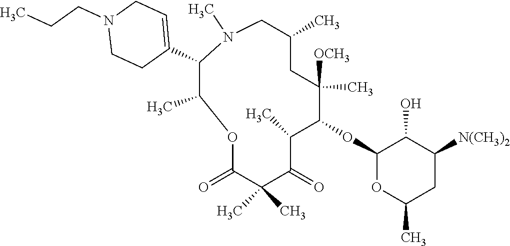 |
| 4 | 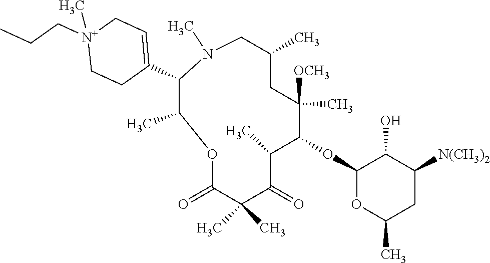 |
| 5 | 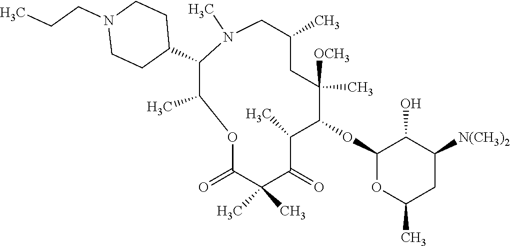 |
| 6 | 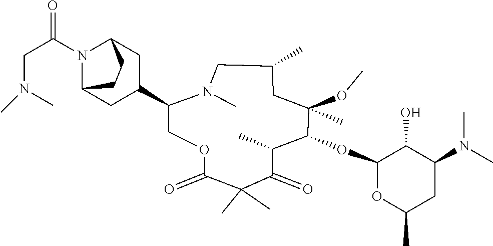 |
| 7 | 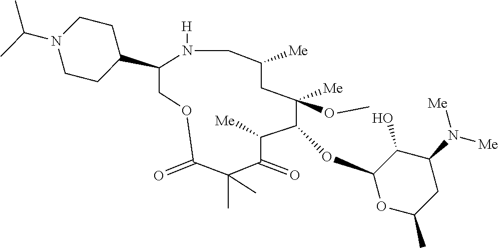 [SMILES: CO[C@]1(C)C[C@@H](C)CN[C@H](C2CCN(C(C)C)CC2)COC(=O)C(C)(C)C(=O)[C@H](C)[C@H]1O[C@@H]1O[C@H](C)C[C@H](N(C)C)[C@H]1O] |
| 8 | 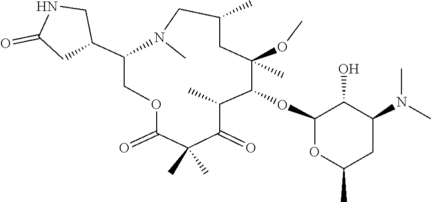 |
| 9 | 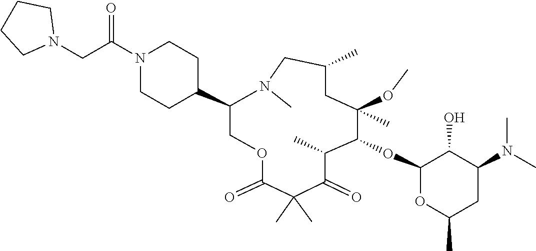 [SMILES: CO[C@]1(C)C[C@@H](C)CN(C)[C@H](C2CCN(C(=O)CN3CCCC3)CC2)COC(=O)C(C)(C)C(=O)[C@H](C)[C@H]1O[C@@H]1O[C@H](C)C[C@H](N(C)C)[C@H]1O] |
| 10 | 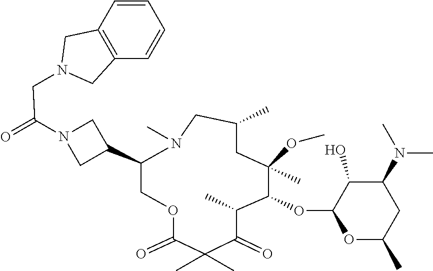 [SMILES: CO[C@]1(C)C[C@@H](C)CN(C)[C@H](C2CN(C(=O)CN3Cc4ccccc4C3)C2)COC(=O)C(C)(C)C(=O)[C@H](C)[C@H]1O[C@@H]1O[C@H](C)C[C@H](N(C)C)[C@H]1O] |
| 11 | 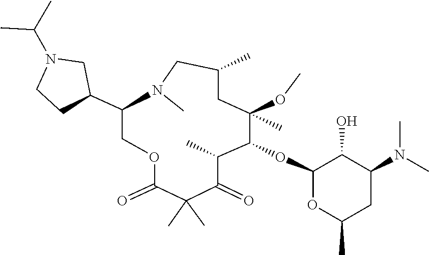 [SMILES: CO[C@]1(C)C[C@@H](C)CN(C)[C@H]([C@H]2CCN(C(C)C)C2)COC(=O)C(C)(C)C(=O)[C@H](C)[C@H]1O[C@@H]1O[C@H](C)C[C@H](N(C)C)[C@H]1O] |
| 12 | 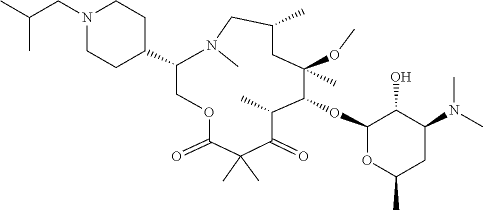 [SMILES: CO[C@]1(C)C[C@@H](C)CN(C)[C@@H](C2CCN(CC(C)C)CC2)COC(=O)C(C)(C)C(=O)[C@H](C)[C@H]1O[C@@H]1O[C@H](C)C[C@H](N(C)C)[C@H]1O] |
| 13 | 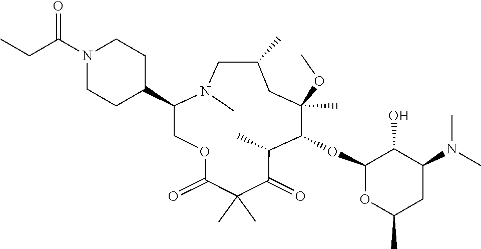 [SMILES: CCC(=O)N1CCC([C@@H]2COC(=O)C(C)(C)C(=O)[C@H](C)[C@@H](O[C@@H]3O[C@H](C)C[C@H](N(C)C)[C@H]3O)[C@](C)(OC)C[C@@H](C)CN2C)CC1] |
| 14 | 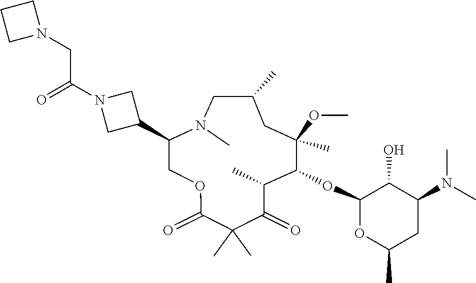 [SMILES: CO[C@]1(C)C[C@@H](C)CN(C)[C@H](C2CN(C(=O)CN3CCC3)C2)COC(=O)C(C)(C)C(=O)[C@H](C)[C@H]1O[C@@H]1O[C@H](C)C[C@H](N(C)C)[C@H]1O] |
| 15 | 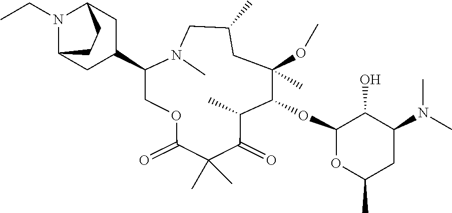 [SMILES: CCN1[C@@H]2CC[C@H]1CC([C@@H]1COC(=O)C(C)(C)C(=O)[C@H](C)[C@@H](O[C@@H]3O[C@H](C)C[C@H](N(C)C)[C@H]3O)[C@](C)(OC)C[C@@H](C)CN1C)C2] |
| 16 | 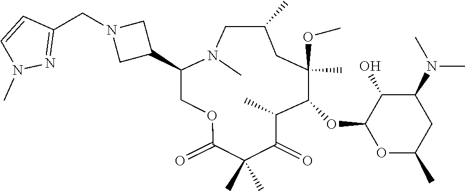 |
| 17 | 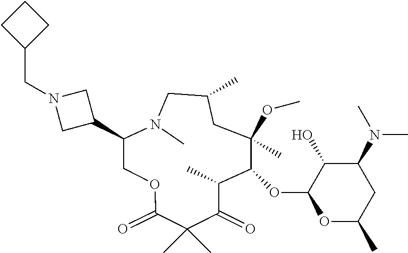 [SMILES: CO[C@]1(C)C[C@@H](C)CN(C)[C@H](C2CN(CC3CCC3)C2)COC(=O)C(C)(C)C(=O)[C@H](C)[C@H]1O[C@@H]1O[C@H](C)C[C@H](N(C)C)[C@H]1O] |
| 18 | 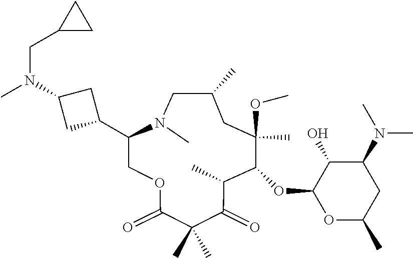 |
| 19 | 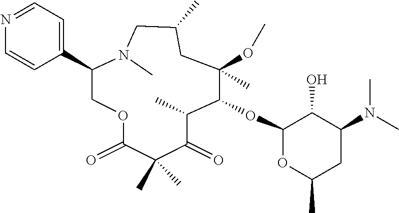 |
| 20 | 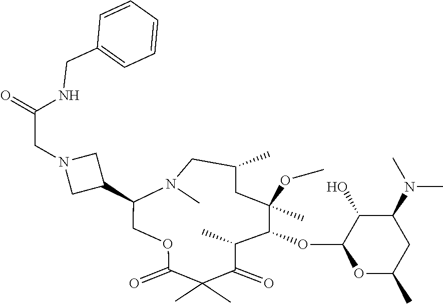 [SMILES: CO[C@]1(C)C[C@@H](C)CN(C)[C@H](C2CN(CC(=O)NCc3ccccc3)C2)COC(=O)C(C)(C)C(=O)[C@H](C)[C@H]1O[C@@H]1O[C@H](C)C[C@H](N(C)C)[C@H]1O] |
| 21 | 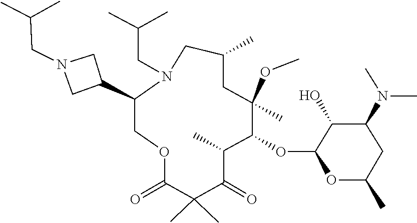 [SMILES: CO[C@]1(C)C[C@@H](C)CN(CC(C)C)[C@H](C2CN(CC(C)C)C2)COC(=O)C(C)(C)C(=O)[C@H](C)[C@H]1O[C@@H]1O[C@H](C)C[C@H](N(C)C)[C@H]1O] |
| 22 | 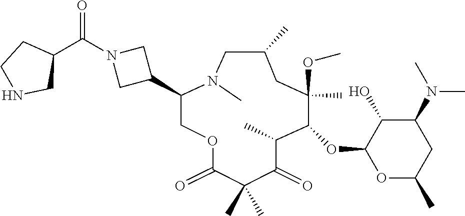 |
| 23 | 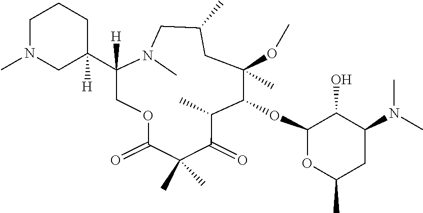 |
| 24 | 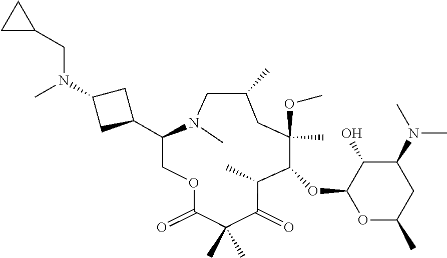 |
| 25 | 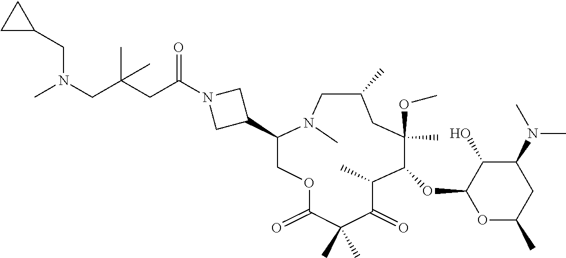 |
| 26 | 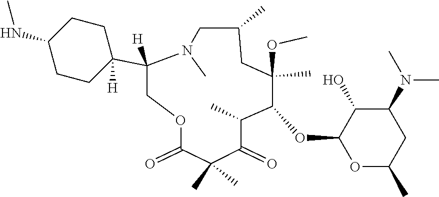 |
| 27 | 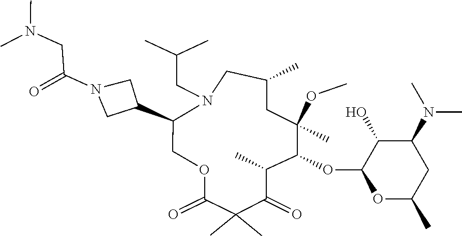 |
| 28 | 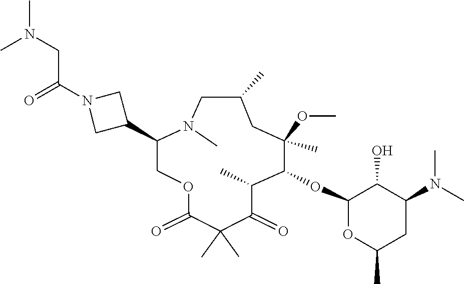 |
| 29 | 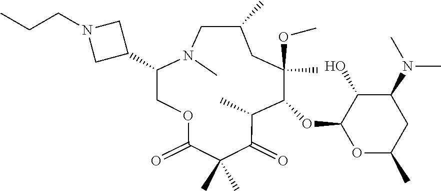 |
| 30 | 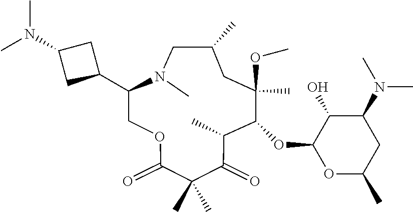 |
| 31 | 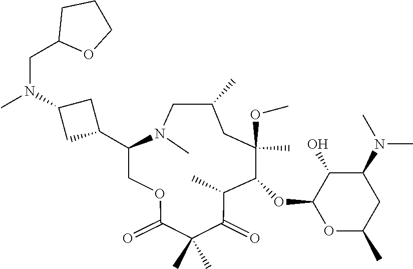 |
| 32 | 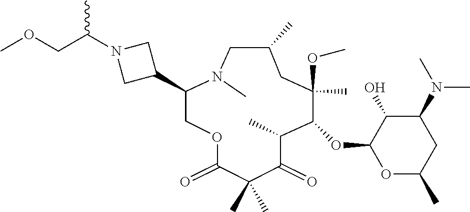 |
| 33 | 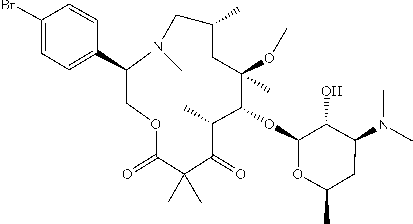 [SMILES: CO[C@]1(C)C[C@@H](C)CN(C)[C@H](c2ccc(Br)cc2)COC(=O)C(C)(C)C(=O)[C@H](C)[C@H]1O[C@@H]1O[C@H](C)C[C@H](N(C)C)[C@H]1O] |
| 34 | 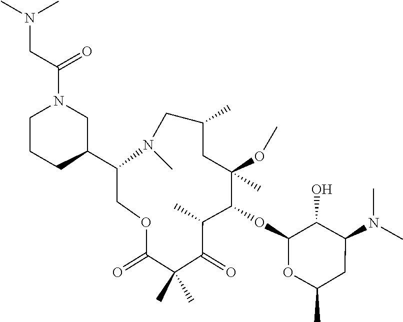 |
| 35 | 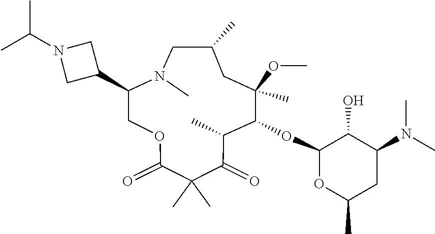 [SMILES: CO[C@]1(C)C[C@@H](C)CN(C)[C@H](C2CN(C(C)C)C2)COC(=O)C(C)(C)C(=O)[C@H](C)[C@H]1O[C@@H]1O[C@H](C)C[C@H](N(C)C)[C@H]1O] |
| 36 | 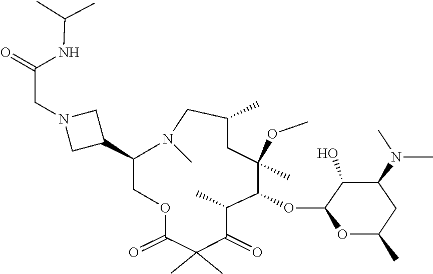 [SMILES: CO[C@]1(C)C[C@@H](C)CN(C)[C@H](C2CN(CC(=O)NC(C)C)C2)COC(=O)C(C)(C)C(=O)[C@H](C)[C@H]1O[C@@H]1O[C@H](C)C[C@H](N(C)C)[C@H]1O] |
| 37 | 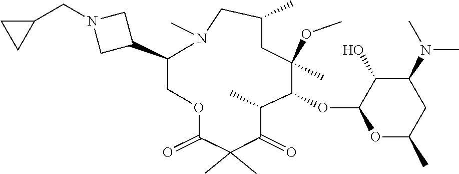 [SMILES: CO[C@]1(C)C[C@@H](C)CN(C)[C@H](C2CN(CC3CC3)C2)COC(=O)C(C)(C)C(=O)[C@H](C)[C@H]1O[C@@H]1O[C@H](C)C[C@H](N(C)C)[C@H]1O] |
| 38 | 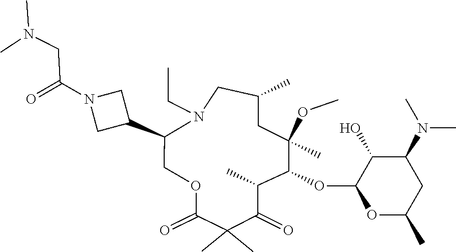 |
| 39 | 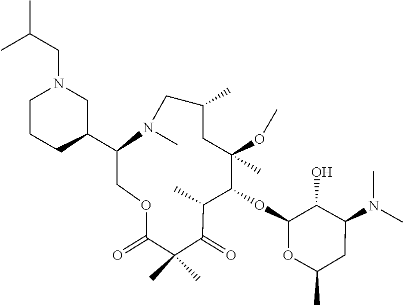 |
| 40 | 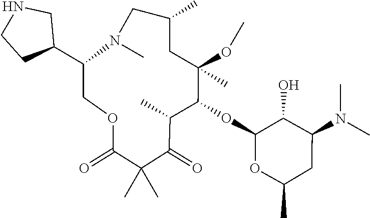 [SMILES: CO[C@]1(C)C[C@@H](C)CN(C)[C@@H]([C@H]2CCNC2)COC(=O)C(C)(C)C(=O)[C@H](C)[C@H]1O[C@@H]1O[C@H](C)C[C@H](N(C)C)[C@H]1O] |
| 41 | 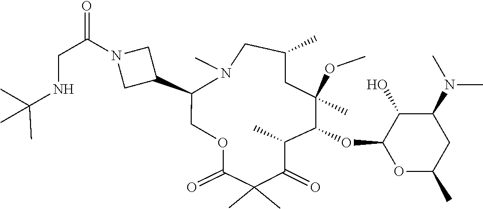 [SMILES: CO[C@]1(C)C[C@@H](C)CN(C)[C@H](C2CN(C(=O)CNC(C)(C)C)C2)COC(=O)C(C)(C)C(=O)[C@H](C)[C@H]1O[C@@H]1O[C@H](C)C[C@H](N(C)C)[C@H]1O] |
| 42 | 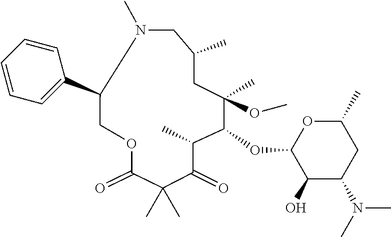 [SMILES: CO[C@]1(C)C[C@@H](C)CN(C)[C@H](c2ccccc2)COC(=O)C(C)(C)C(=O)[C@H](C)[C@H]1O[C@@H]1O[C@H](C)C[C@H](N(C)C)[C@H]1O] |
| 43 | 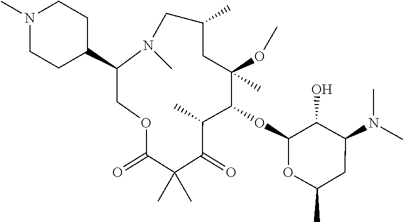 |
| 44 | 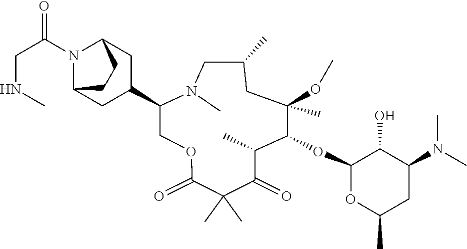 [SMILES: CNCC(=O)N1[C@@H]2CC[C@H]1CC([C@@H]1COC(=O)C(C)(C)C(=O)[C@H](C)[C@@H](O[C@@H]3O[C@H](C)C[C@H](N(C)C)[C@H]3O)[C@](C)(OC)C[C@@H](C)CN1C)C2] |
| 45 | 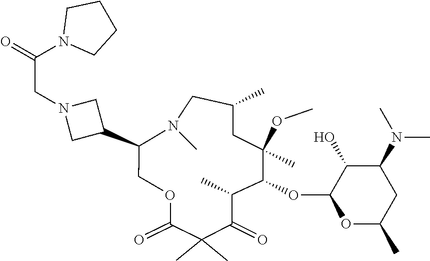 [SMILES: CO[C@]1(C)C[C@@H](C)CN(C)[C@H](C2CN(CC(=O)N3CCCC3)C2)COC(=O)C(C)(C)C(=O)[C@H](C)[C@H]1O[C@@H]1O[C@H](C)C[C@H](N(C)C)[C@H]1O] |
| 46 | 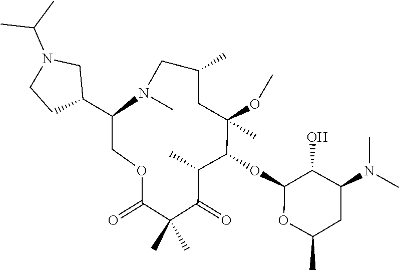 |
| 47 | 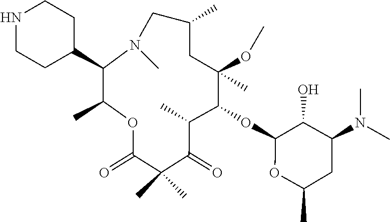 |
| 48 | 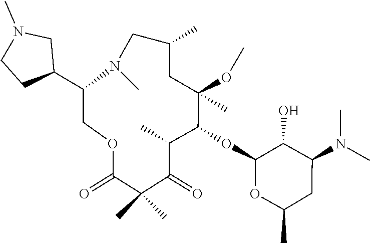 |
| 49 | 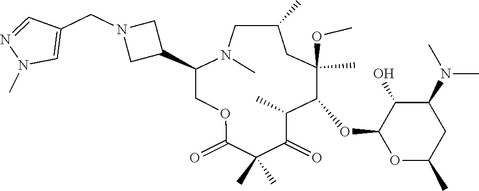 |
| 50 | 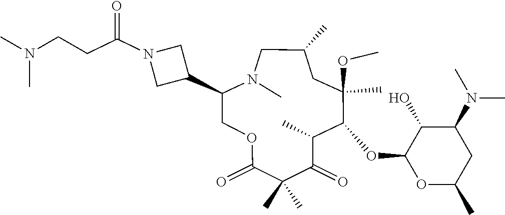 |
| 51 | 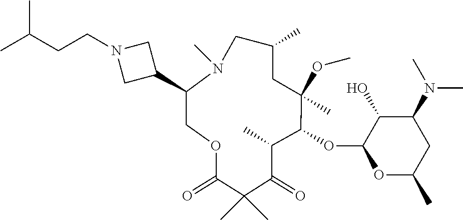 [SMILES: CO[C@]1(C)C[C@@H](C)CN(C)[C@H](C2CN(CCC(C)C)C2)COC(=O)C(C)(C)C(=O)[C@H](C)[C@H]1O[C@@H]1O[C@H](C)C[C@H](N(C)C)[C@H]1O] |
| 52 | 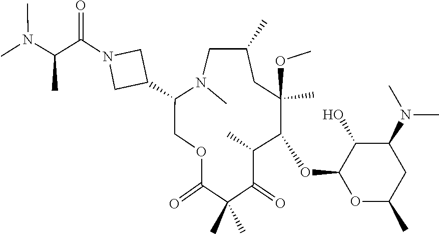 |
| 53 | 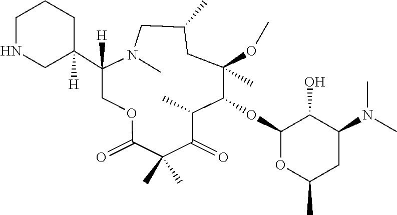 |
| 54 | 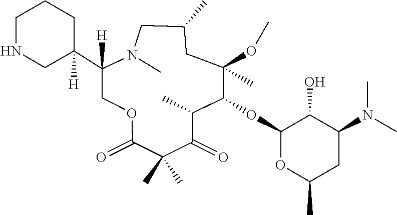 |
| 55 | 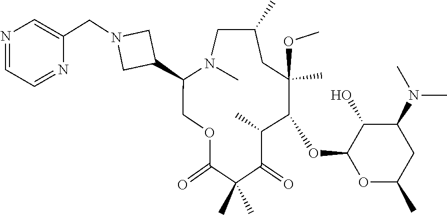 |
| 56 | 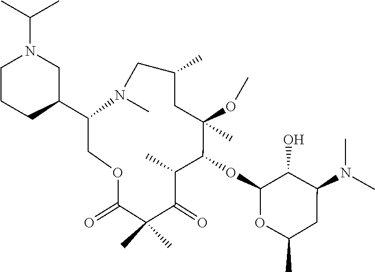 |
| 57 | 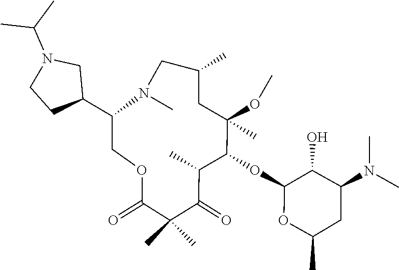 |
| 60 | 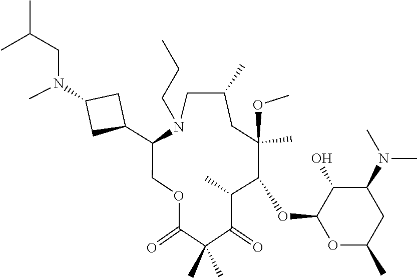 |
| 61 | 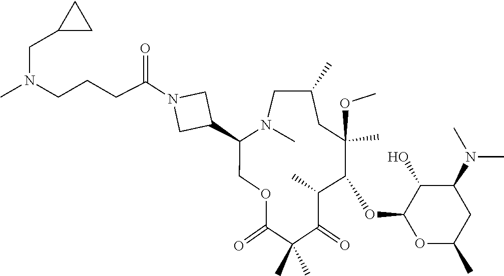 |
| 62 | 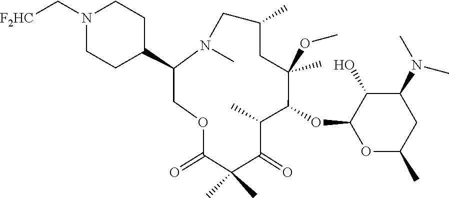 |
| 63 | 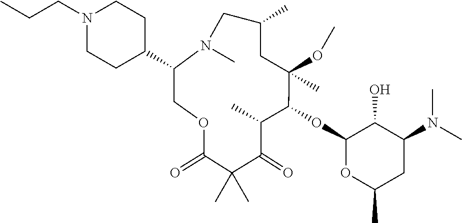 [SMILES: CCCN1CCC([C@H]2COC(=O)C(C)(C)C(=O)[C@H](C)[C@@H](O[C@@H]3O[C@H](C)C[C@H](N(C)C)[C@H]3O)[C@](C)(OC)C[C@@H](C)CN2C)CC1] |
| 64 | 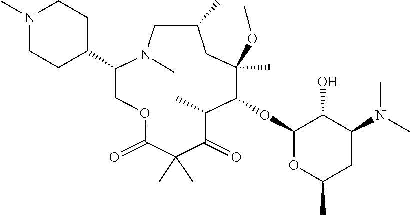 |
| 65 | 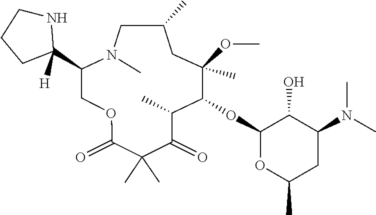 [SMILES: CO[C@]1(C)C[C@@H](C)CN(C)[C@H]([C@@H]2CCCN2)COC(=O)C(C)(C)C(=O)[C@H](C)[C@H]1O[C@@H]1O[C@H](C)C[C@H](N(C)C)[C@H]1O] |
| 66 | 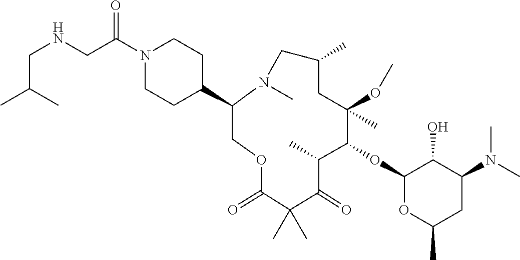 [SMILES: CO[C@]1(C)C[C@@H](C)CN(C)[C@H](C2CCN(C(=O)CNCC(C)C)CC2)COC(=O)C(C)(C)C(=O)[C@H](C)[C@H]1O[C@@H]1O[C@H](C)C[C@H](N(C)C)[C@H]1O] |
| 67 | 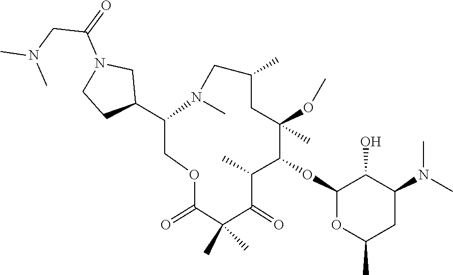 |
| 68 | 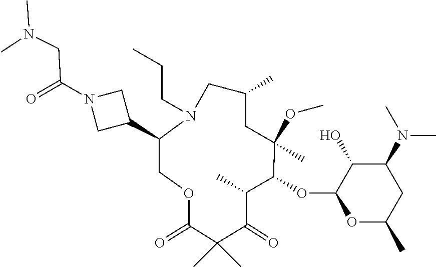 |
| 69 | 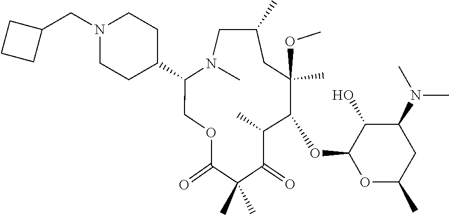 |
| 70 | 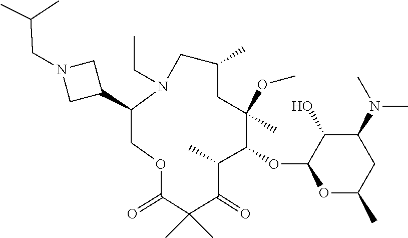 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@H]1C1CN(CC(C)C)C1] |
| 71 | 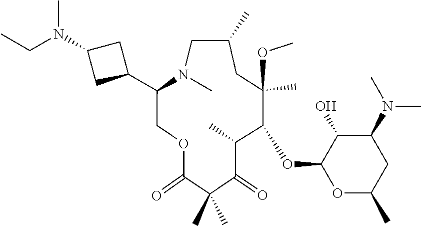 |
| 72 | 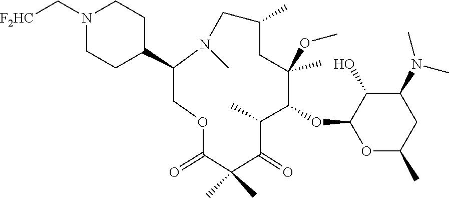 |
| 73 | 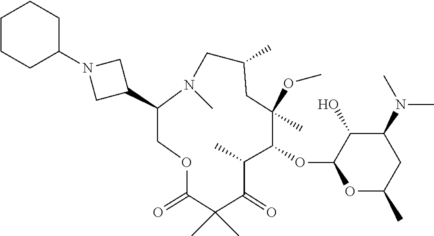 [SMILES: CO[C@]1(C)C[C@@H](C)CN(C)[C@H](C2CN(C3CCCCC3)C2)COC(=O)C(C)(C)C(=O)[C@H](C)[C@H]1O[C@@H]1O[C@H](C)C[C@H](N(C)C)[C@H]1O] |
| 74 | 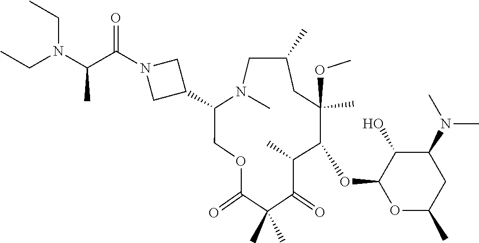 |
| 76 | 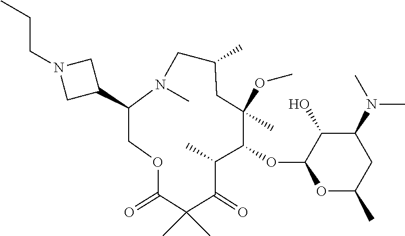 [SMILES: CCCN1CC([C@@H]2COC(=O)C(C)(C)C(=O)[C@H](C)[C@@H](O[C@@H]3O[C@H](C)C[C@H](N(C)C)[C@H]3O)[C@](C)(OC)C[C@@H](C)CN2C)C1] |
| 77 | 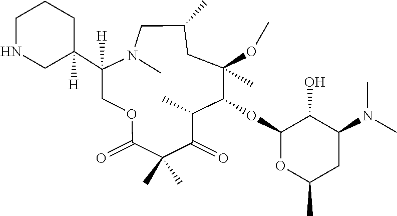 |
| 78 | 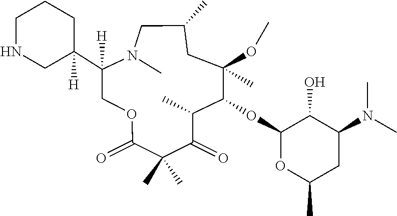 |
| 79 | 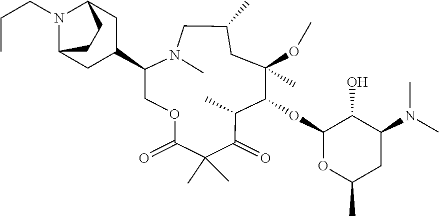 [SMILES: CCCN1[C@@H]2CC[C@H]1CC([C@@H]1COC(=O)C(C)(C)C(=O)[C@H](C)[C@@H](O[C@@H]3O[C@H](C)C[C@H](N(C)C)[C@H]3O)[C@](C)(OC)C[C@@H](C)CN1C)C2] |
| 80 | 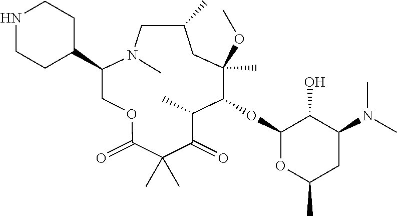 [SMILES: CO[C@]1(C)C[C@@H](C)CN(C)[C@H](C2CCNCC2)COC(=O)C(C)(C)C(=O)[C@H](C)[C@H]1O[C@@H]1O[C@H](C)C[C@H](N(C)C)[C@H]1O] |
| 81 | 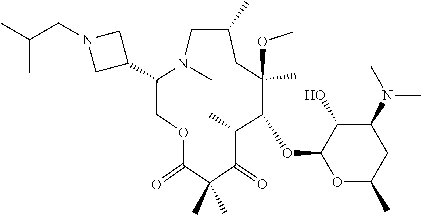 |
| 82 | 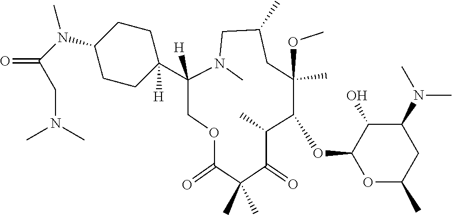 |
| 84 | 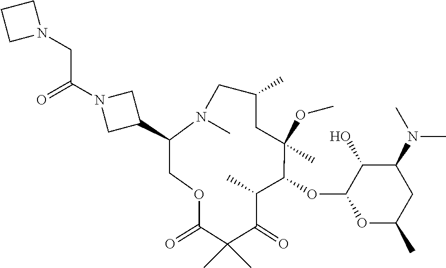 [SMILES: CO[C@]1(C)C[C@@H](C)CN(C)[C@H](C2CN(C(=O)CN3CCC3)C2)COC(=O)C(C)(C)C(=O)[C@H](C)[C@H]1O[C@H]1O[C@H](C)C[C@H](N(C)C)[C@H]1O] |
| 85 | 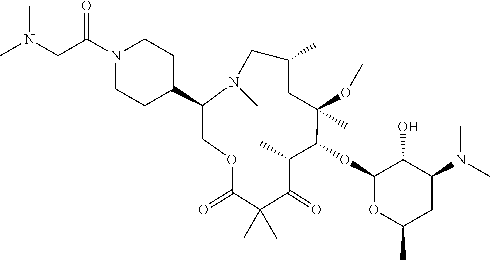 |
| 86 | 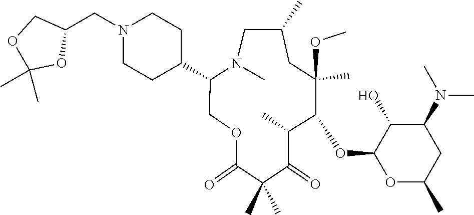 |
| 87 | 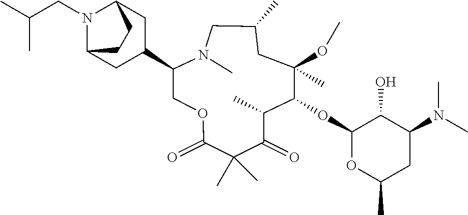 [SMILES: CO[C@]1(C)C[C@@H](C)CN(C)[C@H](C2C[C@H]3CC[C@@H](C2)N3CC(C)C)COC(=O)C(C)(C)C(=O)[C@H](C)[C@H]1O[C@@H]1O[C@H](C)C[C@H](N(C)C)[C@H]1O] |
| 88 | 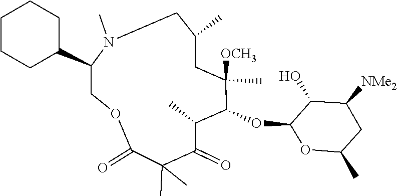 [SMILES: CO[C@]1(C)C[C@@H](C)CN(C)[C@H](C2CCCCC2)COC(=O)C(C)(C)C(=O)[C@H](C)[C@H]1O[C@@H]1O[C@H](C)C[C@H](N(C)C)[C@H]1O] |
| 89 | 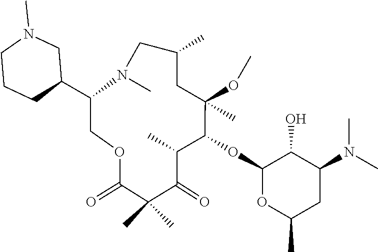 |
| 90 | 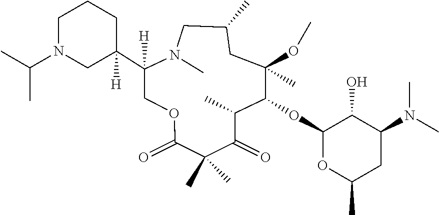 |
| 91 | 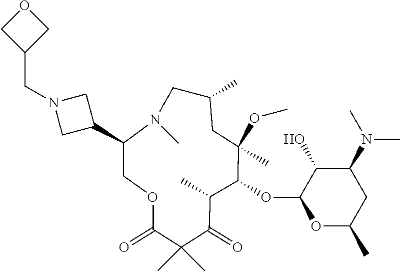 [SMILES: CO[C@]1(C)C[C@@H](C)CN(C)[C@H](C2CN(CC3COC3)C2)COC(=O)C(C)(C)C(=O)[C@H](C)[C@H]1O[C@@H]1O[C@H](C)C[C@H](N(C)C)[C@H]1O] |
| 92 | 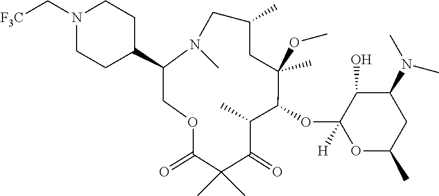 [SMILES: CO[C@]1(C)C[C@@H](C)CN(C)[C@H](C2CCN(CC(F)(F)F)CC2)COC(=O)C(C)(C)C(=O)[C@H](C)[C@H]1O[C@@H]1O[C@H](C)C[C@H](N(C)C)[C@H]1O] |
| 93 | 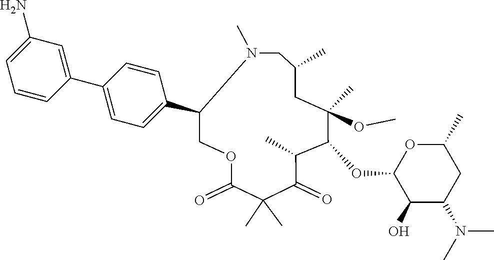 |
| 94 | 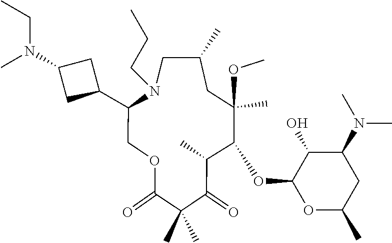 |
| 95 | 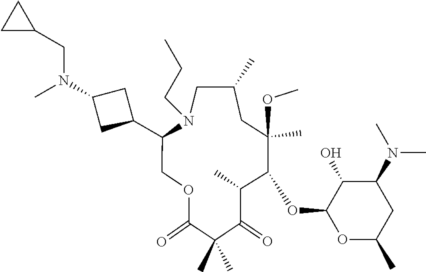 |
| 96 | 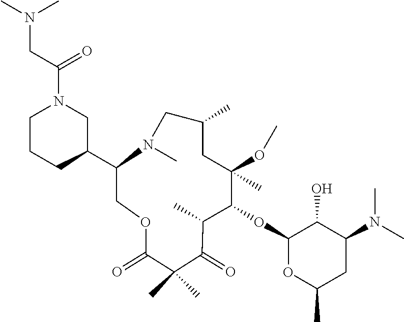 |
| 97 | 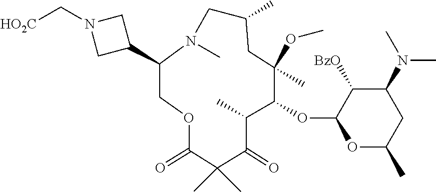 |
| 98 | 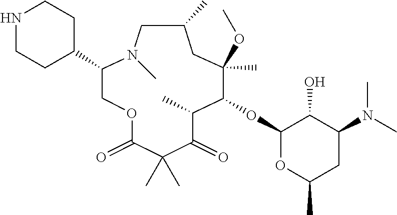 [SMILES: CO[C@]1(C)C[C@@H](C)CN(C)[C@@H](C2CCNCC2)COC(=O)C(C)(C)C(=O)[C@H](C)[C@H]1O[C@@H]1O[C@H](C)C[C@H](N(C)C)[C@H]1O] |
| 99 | 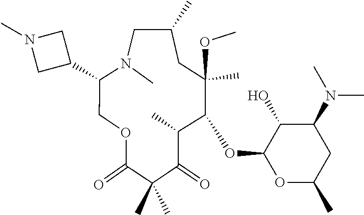 |
| 100 | 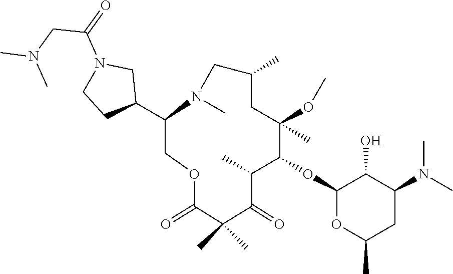 |
| 101 | 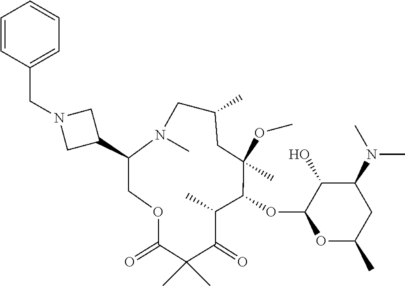 [SMILES: CO[C@]1(C)C[C@@H](C)CN(C)[C@H](C2CN(Cc3ccccc3)C2)COC(=O)C(C)(C)C(=O)[C@H](C)[C@H]1O[C@@H]1O[C@H](C)C[C@H](N(C)C)[C@H]1O] |
| 102 | 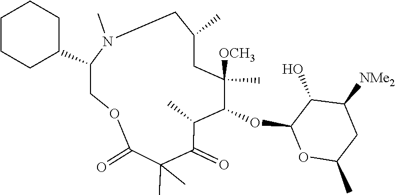 [SMILES: CO[C@]1(C)C[C@@H](C)CN(C)[C@@H](C2CCCCC2)COC(=O)C(C)(C)C(=O)[C@H](C)[C@H]1O[C@@H]1O[C@H](C)C[C@H](N(C)C)[C@H]1O] |
| 103 | 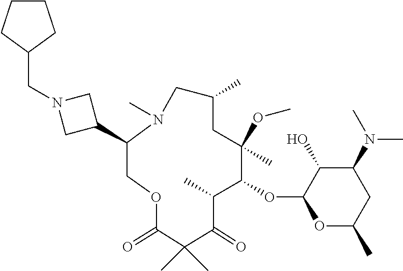 [SMILES: CO[C@]1(C)C[C@@H](C)CN(C)[C@H](C2CN(CC3CCCC3)C2)COC(=O)C(C)(C)C(=O)[C@H](C)[C@H]1O[C@@H]1O[C@H](C)C[C@H](N(C)C)[C@H]1O] |
| 104 | 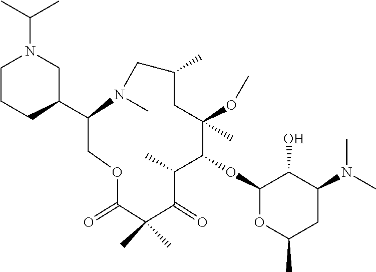 |
| 105 | 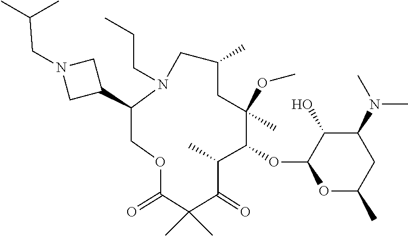 [SMILES: CCCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@H]1C1CN(CC(C)C)C1] |
| 106 | 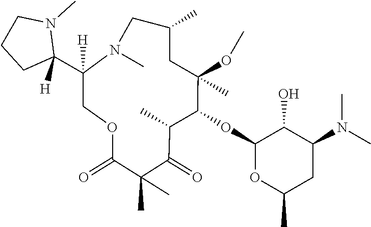 |
| 107 | 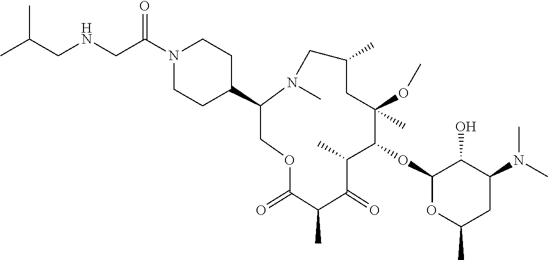 [SMILES: CO[C@]1(C)C[C@@H](C)CN(C)[C@H](C2CCN(C(=O)CNCC(C)C)CC2)COC(=O)[C@H](C)C(=O)[C@H](C)[C@H]1O[C@@H]1O[C@H](C)C[C@H](N(C)C)[C@H]1O] |
| 108 | 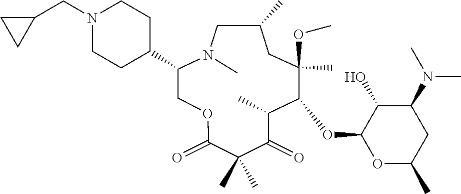 |
| 109 | 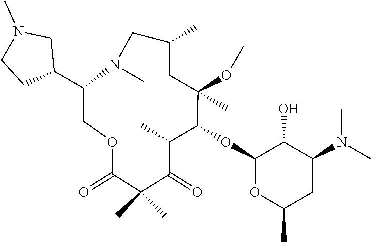 |
| 110 | 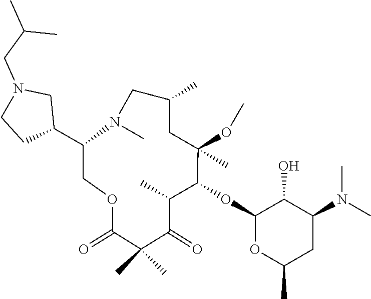 |
| 111 | 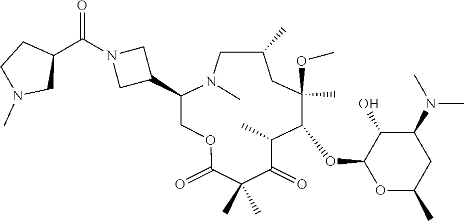 |
| 112 | 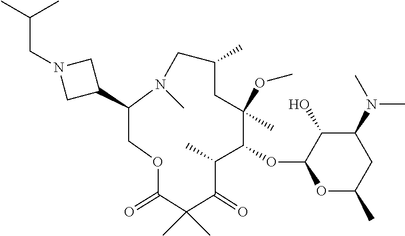 [SMILES: CO[C@]1(C)C[C@@H](C)CN(C)[C@H](C2CN(CC(C)C)C2)COC(=O)C(C)(C)C(=O)[C@H](C)[C@H]1O[C@@H]1O[C@H](C)C[C@H](N(C)C)[C@H]1O] |
| 113 | 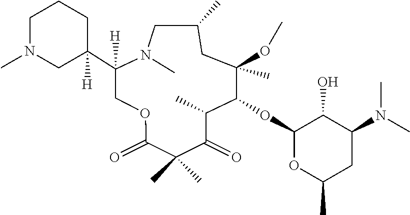 |
| 114 | 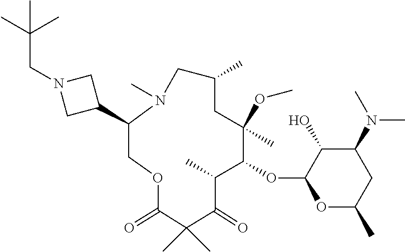 [SMILES: CO[C@]1(C)C[C@@H](C)CN(C)[C@H](C2CN(CC(C)(C)C)C2)COC(=O)C(C)(C)C(=O)[C@H](C)[C@H]1O[C@@H]1O[C@H](C)C[C@H](N(C)C)[C@H]1O] |
| 115 | 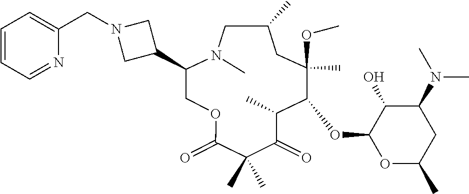 |
| 116 | 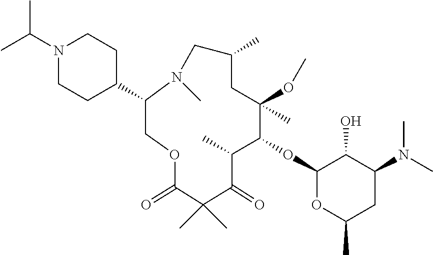 [SMILES: CO[C@]1(C)C[C@@H](C)CN(C)[C@@H](C2CCN(C(C)C)CC2)COC(=O)C(C)(C)C(=O)[C@H](C)[C@H]1O[C@@H]1O[C@H](C)C[C@H](N(C)C)[C@H]1O] |
| 117 | 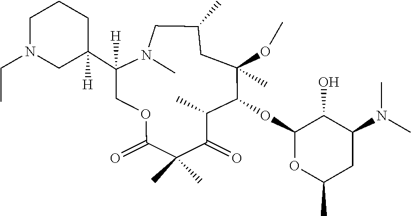 |
| 118 | 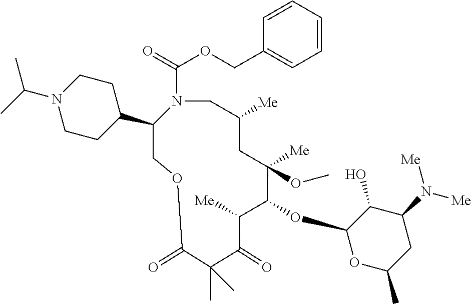 [SMILES: CO[C@]1(C)C[C@@H](C)CN(C(=O)OCc2ccccc2)[C@H](C2CCN(C(C)C)CC2)COC(=O)C(C)(C)C(=O)[C@H](C)[C@H]1O[C@@H]1O[C@H](C)C[C@H](N(C)C)[C@H]1O] |
| 119 | 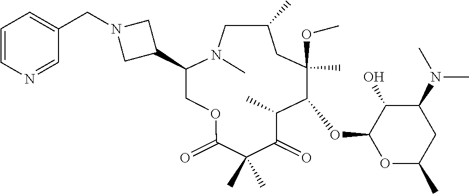 |
| 120 | 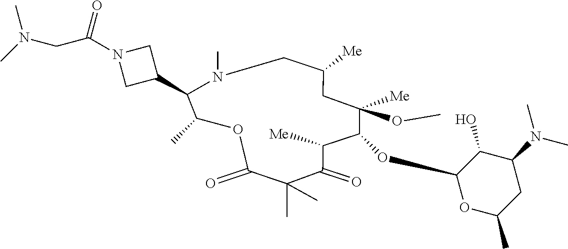 |
| 121 | 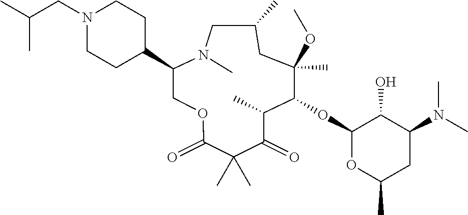 [SMILES: CO[C@]1(C)C[C@@H](C)CN(C)[C@H](C2CCN(CC(C)C)CC2)COC(=O)C(C)(C)C(=O)[C@H](C)[C@H]1O[C@@H]1O[C@H](C)C[C@H](N(C)C)[C@H]1O] |
| 122 | 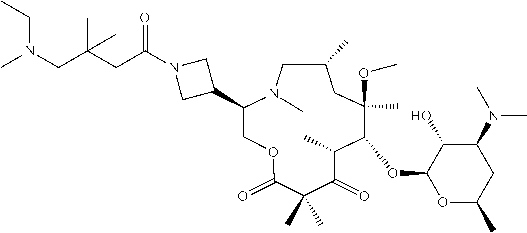 |
| 123 | 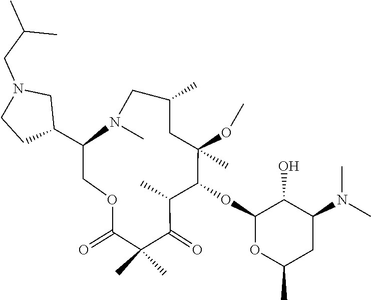 |
| 124 | 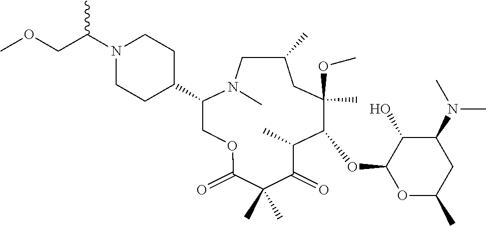 |
| 125 | 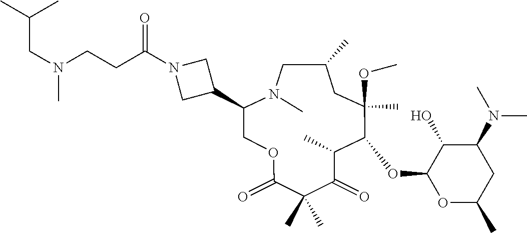 |
| 126 | 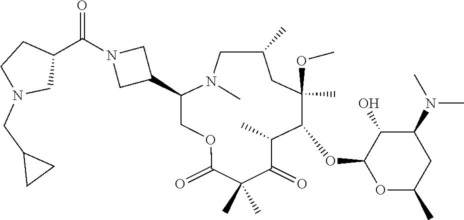 |
| 127 | 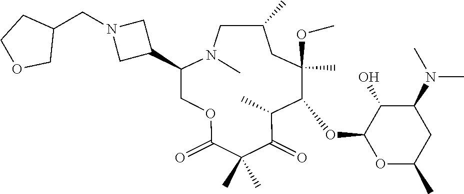 |
| 128 | 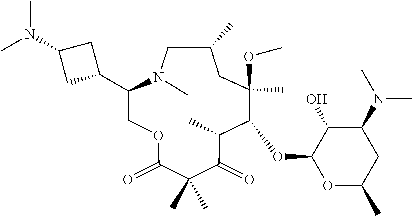 |
| 129 | 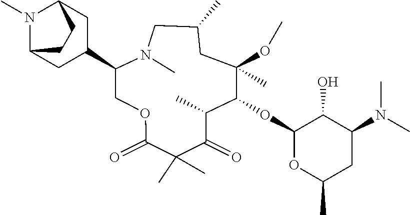 |
| 130 | 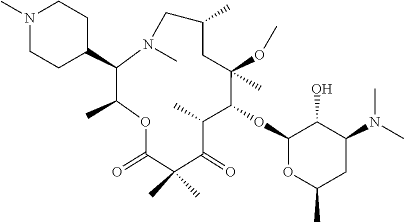 |
| 131 | 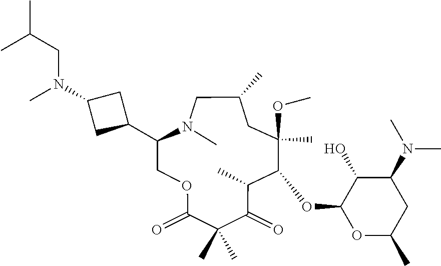 |
| 132 | 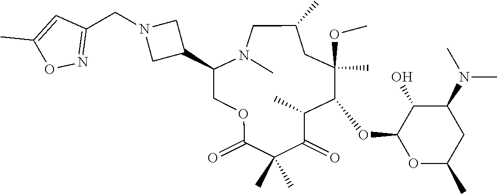 |
| 133 | 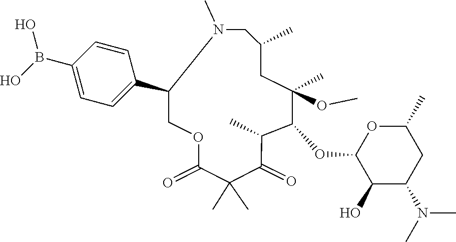 [SMILES: CO[C@]1(C)C[C@@H](C)CN(C)[C@H](c2ccc(B(O)O)cc2)COC(=O)C(C)(C)C(=O)[C@H](C)[C@H]1O[C@@H]1O[C@H](C)C[C@H](N(C)C)[C@H]1O] |
| 134 | 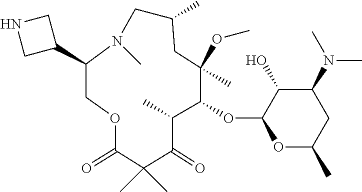 [SMILES: CO[C@]1(C)C[C@@H](C)CN(C)[C@H](C2CNC2)COC(=O)C(C)(C)C(=O)[C@H](C)[C@H]1O[C@@H]1O[C@H](C)C[C@H](N(C)C)[C@H]1O] |
| 135 | 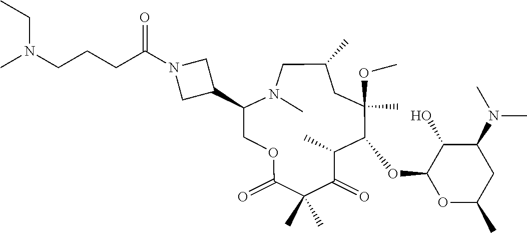 |
| 136 | 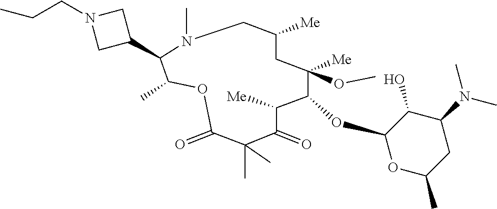 [SMILES: CCCN1CC([C@@H]2[C@@H](C)OC(=O)C(C)(C)C(=O)[C@H](C)[C@@H](O[C@@H]3O[C@H](C)C[C@H](N(C)C)[C@H]3O)[C@](C)(OC)C[C@@H](C)CN2C)C1] |
| 137 | 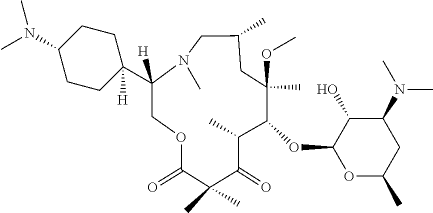 |
| 138 | 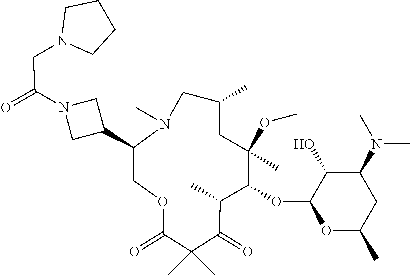 [SMILES: CO[C@]1(C)C[C@@H](C)CN(C)[C@H](C2CN(C(=O)CN3CCCC3)C2)COC(=O)C(C)(C)C(=O)[C@H](C)[C@H]1O[C@@H]1O[C@H](C)C[C@H](N(C)C)[C@H]1O] |
| 139 | 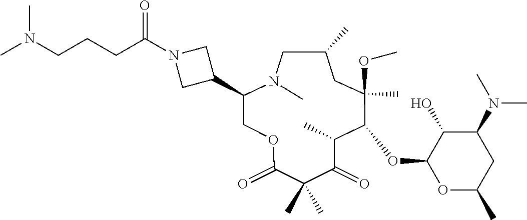 |
| 140 | 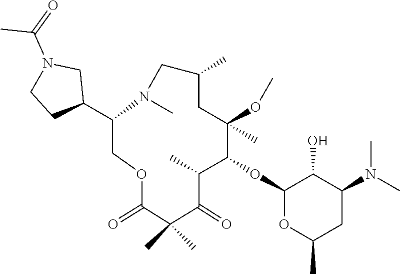 |
| 142 | 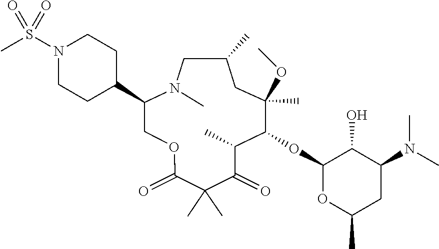 |
| 143 | 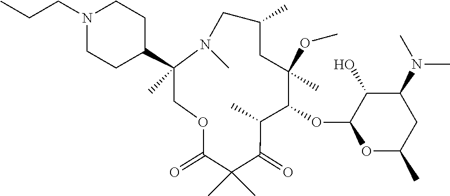 [SMILES: CCCN1CCC([C@]2(C)COC(=O)C(C)(C)C(=O)[C@H](C)[C@@H](O[C@@H]3O[C@H](C)C[C@H](N(C)C)[C@H]3O)[C@](C)(OC)C[C@@H](C)CN2C)CC1] |
| 144 | 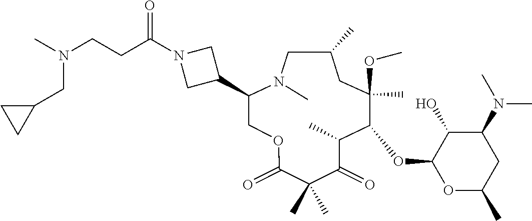 |
| 145 | 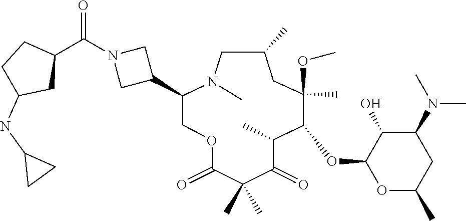 |
| 146 | 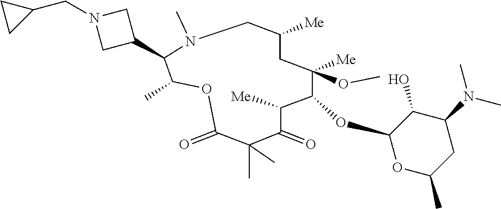 [SMILES: CO[C@]1(C)C[C@@H](C)CN(C)[C@H](C2CN(CC3CC3)C2)[C@@H](C)OC(=O)C(C)(C)C(=O)[C@H](C)[C@H]1O[C@@H]1O[C@H](C)C[C@H](N(C)C)[C@H]1O] |
| 147 | 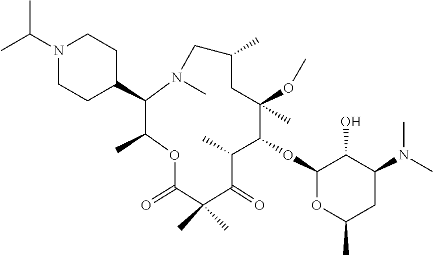 |
| 148 | 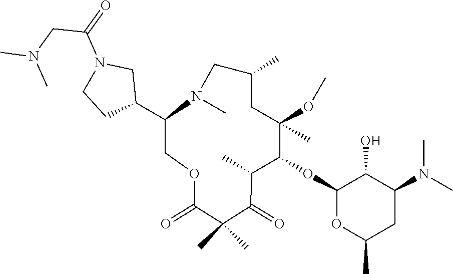 |
| 149 | 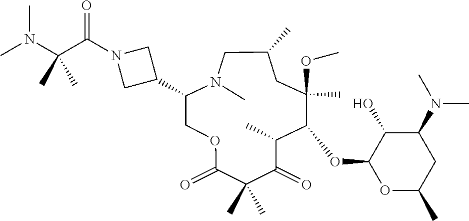 |
| 150 | 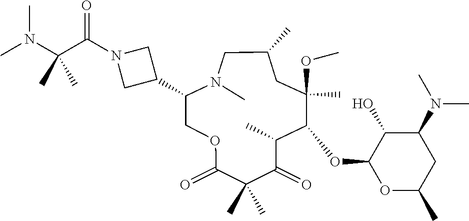 |
| 151 | 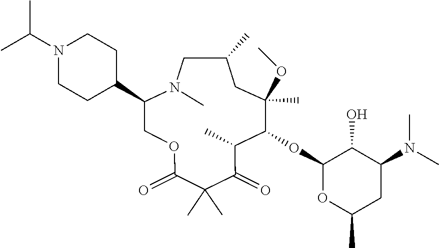 [SMILES: CO[C@]1(C)C[C@@H](C)CN(C)[C@H](C2CCN(C(C)C)CC2)COC(=O)C(C)(C)C(=O)[C@H](C)[C@H]1O[C@@H]1O[C@H](C)C[C@H](N(C)C)[C@H]1O] |
| 154 | 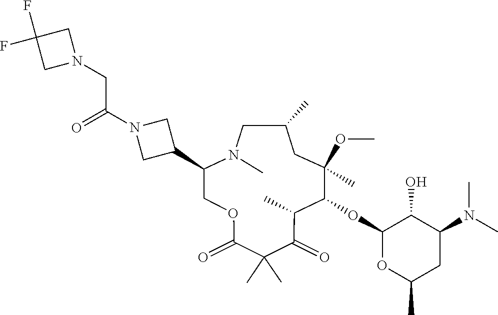 [SMILES: CO[C@]1(C)C[C@@H](C)CN(C)[C@H](C2CN(C(=O)CN3CC(F)(F)C3)C2)COC(=O)C(C)(C)C(=O)[C@H](C)[C@H]1O[C@@H]1O[C@H](C)C[C@H](N(C)C)[C@H]1O] |
| 155 | 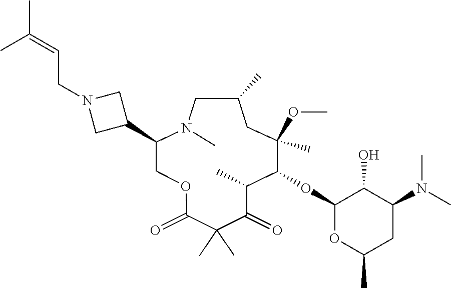 [SMILES: CO[C@]1(C)C[C@@H](C)CN(C)[C@H](C2CN(CC=C(C)C)C2)COC(=O)C(C)(C)C(=O)[C@H](C)[C@H]1O[C@@H]1O[C@H](C)C[C@H](N(C)C)[C@H]1O] |
| 156 | 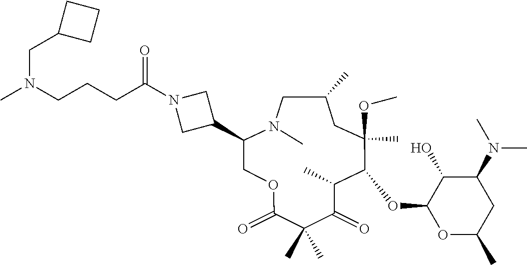 |
| 157 | 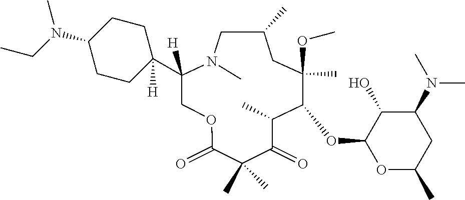 |
| 158 | 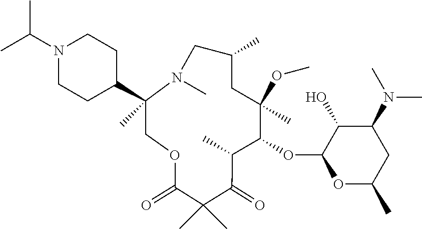 [SMILES: CO[C@]1(C)C[C@@H](C)CN(C)[C@](C)(C2CCN(C(C)C)CC2)COC(=O)C(C)(C)C(=O)[C@H](C)[C@H]1O[C@@H]1O[C@H](C)C[C@H](N(C)C)[C@H]1O] |
| 159 | 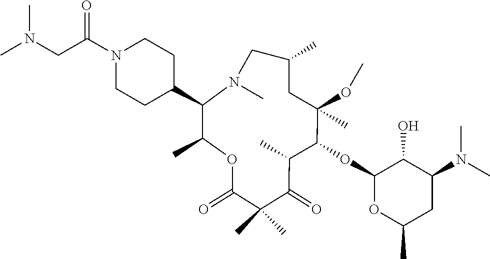 |
| 160 | 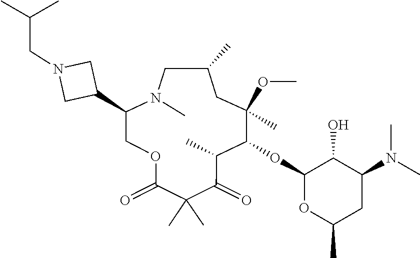 [SMILES: CO[C@]1(C)C[C@@H](C)CN(C)[C@H](C2CN(CC(C)C)C2)COC(=O)C(C)(C)C(=O)[C@H](C)[C@H]1O[C@@H]1O[C@H](C)C[C@H](N(C)C)[C@H]1O] |
| 161 | 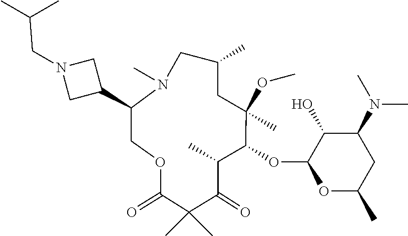 [SMILES: CO[C@]1(C)C[C@@H](C)CN(C)[C@H](C2CN(CC(C)C)C2)COC(=O)C(C)(C)C(=O)[C@H](C)[C@H]1O[C@@H]1O[C@H](C)C[C@H](N(C)C)[C@H]1O] |
| 164 | 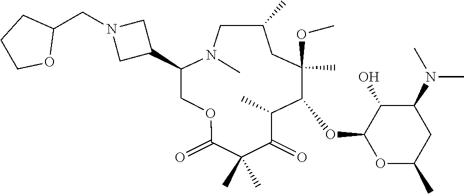 |
| 165 | 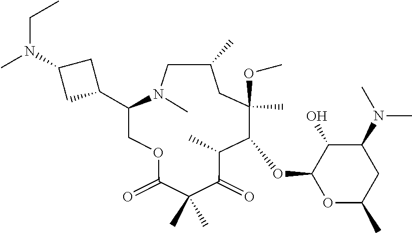 |
| 166 | 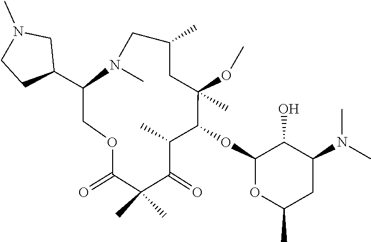 |
| 167 | 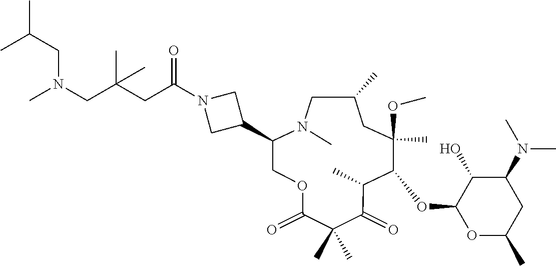 |
| 168 | 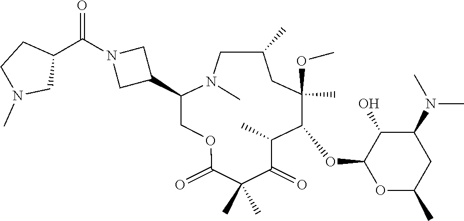 |
| 169 | 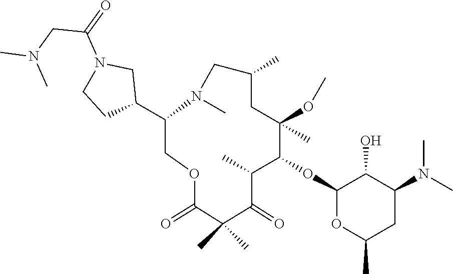 |
| 170 | 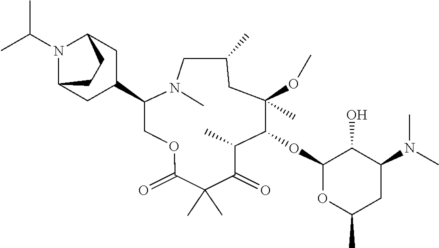 [SMILES: CO[C@]1(C)C[C@@H](C)CN(C)[C@H](C2C[C@H]3CC[C@@H](C2)N3C(C)C)COC(=O)C(C)(C)C(=O)[C@H](C)[C@H]1O[C@@H]1O[C@H](C)C[C@H](N(C)C)[C@H]1O] |
| 171 | 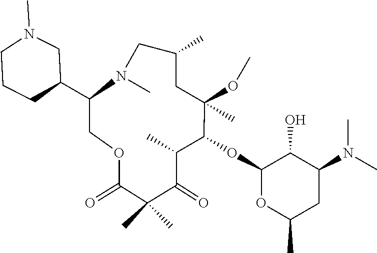 |
| 172 | 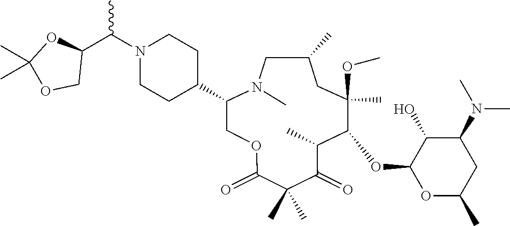 |
| 173 | 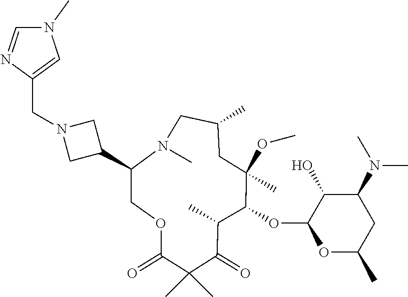 [SMILES: CO[C@]1(C)C[C@@H](C)CN(C)[C@H](C2CN(Cc3cn(C)cn3)C2)COC(=O)C(C)(C)C(=O)[C@H](C)[C@H]1O[C@@H]1O[C@H](C)C[C@H](N(C)C)[C@H]1O] |
| 174 | 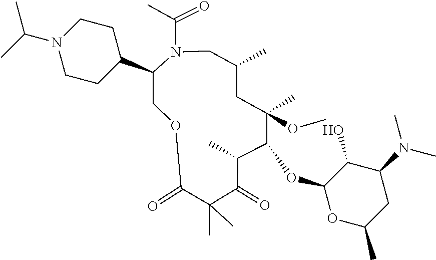 [SMILES: CO[C@]1(C)C[C@@H](C)CN(C(C)=O)[C@H](C2CCN(C(C)C)CC2)COC(=O)C(C)(C)C(=O)[C@H](C)[C@H]1O[C@@H]1O[C@H](C)C[C@H](N(C)C)[C@H]1O] |
| 175 | 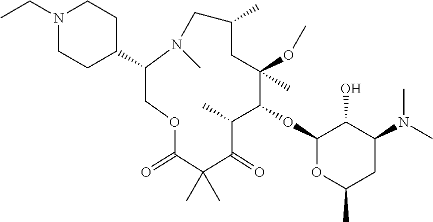 [SMILES: CCN1CCC([C@H]2COC(=O)C(C)(C)C(=O)[C@H](C)[C@@H](O[C@@H]3O[C@H](C)C[C@H](N(C)C)[C@H]3O)[C@](C)(OC)C[C@@H](C)CN2C)CC1] |
| 176 | 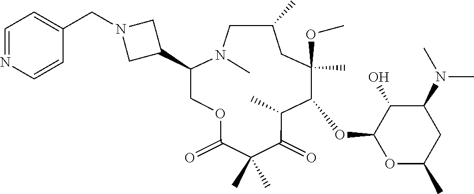 |
| 177 | 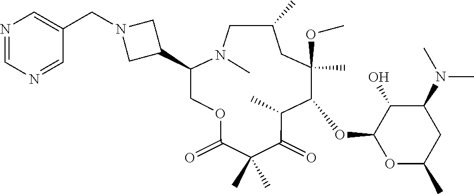 |
| 178 | 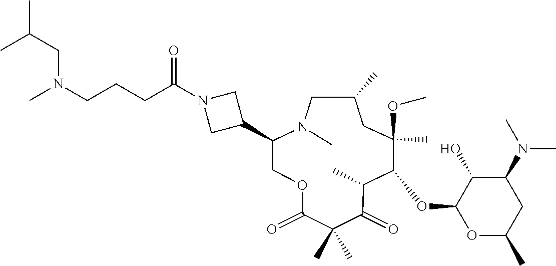 |
| 179 | 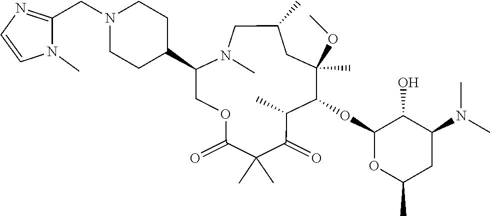 [SMILES: CO[C@]1(C)C[C@@H](C)CN(C)[C@H](C2CCN(Cc3nccn3C)CC2)COC(=O)C(C)(C)C(=O)[C@H](C)[C@H]1O[C@@H]1O[C@H](C)C[C@H](N(C)C)[C@H]1O] |
| 180 | 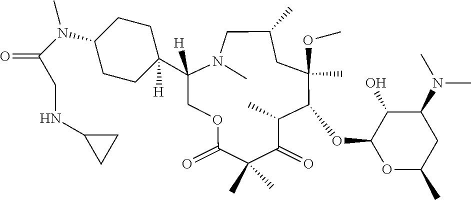 |
| 181 | 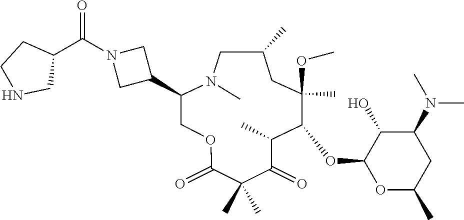 |
| 182 | 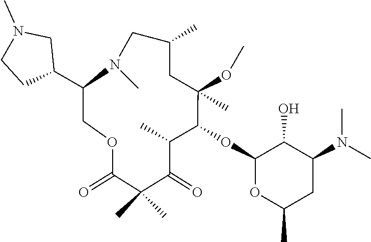 |
| 183 | 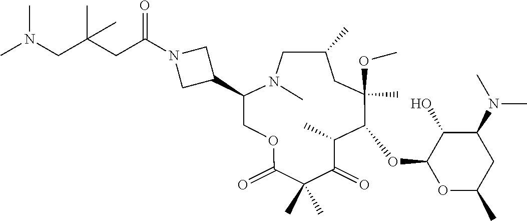 |
| 184 | 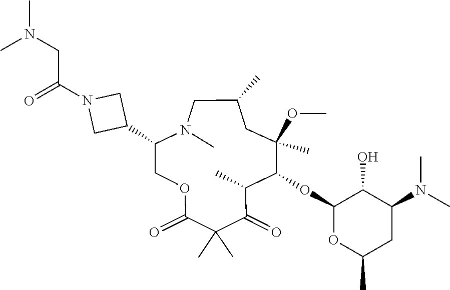 |
| 185 | 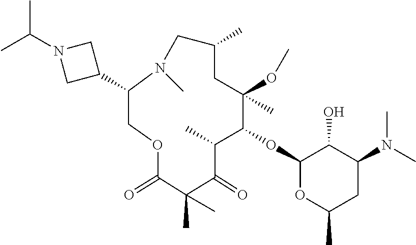 |
| 186 | 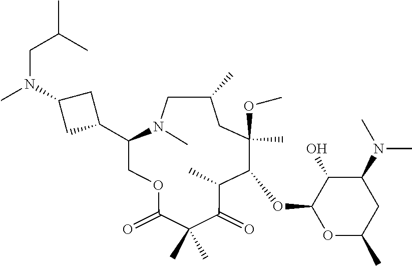 |
| 187 | 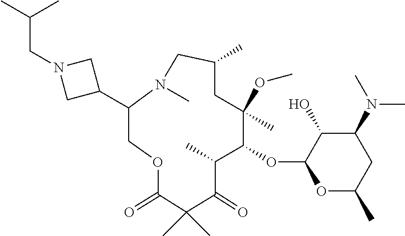 [SMILES: CO[C@]1(C)C[C@@H](C)CN(C)C(C2CN(CC(C)C)C2)COC(=O)C(C)(C)C(=O)[C@H](C)[C@H]1O[C@@H]1O[C@H](C)C[C@H](N(C)C)[C@H]1O] |
| 188 | 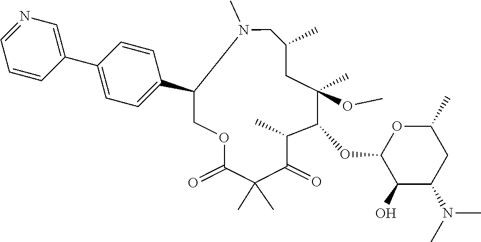 [SMILES: CO[C@]1(C)C[C@@H](C)CN(C)[C@H](c2ccc(-c3cccnc3)cc2)COC(=O)C(C)(C)C(=O)[C@H](C)[C@H]1O[C@@H]1O[C@H](C)C[C@H](N(C)C)[C@H]1O] |
| 189 | 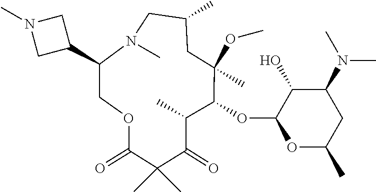 |
| 190 | 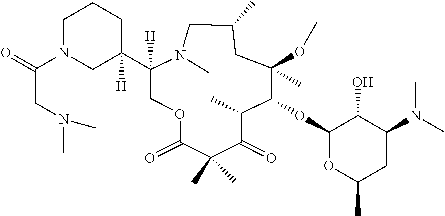 |
| 191 | 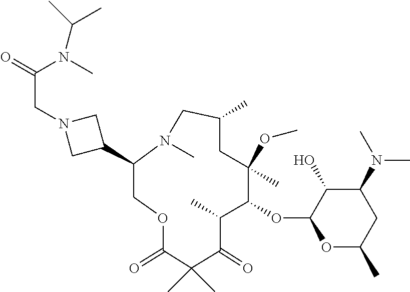 [SMILES: CO[C@]1(C)C[C@@H](C)CN(C)[C@H](C2CN(CC(=O)N(C)C(C)C)C2)COC(=O)C(C)(C)C(=O)[C@H](C)[C@H]1O[C@@H]1O[C@H](C)C[C@H](N(C)C)[C@H]1O] |
| 192 | 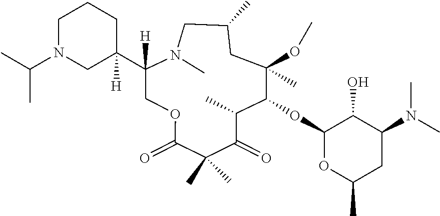 |
| 193 | 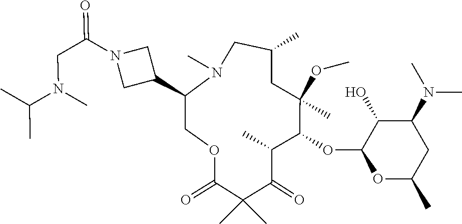 [SMILES: CO[C@]1(C)C[C@@H](C)CN(C)[C@H](C2CN(C(=O)CN(C)C(C)C)C2)COC(=O)C(C)(C)C(=O)[C@H](C)[C@H]1O[C@@H]1O[C@H](C)C[C@H](N(C)C)[C@H]1O] |
| 194 | 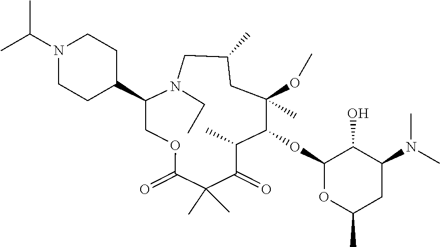 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@H]1C1CCN(C(C)C)CC1] |
| 195 | 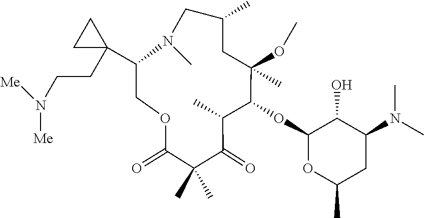 |
| 196 | 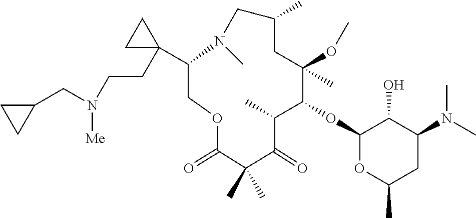 |
| 197 | 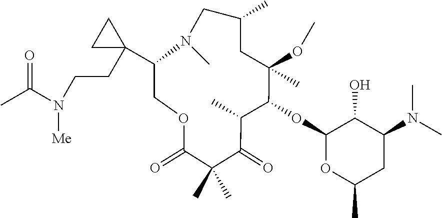 |
| 198 | 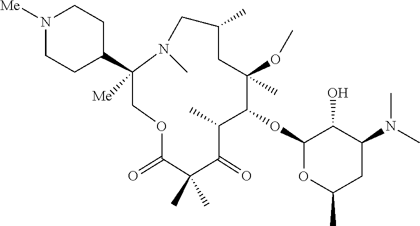 |
| 199 | 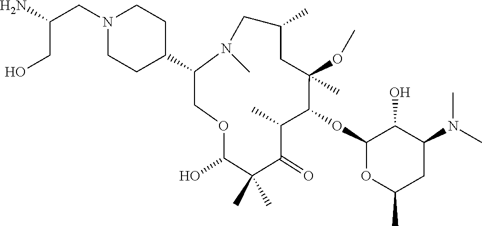 |
| 200 | 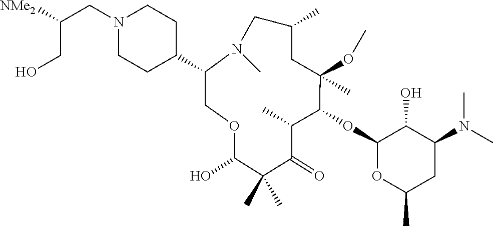 |
| 201 | 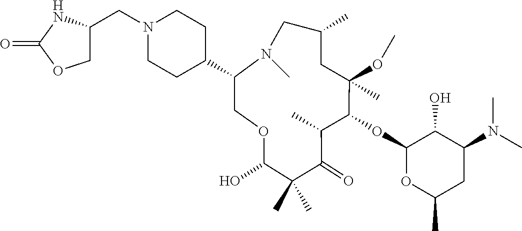 |
| 202 | 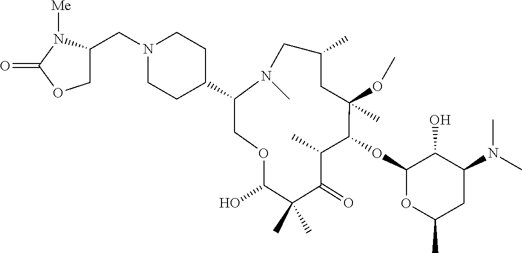 |
| 203 | 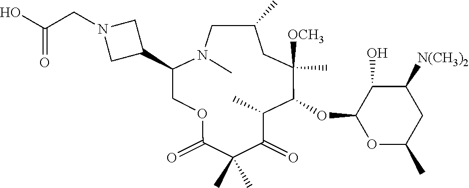 |
| 204 | 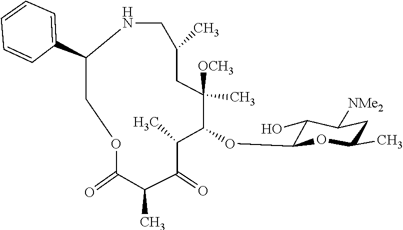 |
| 205 | 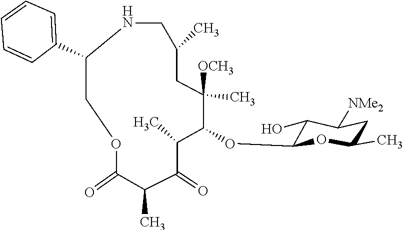 |

143. A pharmaceutical composition comprising the compound of claim 121, or a pharmaceutically acceptable salt thereof, and a pharmaceutically acceptable excipient.

144. A kit comprising the compound of claim 121, or pharmaceutically acceptable salt thereof, and instructions for administration to a subject.

145. A method of treating an infectious disease in a subject in need of such treatment, comprising administering to the subject a compound of claim 121, or pharmaceutically acceptable salt thereof, wherein the infectious disease is a bacterial infection which is a Gram positive bacterial infection or a Gram negative bacterial infection or a parasitic infection.

146. The method of claim 145, wherein the infectious disease is a bacterial infection caused by a bacteria selected from the group consisting of Staphylococcus, Acinetobacter, Klebsiella, Escherichia, and Pseudomonas.

147. A compound of formula N-a: 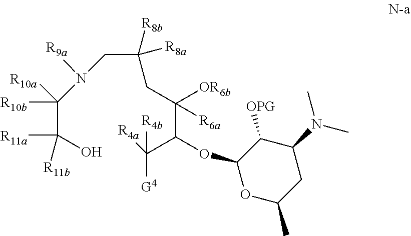
or a salt thereof, wherein R₄ₐ, R_{4b}, R₆ₐ, R_{6b}, R₈ₐ, R_{8b}, R₁₀ₐ, R_{10b}, R₁₁ₐ, and R_{11b} are as defined in claim 1;
G⁴ is 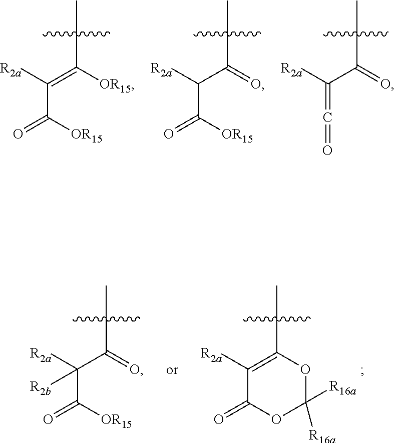
each instance of R¹⁵ is independently silyl, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R¹⁵ groups are joined to form an optionally substituted heterocyclyl or heteroaryl ring; and
each instance of R^{16a} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl; and PG is a protecting group.

148. A process of preparing a compound of formula I of claim 121, comprising the step of preparing a compound of formula N-a, by combining N-1 with N-2 under reductive amination conditions: 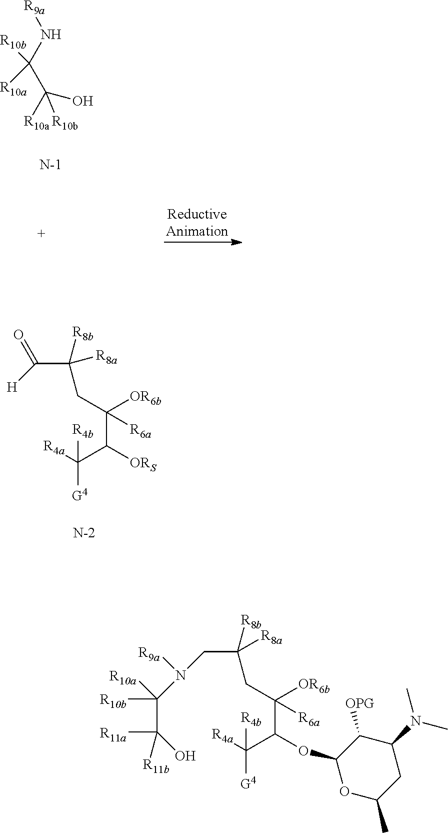
wherein R₅ is 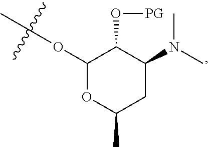
PG is a protecting group, and “ [symbol] ” indicates a point of attachment; and
cyclization of a compound of formula N-a and subsequent deprotection to provide a compound of formula I: [chemical structure]
